(19) United States
(12) Patent Application Publication
(10) Pub. No.: US 2026/0199433 A1
(43) Pub. Date: Jul. 16, 2026

(54) ENHANCED ADOPTIVE CELL THERAPY

(71) Applicant: Werewolf Therapeutics, Inc., Watertown, MA (US)

(72) Inventors: William WINSTON, West Newton, MA (US); Cynthia SEIDEL-DUGAN, Clearwater, FL (US); Jose Andres SALMERON-GARCIA, Acton, MA (US); Randi ISAACS, East Hampton, NY (US); Connor Jude DWYER, Waltham, MA (US); Kristin R. MORRIS, Boston, MA (US)

(21) Appl. No.: 19/629,611

(22) Filed: Mar. 26, 2026

Related U.S. Application Data
(63) Continuation of application No. PCT/US2024/048566, filed on Sep. 26, 2024.
(60) Provisional application No. 63/585,513, filed on Sep. 26, 2023.
(60) Provisional application No. 63/574,929, filed on Apr. 5, 2024.

(51) Int. Cl. A61K 38/20; A61K 31/166; A61K 31/197; A61K 31/396; A61K 31/4178; A61K 31/4745; A61K 31/513; A61K 31/519; A61K 31/52; A61K 31/704; A61K 31/7064; A61K 31/7068; A61K 40/11; A61K 40/31; A61K 40/42

(52) U.S. Cl. CPC A61K 38/208; A61K 31/166; A61K 31/197; A61K 31/396; A61K 31/4178; A61K 31/4745; A61K 31/513; A61K 31/519; A61K 31/52; A61K 31/704; A61K 31/7064; A61K 31/7068; A61K 38/2013; A61K 40/11; A61K 40/31; A61K 40/4211

(57) ABSTRACT

The disclosure relates to combination therapy comprising an inducible cytokine prodrug and an adoptive cell therapy. The disclosure also relates to methods for treating subjects using such combinations.

The present application is a continuation of International Patent Application No. PCT/US2024/048566, filed Sep. 26, 2024, which designated the United States, which claims the benefit of U.S. Provisional Application No. 63/585,513, filed on Sep. 26, 2023, and U.S. Provisional Application No. 63/574,929, filed on Apr. 5, 2024, the entire contents of each of which are incorporated herein by reference in their entireties.

### 1. SEQUENCE LISTING

The instant application contains a Sequence Listing which has been submitted electronically in XML format and is hereby incorporated by reference in its entirety. Said XML copy, created on Sep. 17, 2024, is named 761146_102320_SL.xml and is 464 bytes in size.

### 2. BACKGROUND

Inducible cytokine prodrugs, that are conditionally activated in the tumor microenvironment through protease cleavage to release the fully active, native cytokine within the tumor to stimulate a potent anti-tumor immune response, are described in International Publication Nos.: WO2019/222294, WO2019/222295, WO2019/222296, WO2021/097376, and WO2021/236676. These prodrugs typically include a native cytokine polypeptide that is attached to a cytokine blocking domain and typically a half-life extension domain, through a protease cleavable linker. For example, IL-2 prodrugs can include a native IL-2 molecule attached through a protease cleavable linker to a half-life extension domain (e.g., anti-human serum albumin antibody binding fragment such as a VH domain) and an IL-2 blocking element (e.g., anti-IL-2 antibody binding fragment, such as a Fab) to block binding of IL-2 to IL-2β/γ receptors on normal tissue in the periphery. Upon cleavage of the protease cleavable linker in the tumor microenvironment, fully active native IL-2 is released within the tumor to stimulate a potent anti-tumor immune response.

Adoptive cell therapies have seen some clinical success, but have similarly not reached their full potential. CAR-T therapies targeting CD19 or BCMA have been approved for B cell cancers and multiple myeloma, respectively, but their effectiveness is limited by lack of persistence and antigen escape. CAR-T therapy has overall not been successfully employed against solid tumors, in part as a result of the immunosuppressive tumor microenvironment along with physical barriers and the heterogeneity of solid tumors. CAR-T therapy is also associated with toxicity including cytokine release syndrome (CRS), neurotoxicity, and on target off tumor effects (Siegler et al., Front Immunol (2020) 11:1973). Cell therapies focusing on TCRs or other immune cell receptors have grown in popularity in recent years, and many such programs are in clinical trials. TCR therapies, like CAR-T therapies, are susceptible to loss of T cell persistence and antigen escape. Additional challenges to TCR based therapy include their restriction to MHC presented antigens, the limited availability of tumor-specific T cells and deficiencies in antigen processing or major histocompatibility complex (MHC) expression in cancer cells.

Tumor infiltrating lymphocytes (TILs) have been more successful in treating solid tumors such as metastatic melanoma and cervical cancer than CAR-T therapy or exogenous TCR based treatments. This is thought to be in part a result of TIL populations having multiple TCRs capable of recognizing tumor antigens, lessening the issue of tumor heterogeneity. However, TILs must overcome the immunosuppressive microenvironment and physical barriers associated with solid tumors.

There is a need for methods for improving the effectiveness and persistence of adoptive cell therapies while avoiding or minimizing toxicity.

### 3. SUMMARY

This disclosure relates to combination therapy that includes adoptive cell therapy and an inducible cytokine prodrug, and to compositions for use in such therapy. Adoptive cell therapy can have increased efficacy and/or improved safety when combined with the inducible cytokine prodrugs as described herein. Without wishing to be bound by any particular theory, it is believed that the combination of adoptive cell therapy and an inducible cytokine that is activated in the tumor microenvironment, can lead to greater immune cell recruitment, proliferation and effector function in the tumor microenvironment. This is expected to help limit well-known challenges and adverse events of adoptive cell therapy such as cytokine release syndrome, neurotoxicity, on-target but off tumor toxicity, and immune exhaustion.

This disclosure further relates to the use of inducible cytokines to increase the efficacy of adoptive cell therapies by, for example, increasing the ability of the therapeutic cells to proliferate and/or deliver effector functions at the desired site of activity. This may, for example, overcome the immunosuppressive effect of the tumor microenvironment, increasing the effectiveness of adoptive cell therapies in solid tumors.

Inducible cytokines can interact with endogenous non-engineered immune cells in the tumor microenvironment. When an inducible cytokine is combined with an adoptive cell therapy targeting a specific tumor antigen, activation of the inducible cytokine and targeting the adoptive cell therapy to the tumor microenvironment can lead to activation of the inducible cytokine and enhanced activity of the adoptive cell therapy. Activation of the inducible cytokine can also lead to enhanced effector function of endogenous immune cells which can lead to killing of tumor cells that do not express the specific tumor antigen recognized by the adoptive cell therapy. This can lead to greater efficacy, for example, by mitigating problems such as tumor heterogeneity.

This disclosure relates to methods of treating a subject in need thereof, comprising administering to the subject an effective amount of an inducible cytokine prodrug, or polynucleotide encoding the same, in combination with an adoptive cell therapy, for example an antigen binding protein. Polynucleotides encoding the inducible cytokine prodrug disclosed herein can be provided as a viral vector or can be encapsulated (e.g., encapsulated in a viral particle, encapsulated in a nanoparticle). Polynucleotides encoding the inducible cytokine prodrug can be provided in an immune cell. Polynucleotides encoding the antigen binding protein can be provided as a viral vector or encapsulated (e.g., encapsulated in a viral particle, encapsulated in a nanoparticle). An inducible cytokine prodrug as disclosed herein and an antigen binding protein as disclosed herein, can both be provided as a viral vector or encapsulated (e.g., encapsulated in a viral particle or nanoparticle).

Antigen binding proteins disclosed herein, when expressed in an immune cell, can direct the immune cell and its immune effector functions to cells that express a desired antigen. Antigen binding proteins can confer specificity for an antigen including CD19, CD123, CD22, CD30, CD171, CS-1, CLL-1 (CLECL1), CD33, EGFRvIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

The antigen binding protein can be a CAR (e.g., a CAR comprising a binding domain sequence selected from SEQ ID NO: 562-648). CARs serving as antigen binding proteins can comprise a hinge domain sequence of SEQ ID NO: 649-651, can comprise a transmembrane domain sequence of SEQ ID NO: 652-653, can comprise a costimulatory domain sequence of SEQ ID NO: 654-658, and/or a primary signaling domain sequence of SEQ ID NO: 659-660. This disclosure relates to methods including CARs that can comprise sequences of SEQ ID NO: 661-665. Antigen binding proteins of this disclosure can be exogenous TCR subunits (e.g., subunits of a sequence of SEQ ID NO: 666-702) or TFPs (e.g. with a sequence of SEQ ID NO: 703-705).

The inducible cytokine prodrugs disclosed herein can comprise a cytokine polypeptide, a protease cleavable linker, and a blocking element. The inducible cytokine prodrugs disclosed herein have attenuated cytokine biological activity that can be restored (e.g., not attenuated) upon proteolytic cleavage of a protease cleavable linker by a protease.

Protease cleavable linkers described in this disclosure can comprise any amino acid sequence, such as an amino acid sequence selected from SEQ ID NOs: 195-220 or an amino acid sequence that has at least about 90% identity to SEQ ID NOs: 195-220. Protease cleavable linkers can comprise one cleavable moiety or more than two cleavable moieties, each of which is a substrate for a protease. When protease cleavable linkers comprise more than two cleavable moieties, a first cleavable moiety comprising a first amino acid sequence can be a substrate for a first protease and a second cleavable moiety comprising a second amino acid sequence that can be a substrate for a second protease. The protease cleavable linkers can further comprise a non-cleavable linker sequence (e.g., non-cleavable to a protease). Protease cleavable linkers can be cleaved with greater catalytic efficiency, greater specificity, or both, by one or more proteases (e.g., FAPa, CTSL1, an ADAM selected from ADAM 8, ADAM 9, ADAM 10, ADAM12 ADAM17, and ADAMTS1, and an MMP selected from MMP1, MMP2, MMP9 and MMP14) than a reference polypeptide sequence. The one or more proteases can be selected from MMP1, MMP2, MMP9, MMP4, cathepsin B, cathepsin C, cathepsin D, cathepsin E, cathepsin G, cathepsin K or cathepsin L. The reference polypeptide sequence of a protease cleavable linker sequence can be present in a naturally occurring polypeptide substrate for FAPa, CTSL1, an ADAM selected from ADAM 8, ADAM 9, ADAM 10, ADAM12 ADAM17, and ADAMTS1, and an MMP selected from MMP1, MMP2, MMP9 and MMP14, or a combination thereof.

The inducible cytokine prodrugs can comprise a blocking element. The blocking element can be a steric blocking moiety, a specific blocking moiety, or combinations thereof. The blocking moiety can comprise human serum albumin (HSA) or an anti-HSA antibody.

The inducible cytokine prodrugs can have half-lives of at least about 1 hour, 2 hours, 3 hours, 4 hours, 5 hours, 6 hours, 7 hours, 8 hours, 9 hours, 10 hours, 11 hours, 12 hours, 18 hours, or 24 hours. The inducible cytokine prodrugs can further comprise one or more half-life extension domains.

The inducible cytokine prodrugs described herein can comprise two or more polypeptide chains

The first and second polypeptide chains can be cytokine subunits or functional fragments thereof (e.g., a first cytokine subunit and a second cytokine subunit). The cytokine subunits (or functional fragments thereof) can associate to form a complex that has attenuated biological activity. Cleavage of the protease cleavable linker by a protease can produce a cytokine with biological activity that is not attenuated (e.g., restored biological activity compared to the unblocked cytokine). The cytokine polypeptide (e.g., first cytokine subunit of the fusion protein) can be IL-12 subunit IL-12p35, mutein, or a functional fragment thereof and the second cytokine subunit of the second polypeptide can be IL-12 subunit IL-12p40, mutein, or a functional fragment thereof. The cytokine polypeptide (first cytokine subunit or functional fragment thereof) can be IL-12 subunit p40 or a functional fragment thereof, and the second cytokine subunit or functional fragment thereof can be IL-12 subunit IL-12p35 or a functional fragment thereof. The cytokine polypeptide can comprise both IL-12 subunit IL-12p35, muteins or functional fragments thereof. The cytokine polypeptide can be IL-2, IL-15, IL-21, or a mutein or functional fragment thereof. The second cytokine subunit can be exogenous to the subject. Alternatively, the second cytokine subunit can be endogenous to the subject. The second cytokine subunit of methods described herein can comprise a linker and a half-life extension moiety.

In methods of this disclosure comprising an inducible cytokine prodrug comprising a cytokine polypeptide, a protease cleavable linker, and a first fragment of an antibody specific for the cytokine polypeptide, in combination with a second polypeptide comprising a second fragment of an antibody specific for the cytokine polypeptide of the fusion protein. The first fragment of an antibody specific for the cytokine or cytokine polypeptide and second polypeptide comprising a second fragment of an antibody specific for the cytokine or cytokine polypeptide can be capable of associating to form an antibody specific for the cytokine or cytokine polypeptide. The biological activity of the complex can be attenuated and cleavage of the protease cleavable linker by a protease can produce a cytokine with biological activity that is not attenuated.

The cytokine polypeptides generated by methods disclosed herein can be defined by the formula [A1]-[L5]-[A2]. [A1] can comprise an IL-12 subunit p40, a mutein, or functional fragment thereof. [A2] can comprise an IL-12 subunit IL-12p35, a mutein, or functional fragment thereof. [L5] can comprise a polypeptide linker that is optionally protease cleavable.

Methods described herein can comprise administering adoptive cell therapy to a patient. Adoptive cell therapy of methods of this disclosure can comprise TILs, T cells, NK cells, NKT cells, Tregs, autologous immune cells, allogeneic immune cells, or immune cells that comprise antigen binding proteins (e.g., immune cells comprising a chimeric autoantibody receptor (CAAR)). Allogeneic immune cells of method described herein can have an endogenous TCR gene or MHC gene which has been disrupted.

Subjects treated by methods described herein can have an autoimmune disease (e.g., graft versus host disease, multiple sclerosis, rheumatoid arthritis, myasthenia gravis, Crohn's disease, lupus) or can be at risk of graft rejection.

A subject can be administering 1 dose of the inducible cytokine prodrug, 2 doses of the inducible cytokine prodrug, 3 doses of the inducible cytokine prodrug, 4 doses of the inducible cytokine prodrug, or 5 or more doses of the inducible cytokine prodrug. This disclosure relates to methods of treating a subject by administering 1 dose of the adoptive cell therapy, 2 doses of the adoptive cell therapy, 3 doses of the adoptive cell therapy 4 doses of the adoptive cell therapy, or wherein the subject is administered 5 or more doses of the adoptive cell therapy. Methods described herein can comprise administration of at least one dose of the inducible cytokine prodrug and at least one dose of the adoptive cell therapy administered to the subject simultaneously or with administration of at least one dose of the fusion protein before at least one dose of the immune cell. A patient can be administered about 1 day or more than 1 day (e.g. 2 days, 3 days, 4, days, 5, days, 6, days, 7 days, 2 weeks, 3 weeks, 4 weeks, 5 weeks, or 6 weeks) before at least one dose of the adoptive cell therapy. At least one dose of the inducible cytokine prodrug can be administered after at least one dose of the adoptive cell therapy and the fusion protein can be administered about 1 day or more than 1 day (e.g., 2 days, 3 days, 4, days, 5, days, 6, days, 7 days, 2 weeks, 3 weeks, 4 weeks, 5 weeks, or 6 weeks) after at least one dose of adoptive cell therapy. In methods of this disclosure, at least one dose of the inducible cytokine prodrug can be administered to a subject when the administered immune cell count has decreased by at least about (e.g., decreased by about 10%, about 20%, about 30%, about 40%, about 50%, about 60%, about 70%, about 80%, about 90%) or when immune cells are no longer detectable. This disclosure relates to methods of treatment comprising administering a fusion protein and immune cells that can have an overlap in the timing of their pharmacological or biological activities.

Methods of this disclosure can further include a step of administering a chemotherapeutic agent (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, and Procarabizine) or a checkpoint inhibitor (e.g., an anti-PD-L1 agent, an anti-CTLA4 agent, an anti-PD-1 agent, an anti-CD47 agent, and an anti-GD2 agent). The disclosure further relates to pharmaceutical compositions comprising an inducible cytokine prodrug and an adoptive cell therapy (i.e., an antigen binding protein). The inducible cytokine prodrugs of this disclosure can comprise a cytokine polypeptide (e.g., IL-2, IL-12, IL-15, and IL-21, or a functional fragment, mutein or subunit thereof), a protease cleavable linker (e.g., a linker with a sequence of any one of SEQ ID NOs: 195-220 or an amino acid sequence that has at least about 90% identity to SEQ ID NOs: 195-220), and a blocking element (e.g., steric blocking element, a specific blocking element, or combination thereof).

This disclosure relates to an immune cell comprising a polynucleotide encoding a fusion protein and a polynucleotide encoding an antigen binding protein.

This disclosure further relates to a method of making an immune cell comprising an immune cell with a (one or more) polynucleotide encoding an inducible cytokine prodrug. In methods of making an immune cell of this disclosure, the polynucleotide can further encode an antigen binding protein.

### 4. BRIEF DESCRIPTION OF THE DRAWINGS

FIG. 1 is a schematic diagram depicting the study protocol for pmel-1 CD8+ T cell adoptive cell therapy for B16F10 with pmel-1 in combination with WW0621/0523 or WW0757/636.

FIGS. 2A-2F are a series of graphs showing CD45+CD3+ cells as % of single cells (FIGS. 2A and 33D), CD4+ T cell as % of CD45+CD3+ cells (FIGS. 2B and 2D), and CD8+ T cell as % of CD45+CD3+ cells (FIGS. 2C and 2F) at days 8 and days 15 after treatment of vehicle, 5×10⁶ pmel-1 alone (i.e., without an inducible cytokine prodrug), 10×10⁶ pmel-1 (i.e., without an inducible cytokine prodrug), 5×10⁶ pmel-1 with and WW0621/0523, or 10×10⁶ pmel-1 with WW0621/0523, or WW0621/0523 alone. The graphs show that pmel-1 at 5×10⁶ pmel-1 and 10×10⁶ pmel-1 combination with WW0621/0523 lead to selective expansion of CD8+ T cells.

FIGS. 3A-3C shows engraftment of donor cells 8 days post infusion by detecting pmel-1 CD8+VB13+ TCR by flow cytometry. FIG. 3A are flow cytometric plots depicting the percentage of engraftment of donor cells (Vβ13+CD8+ cells) in mice treated with activated pmel-1 cells but not inducible cytokine or with activated pmel-1 cell plus inducible IL-2 (WW0621/0523). The gates show the population of Vβ13+CD8+ cells. FIG. 3B is a graph showing quantification of CD8+Vβ13+ cells as a percentage of single cells, and FIG. 3C is a graph showing the quantification of CD8+Vβ13+ cells as a percentage of total CD8+ cells, in mice treated with (i) vehicle, (ii) WW0621/0523 alone, (iii) pmel-1 at 5×10⁶ and no inducible cytokine prodrug, (iv) pmel-1 at 5×10⁶ and WW0621/0523, (v) pmel-1 at 10×10⁶ and no inducible cytokine prodrug, (vi) pmel-1 at 10×10⁶ and WW0621/0523.

FIGS. 4A-4C shows engraftment of donor cells 15 days post infusion by detecting pmel-1 CD8+VB13+ TCR by flow cytometry. FIG. 4A is flow cytometric plots depicting the percentage of engraftment of donor cells (Vβ13 expressing CD8 cells) in mice treated with inducible IL-2 prodrug (WW0621/0523) and no pmel-1 cells, or 5×10⁶ or 10×10⁶ pmel-1 cells. The gates show the population of Vβ13+CD8+ cells. FIG. 4B is a graph showing quantification CD8+ Vβ13+ cells as a percentage of single cells, and FIG. 4C is a graph showing quantification of CD8+ Vβ13+ cells as a percentage of total CD8+ cells, in mice treated with (i) vehicle, (ii) WW0621/0523 alone, (iii) pmel-1 at 5×10⁶ and no inducible cytokine prodrug, (iv) pmel-1 at 5×10⁶ and WW0621/0523, (v) pmel-1 at 10×10⁶ and no inducible cytokine prodrug, (vi) pmel-1 at 10×10⁶ and WW0621/0523.

FIGS. 5A-5D is a series of graphs showing the engraftment of donor cells 35 days post infusion. FIGS. 5A and 5B show the percentage of CD8+Vβ13+ as % of single cells (FIG. 5A) or as % of CD8+ cells (FIG. 5B) for vehicle, pmel-1 5×10⁶ alone, and pmel-1 5×10⁶ with WW0621/0523. FIGS. 5C and 5D show the percentage of CD8+Vβ13+ as % of single cells (FIG. 5C) or as % of CD8+ cells (FIG. 5D) for vehicle, pmel-1 10×10⁶ alone, and pmel-1 10×10⁶ with WW0621/0523. The graphs show that infusion with pme-1 in combination with WW0621/0523 leads to long term persistence of donor CD8+T-cells for at least 35 days.

FIGS. 6A-6C are graphs showing the results of combination treatment with pmel-1 and an inducible IL-2 prodrug in a B16F10 melanoma mouse model. FIGS. 6A-6B are graphs showing tumor volume average (mm³) over time in mice treated with pmel-1 CD8+ T cells at two different dose levels of 5×10⁶ (FIG. 6A) and 10×10⁶ (FIG. 6B) with or without inducible cytokine prodrug. The data show tumor better tumor growth control over time in mice treated with a combination of pmel-1 (at 5×10⁶ and 10×10⁶) plus WW0621/0523. FIG. 6C shows that survival of mice is increased in mice treated with a combination of pmel-1 (at 5×10⁶ and 10×10⁶) plus WW0621/0523.

FIGS. 7A-7H shows a series of spider plots showing tumor size (mm³) over time in a B16F10 melanoma mouse model. Mice were treated with WW0621/0523 alone, WW0729/0523 alone, IL-2 alone, vehicle+pmel-1, WW0621/0523+pmel-1, WW0729/0523+pmel-1, or IL-2+pmel-1. WW0729/0523 is uncleavable WW0621/0523. The results show that WW0621/0523+pmel-1 delays B16F10 tumor growth.

FIGS. 8A-8F shows a series of spider plots showing activity of combination treatment with pmel-1 with WW0621/0523 relative to pmel-1 alone or WW0621/0523 alone in a B16F10 mouse syngeneic tumor model corresponding to the data shown in FIG. 1. Each line in the plot is the tumor volume over time for a single mouse. Number of surviving animals out of the group of 7 at end of study is also indicated for each plot. The plots also show the number of animals that made a complete recovery/number of animals per group. Three of seven (3/7) animals treated with the combination of 10×10⁶ pmel cells and WW0621/0523 had a complete recovery.

FIGS. 9A-9C are graphs showing the results of combination treatment with pmel-1 plus inducible IL-2 prodrug in a B16F10 melanoma mouse model. FIG. 9A is a graph showing average tumor size (mm³) over time with WW0621/0523 alone, WW0729/0523 alone, IL-2 alone, and vehicle. FIG. 9B is a graph showing average tumor size (mm³) over time with WW0621/0523 plus pmel, WW0729/0523 plus pmel, IL-2 alone plus pmel, vehicle, and vehicle plus pmel. FIG. 9C shows the percentage of survival post treatment in B16F10 mice. The results show that WW0621/0523+pmel-1 delays B16F10 tumor growth and resulted in about 90% survival at the end of the study. Similarly, WW0729/523+pmel-1 delays B16F10 tumor growth and increases survival of animals at end of study (resulted in about 60% survival at the end of the study).

FIGS. 10A-10D are a series of spider plots showing tumor size (mm³) over time in a B16F10 melanoma mouse model administered with a combination of IL-2+pmel-1 or WW0621/0523+pmel.

FIG. 10E is a graph showing the percentage of survival post treatment of a combination of IL-2+pmel-1 or WW0621/0523+pmel. The results show that tumor volume is decreased and survival percentage is increased when mice are treated with a combination of W0621/0523+pmel relative to IL-2+pmel-1.

FIGS. 11A-11F are graphs showing the quantification of donor CD8+VB13+ T cells as a percentage of single cells (FIGS. 11A-11C) or as a percentage of total CD8+ T-cells (FIGS. 11D-11F) at days 8, 15, and 22 post treatment with vehicle, vehicle+pmel-1, WW0621/0523+pmel, WW0729/0523+pmel, WW0621/0523 alone, WW0729/0523 alone, IL-2 alone, or IL-2+pmel-1.

FIGS. 12A and 12B show engraftment of donor cells 8 days post infusion by detecting pmel-1 CD8+VB13+ TCR by flow cytometry. FIG. 12A is a graph showing quantification of CD8+ Vβ13+ cells as a percentage of single cells, and FIG. 12B is a graph showing quantification of CD8+ Vβ13+ cells as a percentage of total CD8+ cells, in mice treated with (i) vehicle, (ii) pmel-land no inducible cytokine prodrug (e.g., vehicle), (iii) pmel-1 at 10×10⁶ and WW0757/0636, (iv) pmel-1 at 10×10⁶ and noncleavable (NC) WW0757/0636, (v) WW0757/0636 alone (e.g., without pmel-1), and (vi) noncleavable (NC) WW0757/0636 alone (e.g., without pmel-1).

FIGS. 13A-13F show a series of spider plots showing activity of combination treatment with pmel-1 with WW0757/0636 relative to pmel-1 alone or WW0757/0636 alone in a B16F10 mouse syngeneic tumor model. Each line in the plots is the tumor volume over time for a single mouse. Number of surviving animals out of the group of 8 (e.g., complete recovery (CR)) at end of study is also indicated for each plot. FIG. 13A shows the spider plot for vehicle treated group. FIG. 13B shows the spider plot for noncleavable (NC) WW0757/0636 alone treated group. FIG. 13C shows the spider plot for WW0757/0636 alone treated group.

FIG. 13D shows the spider plot for pmel-1 treated group alone. FIG. 13E shows the spider plot for noncleavable (NC) WW0757/0636 and pmel-1 treated group. FIG. 13F shows the spider plot for WW0757/0636 and pmel-1 treated group. Three of seven (3/8) animals treated with 10×10⁶ pmel cells and WW0757/0636 had a complete recovery.

FIGS. 14A and 14B are graphs showing the results of combination treatment with pmel-1 and an inducible IL-12 prodrug in a B16F10 melanoma mouse model. FIG. 14A shows survival in mice treated with a combination of pmel-1 (at 10×10⁶) and/or WW0757/0636. FIG. 14B is a graph showing tumor volume average (mm³) over time in mice treated with pmel-1 and/or inducible IL-12 prodrug (i.e., WW0757/0636). The data show tumor enhanced survival and control of tumor growth over time in mice treated with a combination of pmel-1 (at 10×10⁶) plus WW0757/0636.

FIGS. 15A and 15B show the enumeration of immune cells in B16F10 tumors 9 days after the initiation of therapy. The graphs show vehicle and vehicle pmel treated mice compared to mice receiving WW0621/0523 or WW0621/0523+pmel. FIG. 15A shows the immune cells as a percent of total CD45+ cells within the tumor microenvironment and FIG. 15B shows total immune cell numbers. N=5 mice/group.

FIGS. 16A and 16B show the enumeration of immune cells in B16F10 tumors 9 days after the initiation of therapy. Both graphs show vehicle and vehicle pmel treated mice compared to mice receiving WW0757/636 or WW0757/636+pmel. FIG. 16A shows the immune cells as a percent of total CD45+ cells within the tumor microenvironment and FIG. 16B shows total immune cell numbers. N=5 mice/group.

FIGS. 17A-17D shows the engraftment of the donor pmel cells identified by Vβ13 expression in B16F10 tumors treated with CD8+ pmel cells alone, WW0621/0523 alone, or the combination of both therapies. FIG. 17A are representative flow plots of CD8+ T cells showing the fraction of Vβ13+ donor cells vs Vβ13− host CD8+ T cells. FIGS. 17B-17D are graphs that quantifies the donor Vβ13+ cells by total cell number, as a percent of CD45 and as a percent of CD8+ T cells. N=5 mice/group.

FIGS. 18A-18D show the engraftment of the donor pmel cells identified by Vβ13 expression in B16F10 tumors treated with CD8+pmel cells alone WW0757/636 alone, or the combination of both therapies. FIG. 18A are representative flow plots of CD8+ T cells showing the fraction of Vβ13+ donor cells vs Vβ13− host CD8+ T cells. FIGS. 18B-18D quantifies the donor Vβ13+ cells by total cell number, as a percent of CD45 and as a percent of CD8+ T cells. N=5 mice/group.

FIGS. 19A-19C show the expression of CD25 on donor and host CD8+ T cells treated with pmel, WW0621/0523 alone, WW0757/636 alone, or a combination of pmel and inducible IL-2 prodrug or inducible IL-12 prodrug. FIG. 19A are representative flow plots of donor CD8+ Vβ13+ cells CD25 expression. FIGS. 19B and 19C display the percentage of CD25+ cells in the CD8+ Vβ13+ (B) and CD8+ Vβ13− (C) cells within the tumor. N=5 mice/group.

FIGS. 20A-20C show the expression of Ki67 on donor and host CD8+ T cells treated with pmel, WW0621/0523 alone, WW0757/636 alone, or a combination of pmel and inducible IL-2 prodrug or inducible IL-12 prodrug. FIG. 20A are representative flow plots of donor CD8+ Vβ13+ cells Ki67 expression. FIGS. 20B-20C display the percentage of Ki67+ cells in the CD8+ Vβ13+ (B) and CD8+ Vβ13− (C) cells within the tumor. N=5 mice/group.

FIGS. 21A-21C shows the expression of PD-1 on donor and host CD8+ T cells treated with pmel, WW0621/0523 alone, WW0757/636 alone, or a combination of pmel and inducible IL-2 prodrug or inducible IL-12 prodrug. FIG. 21A are representative flow plots of donor CD8+ Vβ13+ cells PD-1 expression. FIGS. 21B-21C display the percentage of PD-1+ cells in the CD8+ Vβ13+ (B) and CD8+ Vβ13− (C) cells within the tumor. N=5 mice/group.

FIGS. 22A-22D depicts the polyfunctionality of donor CD8+ Vβ13+ cells in the tumor microenvironment treated with or without WW0621/0523 or WW0757/636. FIG. 22A shows the frequency of donor CD8+ Vβ13+ coexpressing one, two or three of IFNγ, TNFα and Granzyme B. FIGS. 22B-2D shows the frequency of donor CD8+ Vβ13+ cells that express IFNγ, TNFα or Granzyme B. N=5 mice/group, one way ANOVA with multiple comparisons.

FIGS. 23A-23D depicts the polyfunctionality of donor CD8+ Vβ13− cells in the tumor microenvironment treated with pmel cells alone, WW0621/0523 alone, WW0757/636 alone, or a combination of pmel cells and inducible IL-2 prodrug or inducible IL-12 prodrug. FIG. 23A shows the frequency of host CD8+ Vβ13− coexpressing one, two or three of IFNγ, TNFα and Granzyme B. FIGS. 23B-23D shows the frequency of donor CD8+ Vβ13− cells that express IFNγ, TNFα or Granzyme B. N=5 mice/group, one way ANOVA with multiple comparisons.

FIG. 24 is flow cytometric plots depicting the percentage of expression of CD19 CAR T cells expressing inducible IL-2 prodrug in CD4+ T cells and CD8+ T cells by flow cytometry prior to infusion into mice.

FIGS. 25A-25E are graphs showing the results of treatment with CD19 CAR-T cells expressing inducible IL-2 prodrug in a Burkitt's lymphoma mouse model. FIG. 25A shows a survival plot for mice treated with 5×10⁶ CD19 CAR+T cells or 5×10⁶ CD19 CAR T cells expressing inducible IL-2 prodrug and untreated mice. FIG. 25B is a graph showing tumor volume average (mm³) over time in mice treated with 5×10⁶ CD19 CAR+T cells or 5×10⁶ CD19 CAR T cells expressing inducible IL-2 prodrug and untreated mice. FIGS. 25C-25E are graphs showing tumor volume (mm³) over time in individual mice that were untreated (FIG. 25C), treated with 5×10⁶ CD19 CAR+T cells (FIG. 25D) or treated with 5×10⁶ CD19 CAR T cells expressing inducible IL-2 prodrug (FIG. 25E). The data show increased control of tumor growth in mice treated with a CD19 CAR T cells expressing inducible IL-2 prodrug compared to untreated mice and mice treated with CD19 CAR+ T cells alone. CR refers to complete remission.

FIGS. 26A-26E are graphs showing the results of treatment with CD19 CAR T cells expressing inducible IL-2 prodrug in a Burkitt's lymphoma mouse model. FIG. 26A shows a survival plot for mice treated with 10×10⁶ CD19 CAR+T cells, or mice treated with 10×10⁶ CD19 CAR T cells expressing inducible IL-2 prodrug and untreated mice. FIG. 26B is a graph showing tumor volume average (mm³) over time in mice treated with 10×10⁶ CD19 CAR+T cells or 10×10⁶ CD19 CAR T cells expressing inducible IL-2 prodrug. FIGS. 26C-26E are graphs showing tumor volume (mm³) over time in individual mice that were untreated (FIG. 26C) mice treated with 10×10⁶ CD19 CAR+T cells (FIG. 26D) or 10×10⁶ CD19 CAR T cells expressing inducible IL-2 prodrug (FIG. 26E). The data show increased control of tumor growth in mice treated with CD19 CAR T cells expressing inducible IL-2 prodrug compared to untreated mice and mice treated with CD19 CART cells alone.

FIG. 27 is a graph showing CD45+CD3+ cells as a percentage of single cells in a Burkitt's lymphoma mouse model in which mice were treated with 5×10⁶ CD19 CAR+ T cells, 5×10⁶ CD19 CAR T cells expressing inducible IL-2 prodrug, 10×10⁶ CD19 CAR+ T cells, or 10×10⁶ CD19 CAR T cells expressing inducible IL-2 prodrug. A one way ANOVA with multiple comparison statistical analysis was performed. The graph shows that CD19 CAR T cells expressing inducible IL-2 prodrug have significantly increased persistence of CD45+CD3+ donor cells relative to CD19 CAR T cells alone.

FIG. 28A-28C shows expression of CD19 CAR T cells expressing inducible IL-2 prodrug in CD4+ T cells and CD8+ T cells by flow cytometry. FIG. 28A are flow cytometric plots depicting the percentage of CD4+ T cells and CD8+ T cells that express inducible IL-2 prodrug tagged with GFP in mice treated with 5×10⁶ CD19 CAR+ T cells, 5×10⁶ CD19 CAR T cells expressing inducible IL-2 prodrug, 10×10⁶ CD19 CAR+ T cells, or 10×10⁶ CD19 CAR T cells expressing inducible IL-2. The gates show the population of CD4+ T cells or CD8+ T cells. FIG. 28B is a graph showing the percentage of CD4+ T cells expressing inducible IL-2 prodrug. FIG. 28C is a graph showing the percentage of CD8+ T cells expressing inducible IL-2 prodrug. The data show that inducible IL-2 prodrug is primarily expressed in CD8+ T cells and shows persistence of the engrafted cells throughout the study period.

FIGS. 29A-29C depicts the IL-2 inducible prodrug expression of human CAR T cells transferred into NSG mice bearing Raji tumors 65 days after initial transfer. FIG. 29A are representative flow plots of CD4+ and CD8+ T cells showing IL-2 inducible prodrug expression marked by GFP expression in both CAR and CAR IL-2 inducible prodrug transduced cell products 65 days post infusion. FIGS. 29B-29C shows the frequency of IL-2 inducible prodrug positive cells in CD4+ or CD8+ T cells. N=3-4 mice/group.

FIG. 30 is a schematic diagram depicting the study protocol for systemic inducible IL-2 prodrug and/or inducible IL-12 prodrug therapy (WW0621/0523 or WW0758/0636) in combination with CD19 CAR T cells to treat NSG mice bearing Raji tumors.

FIG. 31A-31F depicts the tumor growth and survival of NSG mice bearing Raji tumors treated with untransduced T cells or inducible IL-2 prodrug or inducible IL-12 prodrug therapy alone. FIG. 31A shows the tumor growth of Raji tumors treated with untransduced T cells or inducible IL-2 prodrug or inducible IL-12 prodrug therapy alone. FIG. 31B shows the tumor survival of tumor bearing mice treated with untransduced T cells or inducible IL-2 prodrug or inducible IL-12 prodrug therapy alone. FIGS. 31C-31F shows the representative flow plots of each of the groups treated with untransduced T cells or inducible IL-2 prodrug or inducible IL-12 prodrug therapy alone. Each line represents an individual mouse. N=8 mice/group.2

FIGS. 32A-37F depicts the tumor growth and survival of NSG mice bearing Raji tumors treated with CD19 CAR T cells alone, inducible IL-2 prodrug therapy alone or a combination of therapies. FIG. 32A shows the tumor growth of Raji tumors treated with CD19 CAR T cells alone, inducible IL-2 prodrug or inducible IL-12 prodrug therapy alone or a combination of therapies. FIG. 32B shows the tumor survival of tumor bearing mice treated with CD19 CAR T cells alone, inducible IL-2 prodrug or inducible IL-12 prodrug therapy alone or a combination of therapies. FIGS. 32C-32F shows the representative flow plots of each of the groups treated with CD19 CAR T cells alone, inducible IL-2 prodrug or inducible IL-12 prodrug therapy alone or a combination of therapies. Each line represents an individual mouse. N=8 mice/group.

FIGS. 33A and 33B depicts the proportion of human T cells in NSG mice bearing Raji tumors receiving untransduced human T cells or inducible IL-2 prodrug or inducible IL-12 prodrug therapy alone. FIG. 33A are representative flow plots showing the proportion of human T cells marked by CD45+ CD3+ in the peripheral blood of NSG mice 17 days post infusion. FIG. 33B is the frequency of CD45+ CD3+ cells in the peripheral blood of NSG mice bearing Raji tumors. N=8 mice/group.

FIGS. 34A and 34B depicts the proportion of human T cells in NSG mice bearing Raji tumors receiving CD19 CAR T cells alone or in combination with inducible IL-2 prodrug or inducible IL-12 prodrug therapy. FIG. 34A are representative flow plots showing the proportion of human T cells marked by CD45+ CD3+ in the peripheral blood of NSG mice 17 days post infusion. FIG. 34B is the frequency of CD45+ CD3+ cells in the peripheral blood of NSG mice bearing Raji tumors. N=8 mice/group.

FIGS. 35A and 35B depicts the proportion of CD4+ (A) and CD8+ (B) T cells of the adoptively transferred CD45+ CD3+ cells in the peripheral blood of NSG mice 17 days post infusion.

FIGS. 36A-36C depicts the CAR expression of adoptively transferred CD4+ and CD8+ T cells in the peripheral blood of NSG mice 17 days post infusion. FIG. 36A are representative flow plots of peripheral blood CD4+ and CD8+ T cells showing CD19 CAR expression. FIGS. 36B and 36C are the frequency of CD4+ (B) and CD8+ (C) T cells that express the CD19 CAR. N=8 mice/group.

FIGS. 37A-37C depicts the engraftment of CD19 CAR T cells treated with or without inducible IL-2 prodrug or inducible IL-12 prodrug therapy on Days 17 and Day 30 post infusion. Data is depicted as CD45+ CD3+ (% total lymphocyte population), CD4+ T cells (% of CD45+ CD3+) and CD8+ T cells (% of CD45+ CD3+) in NSG mice treated with CD19 CAR T cells (A), CD19 CAR T cells+WW0621/0523 (FIG. 37B) or CD19 CAR T cells+WW0758/0636 (FIG. 37C). N=8 mice/group Day 17 and n=3-7 mice/group Day 30.

FIG. 38 is a schematic diagram depicting the study protocol for systemic inducible IL-21 prodrug (WW50387/WW50394) in combination with CD19 CAR T cells to treat NSG mice bearing Raji tumors.

FIGS. 39A-39C depict the tumor growth inhibition of NSG mice bearing Raji tumors treated with vehicle (FIG. 39A), CD19 CAR T cells plus vehicle (FIG. 39B), or CD 19 CAR T cells plus inducible IL-21 prodrug (FIG. 39C). FIGS. 39D-39F shows body weights of mice treated in FIGS. 39A-39C.

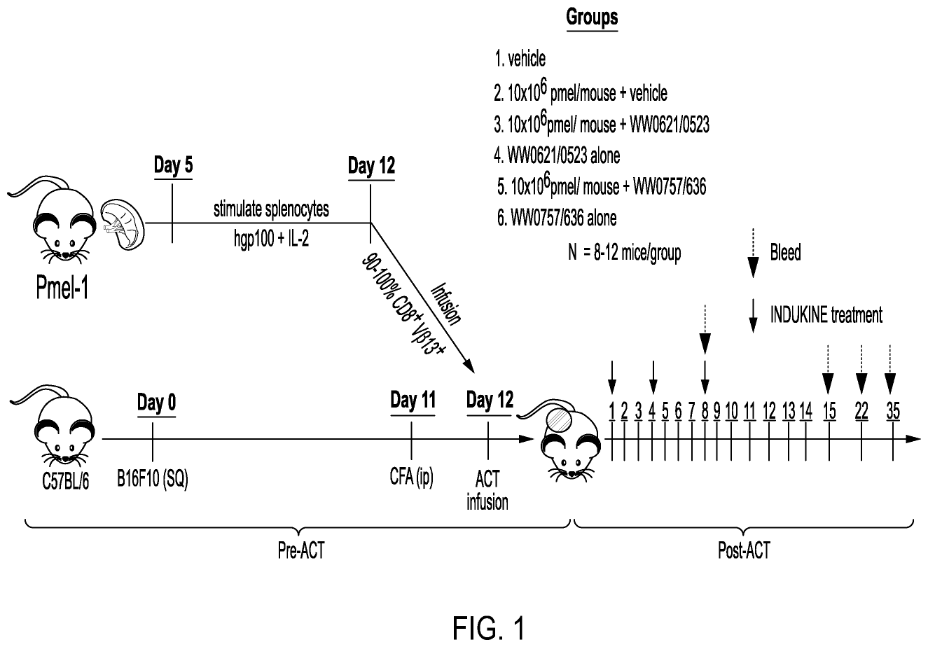

### 5. DETAILED DESCRIPTION

This disclosure relates to combination therapy that includes adoptive cell therapy and an inducible cytokine prodrug, and to compositions for use in such therapy. Adoptive cell therapy can have increased efficacy and/or improved safety when combined with the inducible cytokine prodrugs as described herein. Without wishing to be bound by any particular theory, it is believed that the combination of adoptive cell therapy and an inducible cytokine that is activated in the tumor microenvironment, can lead to greater immune cell recruitment, proliferation and effector function in the tumor microenvironment. This is expected to help limit well-known challenges and adverse events of adoptive cell therapy such as cytokine release syndrome, neurotoxicity, on-target but off tumor toxicity, and immune exhaustion.

This disclosure relates to the use of inducible cytokines to increase the efficacy of adoptive cell therapies by, for example, increasing the ability of the therapeutic cells to proliferate and/or deliver effector functions at the desired site of activity. This may, for example, overcome the immunosuppressive effect of the tumor microenvironment, increasing the effectiveness of adoptive cell therapies in solid tumors.

Inducible cytokines can interact with endogenous non-engineered immune cells in the tumor microenvironment. When an inducible cytokine is combined with an adoptive cell therapy targeting a specific tumor antigen, activation of the inducible cytokine and targeting the adoptive cell therapy to the tumor microenvironment can lead to activation of the inducible cytokine and enhanced activity of the adoptive cell therapy. Activation of the inducible cytokine can also lead to enhanced effector function of endogenous immune cells which can lead to killing of tumor cells that do not express the specific tumor antigen recognized by the adoptive cell therapy. This can lead to greater efficacy, for example, by mitigating problems such as tumor heterogeneity.

These increases in adoptive cell therapy efficacy beneficially also avoid risk of toxicity when compared to combinations with systemic cytokine administration, due to the greatly diminished activity of the inducible cytokine outside of the tumor microenvironment. These increases in efficacy could allow for the design of safer cell therapy compositions or dosing regimens. They could also allow cell therapies to be effective in indications where they have not been previously.

This combination could also be used in place of existing combinations of cell therapy and natural cytokines, such as TILs and IL-2 or TILs and IL-12. The adoptive cell therapy preferably provides therapeutic benefit for cancer and/or inflammation (e.g. autoimmune diseases). For example, the adoptive cell therapy can comprise immune cells that recognize and provide effector function to kill tumor cells. In some embodiments, the adoptive cell therapy comprises immune cells that have been genetically modified, such as to provide specificity for target antigens, for example target antigens that are preferentially expressed by tumors. In some embodiments, such immune cells express chimeric antigen receptors (CAR) or modified T cell receptors (TCR). In some embodiments, the adoptive cell therapy comprises immune cells that are capable of killing tumor cells. In some embodiments, such immune cells can express a inducible cytokine prodrug such as a CAR. In some embodiments, the adoptive cell therapy comprises tumor infiltrating lymphocytes (TILs).

This disclosure further relates to immune cells that are engineered to express a desired inducible cytokine prodrug. For example, a T cell (e.g., TIL, CAR-T, TCR-T) can be engineered to express an inducible cytokine prodrug, such as an inducible IL-2 prodrug or inducible IL-12 prodrug as described herein. Similarly, other immune cells, such as NK cell can be engineered to express an inducible cytokine, and if desired, an antigen binding protein that directs NK cell activity to cells that express a selected antigen.

### A. Inducible Cytokine Prodrug

Typically, an inducible cytokine prodrug contains at least one polypeptide chain, and can comprise two or more polypeptide chains, if desired. The inducible cytokine prodrug comprises a cytokine (e.g., IL-2 or IL-12), a blocking element, a protease cleavable linker, and a half-life extension element. Inducible cytokine prodrugs can be administered in combination with an adoptive cell therapy, e.g., a population of immune cells. The inducible cytokine prodrug and adoptive cell therapy are administered to provide an overlap in their pharmacological or biological activities. Accordingly, the inducible cytokine prodrug disclosed herein can be administered before, concurrently with or after the adoptive cell therapy. Inducible cytokine prodrugs can be expressed by immune cells that are engineered to express the inducible cytokine prodrugs.

Any cytokine of interest can be suitable for the inducible cytokine prodrugs of this disclosure. Exemplary cytokines include, but are not limited to, interleukins such as IL-2, IL-7, IL-12, IL-10, IL-15, IL-18, IL-21IL-23, lymphotoxin, TGF-beta1, TGFbeta2, TGFbeta3, GM-CSF, CXCL10, CCL19, CCL20, CCL21 and functional fragments or muteins of any of the foregoing. Preferred cytokines for use in the inducible cytokine prodrugs disclosed herein are IL-2, IL-12, muteins, functional variants, and functional fragments, or subunits of any of the foregoing. Inducible cytokine (e.g., IL-2 or IL-12) prodrugs of this disclosure have attenuated cytokine receptor agonist activity and the circulating half-life is extended. The inducible cytokine receptor agonist activity is attenuated through the blocking element. The half-life extension element can also contribute to attenuation, for example through steric effects. The blocking element is capable of blocking all or some of the receptor agonist activity of the cytokine by noncovalently binding to the cytokine (e.g., to IL-2 or IL-12) and/or sterically blocking receptor binding. Upon cleavage of the protease cleavable linker a form of the cytokine is released that is active (e.g., more active than the cytokine polypeptide prodrug). Typically, the released cytokine is at least 10× more active than the cytokine polypeptide prodrug. Preferably, the released cytokine is at least 20×, at least 30×, at least 50×, at least 100×, at least 200×, at least 300×, at least 500×, at least 1000×, at least about 10,000× or more active than the inducible cytokine.

The form of cytokine that is released upon cleavage of the inducible cytokine prodrug typically has a short half-life, which is often substantially similar to the half-life of naturally occurring cytokine. Even though the half-life of the inducible cytokine prodrug is extended, toxicity is reduced or eliminated because the agonist activity of the circulating inducible cytokine prodrug is attenuated and active cytokine is targeted to the desired site of activity (e.g., tumor microenvironment).

It will be appreciated by those skilled in the art, that the number of polypeptide chains, and the location of the elements, the half-life extension element, the protease cleavable linker(s), and the blocking element (and components of such elements, such as a VH or VL domain) on the polypeptide chains can vary and is often a matter of design preference. All such variations are encompassed by this disclosure.

The person of ordinary skill in the art is also directed to International Publication Nos.: WO2019/222294, WO2019/222295 WO2019/222296, WO2021097376, and WO2021236676A1, which disclose exemplary inducible cytokines suitable for use in the combination therapy disclosed herein.

The inducible cytokine prodrug can comprise a single polypeptide chain. Typically, the single polypeptide chain inducible cytokine prodrug comprises a cytokine polypeptide [A], a blocking element [D], optionally a half-life extension element [H], and a protease cleavable linker [L]. When the optional half-life is absent, it is preferred that the blocking element can also function as a half-life extending element as described herein and sterically inhibits binding of the cytokine polypeptide in the prodrug to its cognate receptor. The cytokine [A] polypeptide can be operably linked to the blocking element, the half-life extension element (when present) or both the blocking element and the half-life extension element (when present) by a protease cleavable linker. Typically, the single polypeptide can comprise one cytokine polypeptide or two cytokine polypeptides. The cytokine polypeptide can be located at any desired position in the single polypeptide chain.

The single polypeptide can comprise two or more blocking elements that also function as half-life extension elements (e.g., an antibody fragment that binds HSA). When two or more of such blocking elements are present in the inducible IFNalpha prodrug, they can block all or some of the receptor agonist activity of IFNalpha and also extend serum half-life. When two or more such a blocking elements are present in and IFNalpha prodrug, a separate half-life extension element or a separate blocking element are optional and are typically not present.

The inducible cytokine prodrug can be of any of Formulas (I)-(VI).

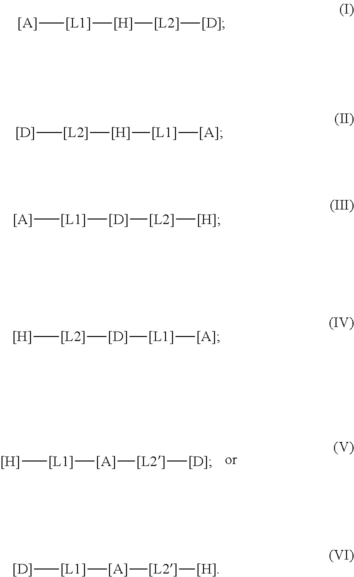

In Formulas (I)-(VI), [A] is a cytokine polypeptide, [D] is a blocking element, [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can have the same or different amino acid sequence and or protease-cleavage site (when L2 is protease-cleavable) as desired. The protease cleavable linker can comprise the sequence GPAGLYAQ (SEQ ID NO: 195) or ALFKSSFP (SEQ ID NO: 198), particularly ALFKSSFP (SEQ ID NO: 198).

SEQ ID NOs: 21-30 are specific examples of inducible IL-2 prodrugs encompassed by Formulas (I)-(VI) and for use according to this disclosure. SEQ ID NOs. 21-30 and additional details regarding their activity is disclosed in International Publication No.: WO2021/097376.

SEQ ID NOs: 31-40 are specific examples of inducible IL-12 prodrugs encompassed by Formulas (I)-(VI) and for use according to this disclosure. SEQ ID NOs.: 31-40 and additional details regarding their activity is disclosed in International Application No.: PCT/US2021/33014 and International Publication No.: WO2019/222295.

In some instances, the single polypeptide chain comprises a cytokine polypeptide [A], a blocking element (i.e., a steric blocking polypeptide) [D], a protease cleavable linker [L], and an optional half-life extension element. The blocking element [D] can be, for example, HSA or an antibody or antibody fragment (e.g. scFv) that binds HSA. As an example, the polypeptide can be of Formula (VII): [D]-[L1]-[A]-[L2]-[D]. SEQ ID NOs: 14-20 are specific examples, of inducible IFN prodrugs for use according to this disclosure. SEQ ID NOs: 14-20 and additional details regarding their activity is disclosed in International Application No.: PCT/US2020/060624.

IFN polypeptide and the blocking element and the half-life extension element are operably linked by the protease-cleavable polypeptide. For example, the polypeptide can be of any of Formulas (I)-(IX).

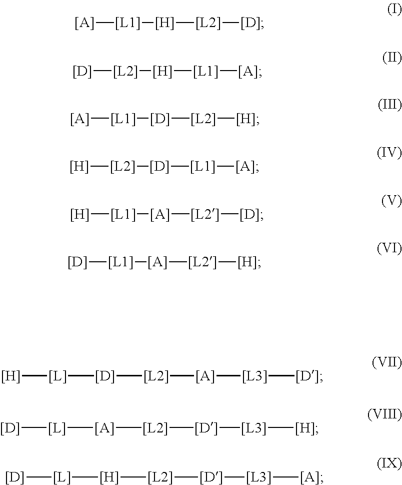

In Formulas (I)-(IX), [A] is a IFN polypeptide, [D] is a IFN blocking element (e.g., extracellular portion of the INFalpha receptor 1 (IFNAR1) or IFNalpha receptor 2 (IFNAR2)), [D′] is either the INFalpha receptor 1 (IFNAR1) or the IFNalpha receptor 2 (IFNAR2) that is not present in [D], [H] is a half-life extension element, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can be the same or different amino acid sequence and or protease-cleavage site (when L2 is protease-cleavable) as desired.

The inducible cytokine prodrugs can contain two or more polypeptide chains. Such inducible cytokine prodrugs comprise a cytokine polypeptide, a half-life extension element that extends the half-life of the inducible cytokine prodrug, typically a blocking domain, and a protease cleavable linker. The components of the inducible cytokine prodrug can be on the same polypeptide chain or on different polypeptide chains. Illustrative of this, and as disclosed and exemplified herein, components of the blocking domain can present on separate polypeptide chains. For example, a first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds to the cytokine (e.g., IL-2, IL-12, or IFN) and attenuates the cytokine activity.

For example, the inducible cytokine prodrug can have a first polypeptide of Formulas (I)-(VI). In Formulas (I)-(VI), [A] is a cytokine polypeptide, [D] is an antibody heavy chain Fab fragment (VH+CH1) or heavy chain variable domain (VH), [H] is a half-life extension element as disclosed herein, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can have the same or different amino acid sequence and or protease-cleavage site (when L2 is protease-cleavable) as desired. A second polypeptide chain comprising antibody light chain (VL+CL) or light chain variable domain (VL) that is complementary to the VH+CH1 or VH. For example, the inducible cytokine prodrug can have a first polypeptide of Formulas (I)-(VI). In Formulas (I)-(VI), [A] is a cytokine polypeptide, [D] is an antibody heavy chain Fab fragment (VL+CL) or light chain variable domain (VL), [H] is a half-life extension element as disclosed herein, [L1] is a protease-cleavable polypeptide linker, [L2] is an polypeptide linker that is optionally protease-cleavable, and [L2′] is a protease-cleavable polypeptide linker. [L1] and [L2] or [L1] and [L2′] can have the same or different amino acid sequence and or protease-cleavage site (when L2 is protease-cleavable) as desired. A second polypeptide an antibody heavy chain Fab fragment (VH+CH1) or heavy chain variable domain (VH) that is complementary to the VL+CL or VL.

For instances, the first polypeptide chain can comprise from the cytokine polypeptide, a protease cleavable linker, a half-life extension element, and a VH and CH1 of an antibody that binds the cytokine (e.g., IL-2, a IL-12 subunit i.e., p35, p40, or the p35p40 heterodimeric complex, or IFN). The second polypeptide chain can comprise a VL and CL of an antibody that binds the cytokine (e.g., IL-2, IL-12 subunit (i.e., p35, p40, or the p35p40 heterodimeric complex), or IFN) and that together with the VH and CH1 of the first polypeptide chain form a Fab that binds the cytokine (e.g., IL-2, IL-12 subunit (i.e., p35, p40, or the p35p40 heterodimeric complex), or IFN) polypeptide.

Cytokines that comprise two subunits, such as IL-12, can also comprise two or more different polypeptides. For instance, the first polypeptide can comprise an cytokine subunit (e.g., IL-12 p35 or il-12 p40), and optionally a blocking domain. The blocking domain, when present, can be operably linked to the cytokine subunit through a first protease cleavable linker. The second polypeptide chain can comprise an cytokine subunit operably linked to a half-life extension element as disclosed herein through a second protease cleavable linker, and optionally a blocking domain. The blocking domain when present can be operably linked to the cytokine subunit through a protease cleavable linker or can be operably linked to the half-life extension element through a linker that is optionally protease cleavable. Only one of the first and second polypeptide contains the blocking domain. Typically, the first polypeptide and second polypeptides contain different cytokine subunits. For instance, when the IL-12 subunit in the first polypeptide is p35, the IL-12 subunit in the second polypeptide is p40, and when the IL-12 subunit in the first polypeptide is p40, the IL-12 subunit in the second polypeptide is p35. A blocking domain can be a single chain antibody that binds the cytokine or an antigen binding fragment thereof. The cleavable linkers in this inducible cytokine prodrug can be the same or different.

The inducible cytokine polypeptide prodrug can comprise three different polypeptides. One polypeptide chain can comprise a cytokine subunit and a second polypeptide can comprise the other cytokine subunit, and the third polypeptide comprises at least a portion (component) of the blocking domain. The first polypeptide can comprise a cytokine subunit, and optionally a half-life extension element. The half-life extension element, when present, can be operably linked to the cytokine subunit through a protease cleavable linker. The second polypeptide can comprise a cytokine subunit, at least an antigen binding portion of an antibody light chain or an antigen binding portion of an antibody heavy chain, and optionally a half-life extension element as disclosed herein. When the half-life extension element is present, it can be operably linked to the cytokine subunit through a protease cleavable linker and the antibody heavy chain or light chain is either a) operably linked to the IL-12 subunit through a second protease cleavable linker, or b) operably linked to the half-life extension element through an optionally cleavable linker. The third polypeptide can comprise can an antigen binding portion of an antibody heavy chain that is complementary to the light chain in the second polypeptide, or an antibody light chain that is complementary to the heavy chain in the second polypeptide and together with said light chain forms a cytokine binding site. In this complex, the cytokine blocking domain is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible cytokine prodrug can be the same or different.

For example, one polypeptide chain comprises either the p35 or p40 IL-12 subunit, but not both, and a second polypeptide comprises the other IL-12 subunit and the third polypeptide comprises at least a portion (component) of the blocking domain. The first polypeptide can comprise a IL-12 subunit, and optionally a half-life extension element. The half-life extension element, when present, can be operably linked to the IL-12 subunit through a protease cleavable linker. The second polypeptide can comprise a IL-12 subunit, at least an antigen binding portion of an antibody light chain or an antigen binding portion of an antibody heavy chain, and optionally a half-life extension element as disclosed herein. When the half-life extension element is present, it can be operably linked to the IL-12 subunit through a protease cleavable linker and the antibody heavy chain or light chain is either a) operably linked to the IL-12 subunit through a second protease cleavable linker, or b) operably linked to the half-life extension element through an optionally cleavable linker. The third polypeptide can comprise can an antigen binding portion of an antibody heavy chain that is complementary to the light chain in the second polypeptide, or an antibody light chain that is complementary to the heavy chain in the second polypeptide and together with said light chain forms an IL-12 binding site. When the IL-12 subunit in the first polypeptide is p35, the IL-12 subunit in the second polypeptide is p40, and when the IL-12 subunit in the first polypeptide is p40, the IL-12 subunit in the second polypeptide is p35. In this inducible cytokine prodrug, the IL-12 blocking domain is preferably an antigen binding fragment of an antibody. The antigen binding fragment comprises as separate components, at least an antigen-binding portion of an antibody light chain and at least an antigen-binding portion of a complementary antibody heavy chain. The protease cleavable linkers in this inducible IL-12 prodrug can be the same or different.

The inducible polypeptide complex can comprise two different polypeptides wherein cytokine subunits (e.g., p35 and p40) are located on the same polypeptide chain. For example, a first polypeptide chain can comprise p35, p40, a half-life extension element and at least an antigen binding portion of an antibody light chain. p35 and p40 can be operably linked, and the half-life extension element can be operably linked to p40 through a first protease cleavable linker and the antigen binding portion of an antibody light chain can be operably linked to p35 through a protease cleavable linker. Alternatively, the half-life extension element can be operably linked to p35 through a protease cleavable linker and the antigen binding portion of an antibody light chain is operably linked to p40 through a protease cleavable linker. The second polypeptide comprises at least an antigen binding portion of an antibody heavy chain that is complementary to the light chain in the second polypeptide and together with said light chain forms and IL-12 binding site. The protease cleavable linkers in this inducible cytokine prodrug can be the same or different.

In an alternative format, a first polypeptide chain can comprise p35, p40, a sdAb and at least an antigen binding portion of an antibody heavy chain. p35 and p40 can be operably linked, and the sdAb can be operably linked to p40 or through a protease cleavable linker and the antigen binding portion of an antibody heavy chain can be operably linked to p35 through a protease cleavable linker. Alternatively, the sdAb can be operably linked to p35 through a protease cleavable linker and the antigen binding portion of an antibody heavy chain can be operably linked to p40 through a second protease cleavable linker. A second polypeptide comprises at least an antigen binding portion of an antibody light chain that is complementary to the heavy chain in the second polypeptide and together with said light chain forms and IL-12 binding site. The protease cleavable linkers in this complex can be the same or different.

The inducible cytokine prodrugs disclosed herein may contain at least one half-life extension element and at least one blocking element, and such elements can contain two or more components that are present on the same polypeptide chain or on different polypeptide chains. The first polypeptide chain can comprise a first half-life extension element, and a second polypeptide chain can comprise a second half-life extension element. The first half-life extension element can comprise a heavy chain polypeptide or portion thereof (e.g., an Fc domain or fragment thereof) that optionally comprises one or more amino acid mutations that creates a “knob,” and the second half-life extension element can comprise a heavy chain polypeptide or portion thereof (e.g., an Fc domain or fragment thereof) that optionally comprises one or more amino acid mutations that create a “hole.” The first half-life extension element can comprise a heavy chain polypeptide or portion thereof (e.g., an Fc domain or fragment thereof) that optionally comprises one or more amino acid mutations that creates a “hole,” and the second half-life extension element can comprise a heavy chain polypeptide or portion thereof (e.g., an Fc domain or fragment thereof) that optionally comprises one or more amino acid mutations that create a “knob.” The first half-life extension element and the second half-life extension element can form a heterodimer (i.e., heterodimerize). The first half-life extension element and the second half-life extension element can form a heterodimer through disulfide bonds or a optionally protease cleavable linker, for example.

The first polypeptide chain and second polypeptide chain can each comprise a half-life extension element. For example, the first polypeptide chain can comprise the first half-life extension element, a cytokine polypeptide, and a blocking element, and the second polypeptide chain can comprise the second half-life extension element. For example, the first polypeptide chain can comprise the first half-life extension element and a cytokine polypeptide, and the second polypeptide chain can comprise the second half-life extension element and a blocking element. For example, the first polypeptide chain can comprise the first half-life extension element and a blocking element, and the second polypeptide chain can comprise the second half-life extension element and a cytokine polypeptide.

In an example, the inducible IL-12 cytokine prodrug comprises a first polypeptide does not comprise a blocking element and the second polypeptide has the formula: [A]-[L1]-[B]-[L3]-[D] or [D]-[L3]-[B]-[L1]-[A] or [B]-[L1]-[A]-[L2]-[D] or [D]-[L1]-[A]-[L2]-[B], wherein, A is the IL-12 subunit; L1 is the first protease-cleavable linker; L2 is the second protease cleavable linker; L3 is the optionally cleavable linker; B is the half-life extension element; and D is the blocking element.

In another example, the first polypeptide comprises the formula: [A]-[L1]-[D] or [D]-[L1]-[A]; and the second polypeptide has the formula: [A′]-[L2]-[B] or [B]-[L2]-[A′], wherein A is either p35 or p40, wherein when A is p35, A′ is p40 and when A is p40, A′ is p35; A′ is either p35 or p40; L1 is the first protease cleavable linker; L2 is the second protease cleavable linker; B is the half-life extension element; and D is the blocking element.

Compounds 1, 2, 3 and 4 are specific examples of inducible IL-2 prodrugs that comprise two polypeptide chains for use according to this disclosure. Compounds 1, 2, 3, and 4 and additional details regarding their activity is disclosed in WO2021/097376.

TABLE 1
Inducible IL-2 prodrugs

| IL-2 Prodrug | First Polypeptide | Second Polypeptide |
|---|---|---|
| Compound 1 | SEQ ID NO: 1 | SEQ ID NO: 5 |
| Compound 2 | SEQ ID NO: 2 | SEQ ID NO: 5 |
| Compound 3 | SEQ ID NO: 3 | SEQ ID NO: 5 |
| Compound 4 | SEQ ID NO: 4 | SEQ ID NO: 5 |

Compounds 5, 6, 7, 8, 9, and 10 are specific examples of inducible IL-12 prodrugs that comprise two polypeptide chains for use according to this disclosure. Compounds 5, 6, 7, 8, 9, and 10 and additional details regarding their activity is disclosed in International Application No.: PCT/US2021/33014.

TABLE 2
Inducible IL-12 prodrugs

| IL-12 Prodrug | First Polypeptide | Second Polypeptide |
|---|---|---|
| Compound 5 | SEQ ID NO: 6 | SEQ ID NO: 12 |
| Compound 6 | SEQ ID NO: 7 | SEQ ID NO: 12 |
| Compound 7 | SEQ ID NO: 8 | SEQ ID NO: 13 |
| Compound 8 | SEQ ID NO: 9 | SEQ ID NO: 13 |
| Compound 9 | SEQ ID NO: 10 | SEQ ID NO: 13 |
| Compound 10 | SEQ ID NO: 11 | SEQ ID NO: 13 |
| Compound 11 | SEQ ID NO: 45 | SEQ ID NO: 12 |

As described above, the cytokine can be a mutein, if desired. The cytokine mutein retains activity, for example intrinsic IL-12/IL-2 receptor agonist activity.

### B. Protease Cleavable Linker

As described herein, a protease cleavable linker may be designed so that it is cleaved with high efficiency by proteases at a desired location (e.g., proteases that have higher activity in the tumor microenvironment and lower activity in other locations) but are stable and not cleaved or cleaved with low efficiency in other locations (e.g., in the periphery, for example healthy tissue or serum).

The linkers disclosed herein can confer functionality, including flexibility as well as the ability to be cleaved. Flexible linkers are usually applied when joined domains requires a certain degree of movement or interaction. Cleavable linkers are introduced to release free and functional domains in vivo at a target site. The linkers can maintain cooperative inter-domain interactions or preserving biological activity. The cleavable linkers can join functional domains (e.g., a payload and half-life extension element) that are released from the cleavable linker at a target site (e.g. a tumor microenvironment).

In a preferred embodiment, the linker is cleavable by a cleaving agent, e.g., an enzyme. Preferably, the linker comprises a protease cleavage site. In some cases, the linker comprises one or more cleavage sites. The linker can comprise a single protease cleavage site. The linker can also comprise 2 or more protease cleavage sites. For example, 2 protease cleavage sites, 3 protease cleavage sites, 4 protease cleavage sites, 5 protease cleavage sites, or more. In some cases, the linker comprises 2 or more protease cleavage sites, and the protease cleavage sites can be cleaved by the same protease or different proteases. A linker comprising two or more cleavage sites is referred to as a “tandem linker.” The two or more cleavage sites can be arranged in any desired orientation, including, but not limited to one cleavage site adjacent to another cleavage site, one cleavage site overlapping another cleavage site, or one cleavage site following another cleavage site with intervening amino acids between the two cleavage sites.

Of particular interest in the present invention are disease specific protease-cleavable linkers. Also preferred are protease-cleavable linkers that are preferentially cleaved at a desired location in the body, such as the tumor microenvironment, relative to the peripheral circulation. For example, the rate at which the protease-cleavable linker is cleaved in the tumor microenvironment can be at least about 10 times, at least about 100 times, at least about 1,000 times or at least about 10,000 times faster in the desired location in the body, e.g., the tumor microenvironment, in comparison to in the peripheral circulation (e.g., in plasma).

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin G, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAPα), dipeptidyl peptidase, meprins, granzymes and dipeptidyl peptidase IV (DPPIV/CD26). Proteases capable of cleaving linker amino acid sequences (which can be encoded by the nucleic acid sequences provided herein) can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2), matrix metalloproteinase 9 (MMP9), matrix metalloproteinase 14 (MMP14). In addition, or alternatively, the linker can be cleaved by a cathepsin, such as, Cathepsin B, Cathepsin C, Cathepsin D, Cathepsin E, Cathepsin G, Cathepsin K and/or Cathepsin L. Preferably, the linker can be cleaved by MMP14 or Cathepsin L.

Proteases useful for cleavage of linkers and for use in the methods disclosed herein are presented in Table 3, and exemplary proteases and their cleavage site are presented in Table 4:

TABLE 3
Proteases relevant to inflammation and cancer

| | | |
|---|---|---|
| Protease | Specificity | Other aspects |

Secreted by killer T cells:

| | | |
|---|---|---|
| Granzyme B (grB) | Cleaves after Asp | Type of serine protease; strongly |
| | residues (asp-ase) | implicated in inducing perforin-dependent |
| | | target cell apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also secreted by |
| | | killer T cells, but not all are present in |
| | | humans |
| Caspase-8 | Cleaves after Asp | Type of cysteine protease; plays essential |
| | residues | role in TCR-induced cellular expansion- |
| | | exact molecular role unclear |
| Mucosa-associated | Cleaves after arginine | Type of cysteine protease; likely acts both |
| lymphoid tissue | residues | as a scaffold and proteolytically active |
| (MALT1) | | enzyme in the CBM-dependent signaling |
| | | pathway |
| Tryptase | Targets: angiotensin I, | Type of mast cell-specific serine protease; |
| | fibrinogen, prourokinase, | trypsin-like; resistant to inhibition by |
| | TGFβ; preferentially | macromolecular protease inhibitors |
| | cleaves proteins after | expressed in mammals due to their |
| | lysine or arginine | tetrameric structure, with all sites facing |
| | residues | narrow central pore; also associated with |
| | | inflammation |

Associated with inflammation:

| | | |
|---|---|---|
| Thrombin | Targets: FGF-2, | Type of serine protease; modulates |
| | HB-EGF, Osteo-pontin, | activity of vascular growth factors, |
| | PDGF, VEGF | chemokines and extracellular proteins; |
| | | strengthens VEGF-induced proliferation; |
| | | induces cell migration; angiogenic factor; |
| | | regulates hemostasis |
| Chymase | Exhibit chymotrypsin- | Type of mast cell-specific serine protease |
| | like specificity, cleaving | |
| | proteins after aromatic | |
| | amino acid residues | |
| Carboxypeptidase A | Cleaves amino acid | Type of zinc-dependent metalloproteinase |
| (MC-CPA) | residues from C-terminal | |
| | end of peptides and | |
| | proteins | |
| Kallikreins | Targets: high molecular | Type of serine protease; modulate |
| | weight | relaxation response; contribute to |
| | kininogen, pro-urokinase | inflammatory response; fibrin degradation |
| Elastase | Targets: E-cadherin, GM- | Type of neutrophil serine protease; |
| | CSF, IL-1, IL-2, IL-6, | degrades ECM components; regulates |
| | IL8, p38^{MAPK}, TNFα, VE- | inflammatory response; activates pro- |
| | cadherin | apoptotic signaling |
| Cathepsin G | Targets: EGF, ENA-78, | Type of serine protease; degrades ECM |
| | IL-8, MCP-1, MMP-2, | components; chemo-attractant of |
| | MT1-MMP, | leukocytes; regulates inflammatory |
| | PAI-1, RANTES, TGFβ, | response; promotes apoptosis |
| | TNFα | |
| PR-3 | Targets: ENA-78, IL-8, | Type of serine protease; promotes |
| | IL-18, JNK, p38^{MAPK}, | inflammatory response; activates pro- |
| | TNFα | apoptotic signaling |
| Granzyme M (grM) | Cleaves after Met and | Type of serine protease; only expressed in |
| | other long, unbranched | NK cells |
| | hydrophobic residues | |
| Calpains | Cleave between Arg and | Family of cysteine proteases; calcium- |
| | Gly | dependent; activation is involved in the |
| | | process of numerous inflammation- |
| | | associated diseases |

TABLE 4
Exemplary Proteases and Protease Recognition Sequences

| Protease | Cleavage Domain Sequence | SEQ ID NO: |
|---|---|---|
| MMP7 | KRALGLPG | 46 |
| MMP7 | (DE); RPLALWRS(DR)₈ | 47 |
| MMP9 | PR(S/T)(L/I)(S/T) | |
| MMP9 | LEATA | 49 |
| MMP11 | GGAANLVRGG | 50 |
| MMP14 | SGRIGFLRTA | 51 |
| MMP | PLGLAG | 52 |
| MMP | PLGLAX | 53 |
| MMP | PLGC(me)AG | 54 |
| MMP | ESPAYYTA | 55 |
| MMP | RLQLKL | 56 |
| MMP | RLQLKAC | 57 |
| MMP2, MMP9, MMP14 | EP(Cit)G(Hof)YL | 58 |
| Urokinase plasminogen activator (uPA) | SGRSA | 59 |
| Urokinase plasminogen activator (uPA) | DAFK | 60 |
| Urokinase plasminogen activator (uPA) | GGGRR | 61 |
| Lysosomal Enzyme | GFLG | 62 |
| Lysosomal Enzyme | ALAL | 63 |
| Lysosomal Enzyme | FK | |
| Cathepsin B | NLL | |
| Cathepsin D | PIC(Et)FF | 66 |
| Cathepsin K | GGPRGLPG | 67 |
| Prostate Specific Antigen | HSSKLQ | 68 |
| Prostate Specific Antigen | HSSKLQL | 69 |
| Prostate Specific Antigen | HSSKLQEDA | 70 |
| Herpes Simplex Virus Protease | LVLASSSFGY | 71 |
| HIV Protease | GVSQNYPIVG | 72 |
| CMV Protease | GVVQASCRLA | 73 |
| Thrombin | F(Pip)RS | |
| Thrombin | DPRSFL | 75 |
| Thrombin | PPRSFL | 76 |
| Caspase-3 | DEVD | 77 |
| Caspase-3 | DEVDP | 78 |
| Caspase-3 | KGSGDVEG | 79 |
| Interleukin 1β converting enzyme | GWEHDG | 80 |
| Enterokinase | EDDDDKA | 81 |
| FAP | KQEQNPGST | 82 |
| Kallikrein 2 | GKAFRR | 83 |
| Plasmin | DAFK | 84 |
| Plasmin | DVLK | 85 |
| Plasmin | DAFK | 86 |
| TOP | ALLLALL | 87 |
| | GPLGVRG | 88 |
| | IPVSLRSG | 89 |
| | VPLSLYSG | 90 |
| | SGESPAYYTA | 91 |

Exemplary protease cleavable linkers include, but are not limited to kallikrein cleavable linkers, thrombin cleavable linkers, chymase cleavable linkers, carboxypeptidase A cleavable linkers, cathepsin cleavable linkers, elastase cleavable linkers, FAP cleavable linkers, ADAM cleavable linkers, PR-3 cleavable linkers, granzyme M cleavable linkers, a calpain cleavable linkers, a matrix metalloproteinase (MMP) cleavable linkers, a plasminogen activator cleavable linkers, a caspase cleavable linkers, a tryptase cleavable linkers, or a tumor cell surface protease. Specifically, MMP9 cleavable linkers, ADAM cleavable linkers, CTSL1 cleavable linkers, FAPa cleavable linkers, and cathepsin cleavable linkers. Some preferred protease-cleavable linkers are cleaved by a MMP and/or a cathepsin.

The protease cleavable linkers disclosed herein are typically less than 100 amino acids. Such protease cleavable linkers can be of different lengths, such as from 1 amino acid (e.g., Gly) to 30 amino acids, from 1 amino acid to 40 amino acids, from 1 amino acid to 50 amino acids, from 1 amino acid to 60 amino acids, from 1 to 70 amino acids, from 1 to 80 amino acids, from 1 to 90 amino acids, and from 1 to 100 amino acids. In some embodiments, the linker is at least about 1, about 2, about 3, about 4, about 5, about 10, about 15, about 20, about 25, about 30, about 35, about 40, about 45, about 50, about 55, about 60, about 65, about 70, about 75, about 80, about 85, about 90, about 95, or about 100 amino acids in length. Preferred linkers are typically from about 5 amino acids to about 30 amino acids.

Preferably the lengths of linkers vary from 2 to 30 amino acids, optimized for each condition so that the linker does not impose any constraints on the conformation or interactions of the linked domains.

In some embodiments, the protease cleavable linker comprises the sequence GPAGLYAQ (SEQ ID NO: 195); GPAGMKGL (SEQ ID NO: 196); PGGPAGIG (SEQ ID NO: 197); ALFKSSFP (SEQ ID NO: 198); ALFFSSPP (SEQ ID NO: 199); LAQRLRSS (SEQ ID NO: 200); LAQKLKSS (SEQ ID NO; 201); GALFKSSFPSGGGPAGLYAQGGSGKGGSGK (SEQ ID NO: 202); RGSGGGPAGLYAQGSGGGPAGLYAQGGSGK (SEQ ID NO: 203); KGGGPAGLYAQGPAGLYAQGPAGLYAQGSR (SEQ ID NO: 204); RGGPAGLYAQGGPAGLYAQGGGPAGLYAQK (SEQ ID NO: 205); KGGALFKSSFPGGPAGIGPLAQKLKSSGGS (SEQ ID NO: 206); SGGPGGPAGIGALFKSSFPLAQKLKSSGGG (SEQ ID NO: 207); RGPLAQKLKSSALFKSSFPGGPAGIGGGGK (SEQ ID NO: 208); GGGALFKSSFPLAQKLKSSPGGPAGIGGGR (SEQ ID NO: 209); RGPGGPAGIGPLAQKLKSSALFKSSFPGGG (SEQ ID NO: 210); RGGPLAQKLKSSPGGPAGIGALFKSSFPGK (SEQ ID NO: 211); RSGGPAGLYAQALFKSSFPLAQKLKSSGGG (SEQ ID NO: 212); GGPLAQKLKSSALFKSSFPGPAGLYAQGGR (SEQ ID NO: 213); GGALFKSSFPGPAGLYAQPLAQKLKSSGGK (SEQ ID NO: 214); RGGALFKSSFPLAQKLKSSGPAGLYAQGGK (SEQ ID NO: 215); RGGGPAGLYAQPLAQKLKSSALFKSSFPGG (SEQ ID NO: 216); SGPLAQKLKSSGPAGLYAQALFKSSFPGSK (SEQ ID NO: 217); KGGPGGPAGIGPLAQRLRSSALFKSSFPGR (SEQ ID NO: 218); KSGPGGPAGIGALFFSSPPLAQKLKSSGGR (SEQ ID NO: 219); or SGGFPRSGGSFNPRTFGSKRKRRGSRGGGG (SEQ ID NO: 220)

Certain preferred protease cleavable linkers comprise the sequence GPAGLYAQ (SEQ ID NO: 195) or ALFKSSFP (SEQ ID NO: 198). The protease cleavable linkers disclosed herein can comprise one or more cleavage motif or functional variants that are the same or different. The protease cleavable linkers can comprise 1, 2, 3, 4, 5, or more cleavage motifs or functional variants. Protease cleavable linkers comprising 30 amino acids can contain 2 cleavage motifs or functional variants, 3 cleavage motifs or functional variants or more. A “functional variant” of a protease cleavable linker retains the ability to be cleaved with high efficiency at a target site (e.g., a tumor microenvironment that expresses high levels of the protease) and are not cleaved or cleaved with low efficiency in the periphery (e.g., serum). For example, the functional variants retain at least about 50%, about 55%, about 60%, about 70%, about 80%, about 85%, about 95% or more of the cleavage efficiency of a protease cleavable linker comprising any one of SEQ ID NOs. 195-220.

The protease cleavable linkers comprising more than one cleavage motif can be selected from SEQ ID NOs: 195-201 and combinations thereof. Preferred protease cleavable linkers comprising more than one cleavage motif comprise the amino acids selected from SEQ ID NO: 202-220.

The protease cleavable linker can comprise both ALFKSSFP (SEQ ID NO: 198) and GPAGLYAQ (SEQ ID NO: 195). The protease cleavable linker can comprise two cleavage motifs that each have the sequence GPAGLYAQ (SEQ ID NO: 195). Alternatively, or additionally, the protease cleavable linker can comprise two cleavage motifs that each have the sequence ALFKSSFP (SEQ ID NO: 198). The protease cleavable linker can comprise a third cleavage motif that is the same or different.

In some embodiments, the protease cleavable linker comprises an amino acid sequence that is at least about 90%, at least about 95%, at least about 96%, at least about 97%, at least about 98%, or at least 99% identical to SEQ ID NOs: 195 to SEQ ID NO: 220 over the full length of SEQ ID NO: 195-220.

The disclosure also relates to functional variants of protease cleavable linkers comprising SEQ ID NOs. 195-220. The functional variants of protease cleavable linkers comprising SEQ ID NOs: 195-220 generally differ from SEQ ID NOs. 195-220 by one or a few amino acids (including substitutions, deletions, insertions, or any combination thereof), and substantially retain their ability to be cleaved by a protease.

The functional variants can contain at least one or more amino acid substitutions, deletions, or insertions relative to the protease cleavable linkers comprising SEQ ID NOs. 195-220. The functional variant can comprise 1, 2, 3, 4, 5, 6, 7, 8, 9, or 10 amino acid alterations comparted to the protease cleavable linkers comprising SEQ ID NOs. 195-220. In some preferred embodiments, the functional variant differs from the protease cleavable linker comprising SEQ ID NOs. 195-220 by less than 10, less, than 8, less than 5, less than 4, less than 3, less than 2, or one amino acid alterations, e.g., amino acid substitutions or deletions. In other embodiments, the functional variant may comprise 1, 2, 3, 4, 5, 6, 7, 8, 9, or 10 amino acid substitutions compared to SEQ ID NOs. 195-220. The amino acid substitution can be a conservative substitution or a non-conservative substitution, but preferably is a conservative substitution.

In other embodiments, the functional variants of the protease cleavable linkers may comprise 1, 2, 3, 4, or 5 or more non-conservative amino acid substitutions compared to the protease cleavable linkers comprising SEQ ID NOs: 195-220. Non-conservative amino acid substitutions could be recognized by one of skill in the art. The functional variant of the protease cleavable linker preferably contains no more than 1, 2, 3, 4, or 5 amino acid deletions.

The amino acid sequences disclosed in the protease cleavable linkers can be described by the relative linear position in the protease cleavable linker with respect to the sissile bond. As will be well-understood by persons skilled in the art, protease cleavable linkers comprising 8 amino acid protease substrates (e.g., SEQ ID Nos: 195-201) contain amino acid at positions P4, P3, P2, P1, P1′, P2′, P3′, P4′, wherein the sissile bond is between P1 and P1′. For example, amino acid positions for the protease cleavable linker comprising the sequence GPAGLYAQ (SEQ ID NO: 195) can be described as follows (SEQ ID NO: 195):

| G | P | A | G | L | Y | A | Q |
|---|---|---|---|---|---|---|---|
| P4 | P3 | P2 | P1 | P1′ | P2′ | P3′ | P4′ |

Amino acids positions for the protease cleavable linker comprising the sequence ALFKSSFP (SEQ ID NO: 198) can be described as follows (SEQ ID NO: 198):

| A | L | F | K | S | S | F | P |
|---|---|---|---|---|---|---|---|
| P4 | P3 | P2 | P1 | P1′ | P2′ | P3′ | P4′ |

Preferably, the amino acids surrounding the cleavage site (e.g., positions P1 and P1′ for SEQ ID NOs: 195-201) are not substituted.

In embodiments, the protease cleavable linker comprises the sequence GPAGLYAQ (SEQ ID NO: 195) or ALFKSSFP (SEQ ID NO: 198) or a functional variant of SEQ ID NO: 195 or a function variant of SEQ ID NO: 198. As described herein, a functional variant of GPAGLYAQ (SEQ ID NO: 195) or ALFKSSFP (SEQ ID NO: 198) can comprise one or more amino acid substitutions, and substantially retain their ability to be cleaved by a protease. Specifically, the functional variants of GPAGLYAQ (SEQ ID NO: 195) is cleaved by MMP14, and the functional variant of ALFKSSFP (SEQ ID NO: 198) is cleaved by Cathepsin L (CTSL1). The functional variants also retain their ability to be cleaved with high efficiency at a target site (e.g., a tumor microenvironment that expresses high levels of the protease). For example, the functional variants of GPAGLYAQ (SEQ ID NO: 195) or ALFKSSFP (SEQ ID NO: 198) retain at least about 50%, about 55%, about 60%, about 70%, about 80%, about 85%, about 95% or more of the cleavage efficiency of a protease cleavable linker comprising amino acid sequence GPAGLYAQ (SEQ ID NO: 195) or ALFKSSFP (SEQ ID NO: 198), respectively.

Preferably, the functional variant of GPAGLYAQ (SEQ ID NO: 195) or ALFKSSFP (SEQ ID NO: 198) comprise no more than 1, 2, 3, 4, or 5 conservative amino acid substitutions compared to GPAGLYAQ (SEQ ID NO: 195) or ALFKSSFP (SEQ ID NO: 198). Preferably, the amino acids at position P1 and P1′ are not substituted. The amino acids at positions P1 and P1′ in SEQ ID NO: 195 are G and L, and the amino acids at positions P1 and P1′ in SEQ ID NO: 198 are K and S.

The functional variant of GPAGLYAQ (SEQ ID NO: 195) can preferably comprise one or more of the following: a) an arginine amino acid substitution at position P4, b) a leucine, valine, asparagine, or proline amino acid substitution at position P3, c) a asparagine amino acid substitution at position P2, d) a histidine, asparagine, or glycine amino acid substitution at position P1, e) a asparagine, isoleucine, or leucine amino acid substitution at position P1′, f) a tyrosine or arginine amino acid substitution at position P2′, g) a glycine, arginine, or alanine amino acid substitution at position P3′, h) or a serine, glutamine, or lysine amino acid substitution at position P4′. The following amino acid substitutions are disfavored in functional variants of GPAGLYAQ (SEQ ID NO: 195): a) arginine or isoleucine at position P3, b) alanine at position P2, c) valine at position P1, d) arginine, glycine, asparagine, or threonine at position P1′, e) aspartic acid or glutamic acid at position P2′, f) isoleucine at position P3′, g) valine at position P4′. In some embodiments, the functional variant of GPAGLYAQ (SEQ ID NO: 195) does not comprise an amino acid substitution at position P1 and/or P1′.

The amino acid substitution of the functional variant of GPAGLYAQ (SEQ ID NO: 195) preferably comprises an amino acid substitution at position P4 and/or P4′. For example, the functional variant of GPAGLYAQ (SEQ ID NO: 195) can comprise a leucine at position P4, or serine, glutamine, lysine, or phenylalanine at position P4. Alternatively, or additionally, the functional variant of GPAGLYAQ (SEQ ID NO: 195) can comprises a glycine, phenylalanine, or a proline at position P4′.

In some embodiments, the amino acid substitutions at position P2 or P2′ of GPAGLYAQ (SEQ ID NO: 195) are not preferred.

In some embodiments, the functional variant of GPAGLYAQ (SEQ ID NO: 195) comprises the amino acid sequence selected from SEQ ID NOs: 258-331. Specific functional variants of GPAGLYAQ (SEQ ID NO: 195) include GPLGLYAQ (SEQ ID NO: 295), and GPAGLKGA (SEQ ID NO: 285).

The functional variants of ALFKSSFP (SEQ ID NO: 198) preferably comprises hydrophobic amino acid substitutions. The functional variant of ALFKSSFP (SEQ ID NO: 198) can preferably comprise one or more of the following: (a) lysine, histidine, serine, glutamine, leucine, proline, or phenylalanine at position P4; (b) lysine, histidine, glycine, proline, asparagine, phenylalanine at position P3; (c) arginine, leucine, alanine, glutamine, or histadine at position P2; (d) phenylalanine, histidine, threonine, alanine, or glutamine at position P1; (e) histidine, leucine, lysine, alanine, isoleucine, arginine, phenylalanine, asparagine, glutamic acid, or glycine at position P1′, (f) phenylalanine, leucine, isoleucine, lysine, alanine, glutamine, or proline at position P2′; (g) phenylalanine, leucine, glycine, serine, valine, histidine, alanine, or asparagine at position P3′; and phenylalanine, histidine, glycine, alanine, serine, valine, glutamine, lysine, or leucine.

The inclusion of aspartic acid and/or glutamic acid in functional variants of SEQ ID NO: 198 are generally disfavored and avoided. The following amino acid substitutions are also disfavored in functional variants of ALFKSSFP (SEQ ID NO: 198): (a) alanine, serine, or glutamic acid at position P3; (b) proline, threonine, glycine, or aspartic acid at position P2; (c) proline at position P1; (d) proline at position P1′; (e) glycine at position P2′; (f) lysine or glutamic acid at position P3′; (g) aspartic acid at position P4′.

The amino acid substitution of the functional variant of ALFKSSFP (SEQ ID NO: 198) preferably comprises an amino acid substitution at position P4 and/or P1. In some embodiments, an amino acid substitution of the functional variant of ALFKSSFP (SEQ ID NO: 198) at position P4′ is not preferred.

In some embodiments, the functional variant of ALFKSSFP (SEQ ID NO: 198) comprises the amino acid sequence selected from SEQ ID NOs: 332-408. Specific functional variants of ALFKSSFP (SEQ ID NO: 198) include ALFFSSPP (SEQ ID NO: 199), ALFKSFPP (SEQ ID NO: 381), ALFKSLPP (SEQ ID NO: 382); ALFKHSPP (SEQ ID NO: 370); ALFKSIPP (SEQ ID NO: 383); ALFKSSLP (SEQ ID NO: 390); or SPFRSSRQ (SEQ ID NO: 333).

The protease cleavable linkers disclosed herein can form a stable complex under physiological conditions with the amino acid sequences (e.g. domains) that they link, while being capable of being cleaved by a protease. For example, the protease cleavable linker is stable (e.g., not cleaved or cleaved with low efficiency) in the circulation and cleaved with higher efficiency at a target site (i.e. a tumor microenvironment). Accordingly, inducible cytokine prodrugs that include the linkers disclosed herein can, if desired, have a prolonged circulation half-life and/or lower biological activity in the circulation in comparison to the components of the inducible cytokine prodrug as separate molecular entities. Yet, when in the desired location (e.g., tumor microenvironment) the linkers can be efficiently cleaved to release the components that are joined together by the linker and restoring or nearly restoring the half-life and biological activity of the components as separate molecular entities.

The protease cleavable linker desirably remains stable in the circulation for at least 2 hours, at least 5, hours, at least 10 hours, at least 15 hours, at least 20 hours, at least 24 hours, at least 30 hours, at least 35 hours, at least 40 hours, at least 45 hours, at least 50 hours, at least 60 hours, at least 65 hours, at least 70 hours, at least 80 hours, at least 90 hours, or longer.

In some embodiments, the protease cleavable linker is cleaved by less than 90%, 80%, 70%, 60%, 50%, 40%, 30%, 20%, 20%, 5%, or 1% in the circulation as compared to the target location. The protease cleavable linker is also stable in the absence of an enzyme capable of cleaving the linker. However, upon expose to a suitable enzyme (i.e., a protease), the protease cleavable linker is cleaved resulting in separation of the linked domain.

The linker sequence can be located between any or all of the cytokine polypeptide, the serum half-life extension element, and/or the blocking element. As described herein, at least one of the linkers is protease cleavable, and contains a (one or more) cleavage site for a (one or more) desired protease. Preferably, the desired protease is enriched or selectively expressed at the desired site of cytokine activity (e.g., the tumor microenvironment). Thus, the inducible cytokine prodrug is preferentially or selectively cleaved at the site of desired cytokine activity.

In some embodiments, the linker comprises glycine-glycine, a sortase-recognition motif, or a sortase-recognition motif and a peptide sequence (Gly₄Ser)ₙ (SEQ ID NO: 238) or (Gly₃Ser)ₙ (SEQ ID NO: 239), wherein n is 1, 2, 3, 4 or 5. In one embodiment, the sortase-recognition motif comprises a peptide sequence LPXTG, where X is any amino acid (SEQ ID NO: 237). In one embodiment, the covalent linkage is between a reactive lysine residue attached to the C-terminal of the cytokine polypeptide and a reactive aspartic acid attached to the N-terminal of the blocking or other moiety. In one embodiment, the covalent linkage is between a reactive aspartic acid residue attached to the N-terminal of the cytokine polypeptide and a reactive lysine residue attached to the C-terminal of the blocking or other moiety. In some embodiments, the blocking element can be attached to the cytokine polypeptide via sortase-mediated conjugation.

### C. Blocking Element

The blocking element can be any element that binds to the cytokine and inhibits the ability of the cytokine polypeptide to bind and activate its receptor. The blocking element can inhibit the ability of the cytokine (e.g. IL-2 or IL-12) to bind and/or activate its receptor e.g., by sterically blocking and/or by noncovalently binding to the cytokine polypeptide. The blocking element disclosed herein can bind to IL-2 or IL-12 (e.g. p35, p40, or the heterodimer).

Examples of suitable blocking elements include the full length or a cytokine-binding fragment or mutein of the cognate receptor of a cytokine (e.g. IL-2 or IL-12). The cognate receptor for IL-2 can be the IL-2 alpha chain, the IL-2 beta chain, the IL-2 gamma chain, or combinations thereof. The cognate receptor for IL-12 can be IL-12RB1 and/or IL-12RB2.

Antibodies and antigen-binding fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind to a cytokine (e.g., IL-2 or IL-12) can also be used. Other suitable antigen-binding domain that bind to the cytokine polypeptide can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocalin and CTLA4 scaffolds. Further examples of suitable blocking polypeptides include polypeptides that sterically inhibit or block binding of the to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides.

Blocking elements that are particularly suitable are single chain variable fragments (scFv) or Fab fragments.

Also disclosed herein is an inducible cytokine prodrug that contains a blocking element having specificity for a cytokine and further contains a half-life extension element.

The blocking element can contain two or more components that are present on the same polypeptide chain or on separate polypeptide chains. A first polypeptide chain can include an antibody light chain (VL+CL) or light chain variable domain (VL) and a second polypeptide can include an antibody heavy chain Fab fragment (VH+CH1) or heavy chain variable domain (VH) that is complementary to the VL+CL or VL on the first polypeptide. In such situations, these components can associate in the peptide complex to form an antigen-binding site, such as a Fab that binds the cytokine (e.g., IL-2 or IL-12) and attenuates cytokine activity.

### D. Half-Life Extension Element

The half-life extension element increases the in vivo half-life and provides altered pharmacodynamics and pharmacokinetics of the inducible cytokine prodrugs. Without being bound by theory, the half-life extension element alters pharmacodynamics properties including alteration of tissue distribution, penetration, and diffusion of the inducible cytokine prodrug. In some embodiments, the half-life extension element can improve tissue targeting, tissue penetration, diffusion within the tissue, and enhanced efficacy as compared with a protein without a half-life extension element. Without being bound by theory, an exemplary way to improve the pharmacokinetics of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments retain FcRn binding and are routinely linked to other polypeptides to extend their serum half-life. HSA may also be directly bound to the pharmaceutical compositions or bound via a short linker. Fragments of HSA may also be used. HSA and fragments thereof can function as both a blocking element and a half-life extension element. Human IgGs and Fc fragments can also carry out a similar function.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

The half-life extension element as provided herein is preferably a human serum albumin (HSA) binding domain, and antigen binding polypeptide that binds human serum albumin or an immunoglobulin Fc or fragment thereof.

The half-life extension element of an inducible cytokine prodrug extends the half-life of the inducible cytokine prodrug by at least about two days, about three days, about four days, about five days, about six days, about seven days, about eight days, about nine days, about 10 days or more.

### F. Adoptive Cell Therapy and Immune Cells

The adoptive cell therapy can comprise any desired immune cells, such as T cells, B cells, NK cells and the like and any combination of immune cells. For example, the cell therapy can be a substantially homogenous population of T cells, such as CAR-T cells. In other examples, the cell therapy can contain one or more cell types, such as primary cells that have been expanded ex-vitro and are administered to the subject in need thereof. A cell therapy for cancer or inflammation (e.g., autoimmune disease) can include, for example, dendritic cells, B cells, T cells (e.g., CD3+ T cells, CD4+ T cells, CD8+ T cells, NKT cells, alpha beta T cells, gamma delta T cells, etc.), NK cells, and/or macrophages.

An adoptive cell therapy can comprise immune cells that are engineered. For example, an immune cell may be engineered to express an antigen binding protein such as a Chimeric Antigen Receptor (CAR) or a T cell receptor (TCR) subunit. An “antigen binding protein” (ABP) is a protein comprising one or more antigen-binding domains that specifically bind to an antigen or epitope. Immune cells such as T cells may be engineered to express a CAR or TCR in order to make the cell specific for an antigen of interest, such as a tumor antigen. Preferably, an antigen binding protein expressed by an immune cell directs the immune cell and its immune effector functions to cells that express a desired antigen. An immune cell may be engineered using any method, such as via viral vectors, CRISPR, TALEN, or meganucleases. Immune cells can be engineered or genetically modified using any suitable approach, in vitro or in vivo. For in vitro engineering, cells are typically cultured and genetically modified using any suitable method, such as viral transduction. Engineered cells can then be selected and if desired expanded and administered to a subject as adoptive cell therapy. For in vivo engineering, typically an engineered genetic construct is administered to the patient to transduce immune cells. A number of suitable approaches can be used such as, for example, viral vectors that infect immune cells and carry a desired transgene, or other suitable nucleic acid delivery technology. Alternatively, an immune cell may not have been engineered. For example, TIL therapy often does not involve engineering the therapeutic cells because they are capable of killing tumor cells. However, TILs may be selected and expanded, for example ex vivo, to produce a population of cells with specificity for a tumor antigen on the subject's tumor.

An adoptive cell therapy can include immune cells that are autologous or allogeneic. An allogeneic immune cell may be engineered to reduce or prevent expression of endogenous proteins that can induce an immune response, such as TCRs or MHC in order to prevent immune reactions against healthy subject cells and/or a host response to the therapeutic immune cells.

In some embodiments, the adoptive cell therapy comprises administering TILs to a subject with a tumor. In some embodiments, an immune cell expresses an antigen binding protein that directs the immune cell and its immune effector functions to cells that express a desired antigen. In some embodiments, an antigen binding protein is a Chimeric Antigen Receptor (CAR). In some embodiments, an antigen binding protein is a T cell Receptor (TCR) or TCR subunit, such as a TCR beta chain or TCR alpha chain. In some embodiments, an antigen binding protein is a T cell receptor inducible cytokine prodrug (TFP). A polynucleotide encoding an antigen binding protein, and optionally regulatory sequences (such as an EF1a promoter) for expression of the antigen binding protein, may be inserted into an immune cell, for example as a nucleic acid that does not integrate into the host genome (e.g., an AAV vector) or that integrates into the host genome, for example, into a genomic locus of interest. In some embodiments, a sequence encoding an antigen binding protein is inserted into the endogenous TRAC gene locus, thereby disrupting expression of the TRAC gene. In some embodiments, an immune cell may further have a disrupted TRAC gene, a disrupted B2M gene, or a combination thereof.

In some embodiments, an antigen binding protein is expressed on an immune cell. In some embodiments, an antigen binding protein is expressed on a T cell, such as an alpha beta T cell, a gamma delta T cell, a CD8+ T cell, or a CD4+ T cell. In some embodiments, an antigen binding protein is expressed on a NK cell, a NKT cell, or a macrophage.

In some embodiments, the antigen binding protein specifically binds to a tumor antigen. In some embodiments, the antigen binding protein specifically binds to one of the following antigens: CD19, CD123, CD22, CD30, CD171, CS-1, CLL-1 (CLECL1), CD33, EGFRVIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

The antigen binding proteins described herein can include components that have the same amino acid sequence of a sequence described herein or can have an amino acid sequence that differs from the sequence described herein so long as the desired function is maintained. It is understood that one way to define any known modifications and derivatives or those that might arise, of the disclosed proteins and nucleic acids that encode them is through defining the sequence variants in terms of identity to specific known reference sequences. Specifically disclosed are polypeptides and nucleic acids which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the sequences provided herein.

i. Immune Cells Expressing Cytokine

If desired, an immune cell can be engineered to express a desired cytokine, or preferably a desired inducible cytokine prodrug. For example, a T cell (e.g., TIL, CAR-T, TCR-T) can be engineered to express an inducible cytokine prodrug, such as an inducible IL-2 prodrug or inducible IL-12 prodrug as described herein. Similarly, other immune cells, such as NK cell can be engineered to express an inducible cytokine, and if desired, an antigen binding protein that directs NK cell activity to cells that express a selected antigen. Preferably, an inducible cytokine prodrug is chosen such that its protease cleavable linker is cleaved by a protease with higher activity in the microenvironment of a tumor in comparison to other locations, and an immune cell capable of killing cells in the same tumor is chosen to express the inducible cytokine prodrug. An immune cell may be engineered to express an inducible cytokine prodrug using any suitable method, such as via one or more viral vectors. An immune cell engineered to express an inducible cytokine prodrug may be autologous or allogeneic with respect to a subject receiving it as a therapy. In the case of an allogeneic cell, it may be advantageous to further engineer the cell such that it lacks functional endogenous TCRs, MHCs, or both. An immune cell may express inducible cytokine prodrugs temporarily (such as if the immune cell is transduced with mRNA encoding the inducible cytokine prodrug) or over the course of its lifespan (such as if the immune cell is transduced with DNA encoding the inducible cytokine prodrug). An immune cell may be engineered to express an inducible cytokine prodrug and an antigen binding protein (such as a CAR) by transducing it with a single polynucleotide encoding both the inducible cytokine prodrug and the antigen binding protein. Alternatively, an immune cell may be engineered to express an inducible cytokine prodrug and an antigen binding protein by transducing it with two or more polynucleotides, for example, one or more polynucleotide encoding the inducible cytokine prodrug and one or more polynucleotide encoding the antigen binding protein.

In some embodiments, an immune cell expresses an inducible cytokine prodrug as described herein. Generally, the inducible cytokine prodrug comprises a cytokine polypeptide, a protease cleavable linker, and a blocking element, as described herein. In some embodiments, a cell contains a single polynucleotide encoding both an antigen binding protein and an inducible cytokine prodrug as described herein. In some embodiments, a cell contains a first polynucleotide encoding an antigen binding protein and a second polynucleotide encoding an inducible cytokine prodrug as described herein. In some embodiments, a cell contains a first polynucleotide encoding an antigen binding protein and a second polynucleotide encoding a inducible cytokine prodrug comprising a cytokine polypeptide, a protease cleavable linker, and a blocking element as described herein. Preferably, an antigen binding protein expressed by an immune cell directs the immune cell and its immune effector functions to cells that express a desired antigen. In some embodiments, a cell contains one or more polynucleotides encoding the inducible cytokine prodrug and one or more polynucleotides encoding the antigen binding protein. The polynucleotides encoding the antigen binding protein and/or the inducible cytokine prodrug are typically engineered for expression in a desired cell and are introduced into the cell using suitable methods, e.g., transduction, transfection. Accordingly, such polynucleotides are not found in the naturally occurring cell type or species, e.g., human. For example, an immune cell can contain at least three or more polynucleotides that collectively encode the antigen binding protein and the inducible cytokine prodrug. The immune cell may contain two or more polynucleotides encoding an inducible cytokine prodrug and at least one polypeptide encoding an antigen binding protein.

The inducible cytokine prodrug can be expressed by the cell as a soluble protein, which is secreted by the cell into the extracellular space. The inducible cytokine prodrug can also be expressed by the cell as a membrane-associated protein, which can optionally be removed from the membrane by proteolytic cleavage. Methods for designing membrane-associated proteins are well-known in the art and include inducible cytokine prodrugs that include a suitable transmembrane or membrane anchoring sequence, and optionally an intracellular domain that is fused to the inducible cytokine prodrug, optionally through a suitable spacer sequence. The spacer sequence can include a cleavage site for a protease that has greater activity in the tumor microenvironment than in other locations. Upon cleavage of such a spacer sequence, the cytokine or inducible cytokine prodrug can be released from the cell membrane. Suitable spacer sequences include the linkers disclosed herein, and amino acid sequences that include cleavage sites disclosed herein. Suitable transmembrane or membrane anchoring sequences are well-known in the art and include the transmembrane segments of a platelet derived growth factor receptor (PDGFR, e.g., PDGFRbeta, PDGFRalpha), CD4, CD8, IL-2R and the like. Preferably, the membrane associate inducible cytokine prodrug lacks a cytoplasmic portion or has a cytoplasmic portion that does not transduce activation signals to the engineered cell. In one example, a CAR-T cell also expresses an inducible cytokine prodrug that can be a secreted molecule or membrane-associated. The engineered cell can be a T cell that expresses an inducible cytokine prodrug. Preferably the engineered T cell is a TIL or a CAR-T cell. The inducible cytokine prodrug can be soluble or membrane tethered. An inducible cytokine prodrug that is membrane tethered can comprise a cytokine polypeptide, a first protease cleavable linker, and a blocking element as described herein. The inducible cytokine prodrug can contain a suitable transmembrane domain or membrane anchoring sequence. Typically, the suitable transmembrane domain or membrane anchoring sequence is linked to the cytokine peptide, directly or indirectly, through a protease cleavable linker and can be released from the membrane upon cleavage of such protease cleavable linker. For example, a membrane tethered inducible cytokine prodrug can comprise from amino to carboxy terminus: the cytokine polypeptide—a protease cleavable linker—a blocking element (e.g., a scFv, a VH and CH1, or VL and CL)—a cleavable linker that is preferably protease cleavable—a suitable transmembrane or membrane anchoring sequence. As another example, a membrane tethered inducible cytokine prodrug can comprise from amino to carboxy terminus: a blocking element (e.g., e.g., a scFv, a VH and CH1, or VL and CL)—a protease cleavable linker—the cytokine polypeptide—a cleavable linker that is preferably protease cleavable—a suitable transmembrane or membrane anchoring sequence. In some preferred embodiments, the inducible cytokine prodrug (e.g., inducible IL-2 prodrug, inducible IL-12 prodrug) is membrane tethered and comprises the transmembrane domain of PDGFR, CD4, CD8 or IL-2R.

The inducible cytokine prodrug can be an inducible IL-2 prodrug. The inducible cytokine prodrug can be an inducible IL-12 prodrug.

The engineered T cell can express an inducible IL-2 prodrug that comprises a) a first polypeptide chain comprising the amino acid selected from SEQ ID NOs. 1-4 and b) a second polypeptide chain comprising the amino acid sequence of SEQ ID NO: 5. The engineered T cell expresses an inducible IL-2 prodrug, as disclosed herein. The inducible IL-2 prodrug comprises a) first polypeptide chain comprising the amino acid selected from any one of SEQ ID NOs: 1-4 and b) a second polypeptide comprising the amino acid sequence of: SEQ ID NO: 5, or a membrane associated for of any of the foregoing in which the half-life extension element is replaced with a suitable transmembrane domain. The engineered T cell can express an inducible IL-2 prodrug that comprises a) a first polypeptide chain that comprises or consists of the amino acid sequence of SEQ ID NO: 1, and b) a second polypeptide chain that comprises or consists of the amino acid sequence of SEQ ID NO: 5. The engineered T cell can express an inducible IL-2 prodrug that comprises a) a first polypeptide chain that comprises or consists of the amino acid sequence of SEQ ID NO: 2, and b) a second polypeptide chain that comprises or consists of the amino acid sequence of SEQ ID NO: 5. The engineered T cell can express an inducible IL-2 prodrug that comprises a) a first polypeptide chain that comprises or consists of the amino acid sequence of SEQ ID NO: 3, and b) a second polypeptide chain that comprises or consists of the amino acid sequence of SEQ ID NO: 5. The engineered T cell can express an inducible IL-2 prodrug that comprises a) a first polypeptide chain that comprises or consists of the amino acid sequence of SEQ ID NO: 4, and b) a second polypeptide chain that comprises or consists of the amino acid sequence of SEQ ID NO: 5.

The engineered T cell can express a membrane bound inducible IL-12 prodrug that comprises a) a first polypeptide chain comprising the amino acid selected from SEQ ID NOs: 6-11 or 45 and b) a second polypeptide chain comprising the amino acid sequence selected from SEQ ID NOs: 12 or 13. The engineered T cell can express a membrane bound inducible IL-12 prodrug that comprises a) a first polypeptide chain that comprises or consists of the amino acid sequence of SEQ ID NO: 6, and b) a second polypeptide chain that comprises or consists of the amino acid sequence of SEQ ID NO: 12. The engineered T cell can express a membrane bound inducible IL-12 prodrug that comprises a) a first polypeptide chain that comprises or consists of the amino acid sequence of SEQ ID NO: 7, and b) a second polypeptide chain that comprises or consists of the amino acid sequence of SEQ ID NO: 12. The engineered T cell can express a membrane bound inducible IL-12 prodrug that comprises a) a first polypeptide chain that comprises or consists of the amino acid sequence of SEQ ID NO: 8, and b) a second polypeptide chain that comprises or consists of the amino acid sequence of SEQ ID NO: 13. The engineered T cell can express a membrane bound inducible IL-12 prodrug that comprises a) a first polypeptide chain that comprises or consists of the amino acid sequence of SEQ ID NO: 9, and b) a second polypeptide chain that comprises or consists of the amino acid sequence of SEQ ID NO: 13. The engineered T cell can express a membrane bound inducible IL-12 prodrug that comprises a) a first polypeptide chain that comprises or consists of the amino acid sequence of SEQ ID NO: 10, and b) a second polypeptide chain that comprises or consists of the amino acid sequence of SEQ ID NO: 13. The engineered T cell can express a membrane bound inducible IL-12 prodrug that comprises a) a first polypeptide chain that comprises or consists of the amino acid sequence of SEQ ID NO: 11, and b) a second polypeptide chain that comprises or consists of the amino acid sequence of SEQ ID NO: 13. The engineered T cell can express a membrane bound inducible IL-12 prodrug that comprises a) a first polypeptide chain that comprises or consists of the amino acid sequence of SEQ ID NO: 45, and b) a second polypeptide chain that comprises or consists of the amino acid sequence of SEQ ID NO: 12.

The engineered T cell can express an inducible IL-2 prodrug that comprises a polypeptide chain comprising the amino acid selected from SEQ ID NO. 41. The engineered T cell can express an inducible IL-2 prodrug that expresses 1) an inducible IL-2 prodrug that comprises a) a first polypeptide chain comprising the amino acid selected from SEQ ID NOs. 1-4 and b) a second polypeptide chain comprising the amino acid sequence selected from SEQ ID NOs: 5, and 2) a binding domain comprising the amino acid sequence selected from SEQ ID NO: 562-648. The engineered T cell can express an inducible IL-2 prodrug that comprises a polypeptide chain comprising SEQ ID NO: 41 and a binding domain that comprises the amino acid sequence selected from SEQ ID NOs: 562-577.

The engineered T cell can express an inducible IL-12 prodrug that comprises a) a first polypeptide chain comprising the amino acid sequence selected from SEQ ID NOs.6-11 or 45, and b) a second polypeptide sequence comprises, for example, an amino acid sequence of SEQ ID NO: 12 or 13. The engineered T cell can express an inducible IL-12 prodrug that can comprise comprises a) a first polypeptide that comprises or consists the amino acid sequence of SEQ ID NO: 6, and b) a second polypeptide can comprise or consist of the amino acid sequence of SEQ ID NO: 12. The engineered T cell can express an inducible IL-12 prodrug that comprises a) a first polypeptide chain that comprises or consists of the amino acid sequence of SEQ ID NO: 7, and b) a second polypeptide that comprises or consists of the amino acid sequence of SEQ ID NO: 12. The engineered T cell can express an inducible IL-12 prodrug can comprise a) a first polypeptide chain that comprises or consists of the amino acid sequence of SEQ ID NO: 8, and b) a second polypeptide that comprises or consists of the amino acid sequence of SEQ ID NO: 13. The engineered T cell can express an inducible IL-12 prodrug that comprises a) a first polypeptide chain of the amino acid sequence of SEQ ID NO: 9, and b) a second polypeptide that comprises or consists of the amino acid sequence of SEQ ID NO: 13. The engineered T cell can express an inducible IL-12 prodrug that comprises a) a first polypeptide chain of the amino acid sequence of SEQ ID NO: 10, and b) a second polypeptide that comprises or consists of the amino acid sequence of SEQ ID NO: 13. The engineered T cell can express an inducible IL-12 prodrug that comprises a) a first polypeptide chain of the amino acid sequence of SEQ ID NO: 11, and b) a second polypeptide that comprises or consists of the amino acid sequence of SEQ ID NO: 13. The engineered T cell can express an inducible IL-12 prodrug that comprises a) a first polypeptide chain of the amino acid sequence of SEQ ID NO: 45, and b) a second polypeptide that comprises or consists of the amino acid sequence of SEQ ID NO: 13.

The engineered T cell can express a membrane associated inducible IL-12 prodrug that comprises a) a first polypeptide chain comprising the amino acid sequence selected from SEQ ID NOs. 6-11, and b) a second polypeptide sequence comprises, for example, an amino acid sequence of SEQ ID NO: 12 or 13. The engineered T cell can express a membrane associated inducible IL-12 prodrug that can comprise a) a first polypeptide that comprises or consists the amino acid sequence of SEQ ID NO: 6, and b) a second polypeptide can comprise or consists of the amino acid sequence of SEQ ID NO: 12. The engineered T cell can express a membrane associated inducible IL-12 prodrug that comprises a) a first polypeptide chain that comprises or consists of the amino acid sequence of SEQ ID NO: 7, and b) a second polypeptide that comprises or consists of the amino acid sequence of SEQ ID NO: 12. The engineered T cell can express a membrane associated inducible IL-12 prodrug can comprise a) a first polypeptide chain that comprises or consists of the amino acid sequence of SEQ ID NO: 8, and b) a second polypeptide that comprises or consists of the amino acid sequence of SEQ ID NO: 13. The engineered T cell can express a membrane associated inducible IL-12 prodrug that comprises a) a first polypeptide chain of the amino acid sequence of SEQ ID NO: 9, and b) a second polypeptide that comprises or consists of the amino acid sequence of SEQ ID NO: 13. The engineered T cell can express a membrane associated inducible IL-12 prodrug that comprises a) a first polypeptide chain of the amino acid sequence of SEQ ID NO: 10, and b) a second polypeptide that comprises or consists of the amino acid sequence of SEQ ID NO: 13. The engineered T cell can express a membrane associated inducible IL-12 prodrug that comprises a) a first polypeptide chain of the amino acid sequence of SEQ ID NO: 11, and b) a second polypeptide that comprises or consists of the amino acid sequence of SEQ ID NO: 13.

i. Chimeric Antigen Receptors

Chimeric antigen receptors (CARs) are genetically engineered receptors. Immune cells, such as T cells, can be engineered to express these receptors in order to enhance their ability to target an antigen, such as a tumor antigen. In some embodiments, a CAR comprises an antigen binding domain that specifically binds to a target antigen, a hinge domain, a transmembrane domain, a costimulatory domain, and a primary signaling domain. In some embodiments, a cell contains multiple CARs targeting different antigens, such as CD19 and CD20. In some embodiments, a CAR contains a sequence listed in Table 15. In some embodiments, a CAR contains a sequence selected from SEQ ID NO: 661-665. See generally international patent applications WO2016014789 and WO2014153270, which are incorporated herein by reference in their entireties.

### Antigen Binding Domain

A CAR may be engineered to bind to a target antigen (such as a cell surface antigen) by incorporating an antigen binding domain specific for the target antigen. In some embodiments, the antigen binding domain is an antibody or a fragment thereof. In some embodiments, an antigen binding domain is a single chain antibody fragment (scFv) comprising an antibody fragment comprising the variable region of a light chain and an antibody fragment comprising the variable region of a heavy chain that are linked and expressed as a single chain polypeptide, and retain the specificity of the antibody from which they are derived. Typically, the heavy chain and light chain are connected by a short polypeptide linker. Unless specified otherwise, the VH and VL may be in either order. In some embodiments, an antigen binding domain is specific for a tumor antigen described herein. In some embodiments, an antigen binding domain contains a sequence listed in Table 12. In some embodiments, an antigen binding domain contains a sequence selected from SEQ ID NO: 562-648.

### Hinge and Transmembrane Domains

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof. In some embodiments, a hinge domain contains a sequence listed in Table 13. In some embodiments, a hinge domain contains a sequence selected from SEQ ID NO: 649-651.

As used herein, a “transmembrane domain” refers to any protein structure that is thermodynamically stable in a cell membrane, preferably a eukaryotic cell membrane. In some embodiments, a transmembrane domain is a hydrophobic alpha helix that spans the membrane. In some embodiments, the transmembrane domain is a CD8 or CD28 transmembrane domain. In some embodiments, a transmembrane domain contains a sequence listed in Table 13. In some embodiments, a transmembrane domain contains a sequence selected from SEQ ID NO: 649-653.

### Costimulatory Domain

In some embodiments, a CAR comprises at least one intracellular costimulatory domain selected from the group CD28, 4-1BB, ICOS, CD27, and OX40. In some embodiments, a costimulatory domain contains a sequence listed in Table 14. In some embodiments, a costimulatory domain contains a sequence selected from SEQ ID NO: 654-658.

### Primary Signaling Domain

CARs typically contain the intracellular signaling domain of CD3zeta. CD3zeta is the cytoplasmic signaling domain of the T cell receptor complex. CD3z contains 3 immunoreceptor tyrosine-based activation motif (ITAM)s, which transmit an activation signal to the T cell after the T cell is engaged with a cognate antigen. In many cases, CD3zeta provides a primary T cell activation signal but not a fully competent activation signal, which requires a co-stimulatory signaling. In some embodiments, a primary signaling domain contains a sequence listed in Table 14. In some embodiments, a primary signaling domain contains a sequence selected from SEQ ID NO: 659-660.

### T Cell Receptors

A TCR is composed of two different and separate protein chains, namely the TCR alpha (a) and the TCR beta (b) chain. The TCR a chain comprises variable (V), joining (J) and constant (C) regions. The TCR b chain comprises variable (V), diversity (D), joining (J) and constant (C) regions. The rearranged V (D) J regions of both the TCR a and the TCR b chain contain hypervariable regions (CDR, complementarity determining regions), among which the CDR3 region determines the specific epitope recognition. At the C-terminal region both the TCR α chain and TCR b chain contain a hydrophobic transmembrane domain and end in a short cytoplasmic tail. Typically, the TCR is a heterodimer of one a chain and one b chain. This heterodimer can bind to MHC molecules presenting a peptide.

In some embodiments, an antigen binding protein is a TCR subunit, such as an alpha, beta, gamma, or delta chain. In some embodiments, an immune cell is engineered to express a single TCR subunit that is incorporated into endogenous TCRs. In some embodiments, an immune cell is engineered to express multiple TCR subunits. In some embodiments, a TCR contains a sequence listed in Table 16. In some embodiments, a TCR contains a sequence selected from SEQ ID NO: 666-702. See generally international patent application WO2019118508, which is incorporated herein by reference in its entirety. In some embodiments, the TCR can bind target antigen and is not MHC restricted.

### T Cell Receptor Inducible Cytokine Prodrugs

As used herein, a “T cell receptor (TCR) inducible cytokine prodrug” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. A “TFP T cell” is a T cell that has been transduced (e.g., according to the methods disclosed herein) and that expresses a TFP, e.g., incorporated into the natural TCR. In some embodiments, a TFP comprises an antigen binding domain linked to a TCR subunit or a portion of a TCR subunit. In some embodiments, a TCR subunit is TCR alpha, TCR beta, CD3 gamma, CD3 epsilon, or CD3 delta. In some embodiments, the antigen binding domain is specific for a tumor antigen described herein. In some embodiments, a TFP contains a sequence listed in Table 17. In some embodiments, a TFP contains a sequence selected from SEQ ID NO: 703-705. See generally international patent application WO2020198033, which is incorporated herein by reference in its entirety. In some embodiments, the TFP can bind target antigen and is not MHC restricted.

### Chimeric Autoantibody Receptors

A Chimeric Autoantibody Receptor (CAAR) is an engineered receptor containing an antigen or fragment thereof that is specific for an autoantibody. CAARs are typically expressed on T cells, for example Treg cells, and these cells are used to treat autoimmune diseases such as pemphigus vulgaris (PV). See generally international patent applications WO2019236593, WO2020231999, and WO2015168613, each of which is incorporated herein by reference in its entirety.

### Tumor Infiltrating Lymphocytes

The terms “tumor infiltrating lymphocytes” or “TILs” refer to a population of cells originally obtained as white blood cells that have left the bloodstream of a subject and migrated into a tumor. TILs typically include CD8+ and CD4+ T cells (e.g., CD8+ cytotoxic T cells, CD4+ Th 1 T cells and/or CD4+Th17 T cells). TIL cell preparations can also include other cell types, such as natural killer cells, dendritic cells and M1 macrophages. TILs include both primary and secondary TILs. “Primary TILs” are those that are obtained from subject tissue samples, and “secondary TILs” are any TIL cell populations that have been expanded or proliferated as discussed herein, including, but not limited to bulk TILs and expanded TILs. TIL cell populations can include genetically modified TILs.

In some embodiments, a TIL has not been genetically engineered. In some embodiments, a TIL has been modified to express an antigen binding protein or an inducible cytokine prodrug. In some embodiments, a subject in need thereof is treated with autologous TILs.

TILs and production of TILs are described, for example, in international patent application PCT/US2018/064135 and/or U.S. patent application Ser. No. 17/041,305, each of which are incorporated by reference in their entirety. In some embodiments, TILs are selected based on their antigen specificity.

### G. Method of Treatment

Described herein are methods of treating a subject in need thereof with an effective amount of an inducible cytokine prodrug, mutein, subunit, or functional fragment thereof, and an effective amount of an adoptive cell therapy. The adoptive cell therapy preferably involves administering immune cells to the subject that have been engineered to provide desired immune function, or that have not been engineered (e.g., TILs). If desired, the adoptive cell therapy can involve administration of a suitable polynucleotide to engineer cells in vivo. A polynucleotide encoding an antigen binding protein, such as a CAR, may be administered to a subject in any suitable form such as a viral vector. A polynucleotide encoding an antigen binding protein, such as a CAR, may be encapsulated in, for example, a viral particle (e.g., AAV capsid, lentiviral envelope) or a nanoparticle such as a lipid nanoparticle. The antigen binding protein, such as a CAR, may be specific for a tumor antigen. An antigen binding protein expressed by an immune cell may direct the immune cell and its immune effector functions to cells that express a desired antigen.

A subject in need thereof may receive two or more inducible cytokine prodrugs, muteins, subunits, or functional fragments thereof, e.g., a combination of inducible IL-2 and IL-12 prodrugs.

The compositions and methods described herein may be used to treat, for example, a proliferative disease or an autoimmune disease. A proliferative disease may be, for example, a cancer or malignancy or a precancerous condition such as a myelodysplasia, a myelodysplastic syndrome or a preleukemia. In some embodiments, the proliferative disease is cancer. In some embodiments, the cancer is selected from the group consisting of one or more acute leukemias including but not limited to B-cell acute lymphoid leukemia, T cell acute lymphoid leukemia, acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia. In some embodiments, the proliferative disorder is a solid tumor cancer, such as adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. The methods are preferably useful for colon cancer, lung cancer, melanoma, renal cell carcinoma, or breast cancer. The cancer can be melanoma, non-small cell lung cancer (NSCLC), small cell lung cancer (SCLC), head and neck squamous cell cancer (HNSCC), classical Hodgkin lymphoma (cHL), primary mediastinal large B cell lymphoma (PMBCL), urothelial carcinoma, microsatellite instability high or mismatch repair deficient cancer, microsatellite instability high or mismatch repair deficient colorectal cancer, gastric cancer, esophageal cancer, cervical cancer, hepatocellular carcinoma (HCC), merkel cell carcinoma (MCC), renal cell carcinoma (RCC), endometrial carcinoma, tumor mutational burden high cancer, cutaneous squamous cell carcinoma (cSCC), triple negative breast cancer (TNBC), urothelial carcinoma, colorectal cancer or oesophageal carcinoma. The cancer can be metastatic, for example, metastatic renal clear cell carcinoma or metastatic cutaneous malignant melanoma. In some embodiments, the solid tumor cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), renal cancer, and renal cell carcinoma. In some embodiments, the proliferative disorder is a hematological malignancy. In some embodiments, the cancer is selected from the group consisting of chronic lymphocytic leukemia, acute lymphoblastic leukemia, diffuse large B cell lymphoma, non-Hodgkin's lymphoma, Hodgkin's lymphoma, follicular lymphoma, and mantle cell lymphoma. In a preferred embodiment, a proliferative disorder is a solid tumor cancer. An autoimmune disease may be, for example, graft versus host disease, multiple sclerosis, rheumatoid arthritis, myasthenia gravis, Crohn's disease, or lupus. The compositions and methods described herein may also be used to prevent graft rejection.

Further provided are methods of treating a subject with or at risk of developing an of a disease or disorder, such as cancer. The methods comprise administering to a subject in need thereof (a) an effective amount of an inducible cytokine prodrug, and (b) an effective amount of an adoptive cell therapy. In some embodiments, the method further comprises selecting a subject with or at risk of developing a disease or disorder. Preferably, the inducible cytokine prodrug comprises a cytokine polypeptide or a fragment or mutein thereof and a serum half-life extension element. In another embodiment, the inducible cytokine prodrug includes a cytokine polypeptide or a fragment or mutein thereof and a blocking element, e.g. a steric blocking polypeptide, wherein the steric blocking polypeptide is capable of sterically blocking the activity of the cytokine polypeptide, fragment or mutein thereof. In another embodiment, the inducible cytokine prodrug comprises a cytokine polypeptide or a fragment or mutein thereof, a blocking element, and a serum half-life extension element.

An effective amount of an inducible cytokine prodrug, mutein, subunit, or functional fragment thereof, and an effective amount of an adoptive cell therapy, e.g. an immune cell or a polynucleotide encoding an antigen binding protein are administered to a subject in need thereof may be administered to a subject simultaneously. More than one inducible cytokine prodrug (e.g., inducible IL-2 in combination with inducible IL-12 prodrug) can be administered to a subject prior to, following, or with (e.g., concurrently) an adoptive cell therapy (e.g., TILs containing therapy administration). An inducible cytokine prodrug, mutein, subunit, or functional fragment thereof may be administered to a subject before, after, concurrently with or periprocedurally with the adoptive cell therapy (e.g., an immune cell or a polynucleotide encoding an antigen binding protein). In embodiments, the inducible cytokine is administered after the adoptive cell therapy. In some embodiments, an inducible cytokine prodrug, mutein, subunit, or functional fragment thereof is administered to a subject when the detectable therapeutic cell count (such as the count of administered immune cells in the peripheral circulation) has decreased by 10%, 20%, 30%, 40%, 50%, 60%, 70%, 80%, or 90%. In some embodiments, an inducible cytokine prodrug, mutein, subunit, or functional fragment thereof is administered to a subject when therapeutic cells are no longer detectable in the peripheral circulation. In some embodiments, an inducible cytokine prodrug, mutein, subunit, or functional fragment thereof is administered to a subject about 1 day, 2 days, 3 days, 4 days, 5 days, 6 days, 7 days, 2 weeks, 3 weeks, 4 weeks, 5 weeks, or 6 weeks after adoptive cell therapy (e.g., an immune cell or a polynucleotide encoding an antigen binding protein) is administered. An inducible cytokine prodrug, mutein, subunit, or functional fragment thereof may be administered before adoptive cell therapy. In some embodiments, an inducible cytokine prodrug, mutein, subunit, or functional fragment thereof is administered to a subject about 1 day, 2 days, 3 days, 4 days, 5 days, 6 days, 7 days, 2 weeks, 3 weeks, 4 weeks, 5 weeks, or 6 weeks before adoptive cell therapy (e.g., an immune cell or a polynucleotide encoding an antigen binding protein) is administered.

A subject in need thereof may be administered multiple doses of adoptive cell therapy (e.g., an immune cell or a polynucleotide encoding an antigen binding protein). For example, a subject in need thereof may receive 1, 2, 3, 4, or more doses of adoptive cell therapy. A subject in need thereof may be administered multiple doses of an inducible cytokine prodrug, mutein, subunit, or functional fragment thereof. For example, a subject in need thereof may receive 1, 2, 3, 4, or more doses of an inducible cytokine prodrug, mutein, subunit, or functional fragment thereof. So long as the inducible cytokine prodrug and adoptive cell therapy have an overlap in their pharmacological or biological activities, the doses can be administered in any order. For example, a subject in need thereof may receive a dose of an adoptive cell therapy, then multiple periodic doses of an inducible cytokine prodrug.

The method can further involve the administration of one or more additional agents to treat cancer, such as chemotherapeutic agents (e.g., cyclophosphamide, mechlorethamine, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mitomycin, diaziquone (AZQ), cisplatin, carboplatin, oxaliplatin, procarbazine, hexamethylmelamine, methotrexate, pemetrexed, fluorouracil (e.g. 5-fluorouracil), capecitabine, cytarabine, gemcitabine, decitabine, azacitidine, fludarabine, nelarabine, cladribine, clofarabine, pentostatin, thioguanine, mercaptopurine, vincristine, vinblastine, vinorelbine, vindesine, vinflunine, paclitaxel, docetaxel, etoposide, teniposide, doxorubicin, daunorubicin, epirubicin, idarubicin, pirarubicin, aclarubicin, mitoxantrone, actinomycin, bleomycin, bisantrene, gemcitabine, cytarabine, and the like), immuno-oncology agents (e.g., anti-PD-L1, anti-CTLA4, anti-PD-1, anti-CD47, anti-GD2), oncolytic viruses and the like. In certain embodiments, the adoptive cell therapy and inducible cytokine prodrugs are administered to a subject in need thereof in combination with an immune checkpoint inhibitor. Immune checkpoint proteins include, for example, PD-1 which binds ligands PD-L1 (B7-H1, CD274) and PD-L2 (B7-DC, CD273), CTLA-4 (CD152) which binds B7-1 (CD80) and B7-2 (CD86), LAG 3 (CD223) which binds Galectin3, LSECtin and FGL1; TIM3 (HAVCR2) which binds ligands Ceacam 1 and Galectin9; TIGIT (VSTM3, WUCAM) which binds CD112 and CD155; BTLA (CD272) which binds HVEM (TNFRSF14), B7-H3 (CD276), B7-H4 (VTCN1), VISTA (B7-H5), KIR, CD44 (2B4), CD160 (BY55) which bind HVEM; CD134 (TNRFSR4, OX40) which binds CD252 (OX-40L). Therapeutic agents, such as antibodies, that bind immune checkpoint proteins and inhibit their immunosuppressive activity include the anti-PD1 antibodies pembrolizumab (KEYTRUDA), dostarlimab (JEMPERLI), cemiplimab-rwlc (LIBATYO), nivolumab (OPDIVO), camrelizumab, tislelizumab, toripalimab, and sintilimab (TYVYT); the anti-PD-L1 antibodies avelumab (BAVENCIO), durvalumab (IMFINZI), and atezolizumab (TECENTRIQ); the anti-CTLA-4 antibody ipilimumab (YERVOY).

A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder.

The methods and agents as described herein are useful for both prophylactic and therapeutic treatment. For prophylactic use, an effective amount of the inducible cytokine prodrugs or polynucleotides encoding the inducible cytokine prodrugs as well as the immune cells described herein are administered to a subject prior to onset (e.g., before obvious signs of cancer or inflammation) or during early onset (e.g., upon initial signs and symptoms of cancer or inflammation). Prophylactic administration can occur for several days to years prior to the manifestation of symptoms of cancer or inflammation. Prophylactic administration can be used, for example, in the preventative treatment of subjects diagnosed with a genetic predisposition to cancer. Therapeutic treatment involves administering to a subject a effective amount of the inducible cytokine prodrug s or polynucleotides encoding the inducible cytokine prodrugs and the immune cells after diagnosis or development of cancer or inflammation (e.g., an autoimmune disease). Prophylactic use may also apply when a subject is undergoing a treatment, e.g., a chemotherapy, in which inflammation is expected.

### H. Pharmaceutical Compositions

Described herein are compositions comprising inducible cytokine prodrugs, polynucleotides encoding inducible cytokine prodrugs, or cells expressing inducible cytokine prodrugs, as well as compositions for adoptive cell therapy.

In embodiments, the disclosure relates to a kit comprising 1) a pharmaceutical composition that comprises an inducible cytokine prodrugs, and 2) a pharmaceutical composition for adoptive cell therapy. The kit can include ancillary reagents, such as water for injection, if desired. The compositions can be combined for concurrent administration or can be administered in any desired sequence as further described herein.

Compositions useful for the delivery of polynucleotides encoding inducible cytokine prodrugs to cells are provided. Compositions useful for the delivery of comprising inducible cytokine prodrugs, polynucleotides encoding inducible cytokine prodrugs, or cells expressing inducible cytokine prodrugs to a subject are also provided.

A therapeutic cell may express an inducible cytokine prodrug. The inducible cytokine prodrug can be expressed as a soluble protein or as a membrane associated protein, which can optionally be releasable from the membrane by proteolytic cleavage, as described herein. For example, a TIL may be engineered to express inducible IL-2 prodrug and/or IL-12, an NK cell may be engineered to express inducible IL-15, or a CAR-T cell may be engineered to express inducible IL-2 prodrug and/or inducible IL-12 prodrug. A polynucleotide (i.e. one or more polynucleotides) may encode an inducible cytokine prodrug and an antigen binding protein, such as a CAR. Provided herein are compositions comprising multiple polynucleotides, wherein a one or more polynucleotide encodes an inducible cytokine prodrug and another polynucleotide encodes an antigen binding protein. Provided herein are viral vectors and nucleic acids (e.g., recombinant viral genomes) encoding an antigen binding protein and an inducible cytokine prodrug. Provided herein are compositions comprising multiple viral vectors, wherein one or more vectors encode an inducible cytokine prodrug and another vector encodes an antigen binding protein. Provided herein are lipid nanoparticle (LNP) compositions comprising polynucleotides encoding an antigen binding protein and an inducible cytokine prodrug. Provided herein are LNP compositions comprising multiple polynucleotides, wherein one or more polynucleotides encode an inducible cytokine prodrug and another polynucleotide encodes an antigen binding protein. A polynucleotide encoding an antigen binding protein and/or an inducible cytokine prodrug may be RNA or DNA. For example, a polynucleotide encoding an antigen binding protein and/or an inducible cytokine prodrug may be circular RNA. A polynucleotide encoding an antigen binding protein and/or an inducible cytokine prodrug may contain modified nucleosides, or, alternatively, may consist only of natural nucleosides. A polynucleotide encoding an inducible cytokine prodrug may comprise elements preventing its expression in tissues where expression is not desired. For example, promoters may be selected, or miRNA sites may be incorporated in order to prevent expression in a particular tissue. Preferably, an antigen binding protein expressed by an immune cell directs the immune cell and its immune effector functions to cells that express a desired antigen.

Further provided herein are pharmaceutical formulations or compositions containing the inducible cytokine prodrug and/or adoptive cell therapy and a pharmaceutically acceptable carrier. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical formulation or composition in which it is contained. The carrier is selected to minimize degradation of the active ingredient and to minimize adverse side effects in the subject. In some embodiments, a inducible cytokine prodrug, mutein, subunit, or functional fragment thereof is formulated together with an immune cell.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically acceptable salt is used in the formulation to render the formulation isotonic, although the formulate can be hypertonic or hypotonic if desired. Examples of the pharmaceutically acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the inducible cytokine prodrugs or nucleic acid sequences encoding the inducible cytokine prodrugs to humans or other subjects.

The pharmaceutical formulations or compositions are administered in a number of ways depending on whether local or systemic treatment is desired, and on the area to be treated. The compositions are administered via any of several routes of administration, including topically, orally, parenterally (e.g., intravenously, intra-articularly, intraperitoneally, intramuscularly, subcutaneously, intracavity, transdermally, intrahepatically, intracranially), nebulization/inhalation, or by installation via bronchoscopy. Parenteral administration is generally preferred. In some embodiments, the compositions are administered locally (non-systemically), including intratumorally, intra-articularly, intrathecally, etc. In some embodiments, a pharmaceutical composition comprises an inducible cytokine prodrug, mutein, subunit, or functional fragment thereof and an engineered immune cell. In some embodiments, an inducible cytokine prodrug, mutein, subunit, or functional fragment thereof is administered with an engineered immune cell.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, antioxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, an inducible cytokine prodrug or antigen binding protein or nucleic acid sequences encoding them are expressed using a suitable vector. There are a number of compositions and methods which can be used to deliver the nucleic acid molecules and/or polypeptides to cells, either in vitro or in vivo via, for example, viral vectors and recombinant expression vectors. These methods and compositions can largely be broken down into two classes: viral based delivery systems and non-viral based delivery systems. Such methods are well known in the art and readily adaptable for use with the compositions and methods described herein. Such compositions and methods can be used to transfect or transduce cells in vitro or in vivo, for example, to produce cell lines that express and preferably secrete the encoded inducible cytokine prodrug or to therapeutically deliver nucleic acids to a subject. The components of the polynucleotides encoding a inducible cytokine prodrug disclosed herein typically are operably linked in frame to encode a inducible cytokine prodrug.

Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Such vectors can also be used to make the inducible cytokine prodrugs by expression is a suitable host cell, such as CHO cells.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

The inducible cytokine prodrugs and nucleic acids encoding the inducible cytokine prodrugs described herein can be made using any suitable method. For example, nucleic acids encoding a inducible cytokine prodrug can be made using recombinant DNA techniques, synthetic chemistry or combinations of these techniques, and expressed in a suitable expression system, such as in CHO cells. Inducible cytokine prodrugs can similarly be made, for example by expression of a suitable nucleic acid, using synthetic or semi-synthetic chemical techniques, and the like.

To form the cytokine-blocking element conjugate, the cytokine polypeptide can be tagged at the N-terminus with a polyglycine sequence, or alternatively, with at the C-terminus with a LPXTG (SEQ ID NO: 237) motif. The blocking element or other element has respective peptides attached that serve as acceptor sites for the tagged polypeptides. For conjugation to domains carrying a LPXTG (SEQ ID NO: 237) acceptor peptide attached via its N-terminus, the polypeptide will be tagged with an N-terminal poly-glycine stretch. For conjugation to domain carrying a poly-glycine peptide attached via its C-terminus, the polypeptide will be tagged at its C-terminus with a LPXTG (SEQ ID NO: 237) sortase recognition sequence. Recognizing poly-glycine and LPXTG (SEQ ID NO: 237) sequences, sortase will form a peptide bond between polymer-peptide and tagged polypeptides. The sortase reaction cleaves off glycine residues as intermediates and occurs at room temperature.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

“Cytokine” is a well-known term of art that refers to any of a class of immunoregulatory proteins (such as interleukin or interferon) that are secreted by cells especially of the immune system and that are modulators of the immune system. Cytokine polypeptides that can be used in the inducible cytokine prodrugs disclosed herein include IL-2, IL-12, IL-15, and IL-21, as well as fragments of such polypeptides that activate the cognate receptors for the cytokine (i.e., functional fragments of the foregoing). “Chemokine” is a term of art that refers to any of a family of small cytokines with the ability to induce directed chemotaxis in nearby responsive cells.

Cytokines are well-known to have short serum half-lives that frequently are only a few minutes. Even forms of cytokines that have altered amino acid sequences intended to extend the serum half-life yet retain receptor agonist activity typically also have short serum half-lives. As used herein, a “short-half-life cytokine” refers to a cytokine that has a substantially brief half-life circulating in the serum of a subject, such as a serum half-life that is less than 10, less than 15, less than 30, less than 60, less than 90, less than 120, less than 240, or less than 480 minutes. As used herein, a short half-life cytokine includes cytokines which have not been modified in their sequence to achieve a longer than usual half-life in the body of a subject and polypeptides that have altered amino acid sequences intended to extend the serum half-life yet retain receptor agonist activity. This latter case is not meant to include the addition of heterologous protein domains, such as a bona fide half-life extension element, such as serum albumin.

As used herein, a “cytokine polypeptide” refers to a cytokine, mutein, subunit, or functional fragment thereof. For example, the p35 and p40 subunits of IL-12 are each a cytokine polypeptide.

A “conservative” amino acid substitution, as used herein, generally refers to substitution of one amino acid residue with another amino acid residue from within a recognized group which can change the structure of the peptide, but biological activity of the peptide is substantially retained. Conservative substitutions of amino acids are known to those skilled in the art. Conservative substitutions of amino acids can include, but not limited to, substitutions made amongst amino acids within the following groups: (a) M, I, L, V; (b) F, Y, W; (c) K, R, H; (d) A, G; (e) S, T; (f) Q, N; and (g) E, D. For instance, a person of ordinary skill in the art reasonably expect that an isolated replacement of a leucine with an isoleucine or valine, an aspartate with a glutamate, a threonine with a serine, or a similar replacement of an amino acid with a structurally related amino acid will not have a major effect on the biological activity of the resulting molecule.

As used herein, a “half-life extension element” is a part of the inducible cytokine prodrug that increases the serum half-life and improve pK, for example, by altering its size (e.g., to be above the kidney filtration cutoff), shape, hydrodynamic radius, charge, or parameters of absorption, biodistribution, metabolism, and elimination.

As used herein, the term “linker” refers to an amino acid sequence typically less than about 100 amino acids that connects or links a first amino acid sequence of interest (e.g., an amino acid sequence that folds to form a first protein domain) to a second amino acid sequence of interest (e.g., an amino acid sequence that folds to form a second protein domain) in a contiguous polypeptide chain. The linker typically includes one or more protease cleavage sites and thus is protease cleavable. A “tandem linker” refers to a linker that comprises two or more protease cleavages sites which can be cleaved by the same or different proteases, and which can be arranged in any desired orientation, such as one cleavage site adjacent to another cleavage site, one cleavage site overlapping another cleavage site, one cleavage site following by another cleavage site with intervening amino acids between the two cleavage sites.

As used herein, the terms “induce,” and “inducible” refer to the ability of a protein, i.e. a cytokine, that is part of a conjugate, to bind its receptor and effectuate activity upon cleavage of additional elements from the conjugate.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, “subject” can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered.

As used herein, “patient” or “subject” may be used interchangeably and can refer to a subject with a disease or disorder (e.g. cancer). The term patient or subject includes human and veterinary subjects.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

An “attenuated cytokine receptor agonist” is a cytokine receptor agonist that has decreased receptor agonist activity as compared to the cytokine receptor's naturally occurring agonist. An attenuated cytokine agonist may have at least about 10×, at least about 50×, at least about 100×, at least about 250×, at least about 500×, at least about 1000× or less agonist activity as compared to the receptor's naturally occurring agonist. When a inducible cytokine prodrug that contains a cytokine polypeptide as described herein is described as “attenuated” or having “attenuated activity”, it is meant that the inducible cytokine prodrug is an attenuated cytokine receptor agonist.

An “intact inducible cytokine prodrug” is a inducible cytokine prodrug in which no domain has been removed from the inducible cytokine prodrug, for example by protease cleavage. A domain may be removable by protease cleavage or other enzymatic activity, but when the inducible cytokine prodrug is “intact”, this has not occurred.

As used herein “moiety” refers to a portion of a molecule that has a distinct function within that molecule, and that function may be performed by that moiety in the context of another molecule. A moiety may be a chemical entity with a particular function, or a portion of a biological molecule with a particular function. For example, a “blocking element” within a inducible cytokine prodrug is a portion of the inducible cytokine prodrug which is capable of blocking the activity of some or all of the inducible cytokine prodrug polypeptide. This may be a protein domain, such as serum albumin.

The term “adoptive cell therapy” is a term of art that that refers to immune cells that are used for therapeutic purposes. Typically, in the practice of adoptive cell therapy, immune cells are administered to a subject. Such cells can be, for example, obtained from a suitable source, such as the subject to be treated, and expanded or activated, and administered back to the subject for therapeutic purposes. The immune cells can be isogeneic or allogeneic and can be modified to include, for example, chimeric antigen receptors or engineered T cell receptors. The immune cells can be unmodified immune cells, such as unmodified tumor infiltrating lymphocytes. Well-known examples of adoptive cell therapy include tumor-infiltrating lymphocyte (TIL) therapy, engineered T cell receptor (TCR) therapy, chimeric antigen receptor T cell (CAR-T) therapy, natural killer (NK) cell therapy, and chimeric antigen receptor NK cell (CAR-NK) therapy, for example.

The term “effective amount” refers to an amount sufficient to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products.

As used herein the terms “treatment”, “treat”, or “treating” refer to a method of reducing the effects of a disease or condition or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms “prevent”, “preventing”, and “prevention” of a disease or disorder refer to an action, for example, administration of the inducible cytokine prodrug or nucleic acid sequence encoding the inducible cytokine prodrug, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

### 6. INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### 7. EXAMPLES

The following are examples of methods and compositions of the invention. It is understood that various other embodiments may be practiced, given the general description provided herein.

### Reference Example A: Inducible Cytokine Prodrugs

Inducible cytokines are described in WO2019/222294, WO2019/222295, WO2019/222296, and WO2020/232305, WO2021/097376, or WO2021/236676. Generation of an inducible cytokine is described in example 6 of WO2019/222295. Inducible IL-2 prodrug anti-tumor effects are described in Example 12 of WO2019/222295. Inducible IL-12 prodrug anti-tumor effects are described in Example 10 of WO2019/222296.

### Example 1: In Vivo Tumor Models

### 1.1 Tumor Infiltrating Lymphocyte Models

Inducible cytokines and adoptive cell therapy will be studied in mouse models of tumor infiltrating lymphocyte adoptive cell therapy (TIL ACT) using the Pmel-1 mouse, which expresses a T cell receptor specific for gp 100 (a transmembrane protein expressed on melanocytes and melanoma) and/or the Trp-1 mouse, which expresses a T cell receptor specific for tyrosinase-related protein 1 (a protein involved in melanin synthesis). Both mice are available from The Jackson Laboratory. See, e.g., Overwijk et al. J. Exp. Med. (2003) 198 (4): 569-580; Klebanoff et al. Clin Cancer Res. (2011) 17 (16): 5343-5352; Gattinoni et al. J. Clin. Invest. (2005) 115 (6): 1616-1626; Klebanoff et al. Proc. Natl. Acad. Sci. U.S.A. (2005) 102 (27): 9571-9576; Dwyer et al. Eur. J. Immunol. (2020) 50 (9): 1386-1399; Muranski et al. Blood (2008) 112 (2): 362-372.

In studies using the pmel-1 mouse, the spleens from pmel-1 mouse will be harvested and a single cell suspension prepared. The spleen cell suspension will be cultured with hgp 100 peptide 25-33 (1 micromolar, available from Genescript) and IL-2. Cells will be cultured to expand T cell for about 1 week. The expanded cells will then be infused into C57BL/6 mice bearing a murine melanoma (cell line B16F10 derived from C57BL/6) via the tail vein.

In studies using the trp-1 mouse, the spleens from trp-1 mouse will be harvested and a single cell suspension prepared. The spleen cell suspension will be cultured with irradiated spleen cells loaded with tyrosine-related protein peptide 106-130 (1 micromolar, available from Genescript) and IL-2. Cells will be cultured to expand T cell for about 1 week. The expanded cells will then be infused into C57BL/6 mice bearing a murine melanoma (cell line B16F10 derived from C57BL/6) via the tail vein.

In both models, treatment groups will include TIL ACT, TIL ACT plus inducible IL-2 or IL-12 prodrugs (that is activated by cleavage in the tumor microenvironment) and may also include TIL ACT with IL-2 or IL-12. Efficacy will be assessed by measuring tumor size and animal survival. The results are expected to demonstrate that treatment TIL ACT and inducible human IL-2 supports extended proliferation of TIL ACT leading to xenograft eradication.

### 1.2 CAR-T Models

Inducible human IL-2 and IL-12 will be tested with human CAR-T in HER2, CD47 BCMA and/or Burkitt's Lymphoma cancer models. NSG or NOG immune-compromises mice (available from The Jackson Laboratory and Taconic Biosciences, respectively) will be used in the study. See, e.g., Forsberg et al. Cancer Research 79 (5) 899-904 (2019). These mice include alterations (e.g., deletion) of the endogenous IL-2Rgamma chain which prevents IL-2 induced signal transduction by endogenous cells. Human tumor xenografts will be grown in the mice, using the HER2 breast cancer cell line BT474, the CD47 pancreatic cancer cell line BxPC3, or the BCMA multiple myeloma cell line RPMI8226. Human CAR-T cells that target human HER2, CD47, CD19 or BCMA will be administered alone or with an inducible form of human IL-2 or IL-12 that is activated by cleavage in the xenograft tumor microenvironment. Efficacy, tolerability and pharmacodynamic changes following treatment will be evaluated, including HER2, CD47, CD19 or BCMA expression in xenografts following CAR-T treatment. The results are expected to demonstrate that treatment with CAR-T and inducible human IL-2 or IL-12 is well tolerated and that inducible human IL-2 or IL-12 supports extended proliferation of CAR-T leading to xenograft eradication.

### Example 2. Adoptive Cell Therapy for B16F10 Melanoma with Inducible IL-2 Prodrug

### 2.1 Preparation of B16F10 Melanoma and Implantation

B16F10 (CRL-6475) were obtained from ATCC. Cells were put in culture and expanded until they reached 60-80% confluency and cultures were amplified until enough cells are obtained for implantation. On the day of implantation, cells were harvested and resuspended at a density of 2×10⁶ cells/mL (2×10⁵ cells/100 μL). The day prior to implantation, the lower back of all the mice was shaved in the location of the tumor implantation. 100 μL subcutaneous injections was performed on the lower back of the C57BL/6 to implant the B16F10 melanoma. Tumors were allowed to grow for 9-14 days on the C57BL/6 mice to an average of approximately 40-120 mm³ before pmel T cell transfer.

### 2.2 Pmel-1 Splenocyte Preparation and Activation

Spleens from Pmel-1 mice (Jackson Labs) were processed to single cell suspension by passing through a 40-70 μM cell strainer over a 50 ml conical tubes. Cells were washed and collected to treat with ACK lysis buffer for erythrocyte elimination. Cells were collected, washed, and resuspended at 1×10⁶ cells/mL in cell culture media to conduct activation. Pmel splenocytes were stimulated by adding 1 μg/mL hgp 10025-33 peptide (Genscript) and 3 ng/mL rhIL-2 to the media. Cells were placed in a 24 well plate (2 mL/well) and incubate at 37° C. for 72 hours. On Day 3, cells were collected and resuspended at 1×10⁶ cells/mL with 3 ng/mL rhIL-2. From Day 4 to 7, cells were kept at a density of 1-2×10⁶ cells/mL and replenish rhIL-2 for new media added. During this time the pmel-1 cell cultures enter logarithmic growth and become mostly CD8+ T cell and may need be split 1:1 daily.

### 2.3 Initiating Pmel-1 ACT to B16F10 Melanoma

The day prior to ACT, all the mouse tumors were measured to ensure they are 3D and are 40-120 mm³ for randomization. All animals were injected with 4 mg/mouse cyclophosphamide monohydrate (Sigma Aldrich) via intraperitoneal injection. On Day 7 of the pmel-1 cultures, cells were collected and washed twice with 20 mL PBS. Cells are centrifugated and resuspended at 100×10⁶ cells/mL (10×10⁶ cells/100 μL) in PBS for IV injection. On day 12 animals were injected with pmel-1 cells (100 μL) intravenously via the tail vein. Extra pmel-1 cells were analyzed by flow cytometry to determine their phenotype.

Following T cell transfer, on days 1, 4, and 8, mice were dosed ip with IL-2 or different forms of inducible IL-2 prodrug according to the treatment protocol as outlined in Tables 5 and 6 below.

TABLE 5
Treatment Protocol Study 1

| Group | N | Agent |
|---|---|---|
| 1 | 7 | No cells + no cytokine |
| 2 | 7 | 5 × 10⁶ pmel/mouse alone |
| 3 | 7 | 10 × 10⁶ pmel/mouse alone |
| 4 | 7 | 5 × 10⁶ pmel/mouse + |
| | | WW0621/0523 (100 μg) |
| 5 | 7 | 10 × 10⁶ pmel/mouse + |
| | | WW0621/0523 (100 μg) |
| 6 | 7 | No cells + WW0621/0523 |
| | | (100 μg) |

TABLE 6
Treatment Protocol Study 2

| Group | N | Agent |
|---|---|---|
| 1 | 12 | No cells + no cytokine |
| 2 | 12 | 10 × 10⁶ pmel/mouse alone |
| 3 | 12 | 10 × 10⁶ pmel/mouse + |
| | | WW0621/0523 (100 μg) |
| 4 | 12 | 10 × 10⁶ pmel/mouse + |
| | | WW0729/0523 (100 μg) |
| 5 | 12 | No cells + WW0621/0523 |
| | | (100 μg) |
| 6 | 12 | No cells + WW0729/0523 |
| | | (100 μg) |
| 7 | 12 | No cells + rhIL-2 (3 μg) |
| 8 | 12 | 10 × 10⁶ pmel/mouse+ rhIL- |
| | | 2 (3 μg) |

### 2.4. Pmel-1 ACT Follow Up

Following the treatment protocol, the tumors were measured using digital calipers twice weekly along with body weight. To follow donor cell engraftment and persistence, submandibular vein bleeding was performed with a lancet to collect 100-200 μL once per week up to one month for flow cytometry analysis. Pmel-1 cells are identified by using the Vβ13 antibody which denotes their TCR that is specific for gp 100 which is a glycoprotein expressed on melanoma and melanocytes. The vehicle group was also stained as there is a host Vβ13 positive population which is approximately 4% of the CD8⁺ T cells. Tumor growth was followed until the tumor size reached 2000 mm³ or an animal has lost 20% of its initial body weight which are our endpoint criteria. Efficacy was determined by plotting tumor volume against time. Survival curves were also generated.

### 2.5 Results

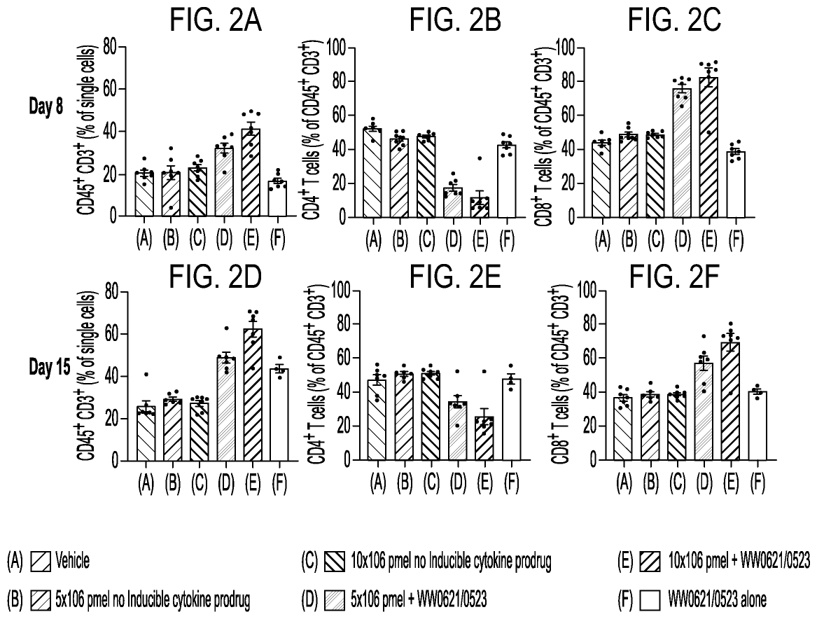

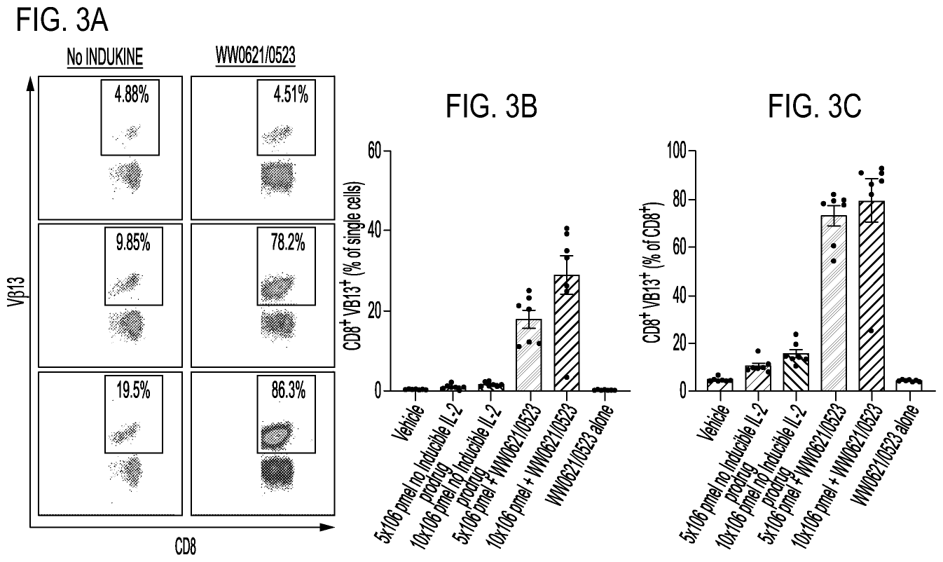

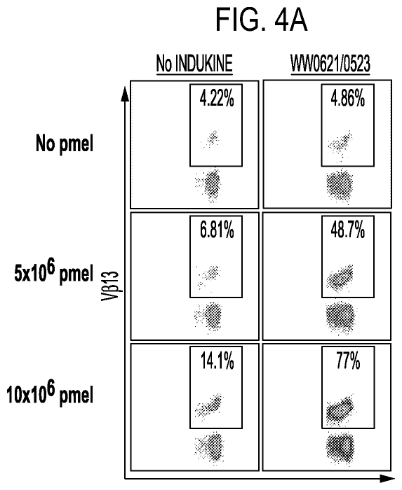

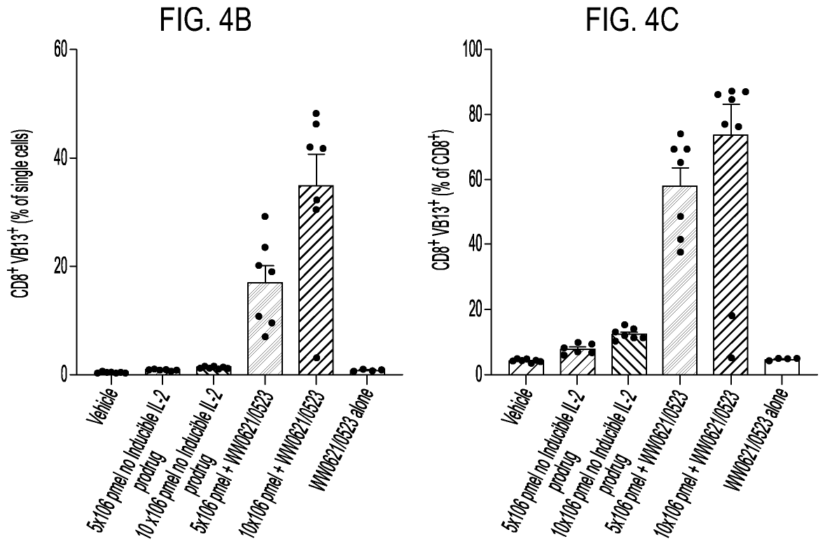

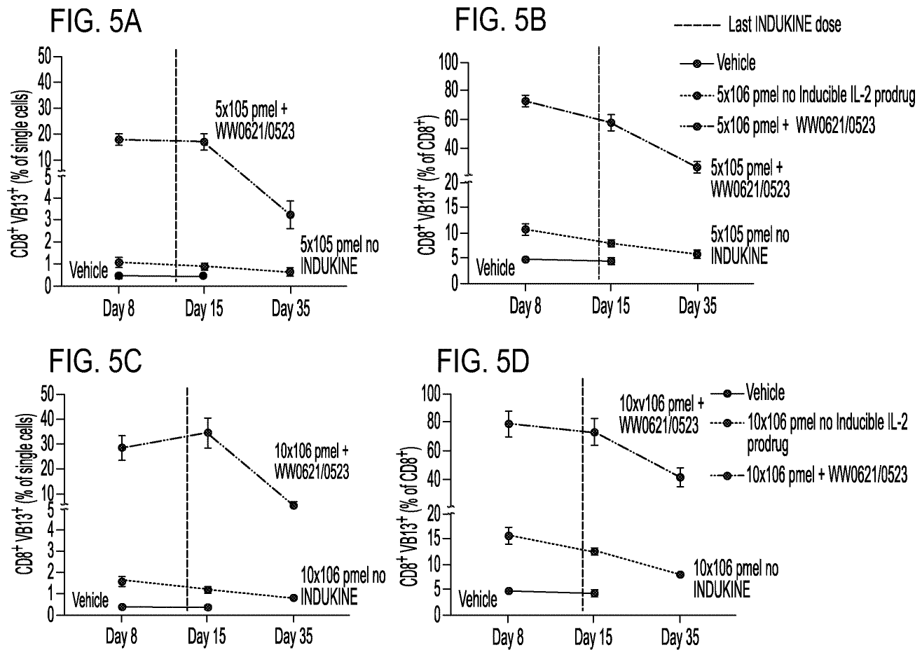

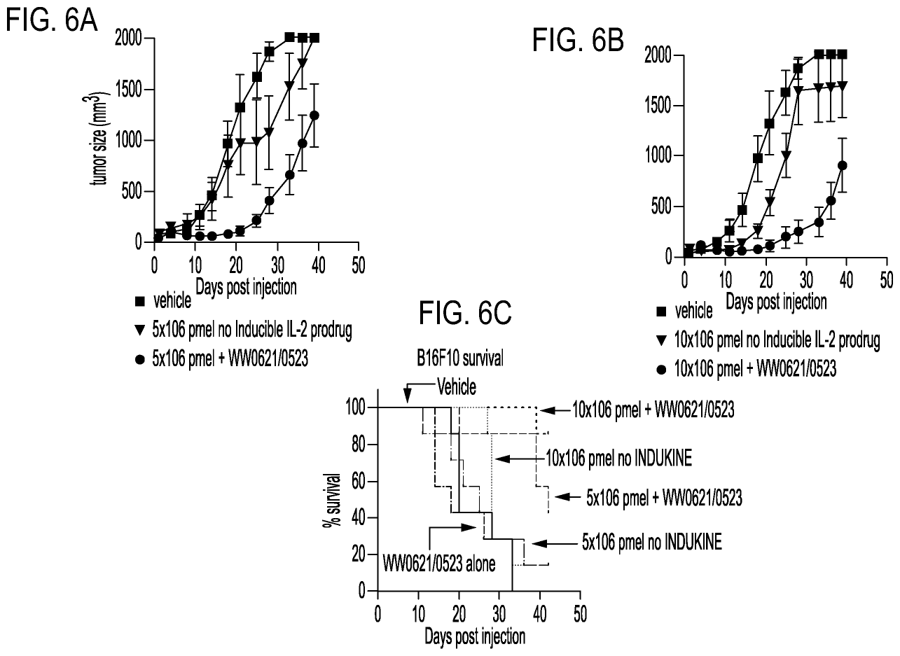

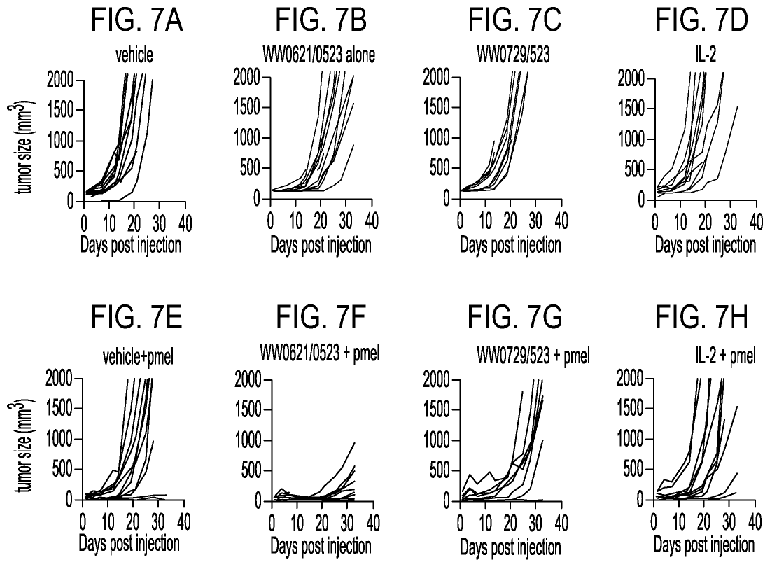

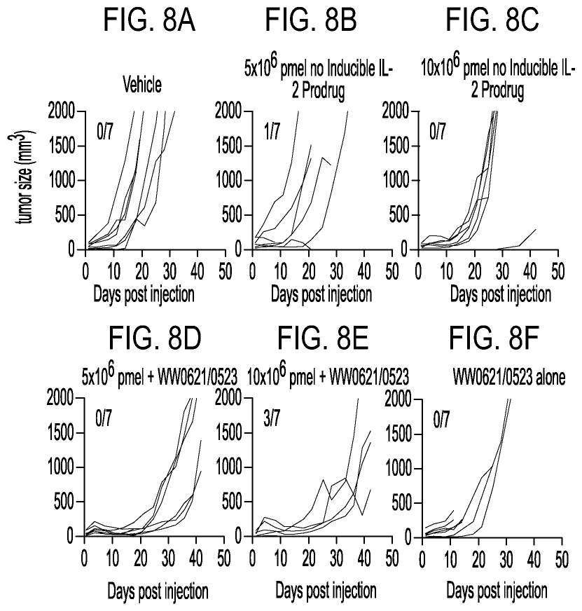

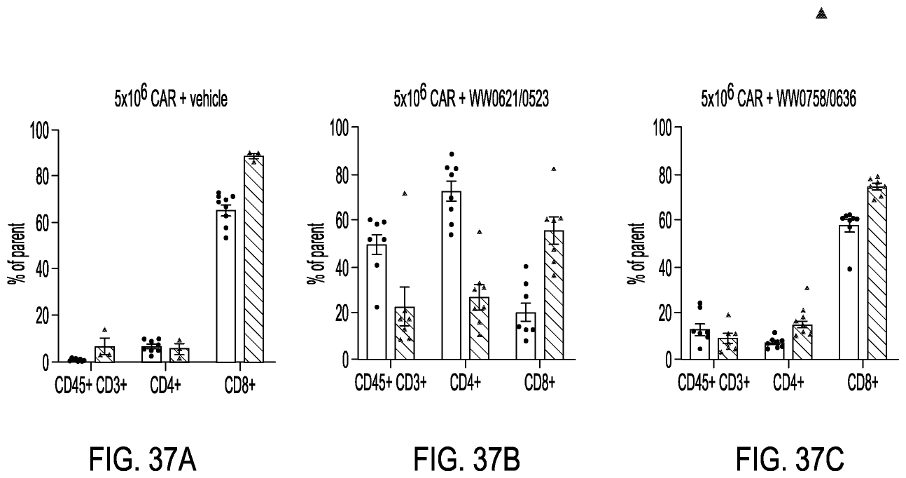

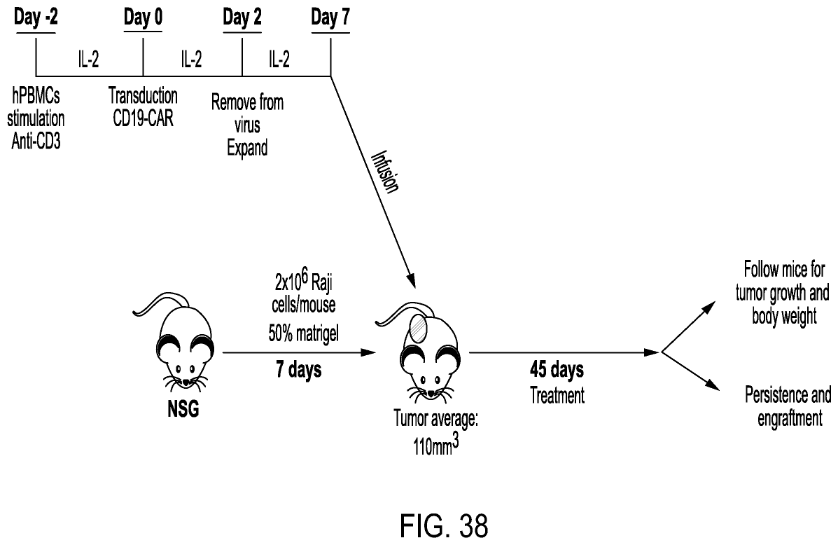

WW0621/0523 in combination with CD8+ pmel-1 ACT in both 5×10⁶ and 10×10⁶ cell groups led to improved engraftment and persistence of donor cells inhibiting tumor growth (e.g., B16F10 growth). See, FIGS. 2A-2F, 3A-3C, 4A-4C, 5A-6D, 6A-6C, 7A-7H, 8A-8F, 9A-9C and 10A-10E. Both dose levels of CD8+pmel plus WW0621/0523 provided improved efficacy controlling B16F10 tumor growth. See, FIG. 37

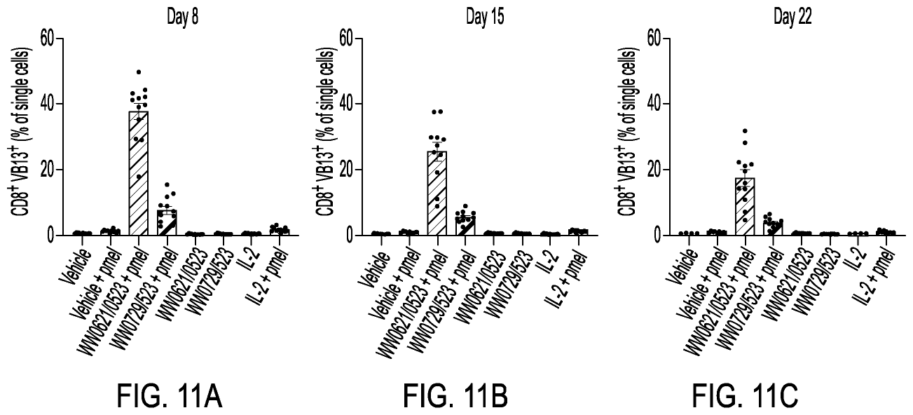

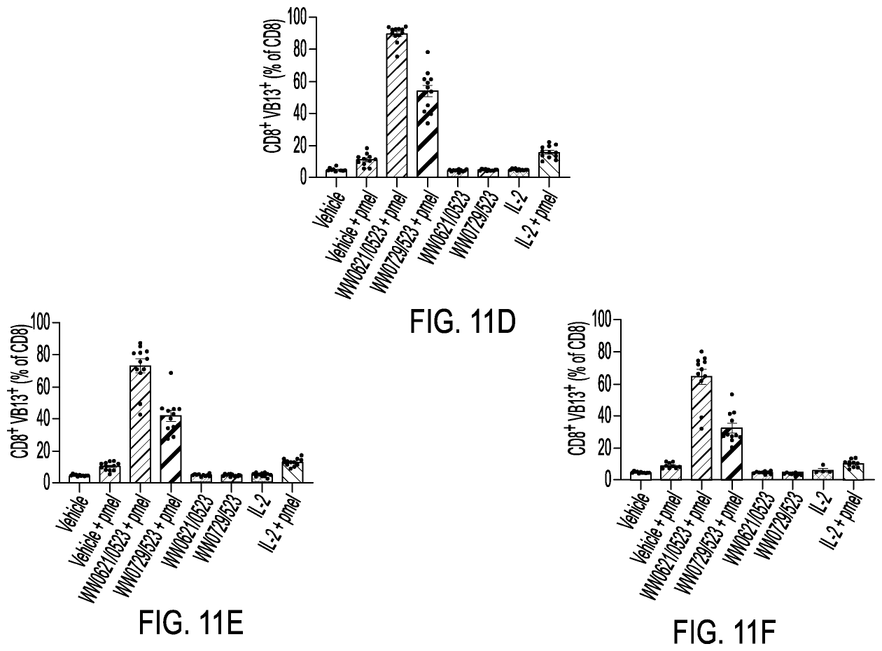

WW0729/0523 lead to some peripheral expansion and tumor growth delay but not to the same extent as WW0621/0523 showing that cleavage of the molecule in the tumor is important. See, FIGS. 11A-11F.

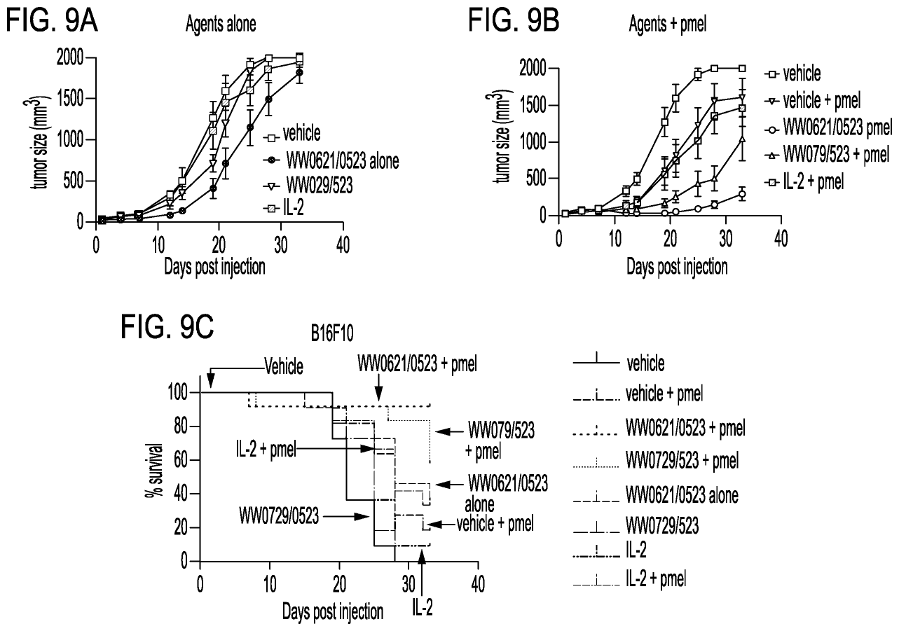

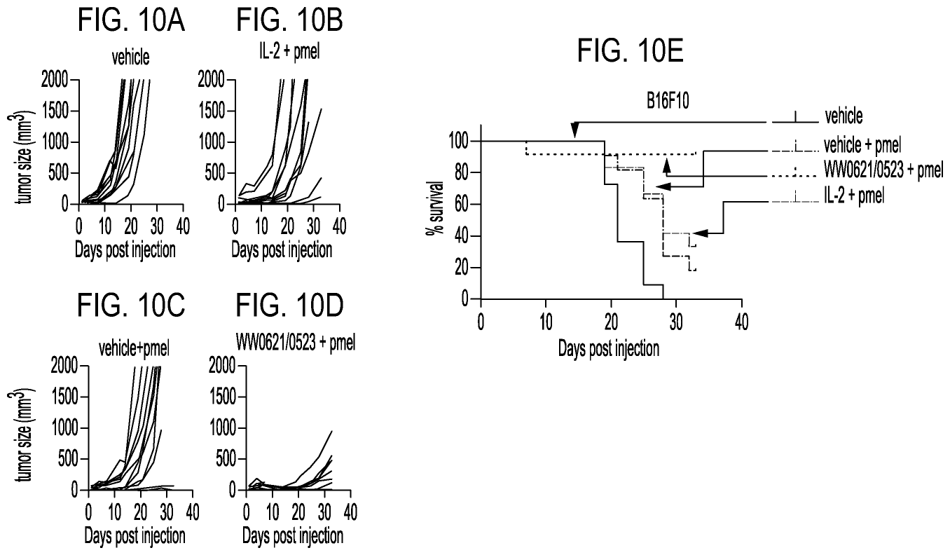

WW0621/0523 in combination with adoptive cell therapy provided a therapeutic benefit against tumor growth (e.g., B16F10 growth). Furthermore, WW0621/0523 in combination with CD8+ pmel-1 ACT provided better tumor growth inhibition compared to rhIL-2 in combination with CD8+ pmel-1 ACT. See, FIGS. 9A-9C, and 10A-10E.

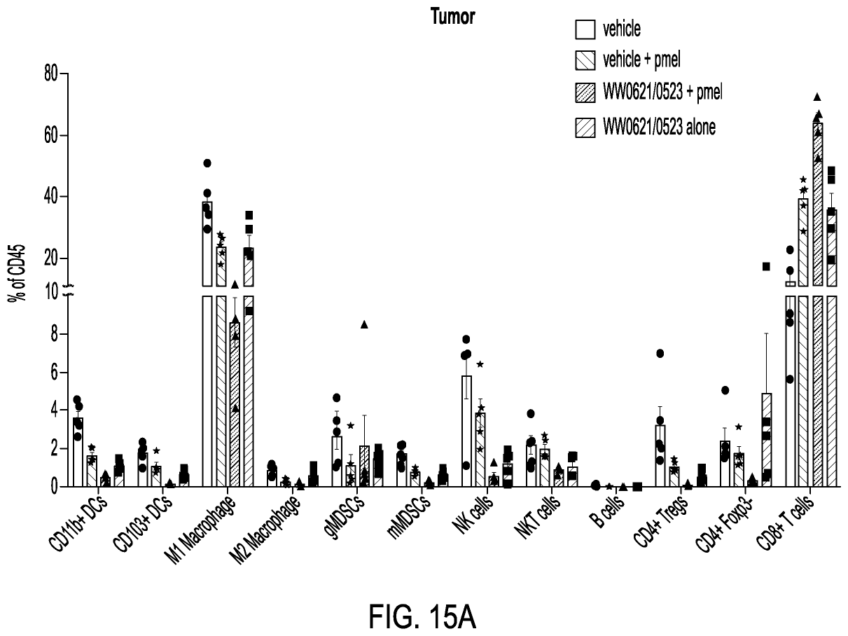

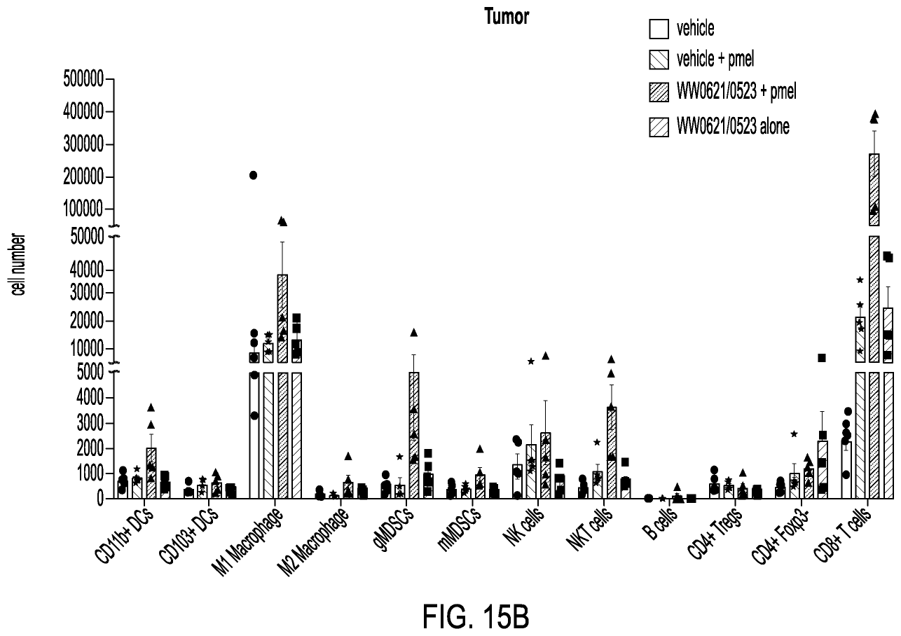

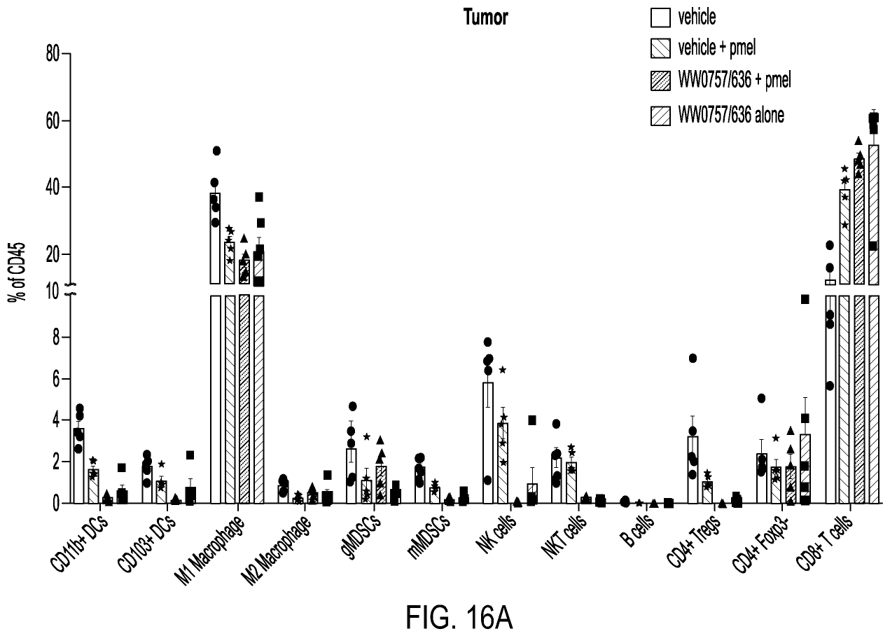

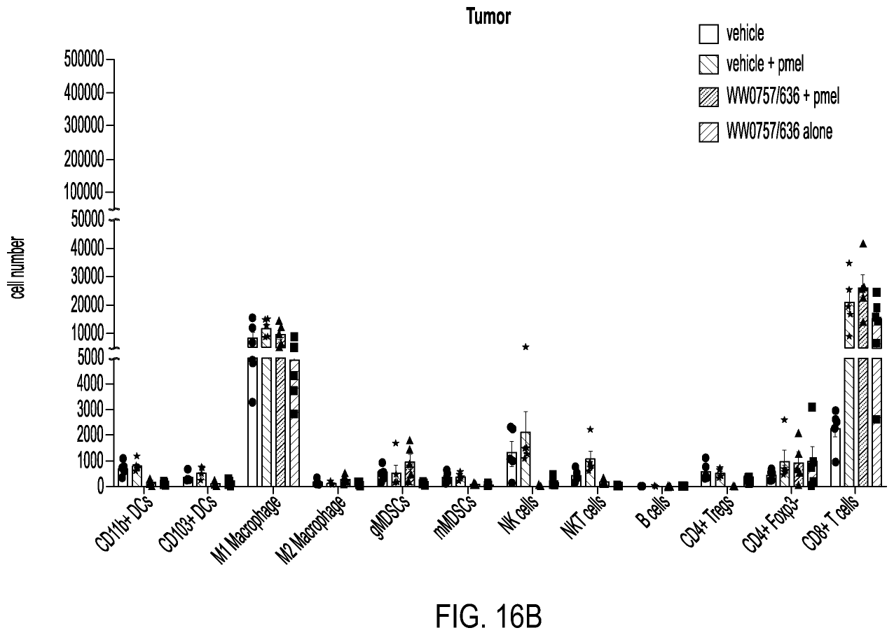

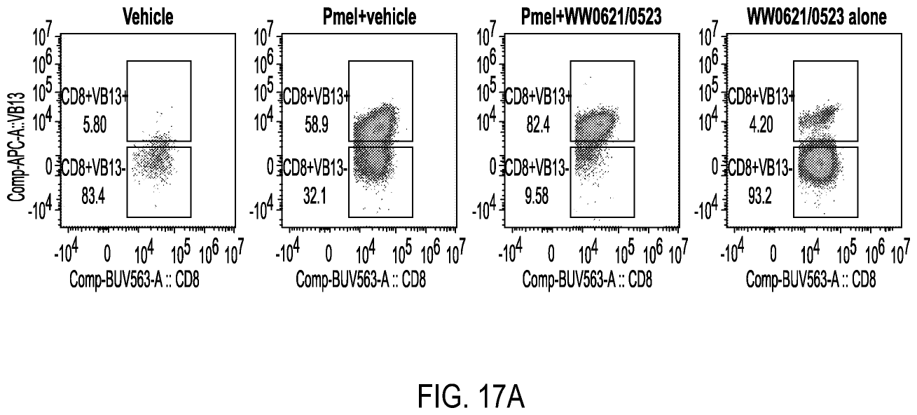

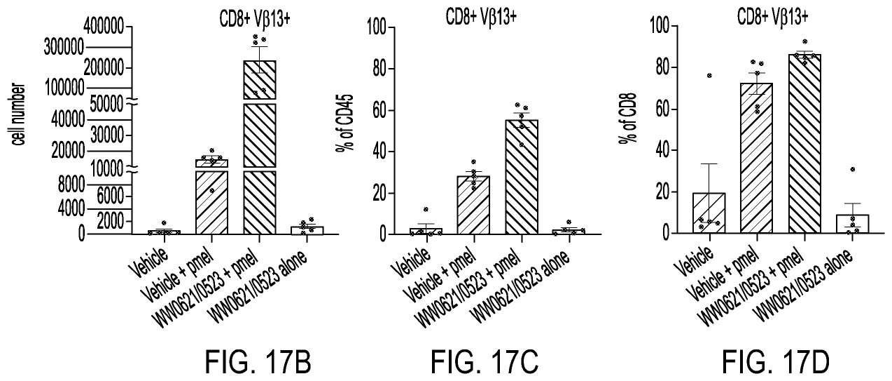

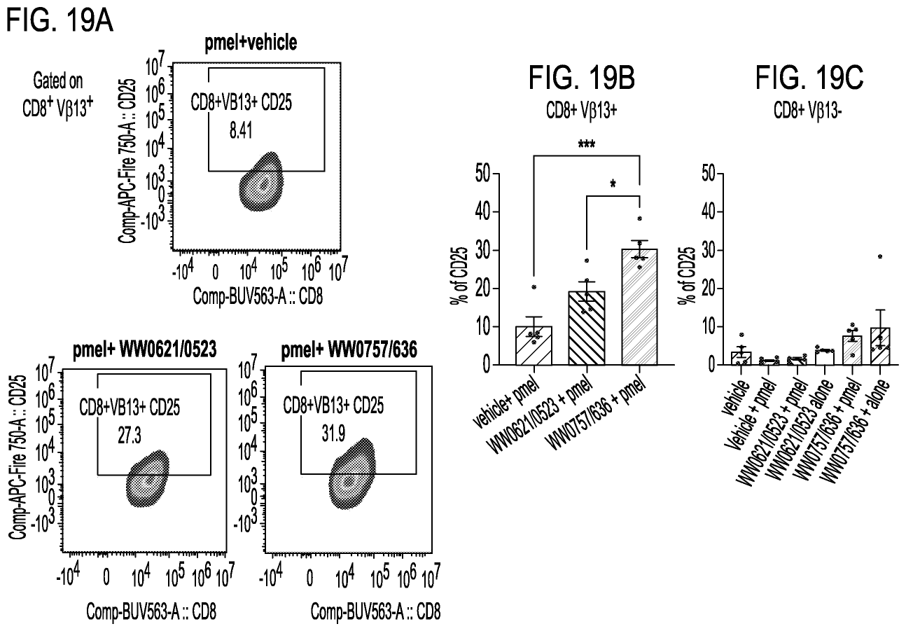

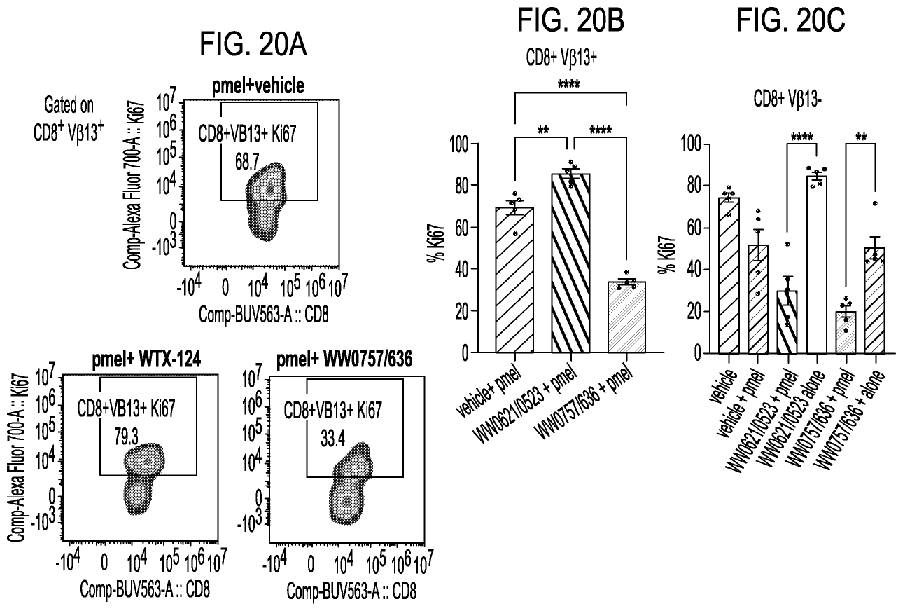

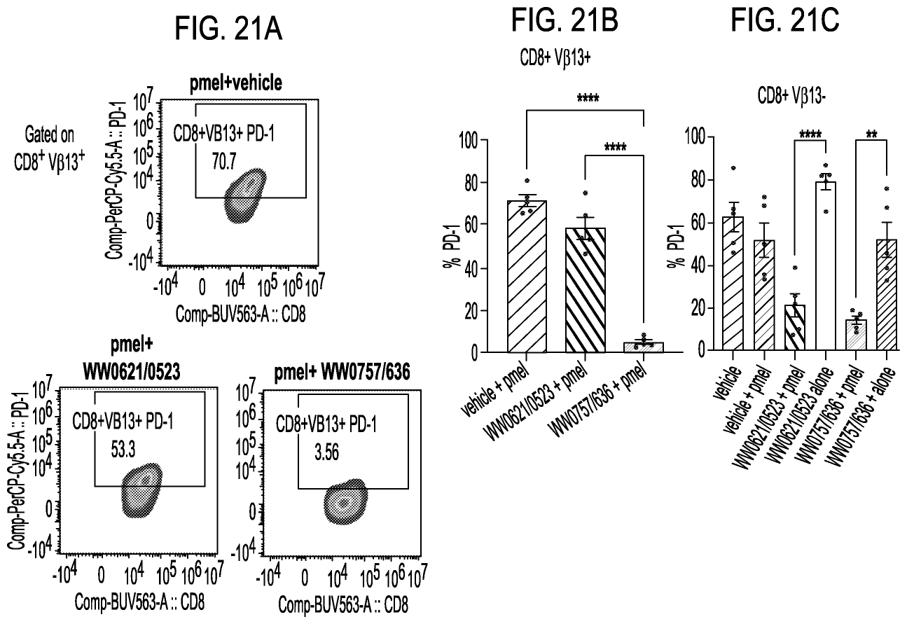

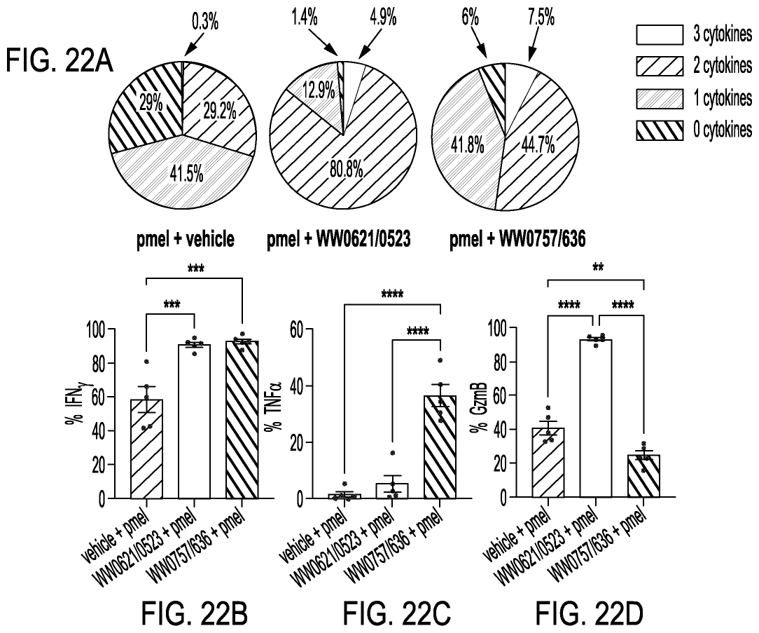

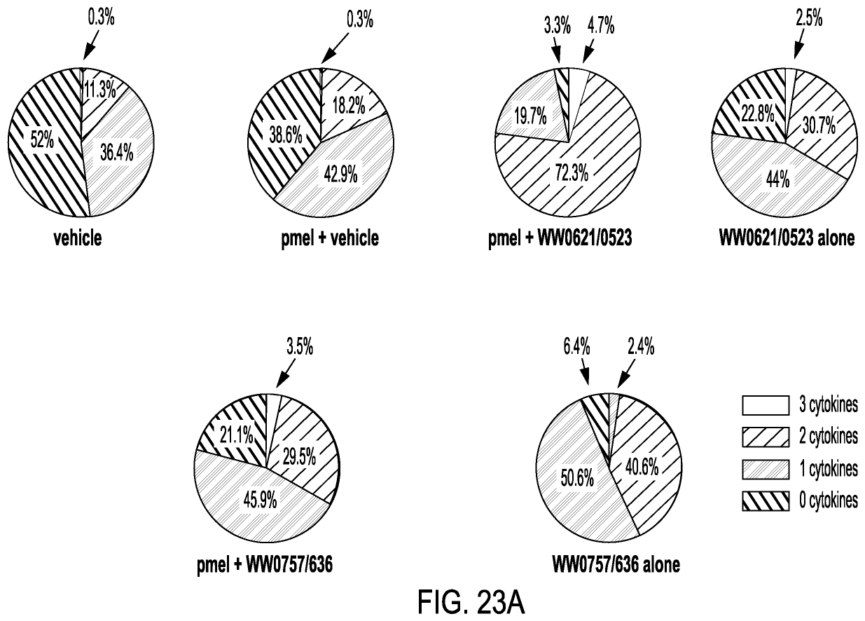

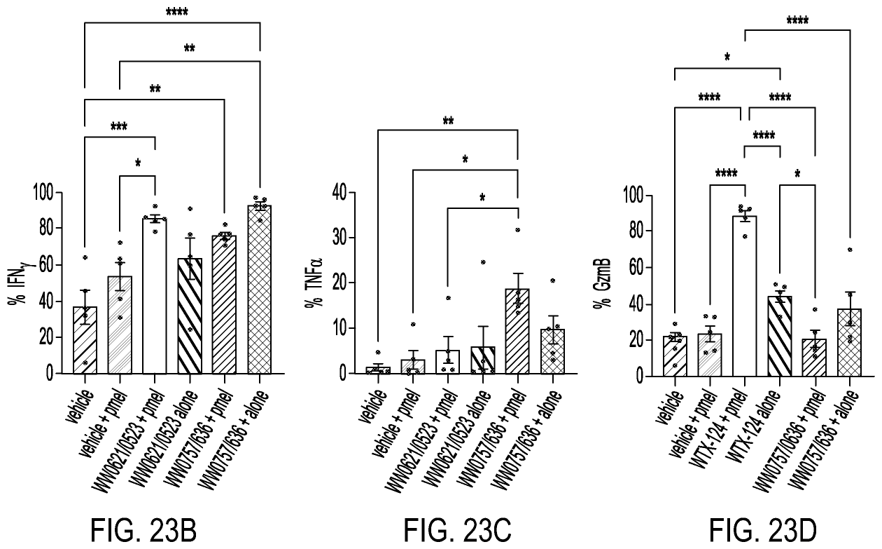

Further results are shown in FIGS. 15A, 15B, 16A, 16B, 17A-17D, 18A-18D, 19A-19C, 20A-20C, 21A-21C, 22A-22D, and 23A-23D.

### Example 3. Engineered Tumor-Infiltrating Lymphocytes that Express Inducible Cytokine Prodrug

Tumor infiltrating lymphocytes (TILs) will be modified to express inducible cytokine prodrugs that are soluble or membrane tethered. The cytokine prodrug expressing TILs will be tested in animal models, including the B16F10 melanoma model.

3.1 Preparation of Engineered TILs that Express Inducible Cytokine Prodrug.

Pmel-1 splenocytes will be prepared and activated as described herein and will be transduced with lentiviral vector encoding inducible cytokine prodrug (either soluble or membrane tethered). Prior to lentiviral transduction, 24 well plates will be coated with 30 μg/mL of Retronectin (Takara) for 2 hrs. Pmel-1 cells will be collected and resuspended at 8×10⁶ cells/mL and 250 μL of cell suspension were added to each well of the Retronectin coated plate. Lentivirus stocks will be diluted to the appropriate MOI for transduction and 250 μL virus will be added to the PBMC. Plates will then be centrifuged for 2 hrs at 2000×g. After centrifugation, the volume of media will be brought up to 2 mLs in 18.5 ng/mL hIL-2 and incubated at 37° C. for 7-10 days. Cells will be kept at a density of 1-2×10⁶ cells/mL and replenished with fresh media containing rhIL-2 every 2 days. Expression of inducible cytokine prodrug by transduced cells will be confirmed using flow cytometry.

### 3.2 Preparation of B16F10 Melanoma and Implantation

B16F10 (CRL-6475) will be obtained from ATCC. Cells will be put in culture and expanded until they reach 60-80% confluency and cultures are amplified until enough cells are obtained for implantation. On the day of implantation, cells will be harvested and resuspended at a density of 2×10⁶ cells/mL (2×10⁵ cells/100 μL). The day prior to implantation, the lower back of all the mice will be shaved in the location of the tumor implantation. 100 μL subcutaneous injections will be performed on the lower back of the C57BL/6 to implant the B16F10 melanoma. Tumors will be allowed to grow for 9-14 days on the C57BL/6 mice to an average of approximately 40-120 mm³ before pmel T cell transfer.

### 3.3 Pmel-1 Splenocyte Preparation and Activation

Cells will be collected, washed, and resuspended at 1×10⁶ cells/mL in cell culture media to conduct activation. Pmel splenocytes will be stimulated by adding 1 μg/mL hgp10025-33 peptide (Genscript) and 3 ng/mL rhIL-2 to the media. Cells will be placed in a 24 well plate (2 mL/well) and incubated at 37° C. for 72 hours. On Day 3, cells will be collected and resuspended at 1×10⁶ cells/mL with 3n g/mL rhIL-2 and transduced with lentivirus containing specific inducible cytokine prodrug sequences. Virus will be maintained in the culture for 2 days. After 2 days, cells will be washed and kept at a density of 1-2×10⁶ cells/mL and replenished with rhIL-2 for new media added for 4 additional days before IV inoculation into animals.

### 3.4 Initiating Pmel-1 ACT to B16F10 Melanoma

The day prior to ACT, all the mouse tumors will be measured to ensure they are 3D and are 40-120 mm³ for randomization. All animals will be injected with 4 mg/mouse cyclophosphamide monohydrate (Sigma Aldritch) via intraperitoneal injection. On day 12, after pmel culture initiation, animals will be injected with pmel-1 cells (100 μL) intravenously via the tail vein. Extra pmel-1 cells will be analyzed by flow cytometry to determine their phenotype.

Following IV transfer of cells, mice will be treated according to the treatment protocol as outlined in Table 7 below.

TABLE 7
Treatment Protocol

| Group | N | Agent |
|---|---|---|
| 1 | 10 | No cells + no cytokine |
| 2 | 10 | 10 × 10⁶ pmel/mouse + |
| | | no cytokine |
| 3 | 10 | 10 × 10⁶ pmel |
| | | WW0621/0523 (soluble |
| | | form)/mouse |
| 4 | 10 | 10 × 10⁶ pmel |
| | | WW0621/0523 |
| | | (membranted |
| | | tethered)/mouse |
| 5. | 10 | 10 × 10⁶ pmel |
| | | WW0621/0523 (100 |
| | | μg)/mouse |

### 3.5. Pmel-1 ACT Follow Up

Following the treatment protocol the tumors will be measured using digital calipers twice weekly along with body weight. To follow donor cell engraftment and persistence, submandibular vein bleeding will be performed with a lancet to collect 100-200 μL once per week up to one month for flow cytometry analysis. Pmel-1 cells will be identified by using the Vβ13 antibody which denotes their TCR that is specific for gp100 which is a glycoprotein expressed on melanoma and melanocytes. The vehicle group will also stained as there is a host Vβ13 positive population which is approximately 4% of the CD8⁺ T cells. Tumor growth will be followed until the tumor size reached 2000 mm³ or an animal has lost 20% of its initial body weight which are our endpoint criteria. Efficacy is determined by plotting tumor volume against time. Survival curves will also be generated.

### Example 4. CAR-T Cell Therapy Expressing Inducible Cytokine Prodrugs

CAR-T cells will be modified to express inducible cytokine prodrugs that are both soluble and membrane tethered. Protocols used for the preparation and efficacy testing using human PBMCs and human tumor cells implanted in NSG animals are described below. CD19 CARTs and tumor cells lines expressing hCD19 will be used as a model.

### 4.1 Preparation of Raji and Implantation on NSG Mice

Raji (CCL-86 ATCC) is a tumor cell line expressing hCD19. Raji cells will be cultured until they reach 2-3×10⁶ cells/mL and cultures were expanded until enough cells are obtained for implantation. The day prior to implantation, the lower back of all the mice will be shaved in the location of the tumor implantation. On the day of implantation, cells will be harvested and resuspended at a density of 2×10⁷ cells/mL (2×10⁶ cells/100 μL in 50% Matrigel (Corning)). 100 μL subcutaneous injections were performed on the lower back of the NSG mice. Tumors will be allowed to grow for 9-14 days to an average of approximately 40-150 mm³ prior to CAR T cell transfer.

### 4.2 CAR T Cell Generation Using Human PBMCs

PBMCs will be isolated from healthy donor leukopaks (BIOIVT) and then cryopreserved until use. Frozen PBMC stocks were thawed, counted and the cell density adjusted to 1×10⁶ cells/mL. PBMCs will be stimulated with anti-CD3 antibody (Clone OKT3, Biolegend) and rhIL-2 in a 24 well plate (2 mL/well) and incubated at 37° C. for 48 hours. Prior to lentiviral transduction, 24 well plates will be coated with 30 μg/mL of Retronectin (Takara) for 2 hrs. Stimulated PBMC will be collected and resuspended at 8×10⁶ cells/mL and 250 μL of cell suspension were added to each well of the Retronectin coated plate. Lentivirus stocks will be diluted to the appropriate MOI for transduction and 250 μL virus will be added to the PBMC. Plates were then centrifuged for 2 hrs at 2000×g. After centrifugation, the volume of media will be brought up to 2 mLs in 18.5 ng/ml hIL-2 and incubated at 37° C. for 7-10 days. Cells will be kept at a density of 1-2×10⁶ cells/mL and replenished with fresh media containing rhIL-2 every 2 days.

To generate inducible cytokine prodrug expressing CAR T cells, CAR T will be transduced with lentivirus expressing inducible cytokine prodrug as described above. Expression of CAR and inducible cytokine prodrug by transduced PBMCs will be confirmed using flow cytometry.

4.3 Efficacy Assessment: Treatment of NSG Animals Bearing Raji Tumors with CAR T Cells or INDUKINE Expressing CART Cells

The day prior to ACT, all the mouse tumors will be measured to ensure they are 3D and are 40-120 mm³ for randomization. On Day 10-15, CAR T cells will be collected and washed twice with 20 mL PBS. Cells will be centrifugated and resuspended at 100×10⁶ cells/mL (10×10⁶ cells/100 μL) in PBS for IV injection. Animals will be injected with CAR T cells (100 μL) intravenously via the tail vein. Different CARTs and combinations will be tested by comparing non-transduced CARTs−/+ systemic inducible cytokine prodrug treatment with CARTs transduced with lentivirus to express different forms of inducible cytokine prodrugs (secreted vs membrane tethered)

To determine efficacy tumors will be measured using digital calipers twice weekly along with body weight. Efficacy will be determined by plotting tumor volume against time (days) CAR T cell engraftment and persistence will be followed; submandibular vein bleeding will be performed with a lancet to collect 100-200 μL once per week up to one month for flow cytometry analysis. Tumor growth will be followed until the tumor size reached 2000 mm³ or animals lost 20% of its initial body weight which are our endpoint criteria.

### Example 5. Adoptive Cell Therapy for B16F10 Melanoma with an Inducible IL-12 Prodrug

### 5.1 Preparation of B16F10 Melanoma and Implantation

B16F10 (CRL-6475) was obtained from ATCC. Cells were put in culture and expanded until they reached 60-80% confluency and cultures were amplified until enough cells were obtained for implantation. On the day of implantation, cells were harvested and resuspended at a density of 2×10⁶ cells/mL (2×10⁵ cells/100 μL). The day prior to implantation, the lower backs of all the mice were shaved in the location of the tumor implantation. 100 μL subcutaneous injections were performed on the lower back of the C57BL/6 to implant the B16F10 melanoma. Tumors were allowed to grow for 9-14 days on the C57BL/6 mice to an average of approximately 40-120 mm³ before pmel T cell transfer.

### 5.2 Pmel-1 Splenocyte Preparation and Activation

Spleens from Pmel-1 mice (Jackson Labs) were processed to single cell suspension by passing through a 40-70 μM cell strainer over a 50 ml conical tubes. Cells were washed and collected to treat with ACK lysis buffer for erythrocyte elimination. Cells were collected, washed, and resuspended at 1×10⁶ cells/mL in cell culture media to conduct activation. Pmel splenocytes were stimulated by adding 1 μg/mL hgp 10025-33 peptide (Genscript) and 3 ng/mL rhIL-2 to the media. Cells were placed in a 24 well plate (2 mL/well) and incubated at 37° C. for 72 hours. On Day 3, cells were collected and resuspended at 1×10⁶ cells/mL with 3 ng/mL rhIL-2. From Day 4 to 7, cells were kept at a density of 1-2×10⁶ cells/mL and were replenished with fresh rhIL-2 in new media added. During this time the pmel-1 cell cultures entered logarithmic growth and became mostly CD8⁺ T cell and needed to be split 1:1 daily.

### 5.3 Initiating Pmel-1 ACT to B16F10 Melanoma

The day prior to ACT, all the mouse tumors were measured to ensure they are 3D and were 40-120 mm³ for randomization. All animals were injected with 4 mg/mouse cyclophosphamide monohydrate (Sigma Aldritch) via intraperitoneal injection. On Day 7 of the pmel-1 cultures, cells were collected and washed twice with 20 mL PBS. Cells were centrifugated and resuspended at 100×10⁶ cells/mL (10×10⁶ cells/100 μL) in PBS for IV injection. On day 12 animals were injected with pmel-1 cells (100 μL) intravenously via the tail vein. Extra pmel-1 cells were analyzed by flow cytometry to determine their phenotype.

Following T cell transfer on days 1, 4, and 8 mice were dosed i.p. with different forms of inducible IL-12 according to the treatment protocol as outlined in Table 8 below.

TABLE 8
Treatment Protocol

| Group | N | Agent |
|---|---|---|
| 1 | 8 | No cells + no cytokine |
| 2 | 8 | 10 × 10⁶ pmel/mouse + |
| | | no cytokine |
| 3 | 8 | 10 × 10⁶ pmel/mouse + |
| | | WW0757/ WW0636 (25 |
| | | μg) |
| 4 | 8 | 10 × 10⁶ pmel/mouse + |
| | | WW0757/ WW0636 (50 |
| | | μg) |
| 5 | 8 | 10 × 10⁶ pmel/mouse + |
| | | WW0825/WW0636 (25 |
| | | μg) |
| 6 | 8 | 10 × 10⁶ pmel/mouse + |
| | | WW0825/WW0636 (50 |
| | | μg) |
| 7 | 8 | No cells + WW0757/ |
| | | WW0636 (25 μg) |
| 8 | 8 | No cells + WW0757/ |
| | | WW0636 (50 μg) |
| 9 | 8 | No cells + |
| | | WW0825/WW0636 (25 |
| | | μg) |
| 10 | 8 | No cells + |
| | | WW0825/WW0636 (50 |
| | | μg) |

### 5.4. Pmel-1 ACT Follow Up

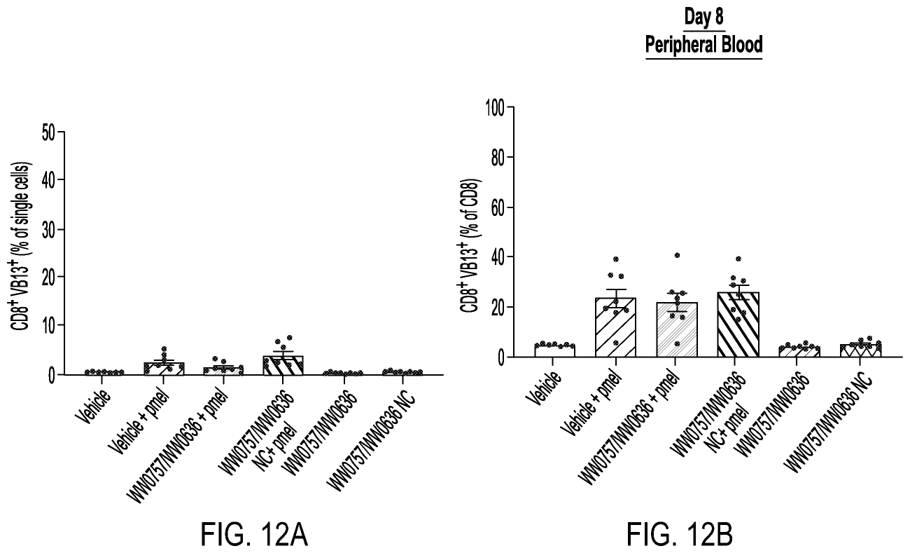

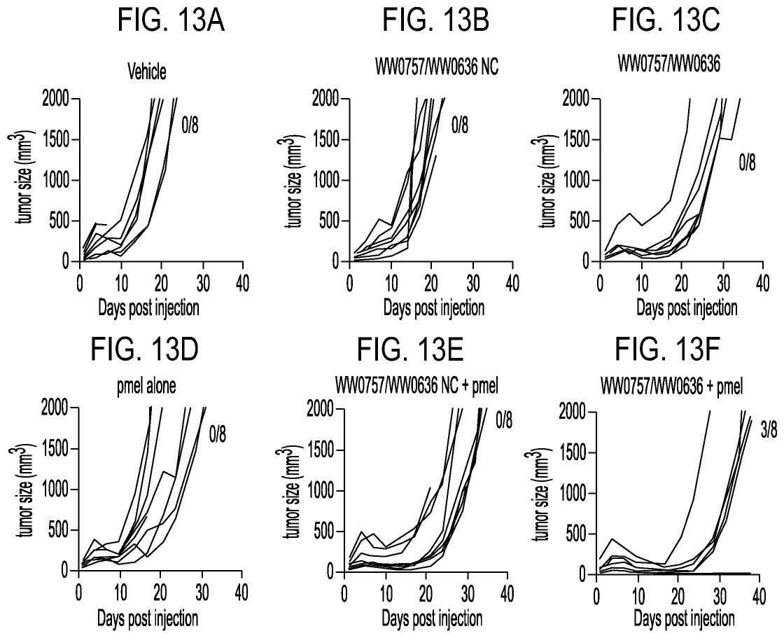

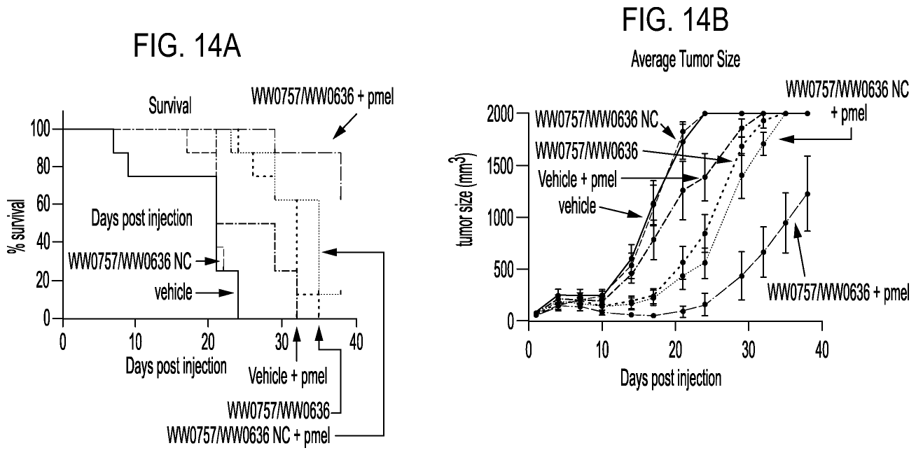

Following the treatment protocol, the tumors were measured using digital calipers twice weekly along with body weight. To follow donor cell engraftment and persistence, submandibular vein bleeding was performed with a lancet to collect 100-200 μL once per week up to one month for flow cytometry analysis. Pmel-1 cells were identified by using the Vβ13 antibody which denotes their TCR that is specific for gp100 which is a glycoprotein expressed on melanoma and melanocytes. The vehicle group was stained as there is a host Vβ13 positive population which is approximately 4% of the CD8⁺ T cells (FIGS. 12A and 12B). Tumor growth was followed until the tumor size reached 2000 mm³ or an animal had lost 20% of its initial body weight which are our endpoint criteria. Efficacy was determined by plotting tumor volume against time (FIGS. 13A-13F, and 14B). Survival curves and average tumor volume curves were also generated (FIG. 14A and FIG. 14B, respectively).

### 5.5 Results

WW0757/0636 in combination with CD8+ pmel-1 ACT led to improved tumor control and animal survival in B16F10 melanoma. See, FIGS. 13A-13F, and 14B. However, WW0757/0636 treatment did not appear to improve engraftment of donor pmel-1 donor CD8+ T cells, but may have improved the functional quality of the donor cells. See, FIGS. 12A-12B.

Further results are shown in FIGS. 15A, 15B, 16A, 16B, 17A-17D, 18A-18D, 19A-19C, 20A-20C, 21A-21C, 22A-22D, and 23A-23D.

### Example 6. CAR T Cell Therapy Expressing an Inducible IL-2 Cytokine Prodrug

CAR-T cells were modified to express an inducible IL-2 prodrug that is membrane tethered.

### 6.1 Inducible Cytokine Prodrug Constructs

A lentiviral construct (pLentiX vector, Thermo Fisher-) encoding a membrane tethered IL-2 under the EF1α promoter was generated. Briefly, the encoded polypeptide, (WW50563), consists of human IL-2, a blocking moiety (an anti-IL2 scFv) and PDGF-R transmembrane domain with a GFP tag. The IL-2, blocking, and transmembrane domains are each linked via a tumor protease cleavable linker, Linker 3 (SEQ ID NO: 198).

### 6.2 Generation of Inducible IL-2 Prodrug Expressing CAR-T Cells

PBMCs were isolated from healthy donor leukopaks (BIOIVT) and then cryopreserved until use. Frozen PBMC stocks were thawed, counted and the cell density adjusted to 1×10⁶ cells/mL. PBMCs were stimulated with 50 ng/mL anti-CD3 antibody (Clone OKT3, Biolegend) and 18.5 ng/mL rhIL-2 in a T175 flask incubated at 37° C. for 48 hours. Prior to lentiviral transduction, 24 well plates were coated with 30 μg/mL of Retronectin (Takara) for 2 hrs at room temperature. Stimulated PBMCs were collected and resuspended at 8×10⁶ cells/mL and 250 μL of cell suspension were added to each well of the Retronectin coated plate. To generate inducible IL-2 prodrug expressing CART cells, inducible IL-2 prodrug lentivirus stocks were diluted to MOI of 20 for transduction in 37 ng/ml of hIL-2 and 250 μL virus was added to the PBMCs. Plates were then centrifuged for 2 hrs at 2000×g. After centrifugation, the volume of media was brought up to 2 mLs in 18.5 ng/ml hIL-2 and incubated at 37° C. for 24 hours. After a 24 hour incubation with the inducible IL-2 prodrug expressing lentivirus, cells were harvested and counted. Cells were then transduced with CD19 CAR Lentivirus (BPS Bioscience, Cat #78601) at an MOI of 3 as described above. Cells were incubated with the CD19 CAR lentivirus for 24 hours. After the 24-hour incubation with the CAR lentivirus, transduced cells were washed clean of lentivirus containing media and resuspended in media containing 18.5 ng/mL rhIL-2. Cells were kept at a density of 1-2×10⁶ cells/mL and replenished with fresh media containing 18.5 ng/ml rhIL-2 every 2 days.

### 6.3 CAR Inducible IL-2 Prodrug Mouse Study Protocol

Female NSG mice (6 weeks old) were purchased from Jackson Laboratories and were kept in the vivarium for one week prior to experimentation. Raji Burkitt's lymphoma was thawed from liquid nitrogen storage and resuspended in culture media. Cells were counted and 5×10⁶ cells were placed in a T75 flask with 10 mL of media for 48 hours at 37° C. After the initial incubation period, Raji cells were cultured for an additional 5 days maintaining a cell density of 1-2×10⁶ cells/mL in T75 flasks to obtain the desired cell number for mouse implantation. After one week of expansion, Raji cells were collected from the flasks, counted, and resuspended at a density of 40×10⁶ cells/mL in PBS. Raji cells were then mixed with 50% Matrigel for a final density of 20×10⁶ cells/mL and 100 μL (2×10⁶ cells) were subcutaneously injected on the lower back of the NSG animals. Tumors were allowed to grow for one week prior to CAR T cell infusion.

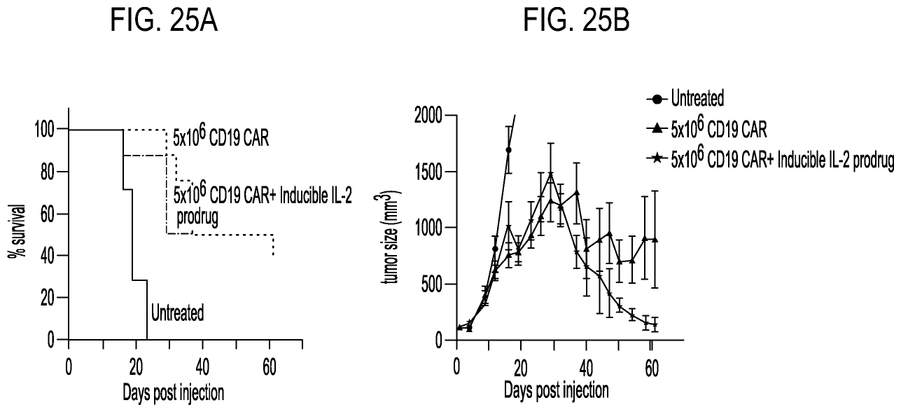

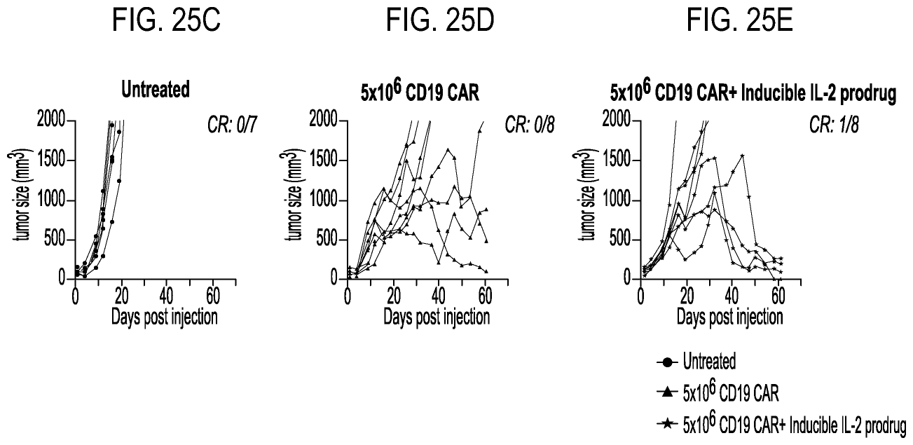

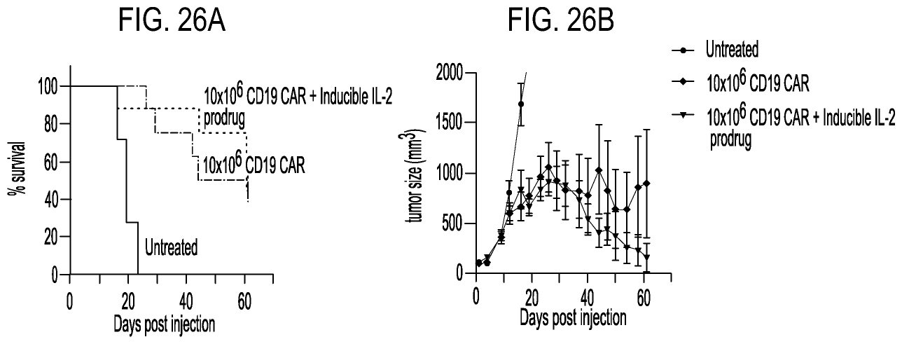

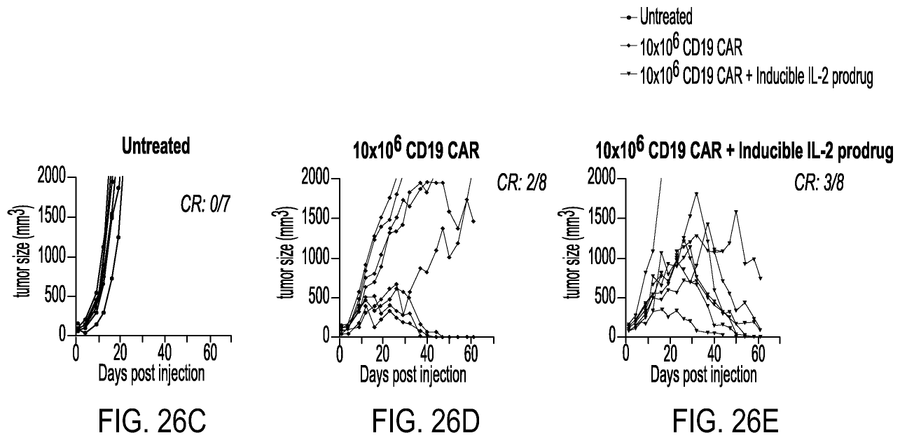

The day before CAR T cell infusion, Raji tumors were measured by digital calipers and mice were randomized to standardize tumor size between the treatment groups. Tumor sizes were 100-120 mm³. CAR T cells and inducible IL-2 prodrug expressing CAR T cells were collected from the 24 well plates and washed with PBS to remove culture media. Cells were counted and resuspended at a density of 100×10⁶ cells/mL (10×10⁶ cells/100 μL) or 50×10⁶ cells/mL (5×10⁶ cells/100 μL) in PBS and 100 μL of cells per mouse were infused intravenously into tumor-bearing NSG mice. For treatment protocol, see Table 9 below. Mice in the study were monitored biweekly for body weight and tumor size by digital calipers until day 61. The criteria for a complete remission (CR) was two or more consecutive tumor measurements under 13 mm³. A partial response (PR) was a tumor that was 50% of the starting tumor volume. The results are shown in FIGS. 25A-25E and 26A-26E.

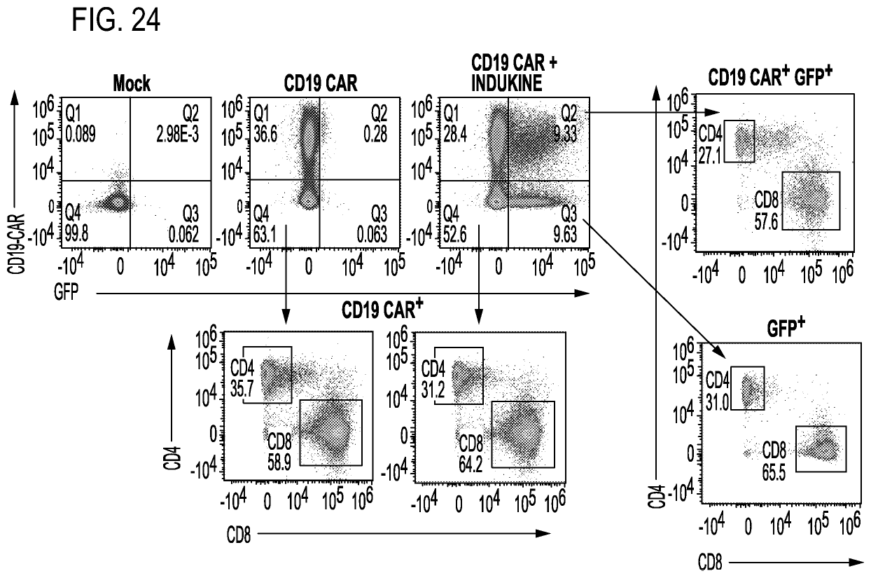

Inducible IL-2 prodrug and CAR expression were validated by flow cytometry analysis of the pre-infusion product. At day 5 pre-infusion, approximately 9.3% of the T cells were CD19 CAR+ Inducible IL-2 prodrug T cells. Of these cells, approximately 27.1% were CD4+ and 57.6% were CD8+ T cells. Results are shown in FIG. 24.

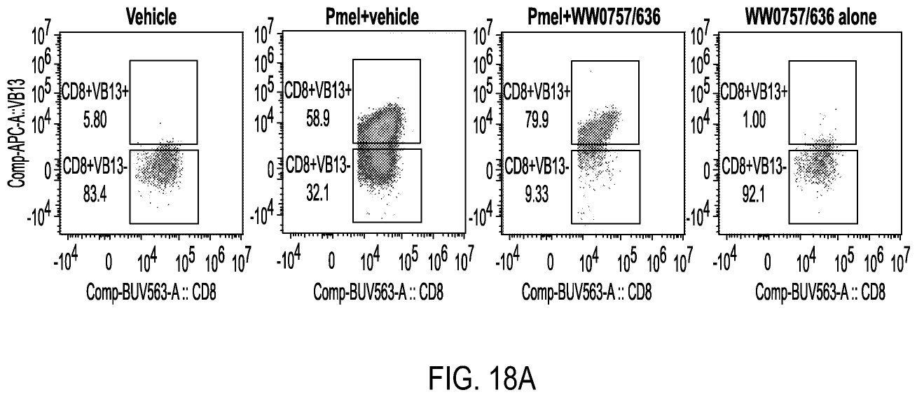

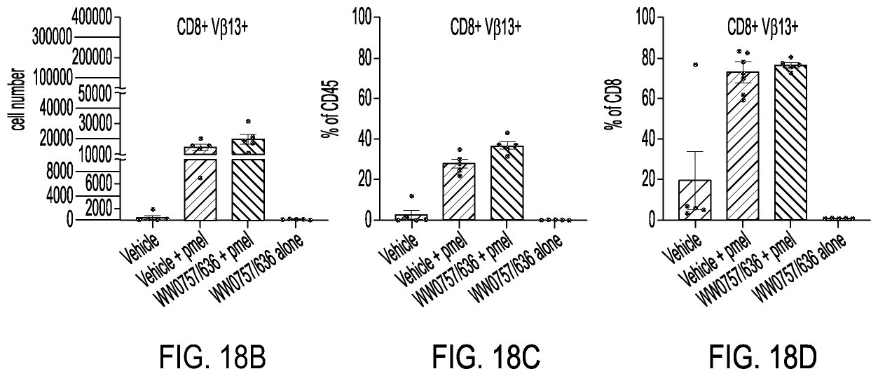

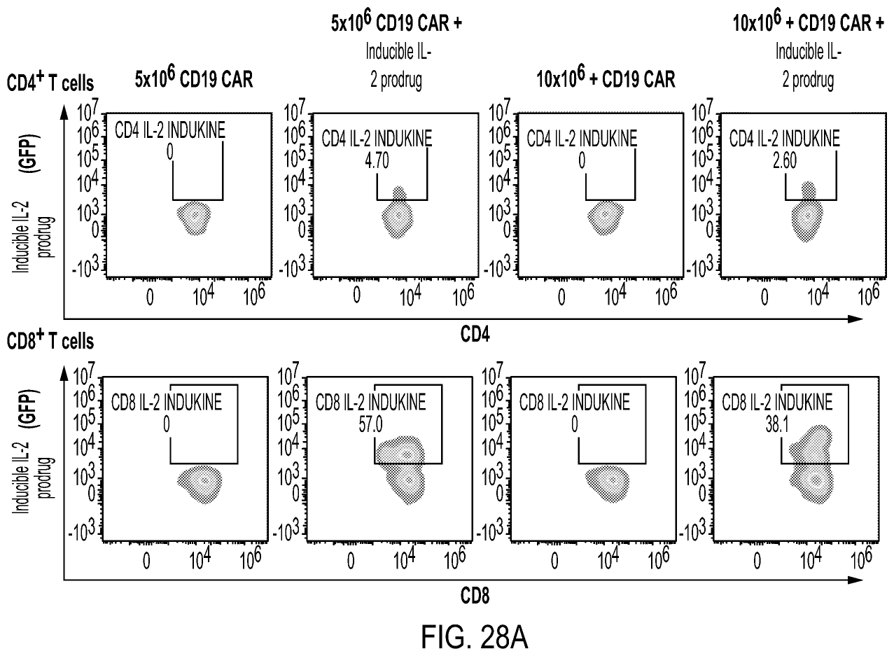

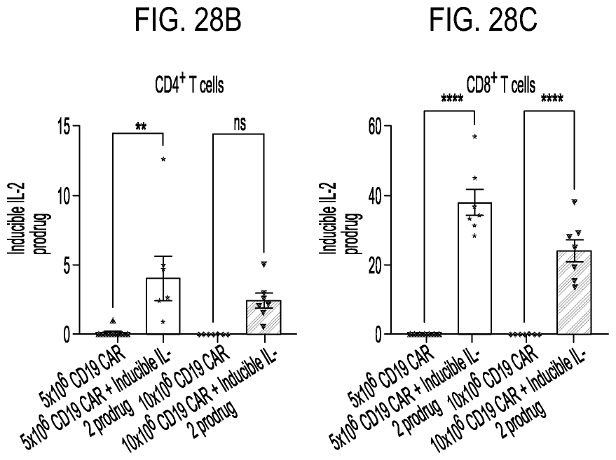

In vivo CAR T cell engraftment and persistence were assessed by flow cytometry of blood collected by submandibular vein bleeds while mice were on study. The results are shown in FIG. 18 and FIGS. 28A-28B.

TABLE 9
Treatment Protocol

| Group | N | Agent | Dose |
|---|---|---|---|
| 1 | 7 | Untreated | No cells + no cytokine |
| 2 | 8 | Mock | 10 × 10⁶ cells |
| 3 | 8 | CD19-CAR | 10 × 10⁶ cells |
| 4 | 8 | CD19-CAR Inducible IL-2 | 10 × 10⁶ cells |
| | | Prodrug (transmembrane | |
| | | tethered) | |
| 5 | 8 | CD19-CAR | 5 × 10⁶ cells |
| 6 | 8 | CD19-CAR Inducible IL-2 | 5 × 10⁶ cells |
| | | Prodrug (transmembrane | |
| | | tethered | |

### 6.4 Results

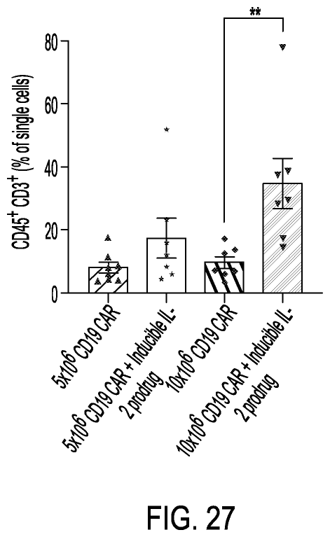

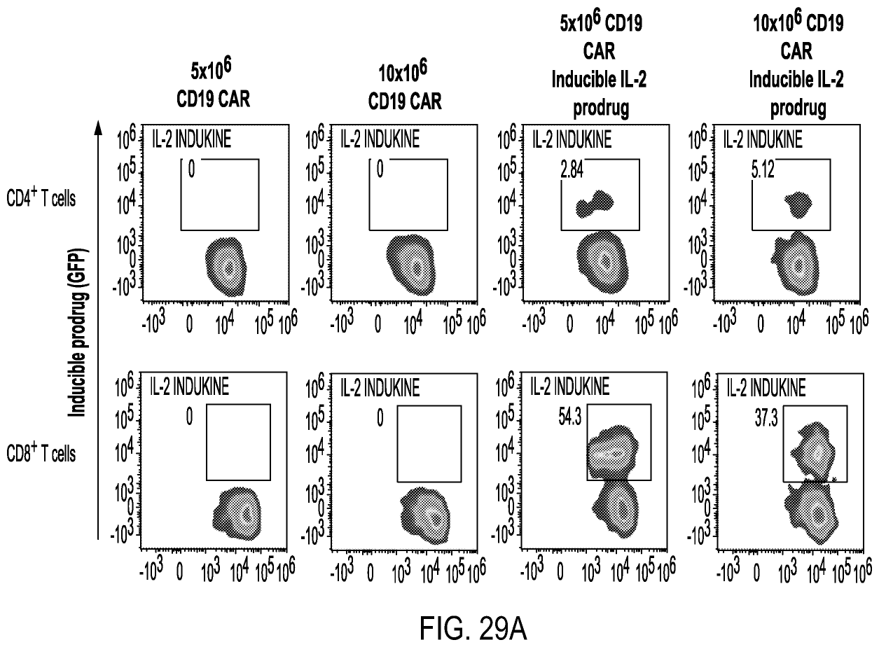

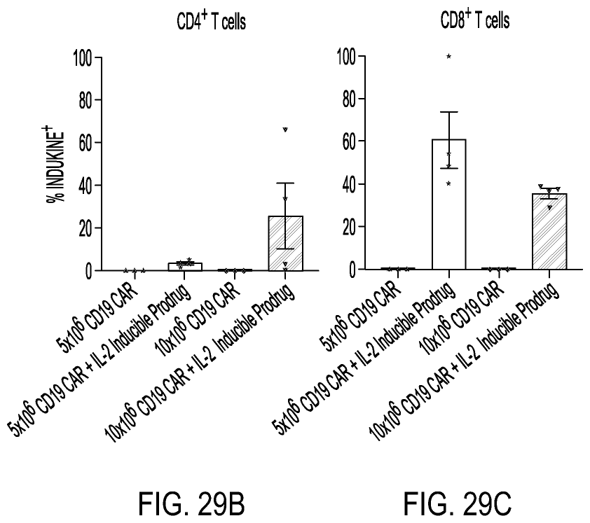

Mice treated with CD19 CAR-T cells expressing inducible IL-2 prodrug led to increased control of tumor growth and animal survival compared to tumor growth in untreated mice or mice treated with CD19 CAR+ T cells alone in a Burkitt's lymphoma mouse model. See, FIGS. 25A-25E and FIGS. 26A-26E. CD19 CAR-T cells expressing inducible IL-2 prodrug also have increased persistence and engraftment relative to CAR-T cells alone. See FIGS. 27, 28A-28C, and 29A-29C.

### Example 7. CAR T Cell Therapy with Systemic IL-2 or IL-12 Prodrug Administration

### 7.1 CAR T Cell Systemic Prodrug Administration Protocol

Female NSG mice (6 weeks old) were purchased from Jackson Laboratories and were kept in the vivarium for one week prior to experimentation. Raji Burkitt's lymphoma was thawed from liquid nitrogen storage and resuspended in culture media. Cells were counted and 5×10⁶ cells were placed in a T75 flask with 10 mL of media for 48 hours at 37° C. After the initial incubation period, Raji cells were cultured for an additional 5 days maintaining a cell density of 1-2×10⁶ cells/mL in T75 flasks to obtain the desired cell number for mouse implantation. After one week of expansion, Raji cells were collected from the flasks, counted, and resuspended at a density of 40×10⁶ cells/mL in PBS. Raji cells were then mixed with 50% Matrigel for a final density of 20×10⁶ cells/mL and 100 μL (2×10⁶ cells) were subcutaneously injected on the lower back of the NSG animals. Tumors were allowed to grow for one week prior to CAR T cell infusion.

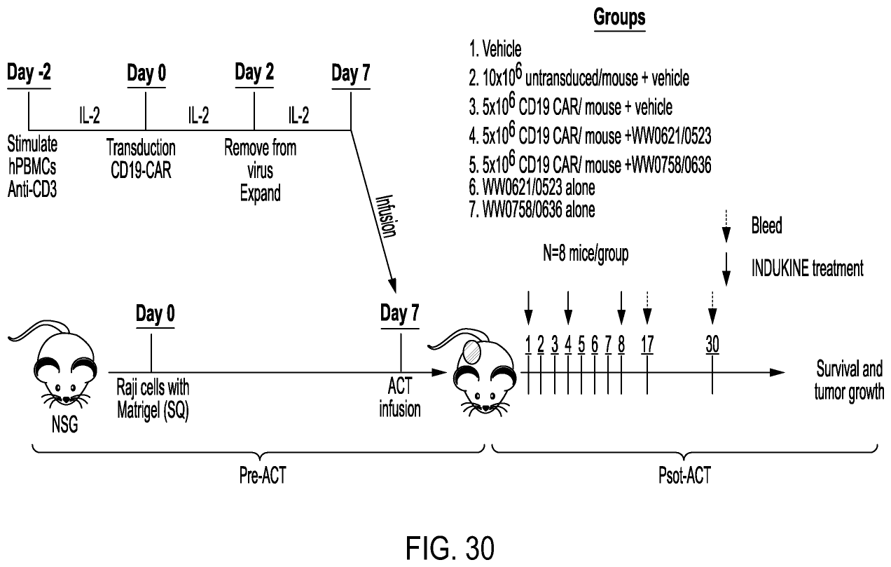

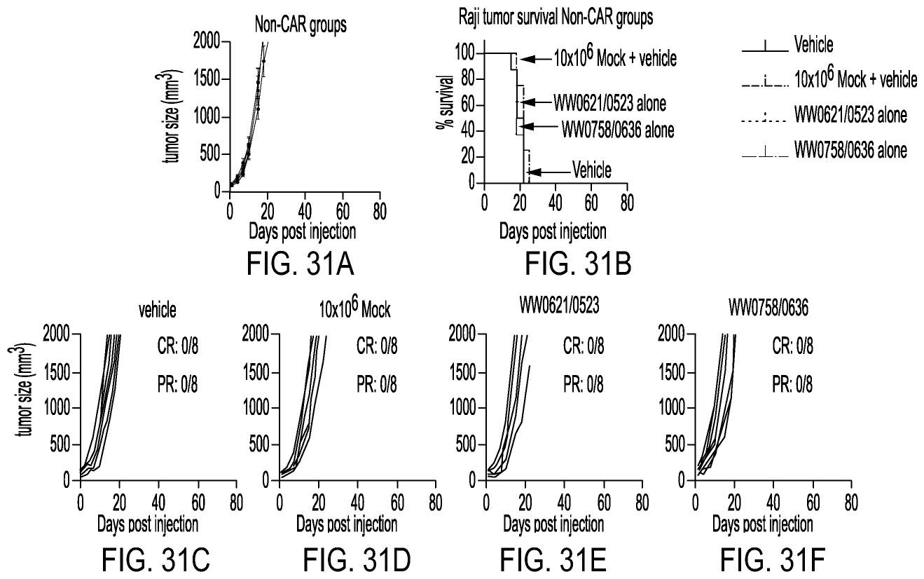

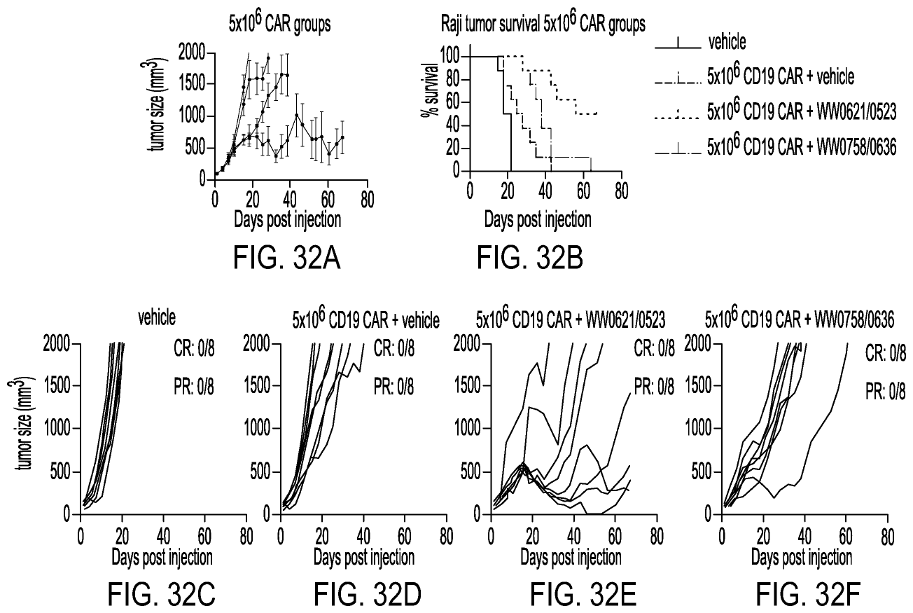

The day before CAR T cell infusion, Raji tumors were measured by digital calipers and mice were randomized to standardize tumor size between the treatment groups. Tumor sizes were 100-120 mm³. CAR T cells were collected from the 24 well plates and washed with PBS to remove culture media. Cells were counted and resuspended at a density of 100×10⁶ cells/mL (10×10⁶ cells/100 μL) or 50×10⁶ cells/mL (5×10⁶ cells/100 μL) in PBS and 100 μL of cells per mouse were infused intravenously into tumor-bearing NSG mice. For treatment protocol, see Table 10 below. After CAR T cell infusion, mice received an ip infusion (100 μL) of vehicle (PBS), inducible IL-2 prodrug (WW0621/0523) (100 μg) or inducible IL-12 prodrug (WW0758/0636) (50 μg). Dosing of prodrugs was conducted with ip injections on days 1, 4 and 8. Mice in the study were monitored biweekly for body weight and tumor size by digital calipers until day 67. The criteria for a complete remission (CR) were two or more consecutive tumor measurements under 13 mm³. A partial response (PR) was a tumor that was 50% of the starting tumor volume. The results are shown in FIGS. 30, 31A-31F, and 32A-32F.

CAR T cell receptor expression was validated by flow cytometry analysis of the pre-infusion product. At day 7 pre-infusion, approximately 50% of the T cells were CD19 CAR+.

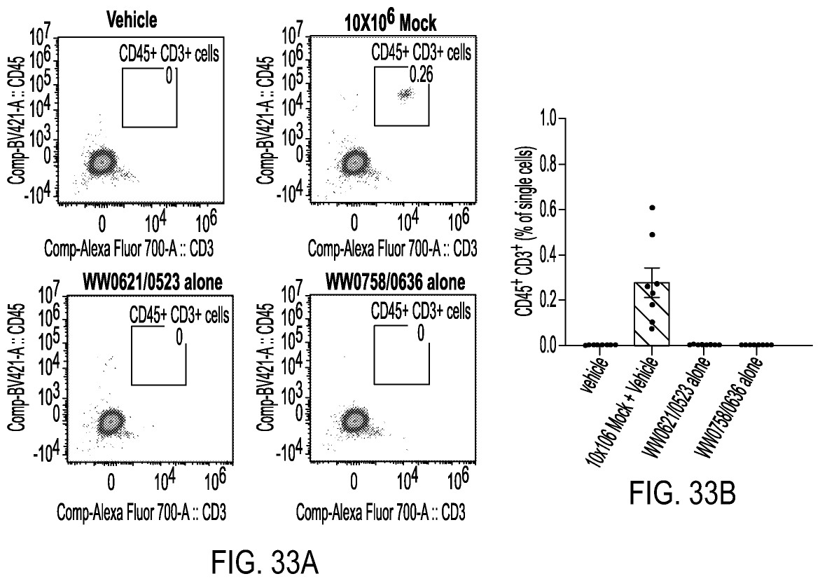

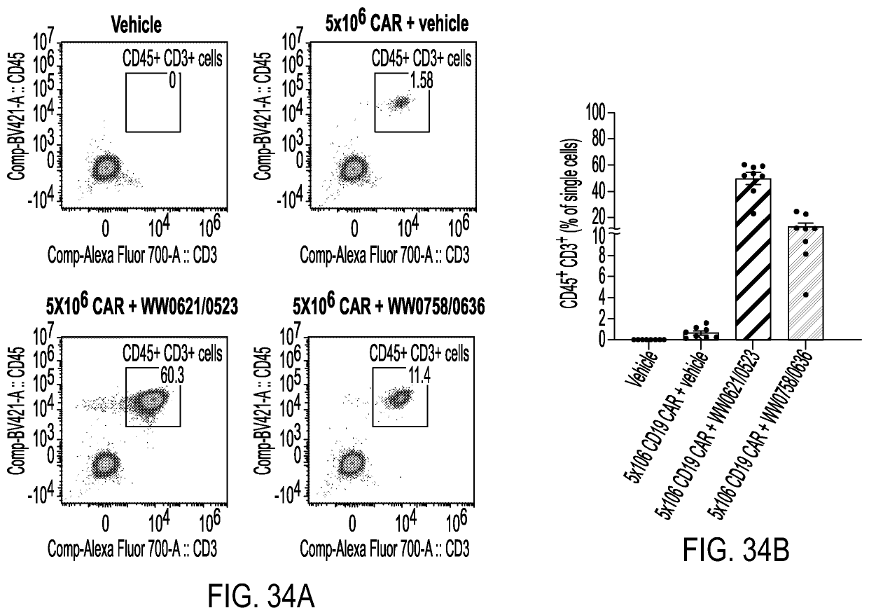

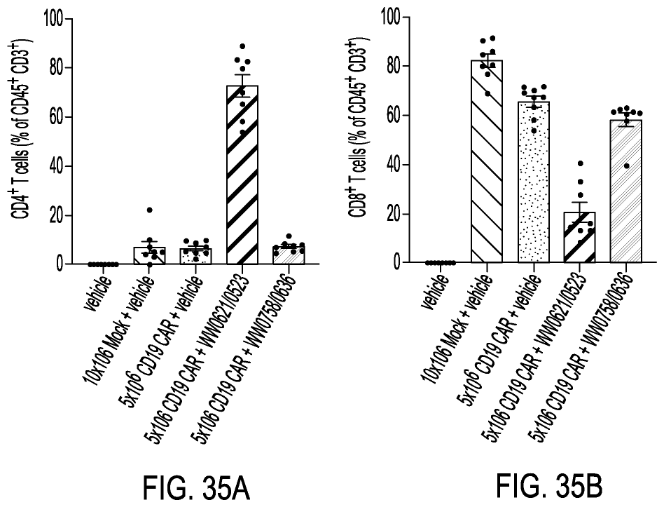

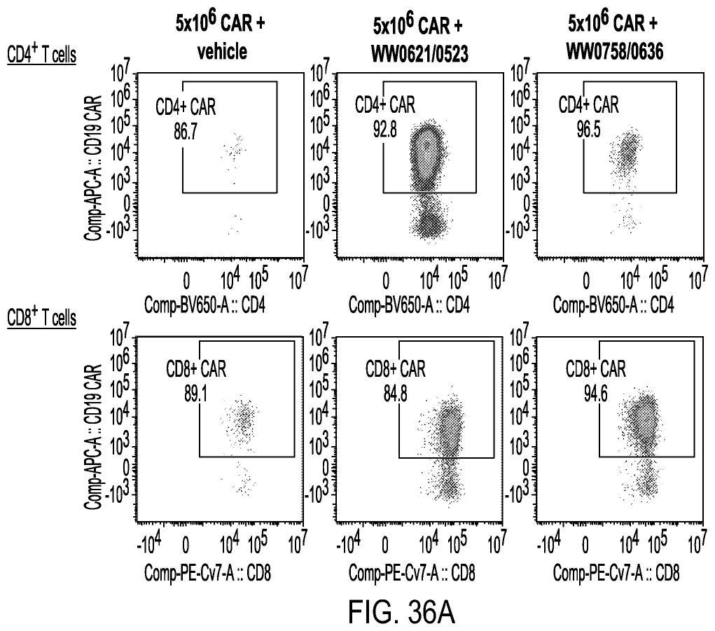

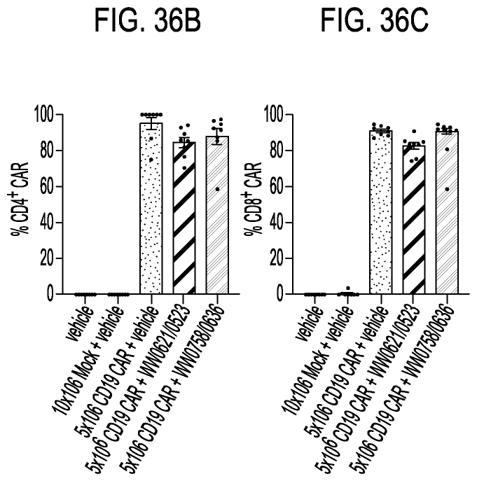

In vivo CAR T cell engraftment and persistence were assessed by flow cytometry of blood collected by submandibular vein bleeds while mice were on study on Day 17 and 30. The results are shown in FIGS. 33A, 33B, 34A, 34B, 35A, 35B, and 36A-36C.

TABLE 10
Treatment Protocol

| Group | N | Agent | Cell Dose |
|---|---|---|---|
| 1 | 8 | Untreated | No cells + no cytokine |
| 2 | 8 | Mock | 10 × 10⁶ cells |
| 3 | 8 | CD19-CAR | 5 × 10⁶ cells |
| 4 | 8 | CD19-CAR + | 5 × 10⁶ cells + 100 μg |
| | | WW0621/0523 | (Day 1, 4, and 8) |
| 5 | 8 | CD19-CAR + | 5 × 10⁶ cells + 50 μg |
| | | WW0758/0636 | (Day 1, 4, and 8) |
| 6 | 8 | WW0621/0523 | no cells + 100 μg |
| | | alone | (Day 1, 4 and 8) |
| 7 | 8 | WW0758/0636 | no cells + 50 μg |
| | | alone | (Day 1, 4 and 8) |

### 7.2 Results

Mice treated with CD19 CAR-T cells and systemic IL-2 or IL-12 prodrug led to increased control of tumor growth and animal survival compared to tumor growth in untreated mice or mice treated with CD19 CAR+ T cells alone in a Burkitt's lymphoma mouse model. IL-2 and IL-12 prodrug administration led to increased engraftment and persistence of the CD19 CAR T cells marked by increased presence in the peripheral blood. The results are shown in FIGS. 30, 31A-31F, 32A-32F and 37A-37C.

### Example 8. CD19 CAR T Cell Therapy with IL-21 Prodrug to Treat NSG Mice Bearing CD19+ Raji Tumors

Twenty-four NOD SCID IL-2Rγ^{-/-} (NSG) mice were subcutaneously implanted with 2×10⁶ Raji tumor cells with 50% Matrigel and tumors were allowed to grow for one week prior to treatment. Human PBMCs were stimulated with 50 ng/mL anti-CD3 (OKT3) and IL-2 for 48 hours prior to lentiviral transduction. Anti-CD3 stimulated PBMCs were lentivirally transduced with a commercial lentivirus expressing a second generation CD19 41BBz CAR and IL-2 for 48 hours. The virus was removed, and CAR T cells were expanded for an additional 5 days prior to infusion into tumor bearing NSG mice.

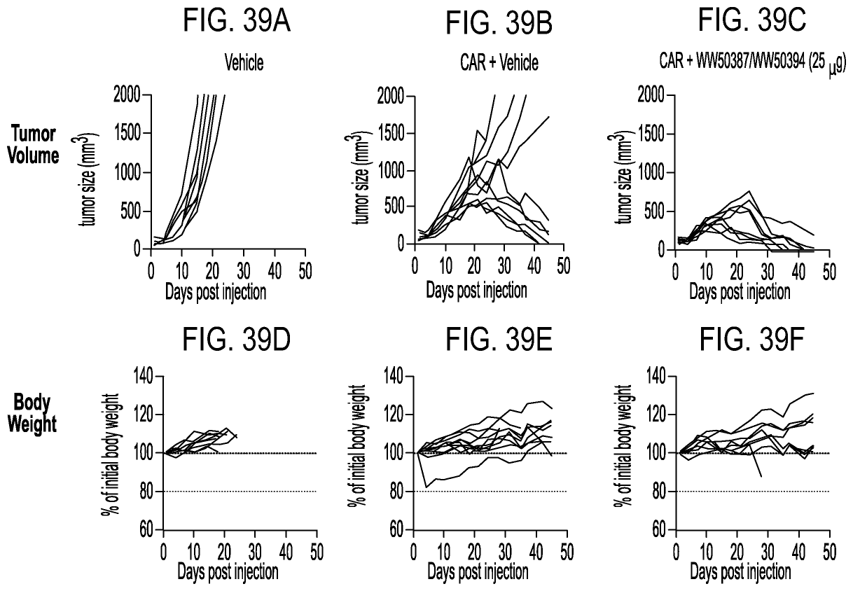

Raji-tumor bearing NSG mice were infused with 5×10⁶ CD19 CAR T cells intravenously through the tail vein. Thirty minutes post infusion, animals received either vehicle (PBS) or 25 ug of WW50387/WW50394 intraperitoneally (Day 1). Animals were treated with vehicle of IL-21 pro-drug on Days 1, 4, 8, 22 and 37 post infusion of the CAR T cells. Tumors and body weight were followed biweekly with a tumor endpoint at 2000 mm³. Results are shown in FIGS. 39A-39C.

### 8. CONSTRUCTS

Exemplary inducible cytokine prodrugs are detailed below in Table 11. While the exemplary inducible cytokine prodrugs contain Linker-2 (GPAGLYAQ, SEQ ID NO: 195), or Linker-3 (ALFKSSFP, SEQ ID NO: 198) or other protease cleavable linkers disclosed herein. For each construct, the disclosed linker can be replaced with either Linker-2 (GPAGLYAQ, SEQ ID NO: 195), or Linker-3 (ALFKSSFP, SEQ ID NO: 198) or other protease cleavable linkers disclosed herein.

The elements of the polypeptide constructs provided in Table 11 contain the abbreviations as follows: “L” refers to a linker. “X” refers to a cleavable linker. Linker 3 refers to a linker that comprises a CTSL-1 substrate motif sequence.

TABLE 11
Exemplary inducible cytokine prodrμg constructs

| Construct # | Construct Description |
|---|---|
| WW0706 | anti-HSA-X-human_p40-L-mouse_p35-XL- |
| | Fab_Lambda_Blocker_(Blocker = Lambda_Fab_R27E_T32D_IGLC2- |
| | 01_X = Linker2) |
| WW0707 | anti-HSA-X-human_p40-L-human_p35-XL- |
| | Fab_Lambda_Blocker_(Blocker = Lambda_Fab_R27E_T32D_IGLC2- |
| | 01_X = Linker2) |
| WW0708 | anti-HSA-X-human_p40-L-mouse_p35-XL- |
| | Fab_Lambda_Blocker_(Blocker = Lambda_Fab_S30E_IGLC2-01_X = Linker2) |
| WW0709 | anti-HSA-X-human_p40-L-human_p35-XL- |
| | Fab_Lambda_Blocker_(Blocker = Lambda_Fab_S30E_IGLC2-01_X = Linker2) |
| WW0710 | anti-HSA-X-human_p40-L-mouse_p35-XL- |
| | Fab_Lambda_Blocker_(Blocker = Lambda_Fab_S30E_N31E_IGLC2- |
| | 01_X = Linker2) |
| WW0711 | anti-HSA-X-human_p40-L-human_p35-XL- |
| | Fab_Lambda_Blocker_(Blocker-Lambda_Fab_S30E_N31E_IGLC2- |
| | 01_X = Linker2) |
| WW0700 | anti-HSA-X-human_p40-L-mouse_p35-XL- |
| | Fab_Lambda_Blocker_(Blocker-Lambda_Fab_N31E_IGLC2-01_X = Linker2) |
| WW0701 | anti-HSA-X-human_p40-L-human_p35-XL- |
| | Fab_Lambda_Blocker_(Blocker-Lambda_Fab_N31E_IGLC2-01_X = Linker2) |
| WW0712 | anti-HSA-X-human_p40-L-mouse_p35-XL- |
| | Fab_Lambda_Blocker_(Blocker-Lambda_Fab_IGLC2-01_X = Linker3) |
| WW0713 | anti-HSA-X-human_p40-L-human_p35-XL- |
| | Fab_Lambda_Blocker_(Blocker-Lambda_Fab_IGLC2-01_X = Linker3) |
| WW0714 | anti-HSA-X-human_p40-L-mouse_p35-XL- |
| | Fab_Lambda_Blocker_(Blocker-Lambda_Fab_N31E_IGLC2-01_X = Linker3) |
| WW0715 | anti-HSA-X-human_p40-L-human_p35-XL- |
| | Fab_Lambda_Blocker_(Blocker-Lambda_Fab_N31E_IGLC2-01_X = Linker3) |
| WW0805 | human_p35-X-anti-HSA-L-Blocker_(Blocker = Opt1_Hv_D53E_D61E_Vl- |
| | Vh_X = Linker3) |
| WW0754 | anti-HSA-X-Human_p35-XL- |
| | Fab_Lambda_Blocker_(Blocker-Lambda_Fab_IGLC2-01_X = Linker2) |
| WW0756 | anti-HSA-X-Human_p35-XL- |
| | Fab_Lambda_Blocker_(Blocker-Lambda_Fab_S30D_N31E_IGLC2- |
| | 01_X = Linker2) |
| WW0762 | anti-HSA-X-Human_p35-XL- |
| | Fab_Lambda_Blocker_(Blocker = Lambda_Fab_IGLC2-01_X = Linker3) |
| WW0770 | human_p40-L-human_p35-X-anti-HSA-L- |
| | Fab_Lambda_Blocker_(Blocker = Lambda_Fab_IGLC2-01_X = Linker2) |
| WW0636 | Human_IL12B_(p40) |
| WW0727 | Fab_Heavy_Blocker_(Blocker = IL-12_Heavy_Fab_D53E_D61E_IgG1_Fab) |
| WW0045 | Blocker2-Linker-(cleav. link.)-IL2-(cleav. link.)-(anti-HSA)-6xHis |
| WW0046 | (anti-HSA)-(cleav. link.)-Blocker2-Linker-(cleav. link.)-IL2-6xHis |
| WW0203 | IL2-(cleav. link.)-(anti-HSA)-linker(cleav. link.)-blocker2 |
| WW0204 | IL2-X-anti-HSA-LX-blocker_(X = Linker4_Blocker = Vh-Vl) |
| WW0205 | IL2-X-anti-HSA-LX-blocker_(X = Linker5_Blocker = Vh-Vl) |
| WW0234 | IL2-X-HSA-LX-blocker_(X = Linker 1; Blocker = 3TOW69) |
| WW0235 | IL2-X-HSA-LX-blocker_(X = Linker 1; Blocker = 3TOW85) |
| WW0236 | IL2-X-HSA-LX-blocker_(X = Linker 1; Blocker = 2TOW91) |
| WW0308 | IL2-X-HSA-LX-blocker(QAPRL_FR2 (“QAPRL” disclosed as SEQ ID NO: |
| | 710))_(X = Linker 1; Blocker = Vh/Vl) |
| WW0415 | IL2-X-anti-HSA-LX-blocker_(Blocker = VHVL.F2.high. A02_Vh/Vl_A46S; |
| | X = Linker 2) |
| WW0621 | IL2-X-anti-HSA-LX-Heavy_blocker_Fab_(Blocker = VH-CH1; X = Linker 3) |
| WW0520 | IL2-X-anti-HSA-LX-Heavy_blocker_Fab_(Blocker = VH-CH1; X = Linker 2) |
| WW0735 | IL2-X-anti-HSA-LL-Heavy_blocker_Fab_(Blocker = VH-CH1_X = Linker2) |
| WW0736 | IL2-X-anti-HSA-LL-Heavy_blocker_Fab_(Blocker = VH-CHI_X = Linker3) |
| WW0523 | Kappa_blocker_Fab_(Blocker = VHVL.F2.high. A02_A46S_Kappa) |
| WW50387 | hIL21(1stQ)-LXL-HSA-X-Fab_Heavy_Blocker (Blocker = ADI-76956_ADI- |
| | 76957_ADI-76958_X = Linker_3_L = G4S_GGS |
| WW50394 | Kappa blocker Fab |

9. SEQUENCE DISCLOSURE

| SEQ | | |
|---|---|---|
| ID NO. | Name | Sequence |
| 42 | Human IL-2 | MYRMQLLSCI ALSLALVTNS APTSSSTKKT |
| | | QLQLEHLLLD LQMILNGINN YKNPKLTRML |
| | | TFKFYMPKKA TELKHLQCLE EELKPLEEVL |
| | | NLAQSKNFHLRPRDLISNIN VIVLELKGSE |
| | | TTFMCEYADE TATIVEFLNR |
| | | WITFCQSIISTLT |
| 43 | Human serum | MKWVTFISLLFLFSSAYSRG VFRRDAHKSE |
| | albumin | VAHRFKDLGE ENFKALVLIA FAQYLQQCPF |
| | | EDHVKLVNEV TEFAKTCVAD ESAENCDKSL |
| | | HTLFGDKLCT VATLRETYGE MADCCAKQEP |
| | | ERNECFLQHK DDNPNLPRLV RPEVDVMCTA |
| | | FHDNEETFLK KYLYEIARRH PYFYAPELLF |
| | | FAKRYKAAFT ECCQAADKAA CLLPKLDELR |
| | | DEGKASSAKQ GLKCASLQKF |
| | | GERAFKAWAV ARLSQRFPKA EFAEVSKLVT |
| | | DLTKVHTECC HGDLLECADD RADLAKYICE |
| | | NQDSISSKLK ECCEKPLLEK SHCIAEVEND |
| | | EMPADLPSLA ADFVGSKDVC |
| | | KNYAEAKDVF LGMFLYEYAR |
| | | RHPDYSVVLLLRLAKTYETT LEKCCAAADP |
| | | HECYAKVFDE FKPLVEEPQN LIKQNCELFE |
| | | QLGEYKFQNA LLVRYTKKVP QVSTPTLVEV |
| | | SRNLGKVGSK CCKHPEAKRM PCAEDCLSVF |
| | | LNQLCVLHEK |
| | | TPVSDRVTKCCTESLVNGRPCFSALEVDETY |
| | | VPKEFNAETFTFHADICTLSEKERQIKKQTAL |
| | | VELVKHK |
| | | PKATKEQLKAVMDDFAAFVEKCCKADDKET |
| | | CFAEEGKKLVAASQAALGL |
| 192 | Blocker 2 | mdmrvpaqllgllllwlrgarcEVQLVESGGGLVQPGG |
| | (IL2 blocker) | SLRLSCAASGFTFSSYTLAWVRQAPGKGLEW |
| | | VAAIDSSSYTYSPDTVRGRFTISRDNAKNSLY |
| | | LQMNSLRAEDTAVYYCARDSNWDALDYWG |
| | | QGTTVTVSSggggsggggsggggsDIQMTQSPSSLS |
| | | ASVGDRVTITCKASQNVGTNVGWYQQKPGK |
| | | APKALIYSASFRYSGVPSRFSGSGSGTDFTLTI |
| | | SSLQPEDFATYYCQQYYTYPYTFGGGTKVEI |
| | | KHHHHHH |
| 193 | Blocker 12 (IL-12 | mdmrvpaqllgllllwlrgarcQSVLTQPPSVSGAPGQR |
| | blocker) | VTISCSGSRSNIGSNTVKWYQQLPGTAPKLLI |
| | | YYNDQRPSGVPDRFSGSKSGTSASLAITGLQA |
| | | EDEADYYCQSYDRYTHPALLFGTGTKVTVLg |
| | | gggsggggsggggsQVQLVESGGGVVQPGRSLRLS |
| | | CAASGFTFSSYGMHWVRQAPGKGLEWVAFI |
| | | RYDGSNKYYADSVKGRFTISRDNSKNTLYLQ |
| | | MNSLRAEDTAVYYCKTHGSHDNWGQGTMV |
| | | TVSSHHHHHH |
| 258 | MMP14 substrate | GPLGLKAQ |
| | motif sequence | |
| 259 | MMP14 substrate | LPLGLKAQ |
| | motif sequence | |
| 260 | MMP14 substrate | SPLGLKAQ |
| | motif sequence | |
| 261 | MMP14 substrate | QPLGLKAQ |
| | motif sequence | |
| 262 | MMP14 substrate | KPLGLKAQ |
| | motif sequence | |
| 263 | MMP14 substrate | FPLGLKAQ |
| | motif sequence | |
| 264 | MMP14 substrate | HPLGLKAQ |
| | motif sequence | |
| 265 | MMP14 substrate | PPLGLKAQ |
| | motif sequence | |
| 266 | MMP14 substrate | APLGLKAQ |
| | motif sequence | |
| 267 | MMP14 substrate | DPLGLKAQ |
| | motif sequence | |
| 268 | MMP14 substrate | GPHGLKAQ |
| | motif sequence | |
| 269 | MMP14 substrate | GPSGLKAQ |
| | motif sequence | |
| 270 | MMP14 substrate | GPQGLKAQ |
| | motif sequence | |
| 271 | MMP14 substrate | GPPGLKAQ |
| | motif sequence | |
| 272 | MMP14 substrate | GPEGLKAQ |
| | motif sequence | |
| 273 | MMP14 substrate | GPFGLKAQ |
| | motif sequence | |
| 274 | MMP14 substrate | GPRGLKAQ |
| | motif sequence | |
| 275 | MMP14 substrate | GPGGLKAQ |
| | motif sequence | |
| 276 | MMP14 substrate | GPAGLKAQ |
| | motif sequence | |
| 277 | MMP14 substrate | LPAGLKGA |
| | motif sequence | |
| 195 | MMP14 substrate | GPAGLYAQ |
| | motif sequence | |
| 278 | MMP14 substrate | GPANLVAQ |
| | motif sequence | |
| 279 | MMP14 substrate | GPAALVGA |
| | motif sequence | |
| 280 | MMP14 substrate | GPANLRAQ |
| | motif sequence | |
| 281 | MMP14 substrate | GPAGLRAQ |
| | motif sequence | |
| 282 | MMP14 substrate | GPAGLVAQ |
| | motif sequence | |
| 283 | MMP14 substrate | GPAGLRGA |
| | motif sequence | |
| 284 | MMP14 substrate | LPAGLVGA |
| | motif sequence | |
| 285 | MMP14 substrate | GPAGLKGA |
| | motif sequence | |
| 286 | MMP14 substrate | GPLALKAQ |
| | motif sequence | |
| 287 | MMP14 substrate | GPLNLKAQ |
| | motif sequence | |
| 288 | MMP14 substrate | GPLHLKAQ |
| | motif sequence | |
| 289 | MMP14 substrate | GPLYLKAQ |
| | motif sequence | |
| 290 | MMP14 substrate | GPLPLKAQ |
| | motif sequence | |
| 291 | MMP14 substrate | GPLELKAQ |
| | motif sequence | |
| 292 | MMP14 substrate | GPLRLKAQ |
| | motif sequence | |
| 293 | MMP14 substrate | GPLLLKAQ |
| | motif sequence | |
| 294 | MMP14 substrate | GPLSLKAQ |
| | motif sequence | |
| 295 | MMP14 substrate | GPLGLYAQ |
| | motif sequence | |
| 296 | MMP14 substrate | GPLGLFAQ |
| | motif sequence | |
| 297 | MMP14 substrate | GPLGLLAQ |
| | motif sequence | |
| 298 | MMP14 substrate | GPLGLHAQ |
| | motif sequence | |
| 299 | MMP14 substrate | GPLGLRAQ |
| | motif sequence | |
| 300 | MMP14 substrate | GPLGLAAQ |
| | motif sequence | |
| 301 | MMP14 substrate | GPLGLEAQ |
| | motif sequence | |
| 302 | MMP14 substrate | GPLGLGAQ |
| | motif sequence | |
| 303 | MMP14 substrate | GPLGLPAQ |
| | motif sequence | |
| 304 | MMP14 substrate | GPLGLQAQ |
| | motif sequence | |
| 305 | MMP14 substrate | GPLGLSAQ |
| | motif sequence | |
| 306 | MMP14 substrate | GPLGLVAQ |
| | motif sequence | |
| 307 | MMP14 substrate | GPLGLKLQ |
| | motif sequence | |
| 308 | MMP14 substrate | GPLGLKFQ |
| | motif sequence | |
| 309 | MMP14 substrate | GPLGLKEQ |
| | motif sequence | |
| 310 | MMP14 substrate | GPLGLKKQ |
| | motif sequence | |
| 311 | MMP14 substrate | GPLGLKQQ |
| | motif sequence | |
| 312 | MMP14 substrate | GPLGLKSQ |
| | motif sequence | |
| 313 | MMP14 substrate | GPLGLKGQ |
| | motif sequence | |
| 314 | MMP14 substrate | GPLGLKHQ |
| | motif sequence | |
| 315 | MMP14 substrate | GPLGLKPQ |
| | motif sequence | |
| 316 | MMP14 substrate | GPLGLKAG |
| | motif sequence | |
| 317 | MMP14 substrate | GPLGLKAF |
| | motif sequence | |
| 318 | MMP14 substrate | GPLGLKAP |
| | motif sequence | |
| 319 | MMP14 substrate | GPLGLKAL |
| | motif sequence | |
| 320 | MMP14 substrate | GPLGLKAE |
| | motif sequence | |
| 321 | MMP14 substrate | GPLGLKAA |
| | motif sequence | |
| 322 | MMP14 substrate | GPLGLKAH |
| | motif sequence | |
| 323 | MMP14 substrate | GPLGLKAK |
| | motif sequence | |
| 324 | MMP14 substrate | GPLGLKAS |
| | motif sequence | |
| 325 | MMP14 substrate | GPLGLFGA |
| | motif sequence | |
| 326 | MMP14 substrate | GPLGLQGA |
| | motif sequence | |
| 327 | MMP14 substrate | GPLGLVGA |
| | motif sequence | |
| 328 | MMP14 substrate | GPLGLAGA |
| | motif sequence | |
| 329 | MMP14 substrate | GPLGLLGA |
| | motif sequence | |
| 330 | MMP14 substrate | GPLGLRGA |
| | motif sequence | |
| 331 | MMP14 substrate | GPLGLYGA |
| | motif sequence | |
| 332 | CTSL1 substrate | ALFKSSPP |
| | motif sequence | |
| 333 | CTSL1 substrate | SPFRSSRQ |
| | motif sequence | |
| 334 | CTSL1 substrate | KLFKSSPP |
| | motif sequence | |
| 335 | CTSL1 substrate | HLFKSSPP |
| | motif sequence | |
| 336 | CTSL1 substrate | SLFKSSPP |
| | motif sequence | |
| 337 | CTSL1 substrate | QLFKSSPP |
| | motif sequence | |
| 338 | CTSL1 substrate | LLFKSSPP |
| | motif sequence | |
| 339 | CTSL1 substrate | PLFKSSPP |
| | motif sequence | |
| 340 | CTSL1 substrate | FLFKSSPP |
| | motif sequence | |
| 341 | CTSL1 substrate | GLFKSSPP |
| | motif sequence | |
| 342 | CTSL1 substrate | VLFKSSPP |
| | motif sequence | |
| 343 | CTSL1 substrate | ELFKSSPP |
| | motif sequence | |
| 344 | CTSL1 substrate | AKFKSSPP |
| | motif sequence | |
| 345 | CTSL1 substrate | AHFKSSPP |
| | motif sequence | |
| 346 | CTSL1 substrate | AGFKSSPP |
| | motif sequence | |
| 347 | CTSL1 substrate | APFKSSPP |
| | motif sequence | |
| 348 | CTSL1 substrate | ANFKSSPP |
| | motif sequence | |
| 349 | CTSL1 substrate | AFFKSSPP |
| | motif sequence | |
| 350 | CTSL1 substrate | AAFKSSPP |
| | motif sequence | |
| 351 | CTSL1 substrate | ASFKSSPP |
| | motif sequence | |
| 352 | CTSL1 substrate | AEFKSSPP |
| | motif sequence | |
| 353 | CTSL1 substrate | ALRKSSPP |
| | motif sequence | |
| 354 | CTSL1 substrate | ALLKSSPP |
| | motif sequence | |
| 355 | CTSL1 substrate | ALAKSSPP |
| | motif sequence | |
| 356 | CTSL1 substrate | ALQKSSPP |
| | motif sequence | |
| 357 | CTSL1 substrate | ALHKSSPP |
| | motif sequence | |
| 358 | CTSL1 substrate | ALPKSSPP |
| | motif sequence | |
| 359 | CTSL1 substrate | ALTKSSPP |
| | motif sequence | |
| 360 | CTSL1 substrate | ALGKSSPP |
| | motif sequence | |
| 361 | CTSL1 substrate | ALDKSSPP |
| | motif sequence | |
| 199 | CTSL1 substrate | ALFFSSPP |
| | motif sequence | |
| 362 | CTSL1 substrate | ALFHSSPP |
| | motif sequence | |
| 363 | CTSL1 substrate | ALFTSSPP |
| | motif sequence | |
| 364 | CTSL1 substrate | ALFASSPP |
| | motif sequence | |
| 365 | CTSL1 substrate | ALFQSSPP |
| | motif sequence | |
| 366 | CTSL1 substrate | ALFLSSPP |
| | motif sequence | |
| 367 | CTSL1 substrate | ALFGSSPP |
| | motif sequence | |
| 368 | CTSL1 substrate | ALFESSPP |
| | motif sequence | |
| 369 | CTSL1 substrate | ALFPSSPP |
| | motif sequence | |
| 370 | CTSL1 substrate | ALFKHSPP |
| | motif sequence | |
| 371 | CTSL1 substrate | ALFKLSPP |
| | motif sequence | |
| 372 | CTSL1 substrate | ALFKKSPP |
| | motif sequence | |
| 373 | CTSL1 substrate | ALFKASPP |
| | motif sequence | |
| 374 | CTSL1 substrate | ALFKISPP |
| | motif sequence | |
| 375 | CTSL1 substrate | ALFKGSPP |
| | motif sequence | |
| 376 | CTSL1 substrate | ALFKNSPP |
| | motif sequence | |
| 377 | CTSL1 substrate | ALFKRSPP |
| | motif sequence | |
| 378 | CTSL1 substrate | ALFKESPP |
| | motif sequence | |
| 379 | CTSL1 substrate | ALFKFSPP |
| | motif sequence | |
| 380 | CTSL1 substrate | ALFKPSPP |
| | motif sequence | |
| 381 | CTSL1 substrate | ALFKSFPP |
| | motif sequence | |
| 382 | CTSL1 substrate | ALFKSLPP |
| | motif sequence | |
| 383 | CTSL1 substrate | ALFKSIPP |
| | motif sequence | |
| 384 | CTSL1 substrate | ALFKSKPP |
| | motif sequence | |
| 385 | CTSL1 substrate | ALFKSAPP |
| | motif sequence | |
| 386 | CTSL1 substrate | ALFKSQPP |
| | motif sequence | |
| 387 | CTSL1 substrate | ALFKSPPP |
| | motif sequence | |
| 388 | CTSL1 substrate | ALFKSEPP |
| | motif sequence | |
| 389 | CTSL1 substrate | ALFKSGPP |
| | motif sequence | |
| 198 | CTSL1 substrate | ALFKSSFP |
| | motif sequence | |
| 390 | CTSL1 substrate | ALFKSSLP |
| | motif sequence | |
| 391 | CTSL1 substrate | ALFKSSGP |
| | motif sequence | |
| 392 | CTSL1 substrate | ALFKSSSP |
| | motif sequence | |
| 393 | CTSL1 substrate | ALFKSSVP |
| | motif sequence | |
| 394 | CTSL1 substrate | ALFKSSHP |
| | motif sequence | |
| 395 | CTSL1 substrate | ALFKSSAP |
| | motif sequence | |
| 396 | CTSL1 substrate | ALFKSSNP |
| | motif sequence | |
| 397 | CTSL1 substrate | ALFKSSKP |
| | motif sequence | |
| 398 | CTSL1 substrate | ALFKSSEP |
| | motif sequence | |
| 399 | CTSL1 substrate | ALFKSSPF |
| | motif sequence | |
| 400 | CTSL1 substrate | ALFKSSPH |
| | motif sequence | |
| 401 | CTSL1 substrate | ALFKSSPG |
| | motif sequence | |
| 402 | CTSL1 substrate | ALFKSSPA |
| | motif sequence | |
| 403 | CTSL1 substrate | ALFKSSPS |
| | motif sequence | |
| 404 | CTSL1 substrate | ALFKSSPV |
| | motif sequence | |
| 405 | CTSL1 substrate | ALFKSSPQ |
| | motif sequence | |
| 406 | CTSL1 substrate | ALFKSSPK |
| | motif sequence | |
| 407 | CTSL1 substrate | ALFKSSPL |
| | motif sequence | |
| 408 | CTSL1 substrate | ALFKSSPD |
| | motif sequence | |

| SEQ ID | | |
|---|---|---|
| NO. | Name | Sequence |
| 1 | WW0621 | APTSSSTKKTQLQLEHLLLDLQMILNGINNYKNPKLTRMLTFKFYMPK |
| | (Heterodimeric | KATELKHLQCLEEELKPLEEVLNLAQSKNFHLRPRDLISNINVIVLELK |
| | inducible IL-2 | GSETTFMCEYADETATIVEFLNRWITFCQSIISTLTSGGPALFKSSFPPGS |
| | prodrug) | EVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVY |
| | | YCTIGGSLSVSSQGTLVTVSSGGGGSGGGGSGGGGSGGGGSGGGGSG |
| | | GGGSSGGPALFKSSFPPGSEVQLVESGGGLVQPGGSLRLSCAASGFTFS |
| | | SYTLAWVRQAPGKGLEWVAAIDSSSYTYSPDTVRGRFTISRDNAKNS |
| | | LYLQMNSLRAEDTAVYYCARDSNWDALDYWGQGTTVTVSSASTKG |
| | | PSVFPLAPSSKSTSGGTAALGCLVKDYFPEPVTVSWNSGALTSGVHTF |
| | | PAVLQSSGLYSLSSVVTVPSSSLGTQTYICNVNHKPSNTKVDKRVEPK |
| | | SC** |
| 2 | WW0520 | APTSSSTKKTQLQLEHLLLDLQMILNGINNYKNPKLTRMLTFKFYMPK |
| | (Heterodimeric | KATELKHLQCLEEELKPLEEVLNLAQSKNFHLRPRDLISNINVIVLELK |
| | inducible IL-2 | GSETTFMCEYADETATIVEFLNRWITFCQSIISTLTSGGPGPAGLYAQP |
| | prodrug) | GSEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAV |
| | | YYCTIGGSLSVSSQGTLVTVSSGGGGSGGGGSGGGGSGGGGSGGGGS |
| | | GGGGSSGGPGPAGLYAQPGSEVQLVESGGGLVQPGGSLRLSCAASGF |
| | | TFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSAST |
| | | KGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEP |
| | | KSC** |
| 3 | WW0735 | APTSSSTKKTQLQLEHLLLDLQMILNGINNYKNPKLTRMLTFKFYMPK |
| | (Heterodimeric | KATELKHLQCLEEELKPLEEVLNLAQSKNFHLRPRDLISNINVIVLELK |
| | inducible IL-2 | GSETTFMCEYADETATIVEFLNRWITFCQSIISTLTSGGPGPAGLYAQP |
| | prodrug) | GSEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAV |
| | | YYCTIGGSLSVSSQGTLVTVSSGGGGSGGGGSGGGGSGGGGSGGGGS |
| | | GGGGSGGGGSGGGGSGGGGSEVQLVESGGGLVQPGGSLRLSCAASG |
| | | FTFSSYTLAWVRQAPGKGLEWVAAIDSSSYTYSPDTVRGRFTISRDNA |
| | | KNSLYLQMNSLRAEDTAVYYCARDSNWDALDYWGQGTTVTVSSAS |
| | | TKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKRV |
| | | EPKSC |
| 4 | WW0736 | APTSSSTKKTQLQLEHLLLDLQMILNGINNYKNPKLTRMLTFKFYMPK |
| | (Heterodimeric | KATELKHLQCLEEELKPLEEVLNLAQSKNFHLRPRDLISNINVIVLELK |
| | inducible IL-2 | GSETTFMCEYADETATIVEFLNRWITFCQSIISTLTSGGPALFKSSFPPGS |
| | prodrug) | EVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVY |
| | | YCTIGGSLSVSSQGTLVTVSSGGGGSGGGGSGGGGSGGGGSGGGGSG |
| | | GGGSGGGGSGGGGSGGGGSEVQLVESGGGLVQPGGSLRLSCAASGFT |
| | | FSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSAST |
| | | KGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKRVEP |
| | | KSC |
| 5 | WW0523 | DIQMTQSPSSLSASVGDRVTITCKAREKLWSAVAWYQQKPGKAPKSL |
| | Kappa_blocker_Fab_ | IYSASFRYSGVPSRFSGSGSGTDFTLTISSLQPEDFATYYCQQYYTYPY |
| | (Blocker = | TFGGGTKVEIKRTVAAPSVFIFPPSDEQLKSGTASVVCLLNNFYPREAK |
| | VHVL.F2.high. | VQWKVDNALQSGNSQESVTEQDSKDSTYSLSSTLTLSKADYEKHKVY |
| | A02_A46S_Kappa) | ACEVTHQGLSSPVTKSFNRGEC |
| 6 | WW0758 | EVQLVESGGGLVQPGNSLRLSCAASGFTFSKFGMSWVRQAPG |
| | Heterodimeric IL- | KGLEWVSSISGSGRDTLYAESVKGRFTISRDNAKTTLYLQMNS |
| | 12 polypeptide, | LRPEDTAVYYCTIGGSLSVSSQGTLVTVSSsggpALFKSSFPpgsrnl |
| | anti-HSA sdAb, | pvatpdpgmfpclhhsqnllravsnmlqkarqtlefypctseeidheditkdktstveaclpleltk |
| | scFv Blocker, 2 | nesclnsretsfitngsclasrktsfmmalclssiyedlkmyqvefktmnakllmdpkrqifldqn |
| | cleavage sites | mlavidelmqalnfnsetvpqkssleepdfyktkiklcillhafriravtidrvmsylnassggpA |
| | | LFKSSFPpgsggggsggggsggggsggggsggggsggggsQSVLTQPPSVSGA |
| | | PGQRVTISCSGSRSNIGSNTVKWYQQLPGTAPKLLIYYNDQRPS |
| | | GVPDRFSGSKSGTSASLAITGLQAEDEADYYCQSYDRYTHPAL |
| | | LFGTGTKVTVLggggsggggsggggsQVQLVESGGGVVQPGRSLRLS |
| | | CAASGFTFSSYGMHWVRQAPGKGLEWVAFIRYeGSNKYYAeS |
| | | VKGRFTISRDNSKNTLYLQMNSLRAEDTAVYYCKTHGSHDNW |
| | | GQGTMVTVSS** |
| 7 | WW0805 | rnlpvatpdpgmfpclhhsqnllravsnmlqkarqtlefypctseeidheditkdktstveaclple |
| | Heterodimeric IL- | ltknesclnsretsfitngsclasrktsfmmalclssiyedlkmyqvefktmnakllmdpkrqifld |
| | 12 polypeptide, | qnmlavidelmqalnfnsetvpqkssleepdfyktkiklcillhafriravtidrvmsylnassggp |
| | anti-HSA sdAb, | ALFKSSFPpgsEVQLVESGGGLVQPGNSLRLSCAASGFTFSKFGM |
| | scFv Blocker, 1 | SWVRQAPGKGLEWVSSISGSGRDTLYAESVKGRFTISRDNAKT |
| | cleavage site | TLYLQMNSLRPEDTAVYYCTIGGSLSVSSQGTLVTVSSggggsggg |
| | | gsggggggggsggggsggggsQSVLTQPPSVSGAPGQRVTISCSGSRSNI |
| | | GSNTVKWYQQLPGTAPKLLIYYNDQRPSGVPDRFSGSKSGTSA |
| | | SLAITGLQAEDEADYYCQSYDRYTHPALLFGTGTKVTVLggggsg |
| | | gggsggggsQVQLVESGGGVVQPGRSLRLSCAASGFTFSSYGMHW |
| | | VRQAPGKGLEWVAFIRYeGSNKYYAeSVKGRFTISRDNSKNTL |
| | | YLQMNSLRAEDTAVYYCKTHGSHDNWGQGTMVTVSS** |
| 8 | WW0754 | EVQLVESGGGLVQPGNSLRLSCAASGFTFSKFGMSWVRQAPG |
| | Heterodimeric IL- | KGLEWVSSISGSGRDTLYAESVKGRFTISRDNAKTTLYLQMNS |
| | 12 polypeptide, | LRPEDTAVYYCTIGGSLSVSSQGTLVTVSSsggpGPAGLYAQpgsr |
| | anti-HSA sdAb, | nlpvatpdpgmfpclhhsqnllravsnmlqkarqtlefypctseeidheditkdktstveaclplel |
| | Fab Blocker, 2 | tknesclnsretsfitngsclasrktsfmmalclssiyedlkmyqvefktmnakllmdpkrqifld |
| | cleavage sites | qnmlavidelmqalnfnsetvpqkssleepdfyktkiklcillhafriravtidrvmsylnassggp |
| | | GPAGLYAQpgsggggggggsggggsggggsggggsggggsQSVLTQPPSVS |
| | | GAPGQRVTISCSGSRSNIGSNTVKWYQQLPGTAPKLLIYYNDQR |
| | | PSGVPDRFSGSKSGTSASLAITGLQAEDEADYYCQSYDRYTHPA |
| | | LLFGTGTKVTVLgqpkaapsvtlfppsseelqankatlvclisdfypgavtvawkadss |
| | | pvkagvetttpskqsnnkyaassylsltpeqwkshrsyscqvthegstvektvaptecs** |
| 9 | WW0756 | EVQLVESGGGLVQPGNSLRLSCAASGFTFSKFGMSWVRQAPG |
| | Heterodimeric IL- | KGLEWVSSISGSGRDTLYAESVKGRFTISRDNAKTTLYLQMNS |
| | 12 polypeptide, | LRPEDTAVYYCTIGGSLSVSSQGTLVTVSSsggpGPAGLYAQpgsr |
| | anti-HSA sdAb, | nlpvatpdpgmfpclhhsqnllravsnmlqkarqtlefypctseeidheditkdktstveaclplel |
| | Fab Blocker, 2 | tknescInsretsfitngsclasrktsfmmalclssiyedlkmyqvefktmnakllmdpkrqifld |
| | cleavage sites | qnmlavidelmqalnfnsetvpqkssleepdfyktkiklcillhafriravtidrvmsylnassggp |
| | | GPAGLYAQpgsggggsggggsggggggggsggggsggggsQSVLTQPPSVS |
| | | GAPGQRVTISCSGSRSNIGdeTVKWYQQLPGTAPKLLIYYNDQR |
| | | PSGVPDRFSGSKSGTSASLAITGLQAEDEADYYCQSYDRYTHPA |
| | | LLFGTGTKVTVLgqpkaapsvtlfppsseelqankatlvclisdfypgavtvawkadss |
| | | pvkagvetttpskqsnnkyaassylsltpeqwkshrsyscqvthegstvektvaptecs** |
| 10 | WW0762 | EVQLVESGGGLVQPGNSLRLSCAASGFTFSKFGMSWVRQAPG |
| | Heterodimeric IL- | KGLEWVSSISGSGRDTLYAESVKGRFTISRDNAKTTLYLQMNS |
| | 12 polypeptide, | LRPEDTAVYYCTIGGSLSVSSQGTLVTVSSsggpALFKSSFPpgsrnl |
| | anti-HSA sdAb, | pvatpdpgmfpclhhsqnllravsnmlqkarqtlefypctseeidheditkdktstveaclpleltk |
| | Fab Blocker, 2 | nesclnsretsfitngsclasrktsfmmalclssiyedlkmyqvefktmnakllmdpkrqifldqn |
| | cleavage sites | mlavidelmqalnfnsetvpqkssleepdfyktkiklcillhafriravtidrvmsylnassggpA |
| | | LFKSSFPpgsggggggggsggggsggggsggggsggggsQSVLTQPPSVSGA |
| | | PGQRVTISCSGSRSNIGSNTVKWYQQLPGTAPKLLIYYNDQRPS |
| | | GVPDRFSGSKSGTSASLAITGLQAEDEADYYCQSYDRYTHPAL |
| | | LFGTGTKVTVLgqpkaapsvtlfppsseelqankatlvclisdfypgavtvawkadsspv |
| | | kagvetttpskqsnnkyaassylsltpeqwkshrsyscqvthegstvektvaptecs** |
| 11 | WW0770 | iwelkkdvyvveldwypdapgemvvltcdtpeedgitwtldqssevlgsgktltiqvkefgdag |
| | Monomeric IL-12 | qytchkggevlshsllllhkkedgiwstdilkdqkepknktflrceaknysgrftcwwlttistdltf |
| | polypeptide, anti- | svkssrgssdpqgvtcgaatlsaervrgdnkeyeysvecqedsacpaaeeslpievmvdavhkl |
| | HSA sdAb, Fab | kyenytssffirdiikpdppknlqlkplknsrqvevsweypdtwstphsyfsltfcvqvqgkskr |
| | Blocker, 1 | ekkdrvftdktsatvicrknasisvraqdryyssswsewasvpcsggggsggggsggggsrnlp |
| | cleavage site | vatpdpgmfpclhhsqnllravsnmlqkarqtlefypctseeidheditkdktstveaclpleltkn |
| | | esclnsretsfitngsclasrktsfmmalclssiyedlkmyqvefktmnakllmdpkrqifldqn |
| | | mlavidelmqalnfnsetvpqkssleepdfyktkiklcillhafriravtidrvmsylnassggpG |
| | | PAGLYAQpgsEVQLVESGGGLVQPGNSLRLSCAASGFTFSKFGM |
| | | SWVRQAPGKGLEWVSSISGSGRDTLYAESVKGRFTISRDNAKT |
| | | TLYLQMNSLRPEDTAVYYCTIGGSLSVSSQGTLVTVSSggggsggg |
| | | gsggggsggggsggggsggggsQSVLTQPPSVSGAPGQRVTISCSGSRSNI |
| | | GSNTVKWYQQLPGTAPKLLIYYNDQRPSGVPDRFSGSKSGTSA |
| | | SLAITGLQAEDEADYYCQSYDRYTHPALLFGTGTKVTVLgqpkaa |
| | | psvtlfppsseelqankatlvclisdfypgavtvawkadsspvkagvetttpskqsnnkyaassyl |
| | | sltpeqwkshrsyscqvthegstvektvaptecs** |
| 12 | WW0636 | iwelkkdvyvveldwypdapgemvvltcdtpeedgitwtldqssevlgsgktltiqvkefgdag |
| | | qytchkggevlshsllllhkkedgiwstdilkdqkepknktflrceaknysgrftcwwlttistdltf |
| | | svkssrgssdpqgvtcgaatlsaervrgdnkeyeysvecqedsacpaaeeslpievmvdavhkl |
| | | kyenytssffirdiikpdppknlqlkplknsrqvevsweypdtwstphsyfsltfcvqvqgkskr |
| | | ekkdrvftdktsatvicrknasisvraqdryyssswsewasvpcs** |
| 13 | WW0727 | QVQLVESGGGVVQPGRSLRLSCAASGFTFSSYGMHWVRQAPG |
| | Monomeric IL-12 | KGLEWVAFIRYeGSNKYYAeSVKGRFTISRDNSKNTLYLQMNS |
| | polypeptide, anti- | LRAEDTAVYYCKTHGSHDNWGQGTMVTVSSastkgpsvfplapsskst |
| | HSA sdAb, Fab | sggtaalgclvkdyfpepvtvswnsgaltsgvhtfpavlqssglyslssvvtvpssslgtqtyicnv |
| | Blocker, 2 | nhkpsntkvdkrvepksc** |
| | cleavage sites | |
| 14 | WW0613 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPALFKSSFPPGS |
| | | CDLPQTHSLGSRRTLMLLAQMRRISLFSCLKDRHDFGFPQEEFG |
| | | NQFQKAETIPVLHEMIQQIFNLFSTKDSSAAWDETLLDKFYTEL |
| | | YQQLNDLEACVIQGVGVTETPLMKEDSILAVRKYFQRITLYLK |
| | | EKKYSPCAWEVVRAEIMRSFSLSTNLQESLRSKESGGPALFKSS |
| | | FPPGSEVQLVESGGGLVQPGNSLRLSCAASGFTFSKFGMSWVR |
| | | QAPGKGLEWVSSISGSGRDTLYAESVKGRFTISRDNAKTTLYLQ |
| | | MNSLRPEDTAVYYCTIGGSLSVSSQGTLVTVSS |
| 15 | WW0614 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPPLAQKLKSSP |
| | | GSCDLPQTHSLGSRRTLMLLAQMRRISLFSCLKDRHDFGFPQEE |
| | | FGNQFQKAETIPVLHEMIQQIFNLFSTKDSSAAWDETLLDKFYT |
| | | ELYQQLNDLEACVIQGVGVTETPLMKEDSILAVRKYFQRITLYL |
| | | KEKKYSPCAWEVVRAEIMRSFSLSTNLQESLRSKESGGPPLAQK |
| | | LKSSPGSEVQLVESGGGLVQPGNSLRLSCAASGFTFSKFGMSW |
| | | VRQAPGKGLEWVSSISGSGRDTLYAESVKGRFTISRDNAKTTL |
| | | YLQMNSLRPEDTAVYYCTIGGSLSVSSQGTLVTVSS |
| 16 | WW0819 | EAHKSEIAHRYNDLGEQHFKGLVLIAFSQYLQKCSYDEHAKLV |
| | | QEVTDFAKTCVADESAANCDKSLHTLFGDKLCAIPNLRENYGE |
| | | LADCCTKQEPERNECFLQHKDDNPSLPPFERPEAEAMCTSFKEN |
| | | PTTFMGHYLHEVARRHPYFYAPELLYYAEQYNEILTQCCAEAD |
| | | KESCLTPKLDGVKEKALVSSVRQRMKCSSMQKFGERAFKAWA |
| | | VARLSQTFPNADFAEITKLATDLTKVNKECCHGDLLECADDRA |
| | | ELAKYMCENQATISSKLQTCCDKPLLKKAHCLSEVEHDTMPAD |
| | | LPAIAADFVEDQEVCKNYAEAKDVFLGTFLYEYSRRHPDYSVS |
| | | LLLRLAKKYEATLEKCCAEANPPACYGTVLAEFQPLVEEPKNL |
| | | VKTNCDLYEKLGEYGFQNAILVRYTQKAPQVSTPTLVEAARNL |
| | | GRVGTKCCTLPEDQRLPCVEDYLSAILNRVCLLHEKTPVSEHVT |
| | | KCCSGSLVERRPCFSALTVDETYVPKEFKAETFTFHSDICTLPEK |
| | | EKQIKKQTALAELVKHKPKATAEQLKTVMDDFAQFLDTCCKA |
| | | ADKDTCFSTEGPNLVTRCKDALASGGPALFKSSFPPGSCDLPQT |
| | | HNLRNKRALTLLVQMRRLSPLSCLKDRKDFGFPQEKVDAQQIK |
| | | KAQAIPVLSELTQQILNIFTSKDSSAAWNTTLLDSFCNDLHQQL |
| | | NDLQGCLMQQVGVQEFPLTQEDALLAVRKYFHRITVYLREKK |
| | | HSPCAWEVVRAEVWRALSSSANVLGRLREEKSGGPALFKSSFP |
| | | PGSEVQLVESGGGLVQPGNSLRLSCAASGFTFSKFGMSWVRQA |
| | | PGKGLEWVSSISGSGRDTLYAESVKGRFTISRDNAKTTLYLQM |
| | | NSLRPEDTAVYYCTIGGSLSVSSQGTLVTVSSHHHHHH |
| 17 | WW0821 | DAHKSEVAHRFKDLGEENFKALVLIAFAQYLQQCPFEDHVKLV |
| | | NEVTEFAKTCVADESAENCDKSLHTLFGDKLCTVATLRETYGE |
| | | MADCCAKQEPERNECFLQHKDDNPNLPRLVRPEVDVMCTAFH |
| | | DNEETFLKKYLYEIARRHPYFYAPELLFFAKRYKAAFTECCQA |
| | | ADKAACLLPKLDELRDEGKASSAKQRLKCASLQKFGERAFKA |
| | | WAVARLSQRFPKAEFAEVSKLVTDLTKVHTECCHGDLLECAD |
| | | DRADLAKYICENQDSISSKLKECCEKPLLEKSHCIAEVENDEMP |
| | | ADLPSLAADFVESKDVCKNYAEAKDVFLGMFLYEYARRHPDY |
| | | SVVLLLRLAKTYETTLEKCCAAADPHECYAKVFDEFKPLVEEP |
| | | QNLIKQNCELFEQLGEYKFQNALLVRYTKKVPQVSTPTLVEVS |
| | | RNLGKVGSKCCKHPEAKRMPCAEDYLSVVLNQLCVLHEKTPV |
| | | SDRVTKCCTESLVNRRPCFSALEVDETYVPKEFNAETFTFHADI |
| | | CTLSEKERQIKKQTALVELVKHKPKATKEQLKAVMDDFAAFV |
| | | EKCCKADDKETCFAEEGKKLVAASQAALGLSGGPALFKSSFPP |
| | | GSCDLPQTHSLGSRRTLMLLAQMRRISLFSCLKDRHDFGFPQEE |
| | | FGNQFQKAETIPVLHEMIQQIFNLFSTKDSSAAWDETLLDKFYT |
| | | ELYQQLNDLEACVIQGVGVTETPLMKEDSILAVRKYFQRITLYL |
| | | KEKKYSPCAWEVVRAEIMRSFSLSTNLQESLRSKESGGPALFKS |
| | | SFPPGSDAHKSEVAHRFKDLGEENFKALVLIAFAQYLQQCPFED |
| | | HVKLVNEVTEFAKTCVADESAENCDKSLHTLFGDKLCTVATLR |
| | | ETYGEMADCCAKQEPERNECFLQHKDDNPNLPRLVRPEVDVM |
| | | CTAFHDNEETFLKKYLYEIARRHPYFYAPELLFFAKRYKAAFTE |
| | | CCQAADKAACLLPKLDELRDEGKASSAKQRLKCASLQKFGER |
| | | AFKAWAVARLSQRFPKAEFAEVSKLVTDLTKVHTECCHGDLL |
| | | ECADDRADLAKYICENQDSISSKLKECCEKPLLEKSHCIAEVEN |
| | | DEMPADLPSLAADFVESKDVCKNYAEAKDVFLGMFLYEYARR |
| | | HPDYSVVLLLRLAKTYETTLEKCCAAADPHECYAKVFDEFKPL |
| | | VEEPQNLIKQNCELFEQLGEYKFQNALLVRYTKKVPQVSTPTL |
| | | VEVSRNLGKVGSKCCKHPEAKRMPCAEDYLSVVLNQLCVLHE |
| | | KTPVSDRVTKCCTESLVNRRPCFSALEVDETYVPKEFNAETFTF |
| | | HADICTLSEKERQIKKQTALVELVKHKPKATKEQLKAVMDDFA |
| | | AFVEKCCKADDKETCFAEEGKKLVAASQAALGL |
| 18 | WW0834 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPALFKSSFPPGS |
| | | CDLPQTHSLGNRRALILLAQMGRISHFSCLKDRYDFGFPQEVFD |
| | | GNQFQKAQAISAFHEMIQQTFNLFSTKDSSAAWDETLLDKFYIE |
| | | LFQQLNDLEACVTQEVGVEEIALMNEDSILAVRKYFQRITLYL |
| | | MGKKYSPCAWEVVRAEIMRSFSFSTNLQKGLRRKDSGGPALFK |
| | | SSFPPGSEVQLVESGGGLVQPGNSLRLSCAASGFTFSKFGMSWV |
| | | RQAPGKGLEWVSSISGSGRDTLYAESVKGRFTISRDNAKTTLYL |
| | | QMNSLRPEDTAVYYCTIGGSLSVSSQGTLVTVSS |
| 19 | WW0742 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPALFKSSFPPGS |
| | | MSYNLLGFLQRSSNFQCQKLLWQLNGRLEYCLKDRMNFDIPEE |
| | | IKQLQQFQKEDAALTIYEMLQNIFAIFRQDSSSTGWNETIVENLL |
| | | ANVYHQINHLKTVLEEKLEKEDFTRGKLMSSLHLKRYYGRILH |
| | | YLKAKEYSHCAWTIVRVEILRNFYFINRLTGYLRNSGGPALFKS |
| | | SFPPGSEVQLVESGGGLVQPGNSLRLSCAASGFTFSKFGMSWV |
| | | RQAPGKGLEWVSSISGSGRDTLYAESVKGRFTISRDNAKTTLYL |
| | | QMNSLRPEDTAVYYCTIGGSLSVSSQGTLVTVSS |
| 20 | WW0612 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPGPAGLYAQPG |
| | | SCDLPQTHSLGSRRTLMLLAQMRRISLFSCLKDRHDFGFPQEEF |
| | | GNQFQKAETIPVLHEMIQQIFNLFSTKDSSAAWDETLLDKFYTE |
| | | LYQQLNDLEACVIQGVGVTETPLMKEDSILAVRKYFQRITLYL |
| | | KEKKYSPCAWEVVRAEIMRSFSLSTNLQESLRSKESGGPGPAGL |
| | | YAQPGSEVQLVESGGGLVQPGNSLRLSCAASGFTFSKFGMSWV |
| | | RQAPGKGLEWVSSISGSGRDTLYAESVKGRFTISRDNAKTTLYL |
| | | QMNSLRPEDTAVYYCTIGGSLSVSSQGTLVTVSS |
| 21 | WW0045 | EVQLVESGGGLVQPGGSLRLSCAASGFTFSSYTLAWVRQAPGK |
| | (Monomeric IL-2 | GLEWVAAIDSSSYTYSPDTVRGRFTISRDNAKNSLYLQMNSLR |
| | inducible prodrug) | AEDTAVYYCARDSNWDALDYWGQGTTVTVSSGGGGSGGGGS |
| | | GGGGSDIQMTQSPSSLSASVGDRVTITCKASQNVGTNVGWYQ |
| | | QKPGKAPKALIYSASFRYSGVPSRFSGSGSGTDFTLTISSLQPED |
| | | FATYYCQQYYTYPYTFGGGTKVEIKGGGGSGGGGSGGGGSGG |
| | | GGSGGGGSGGGGSSGGPGPAGMKGLPGSAPTSSSTKKTQLQLE |
| | | HLLLDLQMILNGINNYKNPKLTRMLTFKFYMPKKATELKHLQC |
| | | LEEELKPLEEVLNLAQSKNFHLRPRDLISNINVIVLELKGSETTF |
| | | MCEYADETATIVEFLNRWITFCQSIISTLTSGGPGPAGMKGLPGS |
| | | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSHHHHHH |
| 22 | WW0046 | EVQLVESGGGLVQPGNSLRLSCAASGFTFSKFGMSWVRQAPG |
| | (Monomeric IL-2 | KGLEWVSSISGSGRDTLYAESVKGRFTISRDNAKTTLYLQMNS |
| | inducible prodrug) | LRPEDTAVYYCTIGGSLSVSSQGTLVTVSSSGGPGPAGMKGLPG |
| | | SEVQLVESGGGLVQPGGSLRLSCAASGFTFSSYTLAWVRQAPG |
| | | KGLEWVAAIDSSSYTYSPDTVRGRFTISRDNAKNSLYLQMNSL |
| | | RAEDTAVYYCARDSNWDALDYWGQGTTVTVSSGGGGSGGGG |
| | | SGGGGSDIQMTQSPSSLSASVGDRVTITCKASQNVGTNVGWYQ |
| | | QKPGKAPKALIYSASFRYSGVPSRFSGSGSGTDFTLTISSLQPED |
| | | FATYYCQQYYTYPYTFGGGTKVEIKGGGGSGGGGSGGGGSGG |
| | | GGSGGGGSGGGGSSGGPGPAGMKGLPGSAPTSSSTKKTQLQLE |
| | | HLLLDLQMILNGINNYKNPKLTRMLTFKFYMPKKATELKHLQC |
| | | LEEELKPLEEVLNLAQSKNFHLRPRDLISNINVIVLELKGSETTF |
| | | MCEYADETATIVEFLNRWITFCQSIISTLTHHHHHH |
| 23 | WW0203 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPALFKSSFPPGSEVQLVESGGGLVQPGNSLRLSCAASGFTFS |
| | | KFGMSWVRQAPGKGLEWVSSISGSGRDTLYAESVKGRFTISRD |
| | | NAKTTLYLQMNSLRPEDTAVYYCTIGGSLSVSSQGTLVTVSSG |
| | | GGGSGGGGSGGGGSGGGGSGGGGSGGGGSSGGPALFKSSFPPG |
| | | SEVQLVESGGGLVQPGGSLRLSCAASGFTFSSYTLAWVRQAPG |
| | | KGLEWVAAIDSSSYTYSPDTVRGRFTISRDNAKNSLYLQMNSL |
| | | RAEDTAVYYCARDSNWDALDYWGQGTTVTVSSGGGGSGGGG |
| | | SGGGGSDIQMTQSPSSLSASVGDRVTITCKASQNVGTNVGWYQ |
| | | QKPGKAPKALIYSASFRYSGVPSRFSGSGSGTDFTLTISSLQPED |
| | | FATYYCQQYYTYPYTFGGGTKVEIKHHHHHHEPEA |
| 24 | WW0204 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPPLAQKLKSSPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGSGGGGSGGGGSGGGGSSGGPPLAQKLKS |
| | | SPGSEVQLVESGGGLVQPGGSLRLSCAASGFTFSSYTLAWVRQ |
| | | APGKGLEWVAAIDSSSYTYSPDTVRGRFTISRDNAKNSLYLQM |
| | | NSLRAEDTAVYYCARDSNWDALDYWGQGTTVTVSSGGGGSG |
| | | GGGSGGGGSDIQMTQSPSSLSASVGDRVTITCKASQNVGTNVG |
| | | WYQQKPGKAPKALIYSASFRYSGVPSRFSGSGSGTDFTLTISSL |
| | | QPEDFATYYCQQYYTYPYTFGGGTKVEIKHHHHHHEPEA |
| 25 | WW0205 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPPGGPAGIGALFKSSFPPLAQKLKSSPGSEVQLVESGGGLVQ |
| | | PGNSLRLSCAASGFTFSKFGMSWVRQAPGKGLEWVSSISGSGR |
| | | DTLYAESVKGRFTISRDNAKTTLYLQMNSLRPEDTAVYYCTIG |
| | | GSLSVSSQGTLVTVSSGGGGSGGGGSGGGGSGGGGSGGGGSG |
| | | GGGSSGGPPGGPAGIGALFKSSFPPLAQKLKSSPGSEVQLVESG |
| | | GGLVQPGGSLRLSCAASGFTFSSYTLAWVRQAPGKGLEWVAAI |
| | | DSSSYTYSPDTVRGRFTISRDNAKNSLYLQMNSLRAEDTAVYY |
| | | CARDSNWDALDYWGQGTTVTVSSGGGGSGGGGSGGGGSDIQ |
| | | MTQSPSSLSASVGDRVTITCKASQNVGTNVGWYQQKPGKAPK |
| | | ALIYSASFRYSGVPSRFSGSGSGTDFTLTISSLQPEDFATYYCQQ |
| | | YYTYPYTFGGGTKVEIKHHHHHHEPEA |
| 26 | WW0234 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPGPAGMKGLPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGSGGGGSGGGGSGGGGSSGGPGPAGMKG |
| | | LPGQVQLQESGGGLVQTGGSLRLSCTTSGTIFSGYTMGWYRQA |
| | | PGEQRELVAVISGGGDTNYADSVKGRFTISRDNTKDTMYLQM |
| | | NSLKPEDTAVYYCYSREVTPPWKLYWGQGTQVTVSSAAAYPY |
| | | DVPDYGSHHHHHH |
| 27 | WW0235 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPGPAGMKGLPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGSGGGGSGGGGSGGGGSSGGPGPAGMKG |
| | | LPGQVQLQESGGGLVQEGGSLRLSCAASERIFSTDVMGWYRQ |
| | | AAEKQRELVAVVSARGTTNYLDAVKGRFTISRDNARNTLTLQ |
| | | MNDLKPEDTASYYCYVRETTSPWRIYWGQGTQVTVSSAAAYP |
| | | YDVPDYGSHHHHHH |
| 28 | WW0236 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPGPAGMKGLPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGSGGGGSGGGGSGGGGSSGGPGPAGMKG |
| | | LPGQVQLQESGGGLVQAGGSLRLSCAASGSIFSANAMGWYRQ |
| | | APGKQRELVAVISSGGSTNYADSVKGRFTISRDNAKNTVYLQM |
| | | NSLKPEDTAVYYCMYSGSYYYTPNDYWGQGTQVTVSSAAAY |
| | | PYDVPDYGSHHHHHH |
| 29 | WW0308 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPGPAGMKGLPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGGGGGSGGGGSGGGGSSGGPGPAGMKG |
| | | LPGSEVQLVESGGGLVQPGGSLRLSCAASGFTFSSYTLAWVRQ |
| | | APGKGLEWVAAIDSSSYTYSPDTVRGRFTISRDNAKNSLYLQM |
| | | NSLRAEDTAVYYCARDSNWDALDYWGQGTTVTVSSGGGGSG |
| | | GGGSGGGGSDIQMTQSPSSLSASVGDRVTITCKASQNVGTNVG |
| | | WYQQKPGQAPRLLIYSASFRYSGVPSRFSGSGSGTDFTLTISSLQ |
| | | PEDFATYYCQQYYTYPYTFGGGTKVEIKHHHHHH |
| 30 | WW0415 | APTSSSTKKTQLQLEHLLLDLQMILNGINNYKNPKLTRMLTFKF |
| | (Monomeric IL-2 | YMPKKATELKHLQCLEEELKPLEEVLNLAQSKNFHLRPRDLISN |
| | inducible prodrug) | INVIVLELKGSETTFMCEYADETATIVEFLNRWITFCQSIISTLTS |
| | | GGPGPAGLYAQPGSEVQLVESGGGLVQPGNSLRLSCAASGFTF |
| | | SKFGMSWVRQAPGKGLEWVSSISGSGRDTLYAESVKGRFTISR |
| | | DNAKTTLYLQMNSLRPEDTAVYYCTIGGSLSVSSQGTLVTVSS |
| | | GGGGSGGGGSGGGGSGGGGSGGGGSGGGGSSGGPGPAGLYA |
| | | QPGSEVQLVESGGGLVQPGGSLRLSCAASGFTFSSYTLAWVRQ |
| | | APGKGLEWVAAIDSSSYTYSPDTVRGRFTISRDNAKNSLYLQM |
| | | NSLRAEDTAVYYCARDSNWDALDYWGQGTTVTVSSGGGGSG |
| | | GGGSGGGGSDIQMTQSPSSLSASVGDRVTITCKAREKLWSAVA |
| | | WYQQKPGKAPKSLIYSASFRYSGVPSRFSGSGSGTDFTLTISSLQ |
| | | PEDFATYYCQQYYTYPYTFGGGTKVEIK |
| 31 | WW0706 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | (chimeric) | LRPEDTAVYYCTIGGSLSVSSQGTLVTVSSsggpGPAGLYAQpgsi |
| | polypeptide, anti- | welkkdvyvveldwypdapgemvvltcdtpeedgitwtldqssevlgsgktltiqvkefgdag |
| | HSA sdAb, Fab | qytchkggevlshsllllhkkedgiwstdilkdqkepknktflrceaknysgrftcwwlttistdltf |
| | Blocker, 2 | svkssrgssdpqgvtcgaatlsaervrgdnkeyeysvecqedsacpaaeeslpievmvdavhkl |
| | cleavage sites | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrvip |
| | | vsgparclsqsrnllkttddmvktareklkhysctaedidheditrdqtstlktclplelhknesclatr |
| | | etssttrgsclppqktslmmtlclgsiyedlkmyqtefqainaalqnhnhqqiildkgmlvaidel |
| | | mqslnhngetlrqkppvgeadpyrvkmklcillhafstrvvtinrvmgylssasggpGPAGL |
| | | YAQpgsggggsggggsggggsggggsggggsQSVLTQPPSVSGAPGQ |
| | | RVTISCSGSeSNIGSNdVKWYQQLPGTAPKLLIYYNDQRPSGVP |
| | | DRFSGSKSGTSASLAITGLQAEDEADYYCQSYDRYTHPALLFGT |
| | | GTKVTVLgqpkaapsvtlfppsseelqankatlvclisdfypgavtvawkadsspvkagve |
| | | tttpskqsnnkyaassylsltpeqwkshrsyscqvthegstvektvaptecs** |
| 32 | WW0707 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | polypeptide, anti- | LRPEDTAVYYCTIGGSLSVSSQGTLVTVSSsggpGPAGLYAQpgsi |
| | HSA sdAb, Fab | welkkdvyvveldwypdapgemvvltcdtpeedgitwtldqssevlgsgktltiqvkefgdag |
| | Blocker, 2 | qytchkggevlshsllllhkkedgiwstdilkdqkepknktflrceaknysgrftcwwlttistdltf |
| | cleavage sites | svkssrgssdpqgvtcgaatlsaervrgdnkeyeysvecqedsacpaaeeslpievmvdavhkl |
| | | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrnlp |
| | | vatpdpgmfpclhhsqnllravsnmlqkarqtlefypctseeidheditkdktstveaclpleltkn |
| | | esclnsretsfitngsclasrktsfmmalclssiyedlkmyqvefktmnakllmdpkrqifldqn |
| | | mlavidelmqalnfnsetvpqkssleepdfyktkiklcillhafriravtidrvmsylnassggpG |
| | | PAGLYAQpgsggggsggggggggsggggggggsggggsQSVLTQPPSVSG |
| | | APGQRVTISCSGSeSNIGSNdVKWYQQLPGTAPKLLIYYNDQRP |
| | | SGVPDRFSGSKSGTSASLAITGLQAEDEADYYCQSYDRYTHPA |
| | | LLFGTGTKVTVLgqpkaapsvtlfppsseelqankatlvclisdfypgavtvawkadss |
| | | pvkagvetttpskqsnnkyaassylsltpeqwkshrsyscqvthegstvektvaptecs** |
| 33 | WW0708 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | (chimeric) | LRPEDTAVYYCTIGGSLSVSSQGTLVTVSSsggpGPAGLYAQpgsi |
| | polypeptide, anti- | welkkdvyvveldwypdapgemvvltcdtpeedgitwtldqssevlgsgktltiqvkefgdag |
| | HSA sdAb, Fab | qytchkggevlshsllllhkkedgiwstdilkdqkepknktflrceaknysgrftcwwlttistdltf |
| | Blocker, 2 | svkssrgssdpqgvtcgaatlsaervrgdnkeyeysvecqedsacpaaeeslpievmvdavhkl |
| | cleavage sites | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrvip |
| | | vsgparclsqsrnllkttddmvktareklkhysctaedidheditrdqtstlktclplelhknesclatr |
| | | etssttrgsclppqktslmmtlclgsiyedlkmyqtefqainaalqnhnhqqiildkgmlvaidel |
| | | mqslnhngetlrqkppvgeadpyrvkmklcillhafstrvvtinrvmgylssasggpGPAGL |
| | | YAQpgsggggggggsggggggggggggsggggsQSVLTQPPSVSGAPGQ |
| | | RVTISCSGSRSNIGeNTVKWYQQLPGTAPKLLIYYNDQRPSGVP |
| | | DRFSGSKSGTSASLAITGLQAEDEADYYCQSYDRYTHPALLFGT |
| | | GTKVTVLgqpkaapsvtlfppsseelqankatlvclisdfypgavtvawkadsspvkagve |
| | | tttpskqsnnkyaassylsltpeqwkshrsyscqvthegstvektvaptecs** |
| 34 | WW0709 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | polypeptide, anti- | LRPEDTAVYYCTIGGSLSVSSQGTLVTVSSsggpGPAGLYAQpgsi |
| | HSA sdAb, Fab | welkkdvyvveldwypdapgemvvltcdtpeedgitwtldqssevlgsgktltiqvkefgdag |
| | Blocker, 2 | qytchkggevlshsllllhkkedgiwstdilkdqkepknktflrceaknysgrftcwwlttistdltf |
| | cleavage sites | svkssrgssdpqgvtcgaatlsaervrgdnkeyeysvecqedsacpaaeeslpievmvdavhkl |
| | | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrnlp |
| | | vatpdpgmfpclhhsqnllravsnmlqkarqtlefypctseeidheditkdktstveaclpleltkn |
| | | esclnsretsfitngsclasrktsfmmalclssiyedlkmyqvefktmnakllmdpkrqifldqn |
| | | mlavidelmqalnfnsetvpqkssleepdfyktkiklcillhafriravtidrvmsylnassggpG |
| | | PAGLYAQpgsggggsggggsggggsggggsggggsggggsQSVLTQPPSVSG |
| | | APGQRVTISCSGSRSNIGeNTVKWYQQLPGTAPKLLIYYNDQRP |
| | | SGVPDRFSGSKSGTSASLAITGLQAEDEADYYCQSYDRYTHPA |
| | | LLFGTGTKVTVLgqpkaapsvtlfppsseelqankatlvclisdfypgavtvawkadss |
| | | pvkagvetttpskqsnnkyaassylsltpeqwkshrsyscqvthegstvektvaptecs** |
| 35 | WW0710 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | (chimeric) | LRPEDTAVYYCTIGGSLSVSSQGTLVTVSSsggpGPAGLYAQpgsi |
| | polypeptide, anti- | welkkdvyvveldwypdapgemvvltcdtpeedgitwtldqssevlgsgktltiqvkefgdag |
| | HSA sdAb, Fab | qytchkggevlshsllllhkkedgiwstdilkdqkepknktflrceaknysgrftcwwlttistdltf |
| | Blocker, 2 | svkssrgssdpqgvtcgaatlsaervrgdnkeyeysvecqedsacpaaeeslpievmvdavhkl |
| | cleavage sites | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrvip |
| | | vsgparclsqsrnllkttddmvktareklkhysctaedidheditrdqtstlktclplelhknesclatr |
| | | etssttrgsclppqktslmmtlclgsiyedlkmyqtefqainaalqnhnhqqiildkgmlvaidel |
| | | mqslnhngetlrqkppvgeadpyrvkmklcillhafstrvvtinrvmgylssasggpGPAGL |
| | | YAQpgsggggsggggsggggsggggsggggsggggsQSVLTQPPSVSGAPGQ |
| | | RVTISCSGSRSNIGeeTVKWYQQLPGTAPKLLIYYNDQRPSGVPD |
| | | RFSGSKSGTSASLAITGLQAEDEADYYCQSYDRYTHPALLFGTG |
| | | TKVTVLgqpkaapsvtlfppsseelqankatlvclisdfypgavtvawkadsspvkagvettt |
| | | pskqsnnkyaassylsltpeqwkshrsyscqvthegstvektvaptecs** |
| 36 | WW0711 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | polypeptide, anti- | LRPEDTAVYYCTIGGSLSVSSQGTLVTVSSsggpGPAGLYAQpgsi |
| | HSA sdAb, Fab | welkkdvyvveldwypdapgemvvltcdtpeedgitwtldqssevlgsgktltiqvkefgdag |
| | Blocker, 2 | qytchkggevlshsllllhkkedgiwstdilkdqkepknktflrceaknysgrftcwwlttistdltf |
| | cleavage sites | svkssrgssdpqgvtcgaatlsaervrgdnkeyeysvecqedsacpaaeeslpievmvdavhkl |
| | | kyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrnlp |
| | | vatpdpgmfpclhhsqnllravsnmlqkarqtlefypctseeidheditkdktstveaclpleltkn |
| | | esclnsretsfitngsclasrktsfmmalclssiyedlkmyqvefktmnakllmdpkrqifldqn |
| | | mlavidelmqalnfnsetvpqkssleepdfyktkiklcillhafriravtidrvmsylnassggpG |
| | | PAGLYAQpgsggggsggggsggggsggggsggggsggggsQSVLTQPPSVSG |
| | | APGQRVTISCSGSRSNIGeeTVKWYQQLPGTAPKLLIYYNDQRPS |
| | | GVPDRFSGSKSGTSASLAITGLQAEDEADYYCQSYDRYTHPAL |
| | | LFGTGTKVTVLgqpkaapsvtlfppsseelqankatlvclisdfypgavtvawkadsspv |
| | | kagvetttpskqsnnkyaassylsltpeqwkshrsyscqvthegstvektvaptecs** |
| 37 | WW0712 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | (chimeric) | LRPEDTAVYYCTIGGSLSVSSQGTLVTVSSsggpALFKSSFPpgsiw |
| | polypeptide, anti- | elkkdvyvveldwypdapgemvvltcdtpeedgitwtldqssevlgsgktltiqvkefgdagqy |
| | HSA sdAb, Fab | tchkggevlshsllllhkkedgiwstdilkdqkepknktflrceaknysgrftcwwlttistdltfsv |
| | Blocker, 2 | kssrgssdpqgvtcgaatlsaervrgdnkeyeysvecqedsacpaaeeslpievmvdavhklky |
| | cleavage sites | enytssffirdiikpdppknlqlkplknsrqvevsweypdtwstphsyfsltfcvqvqgkskrek |
| | | kdrvftdktsatvicrknasisvraqdryyssswsewasvpcsggggsggggsggggsrvipvs |
| | | gparclsqsrnllkttddmvktareklkhysctaedidheditrdqtstlktclplelhknesclatret |
| | | ssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssasggpALFKSS |
| | | FPpgsggggsggggsggggsggggsggggsggggsQSVLTQPPSVSGAPGQRV |
| | | TISCSGSRSNIGSNTVKWYQQLPGTAPKLLIYYNDQRPSGVPDR |
| | | FSGSKSGTSASLAITGLQAEDEADYYCQSYDRYTHPALLFGTGT |
| | | KVTVLgqpkaapsvtlfppsseelqankatlvclisdfypgavtvawkadsspvkagvetttp |
| | | skqsnnkyaassylsltpeqwkshrsyscqvthegstvektvaptecs** |
| 38 | WW0713 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | polypeptide, anti- | LRPEDTAVYYCTIGGSLSVSSQGTLVTVSSsggpALFKSSFPpgsiw |
| | HSA sdAb, Fab | elkkdvyvveldwypdapgemvvltcdtpeedgitwtldqssevlgsgktltiqvkefgdagqy |
| | Blocker, 2 | tchkggevlshsllllhkkedgiwstdilkdqkepknktflrceaknysgrftcwwlttistdltfsv |
| | cleavage sites | kssrgssdpqgvtcgaatlsaervrgdnkeyeysvecqedsacpaaeeslpievmvdavhklky |
| | | enytssffirdiikpdppknlqlkplknsrqvevsweypdtwstphsyfsltfcvqvqgkskrek |
| | | kdrvftdktsatvicrknasisvraqdryyssswsewasvpcsggggsggggsggggsrnlpvat |
| | | pdpgmfpclhhsqnllravsnmlqkarqtlefypctseeidheditkdktstveaclpleltknesc |
| | | lnsretsfitngsclasrktsfmmalclssiyedlkmyqvefktmnakllmdpkrqifldqnmla |
| | | videlmqalnfnsetvpqkssleepdfyktkiklcillhafriravtidrvmsylnassggpALF |
| | | KSSFPpgsggggggggsggggggggsggggggggsQSVLTQPPSVSGAPG |
| | | QRVTISCSGSRSNIGSNTVKWYQQLPGTAPKLLIYYNDQRPSGV |
| | | PDRFSGSKSGTSASLAITGLQAEDEADYYCQSYDRYTHPALLFG |
| | | TGTKVTVLgqpkaapsvtlfppsseelqankatlvclisdfypgavtvawkadsspvkag |
| | | vetttpskqsnnkyaassylsltpeqwkshrsyscqvthegstvektvaptecs** |
| 39 | WW0714 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | (chimeric) | LRPEDTAVYYCTIGGSLSVSSQGTLVTVSSsggpALFKSSFPpgsiw |
| | polypeptide, anti- | elkkdvyvveldwypdapgemvvltcdtpeedgitwtldqssevlgsgktltiqvkefgdagqy |
| | HSA sdAb, Fab | tchkggevlshsllllhkkedgiwstdilkdqkepknktflrceaknysgrftcwwlttistdltfsv |
| | Blocker, 2 | kssrgssdpqgvtcgaatlsaervrgdnkeyeysvecqedsacpaaeeslpievmvdavhklky |
| | cleavage sites | enytssffirdiikpdppknlqlkplknsrqvevsweypdtwstphsyfsltfcvqvqgkskrek |
| | | kdrvftdktsatvicrknasisvraqdryyssswsewasvpcsggggsggggsggggsrvipvs |
| | | gparclsqsrnllkttddmvktareklkhysctaedidheditrdqtstlktclplelhknesclatret |
| | | ssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssasggpALFKSS |
| | | FPpgsggggsggggsggggsggggsggggggggsQSVLTQPPSVSGAPGQRV |
| | | TISCSGSRSNIGSeTVKWYQQLPGTAPKLLIYYNDQRPSGVPDRF |
| | | SGSKSGTSASLAITGLQAEDEADYYCQSYDRYTHPALLFGTGT |
| | | KVTVLgqpkaapsvtlfppsseelqankatlvclisdfypgavtvawkadsspvkagvetttp |
| | | skqsnnkyaassylsltpeqwkshrsyscqvthegstvektvaptecs** |
| 40 | WW0715 | EVQLVESGGGLVQPGNSLRLSCAASGFTFSKFGMSWVRQAPG |
| | Monomeric IL-12 | KGLEWVSSISGSGRDTLYAESVKGRFTISRDNAKTTLYLQMNS |
| | polypeptide, anti- | LRPEDTAVYYCTIGGSLSVSSQGTLVTVSSsggpALFKSSFPpgsiw |
| | HSA sdAb, Fab | elkkdvyvveldwypdapgemvvltcdtpeedgitwtldqssevlgsgktltiqvkefgdagqy |
| | Blocker, 2 | tchkggevlshsllllhkkedgiwstdilkdqkepknktflrceaknysgrftcwwlttistdltfsv |
| | cleavage sites | kssrgssdpqgvtcgaatlsaervrgdnkeyeysvecqedsacpaaeeslpievmvdavhklky |
| | | enytssffirdiikpdppknlqlkplknsrqvevsweypdtwstphsyfsltfcvqvqgkskrek |
| | | kdrvftdktsatvicrknasisvraqdryyssswsewasvpcsggggsggggsggggsrnlpvat |
| | | pdpgmfpclhhsqnllravsnmlqkarqtlefypctseeidheditkdktstveaclpleltknesc |
| | | lnsretsfitngsclasrktsfmmalclssiyedlkmyqvefktmnakllmdpkrqifldqnmla |
| | | videlmqalnfnsetvpqkssleepdfyktkiklcillhafriravtidrvmsylnassggpALF |
| | | KSSFPpgsggggggggggggsggggggggsggggsQSVLTQPPSVSGAPG |
| | | QRVTISCSGSRSNIGSeTVKWYQQLPGTAPKLLIYYNDQRPSGV |
| | | PDRFSGSKSGTSASLAITGLQAEDEADYYCQSYDRYTHPALLFG |
| | | TGTKVTVLgqpkaapsvtlfppsseelqankatlvclisdfypgavtvawkadsspvkag |
| | | vetttpskqsnnkyaassylsltpeqwkshrsyscqvthegstvektvaptecs** |
| 41 | WW50563 | mdmrvpaqllllllwlrgarcaptssstkktqlqlehllldlqmilnginnyknpkltrmltfkfy |
| | Inducible IL-2 | mpkkatelkhlqcleeelkpleevlnlaqsknfhlrprdlisninvivlelkgsettfmceyadetat |
| | Prodrug | iveflnrwitfcqsiistltsggpALFKSSFPpgsgsgg |
| | Human IL-2, | ggsEVQLVESGGGLVQPGGSLRLSCAASGFTFSSYTLAWVRQAP |
| | blocking moiety, | GKGLEWVAAIDSSSYTYSPDTVRGRFTISRDNAKNSLYLQMNS |
| | protease cleavable | LRAEDTAVYYCARDSNWDALDYWGQGTTVTVSSGGGGSGGG |
| | linker, and PDGFR | GSGGGGSDIQMTQSPSSLSASVGDRVTITCKASQNVGTNVGWY |
| | transmembrane | QQKPGKAPKALIYSASFRYSGVPSRFSGSGSGTDFTLTISSLQPE |
| | domain | DFATYYCQQYYTYPYTFGGGTKVEIKAVGQDTQEVIVVPHSLP |
| | | FKVVVISAILALVVLTIISLIILIMLWQKKPR |
| 44 | WW0757 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSsggpALFKSSFPpgsrvi |
| | | pvsgparclsqsrnllkttddmvktareklkhysctaedidheditrdqtstlktclplelhknescla |
| | | tretssttrgsclppqktslmmtlclgsiyedlkmyqtefqainaalqnhnhqqiildkgmlvaide |
| | | lmqslnhngetlrqkppvgeadpyrvkmklcillhafstrvvtinrvmgylssasggpALFKS |
| | | SFPpgsggggsggggsggggsggggsggggsggggsQSVLTQPPSVSGAPGQR |
| | | VTISCSGSRSNIGSNTVKWYQQLPGTAPKLLIYYNDQRPSGVPD |
| | | RFSGSKSGTSASLAITGLQAEDEADYYCQSYDRYTHPALLFGTG |
| | | TKVTVLggggsggggggggsQVQLVESGGGVVQPGRSLRLSCAASG |
| | | FTFSSYGMHWVRQAPGKGLEWVAFIRYeGSNKYYAeSVKGRFT |
| | | ISRDNSKNTLYLQMNSLRAEDTAVYYCKTHGSHDNWGQGTM |
| | | VTVSS |
| 45 | WW0610 | EVQLVESGGGLVQPGNSLRLSCAASGFTFSKFGMSWVRQAPG |
| | | KGLEWVSSISGSGRDTLYAESVKGRFTISRDNAKTTLYLQMNS |
| | | LRPEDTAVYYCTIGGSLSVSSQGTLVTVSSSGGPALFKSSFPPGS |
| | | CDLPQTHNLRNKRALTLLVQMRRLSPLSCLKDRKDFGFPQEKV |
| | | DAQQIKKAQAIPVLSELTQQILNIFTSKDSSAAWNTTLLDSFCN |
| | | DLHQQLNDLQGCLMQQVGVQEFPLTQEDALLAVRKYFHRITV |
| | | YLREKKHSPCAWEVVRAEVWRALSSSANVLGRLREEKSGGPA |
| | | LFKSSFPPGSEVQLVESGGGLVQPGNSLRLSCAASGFTFSKFGM |
| | | SWVRQAPGKGLEWVSSISGSGRDTLYAESVKGRFTISRDNAKT |
| | | TLYLQMNSLRPEDTAVYYCTIGGSLSVSSQGTLVTVSS |
| 707 | WW50394 | QGQDRHMIRMRQLIDIVDQLKNYVNDLVPEFLPAPEDVETNCE |
| | | WSAFSCFQKAQLKSANTGNNERIINVSIKKLKRKPPSTNAGRRQ |
| | | KHRLTCPSCDSYEKKPPKEFLERFKSLLQKMIHQHLSSRTHGSE |
| | | DSggggsggssggpALFKSSFPpgsggggsggsEVQLVESGGGLVQPGNS |
| | | LRLSCAASGFTFSKFGMSWVRQAPGKGLEWVSSISGSGRDTLY |
| | | AESVKGRFTISRDNAKTTLYLQMNSLRPEDTAVYYCTIGGSLSV |
| | | SSQGTLVTVSSsggpALFKSSFPpgsEVQLLESGGGLVQPGGSLRL |
| | | SCAASGFTFSSYAMSWVRQAPGKGLEWVSAISGSGGSTYYADS |
| | | VKGRFTISRDNSKNTLYLQMNSLRAEDTAVYYCARDPVYDLF |
| | | DVWGQGTTVTVSSastkgpsvfplapsskstsggtaalgclvkdyfpepvtvswnsga |
| | | ltsgvhtfpavlqssglyslssvvtvpssslgtqtyicnvnhkpsntkvdkrvepksc** |
| 708 | WW50394 | DIQLTQSPSTLSASVGDRVTITCRASQAISSWLAWYQQKPGKAP |
| | | KLLIYDASSLESGVPSRFSGSGSGTEFTLTISSLQPDDFATYYCQ |
| | | QYDPLRTFGGGTKVEIKrtvaapsvfifppsdeqlksgtasvvcllnnfypreakvq |
| | | wkvdnalqsgnsqesvteqdskdstyslsstltlskadyekhkvyacevthqglsspvtksfnrge |
| | | c** |

TABLE 12
CAR binding domain sequences

| SEQ ID NO | Name | Sequence |
|---|---|---|
| 562 | Anti CD19 | DIQMTQTTSSLSASLGDRVTISCRASQDISKYLNWYQQKPDGTVKLLIY |
| | scFv 1 | HTSRLHSGVPSRFSGSGSGTDYSLTISNLEQEDIATYFCQQGNTLPYTFG |
| | | GGTKLEITGGGGSGGGGSGGGGSEVKLQESGPGLVAPSQSLSVTCTVS |
| | | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| 563 | Anti CD19 | EIVMTQSPATLSLSPGERATLSCRASQDISKYLNWYQQKPGQAPRLLIY |
| | scFv 2 | HTSRLHSGIPARFSGSGSGTDYTLTISSLQPEDFAVYFCQQGNTLPYTFG |
| | | QGTKLEIKGGGGSGGGGSGGGGSQVQLQESGPGLVKPSETLSLTCTVS |
| | | GVSLPDYGVSWIRQPPGKGLEWIGVIWGSETTYYSSSLKSRVTISKDNS |
| | | KNQVSLKLSSVTAADTAVYYCAKHYYYGGSYAMDYWGQGTLVTVSS |
| 564 | Anti CD19 | EIVMTQSPATLSLSPGERATLSCRASQDISKYLNWYQQKPGQAPRLLIY |
| | scFv 3 | HTSRLHSGIPARFSGSGSGTDYTLTISSLQPEDFAVYFCQQGNTLPYTFG |
| | | QGTKLEIKGGGGSGGGGSGGGGSQVQLQESGPGLVKPSETLSLTCTVS |
| | | GVSLPDYGVSWIRQPPGKGLEWIGVIWGSETTYYQSSLKSRVTISKDNS |
| | | KNQVSLKLSSVTAADTAVYYCAKHYYYGGSYAMDYWGQGTLVTVSS |
| 565 | Anti CD19 | QVQLQESGPGLVKPSETLSLTCTVSGVSLPDYGVSWIRQPPGKGLEWIG |
| | scFv 4 | VIWGSETTYYSSSLKSRVTISKDNSKNQVSLKLSSVTAADTAVYYCAK |
| | | HYYYGGSYAMDYWGQGTLVTVSSGGGGSGGGGSGGGGSEIVMTQSP |
| | | ATLSLSPGERATLSCRASQDISKYLNWYQQKPGQAPRLLIYHTSRLHSG |
| | | IPARFSGSGSGTDYTLTISSLQPEDFAVYFCQQGNTLPYTFGQGTKLEIK |
| 566 | Anti CD19 | QVQLQESGPGLVKPSETLSLTCTVSGVSLPDYGVSWIRQPPGKGLEWIG |
| | scFv 5 | VIWGSETTYYQSSLKSRVTISKDNSKNQVSLKLSSVTAADTAVYYCAK |
| | | HYYYGGSYAMDYWGQGTLVTVSSGGGGSGGGGSGGGGSEIVMTQSP |
| | | ATLSLSPGERATLSCRASQDISKYLNWYQQKPGQAPRLLIYHTSRLHSG |
| | | IPARFSGSGSGTDYTLTISSLQPEDFAVYFCQQGNTLPYTFGQGTKLEIK |
| 567 | Anti CD19 | EIVMTQSPATLSLSPGERATLSCRASQDISKYLNWYQQKPGQAPRLLIY |
| | scFv 6 | HTSRLHSGIPARFSGSGSGTDYTLTISSLQPEDFAVYFCQQGNTLPYTFG |
| | | QGTKLEIKGGGGSGGGGSGGGGSGGGGSQVQLQESGPGLVKPSETLSL |
| | | TCTVSGVSLPDYGVSWIRQPPGKGLEWIGVIWGSETTYYSSSLKSRVTI |
| | | SKDNSKNQVSLKLSSVTAADTAVYYCAKHYYYGGSYAMDYWGQGT |
| | | LVTVSS |
| 568 | Anti CD19 | EIVMTQSPATLSLSPGERATLSCRASQDISKYLNWYQQKPGQAPRLLIY |
| | scFv 7 | HTSRLHSGIPARFSGSGSGTDYTLTISSLQPEDFAVYFCQQGNTLPYTFG |
| | | QGTKLEIKGGGGSGGGGSGGGGSGGGGSQVQLQESGPGLVKPSETLSL |
| | | TCTVSGVSLPDYGVSWIRQPPGKGLEWIGVIWGSETTYYQSSLKSRVTI |
| | | SKDNSKNQVSLKLSSVTAADTAVYYCAKHYYYGGSYAMDYWGQGT |
| | | LVTVSS |
| 569 | Anti CD19 | QVQLQESGPGLVKPSETLSLTCTVSGVSLPDYGVSWIRQPPGKGLEWIG |
| | scFv 8 | VIWGSETTYYSSSLKSRVTISKDNSKNQVSLKLSSVTAADTAVYYCAK |
| | | HYYYGGSYAMDYWGQGTLVTVSSGGGGSGGGGSGGGGSGGGGSEIV |
| | | MTQSPATLSLSPGERATLSCRASQDISKYLNWYQQKPGQAPRLLIYHTS |
| | | RLHSGIPARFSGSGSGTDYTLTISSLQPEDFAVYFCQQGNTLPYTFGQGT |
| | | KLEIK |
| 570 | Anti CD19 | QVQLQESGPGLVKPSETLSLTCTVSGVSLPDYGVSWIRQPPGKGLEWIG |
| | scFv 9 | VIWGSETTYYQSSLKSRVTISKDNSKNQVSLKLSSVTAADTAVYYCAK |
| | | HYYYGGSYAMDYWGQGTLVTVSSGGGGSGGGGSGGGGSGGGGSEIV |
| | | MTQSPATLSLSPGERATLSCRASQDISKYLNWYQQKPGQAPRLLIYHTS |
| | | RLHSGIPARFSGSGSGTDYTLTISSLQPEDFAVYFCQQGNTLPYTFGQGT |
| | | KLEIK |
| 571 | Anti CD19 | EIVMTQSPATLSLSPGERATLSCRASQDISKYLNWYQQKPGQAPRLLIY |
| | scFv 10 | HTSRLHSGIPARFSGSGSGTDYTLTISSLQPEDFAVYFCQQGNTLPYTFG |
| | | QGTKLEIKGGGGSGGGGSGGGGSGGGGSQVQLQESGPGLVKPSETLSL |
| | | TCTVSGVSLPDYGVSWIRQPPGKGLEWIGVIWGSETTYYNSSLKSRVTI |
| | | SKDNSKNQVSLKLSSVTAADTAVYYCAKHYYYGGSYAMDYWGQGT |
| | | LVTVSS |
| 572 | Anti CD19 | QVQLQESGPGLVKPSETLSLTCTVSGVSLPDYGVSWIRQPPGKGLEWIG |
| | scFv 11 | VIWGSETTYYNSSLKSRVTISKDNSKNQVSLKLSSVTAADTAVYYCAK |
| | | HYYYGGSYAMDYWGQGTLVTVSSGGGGSGGGGSGGGGSGGGGSEIV |
| | | MTQSPATLSLSPGERATLSCRASQDISKYLNWYQQKPGQAPRLLIYHTS |
| | | RLHSGIPARFSGSGSGTDYTLTISSLQPEDFAVYFCQQGNTLPYTFGQGT |
| | | KLEIK |
| 573 | Anti CD19 | EIVMTQSPATLSLSPGERATLSCRASQDISKYLNWYQQKPGQAPRLLIY |
| | scFv 12 | HTSRLHSGIPARFSGSGSGTDYTLTISSLQPEDFAVYFCQQGNTLPYTFG |
| | | QGTKLEIKGGGGSGGGGSGGGGSQVQLQESGPGLVKPSETLSLTCTVS |
| | | GVSLPDYGVSWIRQPPGKGLEWIGVIWGSETTYYNSSLKSRVTISKDNS |
| | | KNQVSLKLSSVTAADTAVYYCAKHYYYGGSYAMDYWGQGTLVTVSS |
| 574 | Anti CD19 | QVQLQESGPGLVKPSETLSLTCTVSGVSLPDYGVSWIRQPPGKGLEWIG |
| | scFv 13 | VIWGSETTYYNSSLKSRVTISKDNSKNQVSLKLSSVTAADTAVYYCAK |
| | | HYYYGGSYAMDYWGQGTLVTVSSGGGGSGGGGSGGGGSEIVMTQSP |
| | | ATLSLSPGERATLSCRASQDISKYLNWYQQKPGQAPRLLIYHTSRLHSG |
| | | IPARFSGSGSGTDYTLTISSLQPEDFAVYFCQQGNTLPYTFGQGTKLEIK |
| 575 | Anti CD19 | QVQLLESGAELVRPGSSVKISCKASGYAFSSYWMNWVKQRPGQGLEW |
| | scFv 14 | IGQIYPGDGDTNYNGKFKGQATLTADKSSSTAYMQLSGLTSEDSAVYS |
| | | CARKTISSVVDFYFDYWGQGTTVTGGGSGGGSGGGSGGGSELVLTQS |
| | | PKFMSTSVGDRVSVTCKASQNVGTNVAWYQQKPGQSPKPLIYSATYR |
| | | NSGVPDRFTGSGSGTDFTLTITNVQSKDLADYFCQYNRYPYTSFFFTKL |
| | | EIKRRS |
| 576 | Anti CD19 | DIQMTQTTSSLSASLGDRVTISCRASQDISKYLNWYQQKPDGTVKLLIY |
| | scFv 15 | HTSRLHSGVPSRFSGSGSGTDYSLTISNLEQEDIATYFCQQGNTLPYTFG |
| | | GGTKLEITGSTSGSGKPGSGEGSTKGEVKLQESGPGLVAPSQSLSVTCT |
| | | VSGVSLPDYGVSWIRQPPRKGLEWLGVIWGSETTYYNSALKSRLTIIKD |
| | | NSKSQVFLKMNSLQTDDTAIYYCAKHYYYGGSYAMDYWGQGTSVTV |
| | | SSE |
| 577 | Anti CD19 | DIQMTQTTSSLSASLGDRVTISCRASQDISKYLNWYQQKPDGTVKLLIY |
| | scFv 16 | HTSRLHSGVPSRFSGSGSGTDYSLTISNLEQEDIATYFCQQGNTLPYTFG |
| | | GGTKLEITGSTSGSGKPGSGEGSTKGEVKLQESGPGLVAPSQSLSVTCT |
| | | VSGVSLPDYGVSWIRQPPRKGLEWLGVIWGSETTYYNSALKSRLTIIKD |
| | | NSKSQVFLKMNSLQTDDTAIYYCAKHYYYGGSYAMDYWGQGTSVTV |
| | | SS |
| 578 | Anti CD19 | EVKLQESGPGLVAPSQSLSVTCTVSGVSLPDYGVSWIRQPPRKGLEWL |
| | CAR 17 HC | GVIWGSETTYYNSALKSRLTIIKDNSKSQVFLKMNSLQTDDTAIYYCA |
| | | KHYYYGGSYAMDYWGQGTSVTVSS |
| 579 | Anti CD19 | DIQMTQTTSSLSASLGDRVTISCRASQDISKYLNWYQQKPDGTVKLLIY |
| | CAR 17 LC | HTSRLHSGVPSRFSGSGSGTDYSLTISNLEQEDIATYFCQQGNTLPYTFG |
| | | GGTKLEIT |
| 580 | Anti BCMA | DIVLTQSPPSLAMSLGKRATISCRASESVTILGSHLIHWYQQKPGQPPTL |
| | CAR 1 LC | LIQLASNVQTGVPARFSGSGSRTDFTLTIDPVEEDDVAVYYCLQSRTIPR |
| | variable | TFGGGTKLEIK |
| | region | |
| 581 | Anti BCMA | QIQLVQSGPELKKPGETVKISCKASGYTFTDYSINWVKRAPGKGLKWM |
| | CAR 1 HC | GWINTETREPAYAYDFRGRFAFSLETSASTAYLQINNLKYEDTATYFC |
| | variable | ALDYSYAMDYWGQGTSVTVSS |
| | region | |
| 582 | Anti CD20 | QVQLQQWGAGLLKPSETLSLTCAVYGGSFSGYYWSWIRQPPGKGLEW |
| | CAR 1 HC | IGEIDHSGSTNYNPSLKSRVTISVDTSKNQFSLKLSSVTAADTAVYYCA |
| | variable | RGGGSWYSNWFDPWGQGTMVTVSS |
| | region | |
| 583 | Anti CD20 | DIQMTQSPSTLSASVGDRVTITCRASQSISSWLAWYQQKPGKAPKLLIY |
| | CAR 1 LC | DASSLESGVPSRFSGSGSGTEFTLTISSLQPDDFATYYCQQDRSLPPTFG |
| | variable | GGTKVEIK |
| | region | |
| 584 | Anti CD20 | QVQLQQWGAGLLKPSETLSLTCAVYGGSFSGIHWNWIRQPPGKGLEW |
| | CAR 2 HC | IGDIDTSGSTNYNPSLKSRVTISVDTSKNQFSLKLSSVTAADTAVYYCA |
| | variable | RLGQESATYLGMDVWGQGTTVTVSS |
| | region | |
| 585 | Anti CD20 | DIVMTQSPDSLAVSLGERATINCKSSQSVLYSSNNKNYLAWYQQKPGQ |
| | CAR 2 LC | PPKLLIYWASTRESGVPDRFSGSGSGTDFTLTISSLQAEDVAVYYCQQL |
| | variable | YTYPFTFGGGTKVEIK |
| | region | |
| 586 | Anti CD20 | QLQLQESGPGLVKPSETLSLTCTVSGGSISSSSYYWGWIRQPPGKGLEW |
| | CAR 3 HC | IGSIYYSGSTYYNPSLKSRVTISVDTSKNQFSLKLSSVTAADTAVYYCA |
| | variable | RETDYSSGMGYGMDVWGQGTTVTVSS |
| | region | |
| 587 | Anti CD20 | DIQMTQSPSSLSASVGDRVTITCRASQSINSYLNWYQQKPGKAPKLLIY |
| | CARs 3 and | AASSLQSGVPSRFSGSGSGTDFTLTISSLQPEDFATYYCQQSLADPFTFG |
| | 5 LC variable | GGTKVEIK |
| | region | |
| 588 | Anti CD20 | QVQLVQSGAEVKKPGASVKVSCKASGYTFKEYGISWVRQAPGQGLE |
| | CAR 4 HC | WMGWISAYSGHTYYAQKLQGRVTMTTDTSTSTAYMELRSLRSDDTA |
| | variable | VYYCARGPHYDDWSGFIIWFDPWGQGTLVTVSS |
| | region | |
| 589 | Anti CD20 | DIQMTQSPSSLSASVGDRVTITCRASQSISSYLNWYQQKPGKAPKLLIY |
| | CAR 4 LC | AASSLQSGVPSRFSGSGSGTDFTLTISSLQPEDFATYYCQQSYRFPPTFG |
| | variable | QGTKVEIK |
| | region | |
| 590 | Anti CD20 | QVQLQESGPGLVKPSETLSLTCTVSGGSISSPDHYWGWIRQPPGKGLE |
| | CAR 5 HC | WIGSIYASGSTFYNPSLKSRVTISVDTSKNQFSLKLSSVTAADTAVYYC |
| | variable | ARETDYSSGMGYGMDVWGQGTTVTVSS |
| | region | |
| 591 | Anti CD20 | QITLKESGPTLVKPTQTLTLTCTFSGFSLDTEGVGVGWIRQPPGKALEW |
| | CAR 6 HC | LALIYFNDQKRYSPSLKSRLTITKDTSKNQVVLTMTNMDPVDTAVYYC |
| | variable | ARDTGYSRWYYGMDVWGQGTTVTVSS |
| | region | |
| 592 | Anti CD20 | DIQMTQSPSSVSASVGDRVTITCRASQGISSWLAWYQQKPGKAPKLLIY |
| | CAR 6 LC | AASSLQSGVPSRFSGSGSGTDFTLTISSLQPEDFATYYCQQAYAYPITFG |
| | variable | GGTKVEIK |
| | region | |
| 593 | Anti CD20 | QVQLQQWGAGLLKPSETLSLTCAVYGGSFEKYYWSWIRQPPGKGLEW |
| | CAR 7 HC | IGEIYHSGLTNYNPSLKSRVTISVDTSKNQFSLKLSSVTAADTAVYYCA |
| | variable | RVRYDSSDSYYYSYDYGMDVWGQGTTVTVSS |
| | region | |
| 594 | Anti CD20 | DIVLTQSPDSLAVSLGERATINCKSSQSVLYSSNNKNYLAWYQQKPGQ |
| | CARs 7, 8, | PPKLLIYWASSRESGVPDRFSGSGSGTDFTLTISSLQAEDVAVYYCQQS |
| | and 9 LC | YSFPWTFGGGTKVEIK |
| | variable | |
| | region | |
| 595 | Anti CD20 | QVQLQQWGAGLLKPSETLSLTCAVYGGSFSRYVWSWIRQPPGKGLEW |
| | CAR 8 HC | IGEIDSSGKTNYNPSLKSRVTISVDTSKNQFSLKLSSVTAADTAVYYCA |
| | variable | RVRYDSSDSYYYSYDYGMDVWGQGTTVTVSS |
| | region | |
| 596 | Anti CD20 | QVQLQQWGAGLLKPSETLSLTCAVYGGSFSGYAWSWIRQPPGKGLEW |
| | CAR 9 HC | IGEIDHRGFTNYNPSLKSRVTISVDTSKNQFSLKLSSVTAADTAVYYCA |
| | variable | RVRYDSSDSYYYSYDYGMDVWGQGTTVTVSS |
| | region | |
| 597 | Anti CD20 | QVQLQQWGAGLLKPSETLSLTCAVYGGSFQKYYWSWIRQPPGKGLE |
| | CAR 10 HC | WIGEIDTSGFTNYNPSLKSRVTISVDTSKNQFSLKLSSVTAADTAVYYC |
| | variable | ARVGRYSYGYYITAFDIWGQGTTVTVSS |
| | region | |
| 598 | Anti CD20 | DIVMTQSPDSLAVSLGERATINCKSSQSVLYSSNNKNYLAWYQQKPGQ |
| | CAR 10 LC | PPKLLIYWASTRESGVPDRFSGSGSGTDFTLTISSLQAEDVAVYYCQQH |
| | variable | YSFPFTFGGGTKVEIK |
| | region | |
| 599 | Anti CD22 | QVQLQQSGPGLVKPSQTLSLTCAISGDSVSSNSAAWNWIRQSPSRGLE |
| | CAR 1 HC | WLGRTYYRSTWYNDYAVSVKSRITINPDTSKNQFSLQLNSVTPEDTAV |
| | variable | YYCAREVSGTSAFDIWGQGTMVTVS |
| | region | |
| 600 | Anti CD22 | DIQMTQSPSSLSASVGDRVTITCRASQSISSYLNWYQQKPGKAPKLLIY |
| | CAR 1 LC | AASSLQSGVPSRFSGSGSGTDFTLTISSLQPEDFATYYCQQSYSTPLTFG |
| | variable | GGTKLEIK |
| | region | |
| 601 | Anti CD22 | QVQLQQSGPGLVKPSQTLSLTCAISGDSVSSNSAAWNWIRQSPSRGLE |
| | CAR 2 HC | WLGRTYYRSKWYNDYAVSVKSRITINPDTSKNQFSLQLNSVTPEDTAV |
| | variable | YYCARASMTGGYSYGDAFDIWGQGTLVTVS |
| | region | |
| 602 | Anti CD22 | AIRMTQSPSSLSASVGDRVTITCRASQSISSYLNWYQQKPGKAPKLLIY |
| | CAR 2 LC | AASSLQSGVPSRFSGSGSGTDFSLTISSLQPEDSATYYCQQTYSTPLTFG |
| | variable | QGTKVEIK |
| | region | |
| 603 | Anti CD22 | QVQLQQSGPGLVEPSQTLSLTCAISGDSVSSDSVAWNWIRQSPSRGLE |
| | CAR 3 HC | WLGRTYYRSTWYNDYAGSVKSRITINPDTSKNQFSLQLTSVTPEDTAV |
| | variable | YYCTRSRHNTFRGMDVWGQGTTVTVS |
| | region | |
| 604 | Anti CD22 | DIVMTQSPSSLSASVGDRVTITCRASQTISSYLNWYQQKPGKAPKLLIY |
| | CAR 3 LC | DASSLQSGVPSRFSGSGSGTDFTLTINSLQPEDFATYYCQQSYTTPITFG |
| | variable | QGTRLEIK |
| | region | |
| 605 | Anti CD22 | QVQLQQSGPGLVEPSQTLSLTCAISGDSVSSNSAAWNWIRQSPSRGLE |
| | CAR 4 HC | WLGRTYYRSTWYNDYAGSVKSRITINPDTSKNQFSLQLTSVTPEDTAV |
| | variable | YYCTRSRHNTFRGMDVWGQGTLVTVS |
| | region | |
| 606 | Anti CD22 | DIQLTQSPSSLSASVGDRVTITCRASQSISSYLNWYQQKPGKAPKLLIYA |
| | CAR 4 LC | ASSLQSGVPSRFSGSGSGTDFTLTISSLQPEDFATYYCQQSYSTPLTFGG |
| | variable | GTKVEIK |
| | region | |
| 607 | Anti CD22 | QVQLQQSGPGLVEPSQTLSLTCAISGDSVSSDSVAWNWIRQSPSRGLE |
| | CAR 5 HC | WLGRTYYRSTWYNDYAGSVKSRITINPDTSKNQFSLQLNSVTPEDTAV |
| | variable | YYCARDRNGMDVWGQGTMVTVS |
| | region | |
| 608 | Anti CD22 | DIVMTQSPSSLSASVGDRVTITCRASQSISSYLNWYQQKPGKAPKLLIY |
| | CAR 5 LC | DASNLETGVPSRFSGSGSGTDFTFTITSLQPEDFATYYCQQSYTTPLTFG |
| | variable | GGTKVEIK |
| | region | |
| 609 | Anti CD22 | QVQLQQSGPGLVKPSQTLSLTCAISGDSVSSSSAAWNWIRQSPSRGLE |
| | CAR 6 HC | WLGRTYYRSAWYNDYAVSVKSRITINPDTSKNQFSLQLNSVTPEDTAV |
| | variable | YYCARESVLLDGMDVWGRGTTVTVS |
| | region | |
| 610 | Anti CD22 | AIRMTQSPSTLSASVGDRVTITCRASQSISTYLNWYQQKAGKAPRLLIH |
| | CAR 6 LC | DASSLQSGVPSRFSGSGSGTDFTLTISSLQPEDFATYYCQQSYSTPLTFG |
| | variable | GGTKVEIK |
| | region | |
| 611 | Anti CD22 | QVQLQQSGPGLVQPSQTLSLTCVISGDSVSSNSATWNWIRQSPSRGLE |
| | CAR 7 HC | WLGRTYYRSKWYNDYAVSVKSRITINPDTSKNQFSLQLNSVTPEDTAV |
| | variable | YYCARDGDGGSYYDYYYYGMDVWGQGTTVTVS |
| | region | |
| 612 | Anti CD22 | DIQLTQSPSSLSTSVGDRVTITCRASQSISTYLNWYQQKPGKAPKLLIYA |
| | CAR 7 LC | ASNLQSGVPSRFSGSGSGTDFTLTISSLQPEDFATYFCQQSYTTPITFGQ |
| | variable | GTRLEIK |
| | region | |
| 613 | Anti CD22 | QVQLQQSGPGLVKPSQTLSLTCAISGDSVSSNSAAWNWIRQSPSRGLE |
| | CAR 8 HC | WLGRTYYRSAWYNDYAVSVKSRITINPDTSKNQFSLQLSSVTPEDTAV |
| | variable | YYCARDVEGFDYWGQGTLVTVS |
| | region | |
| 614 | Anti CD22 | DIVMTQTPSSLSASVGDRVTITCRASQSISSYLNWYQQKPGKAPKLLIY |
| | CAR 8 LC | AASSLQSGVPSRFSGSGSGTDFTLTISSLQPEDFATYYCQQSYSTPITFG |
| | variable | QGTRLEIK |
| | region | |
| 615 | Anti CD22 | QVQLQQSGPGLVKPSQTLSLTCAISGDSVSGNRATWNWIRQSPSRGLE |
| | CAR 9 HC | WLGRTYYRSAWYNDYAVSVKGRITFNPDTSKNQFSLQLNSVTPEDTA |
| | variable | VYYCARGESGAAADAFDIWGQGTTVTVS |
| | region | |
| 616 | Anti CD22 | DIQLTQSPPSLSASVGDRVTITCRASQSISSYLNWYQQKPGKAPKLLIYA |
| | CAR 9 LC | ASSLQSGVPSRFSGSGFGTDFTLTISSLQPEDFATYYCQQSYSTPQTFGQ |
| | variable | GTKVDIK |
| | region | |
| 617 | Anti CD22 | QVQLVQSGAEVKKPGASVKVSCKASGYTFTSYYMHWVRQAPGQGLE |
| | CAR 10 HC | WMGIINPSGGSTSYAQKFQGRVTMTRDTSTSTVYMELSSLRSEDTAVY |
| | variable | YCAREDSGSGAFDIWGQGTLVTVS |
| | region | |
| 618 | Anti CD22 | EIVLTQSPLSLPVTPGEPASISCRSSRSLLSYHGYNYLDWYLQKPGQSPQ |
| | CAR 10 LC | LLIFVGSNRAPGVPDRFSGSGSGTDFTLNISRVEAEDVGVYYCMQSLQT |
| | variable | PRTFGQGTKVEIK |
| | region | |
| 619 | Anti CD22 | EVQLVQSGAEVKKPGASVKVSCKASGYRFTNYWIHWVRQAPGQGLE |
| | CAR 11 HC | WIGGINPGNNYATYRRKFQGRVTMTADTSTSTVYMELSSLRSEDTAV |
| | variable | YYCTREGYGNYGAWFAYWGQGTLVTVSS |
| | region | |
| 620 | Anti CD22 | DVQVTQSPSSLSASVGDRVTITCRSSQSLANSYGNTFLSWYLHKPGKA |
| | CAR 11 LC | PQLLIYGISNRFSGVPDRFSGSGSGTDFTLTISSLQPEDFATYYCLQGTH |
| | variable | QPYTFGQGTKVEIK |
| | region | |
| 621 | Anti CD22 | EVQLQQSGPGLVKPSQTLSLTCAISGDSVSNNNAAWNWIRQSPSRGLE |
| | CAR 12 HC | WLGRTYHRSTWYNDYVGSVKSRITINPDTSKNQFSLQLNSVTPEDTAV |
| | variable | YYCARETDYGDYGAFDIWGQGTTVTVSS |
| | region | |
| 622 | Anti CD22 | QSALTQPASVSGSPGQSITISCTGSRNDIGAYESVSWYQQHPGNAPKLII |
| | CAR 12 LC | HGVNNRPSGVFDRFSVSQSGNTASLTISGLQAEDEADYYCSSHTTTSTL |
| | variable | YVFGTGTKVTVL |
| | region | |
| 623 | Anti CD22 | EVQLQQSGPGLVNPSQTLSITCAISGDSVSSNSAAWNWIRQSPSRGLEW |
| | CAR 13 HC | LGRTFYRSKWYNDYAVSVKGRITISPDTSKNQFSLQLNSVTPEDTAVY |
| | variable | YCAGGDYYYGLDVWGQGTTVTVSS |
| | region | |
| 624 | Anti CD22 | QSALTQPASVSGSPGQSITISCTGSSSDVGGYNSVSWYQQHPGKAPKL |
| | CAR 13 LC | MIYEVINRPSGVSHRFSGSKSGNTASLTISGLQAEDEADYYCSSYTSSST |
| | variable | YVFGTGTKVTVL |
| | region | |
| 625 | Anti CD22 | EVQLQQSGPGLVKPSQTLSLTCAISGDSVLSNSDTWNWIRQSPSRGLE |
| | CAR 14 HC | WLGRTYHRSTWYDDYASSVRGRVSINVDTSKNQYSLQLNAVTPEDTG |
| | variable | VYYCARDRLQDGNSWSDAFDVWGQGTMVTVSS |
| | region | |
| 626 | Anti CD22 | QSALTQPASVSGSPGQSITISCTGSSSDIGGFNYVSWYQQHAGEAPKLM |
| | CAR 14 LC | IYEVTNRPSGVSDRFSGSKSDNTASLTISGLQAEDEADYYCSSYASGSP |
| | variable | LYVFGTGTKVTVL |
| | region | |
| 627 | Anti CD22 | EVQLQQSGPGLVKPSQTLSLTCAISGDSVLSNSDTWNWIRQSPSRGLE |
| | CAR 15 HC | WLGRTYHRSTWYDDYASSVRGRVSINVDTSKNQYSLQLNAVTPEDTG |
| | variable | VYYCARDRLQDGNSWSDAFDVWGQGTMVTVSS |
| | region | |
| 628 | Anti CD22 | QSALTQPASVSGSPGQSITFSCTGTSSDIGGYNYVSWYQQHPGKAPKL |
| | CAR 15 LC | MIYEVSNRPSGVSNRFSGTKSGNTASLTISGLQAEDEADYYCSSYTSSS |
| | variable | TLYVFGTGTKLTVL |
| | region | |
| 629 | Anti CD22 | QVQLQESGPGLVKPSQTLSLTCAISGDSVLSNSDTWNWIRQSPSRGLE |
| | CAR 16 HC | WLGRTYHRSTWYDDYASSVRGRVSINVDTSKNQYSLQLNAVTPEDTG |
| | variable | VYYCARDRLQDGNSWSDAFDVWGQGTMVTVSS |
| | region | |
| 630 | Anti CD22 | QSALTQPASVSGSPGQSITISCTGTSSDVGGYNYVSWYQQHPGKAPKL |
| | CAR 16 LC | MIYEVSNRPSGVSNRFSGSKSGNTASLTISGLQAEDEADYYCSSYTSSS |
| | variable | TLYVFGTGTKVTVL |
| | region | |
| 631 | Anti CD22 | EVQLQQSGPGLVKPSQTLSLTCAISGDSVLSNSDTWNWIRQSPSRGLE |
| | CAR 17 HC | WLGRTYHRSTWYDDYASSVRGRVSINVDTSKNQYSLQLNAVTPEDTG |
| | variable | VYYCARDRLQDGNSWSDAFDVWGQGTMVTVSS |
| | region | |
| 632 | Anti CD22 | QSALTQPASVSGSPGQSITISCTGTSSDVGGYNYVSWYQQHPGKAPKL |
| | CAR 17 LC | MIYDVSNRPSGVSNRFSGSKSGNTASLTISGLQAEDEADYYCSSYTSSS |
| | variable | TLYVFGTGTKVTVL |
| | region | |
| 633 | Anti CD22 | EVQLQQSGPGLVKPSQTLSLTCAISGDSVLSNSDTWNWIRQSPSRGLE |
| | CAR 18 HC | WLGRTYHRSTWYDDYASSVRGRVSINVDTSKNQYSLQLNAVTPEDTG |
| | variable | VYYCARDRLQDGNSWSDAFDVWGQGTMVTVSS |
| | region | |
| 634 | Anti CD22 | QSALTQPASVSGSPGQSITISCTGTSSDVGGYNYVSWYQQHPGKAPKL |
| | CAR 18 LC | MIYEVSNRPSGVSNRFSGSKSGNTASLTISGLQAEDEADYYCSSYTSSS |
| | variable | TLYIFGTGTKVTVL |
| | region | |
| 635 | Anti CD22 | EVQLQQSGPGLVKPSQTLPLTCAISGDSVLSNSDTWNWIRQSPSRGLE |
| | CAR 19 HC | WLGRTYHRSTWYDDYASSVRGRVSINVDTSKNQYSLQLNAVTPEDTG |
| | variable | VYYCARVRLQDGNSWSDAFDVWGQGTMVTVSS |
| | region | |
| 636 | Anti CD22 | QSALTQPASASGSPGQSVTISCTGTSSDVGGYNYVSWYQQHPGKAPKL |
| | CAR 19 LC | MIYDVSNRPSGVSNRFSGSKSGNTASLTISGLQAEDEADYYCSSYTSSS |
| | variable | TLYVFGTGTQLTVL |
| | region | |
| 637 | Anti CD22 | EVQLQQSGPGLVKPSQTLSLTCAISGDSMLSNSDTWNWIRQSPSRGLE |
| | CAR 20 HC | WLGRTYHRSTWYDDYASSVRGRVSINVDTSKNQYSLQLNAVTPEDTG |
| | variable | VYYCARVRLQDGNSWSDAFDVWGQGTMVTVSS |
| | region | |
| 638 | Anti CD22 | QSALTQPASASGSPGQSVTISCTGTSSDVGGYNYVSWYQQHPGKAPKL |
| | CAR 20 LC | MIYDVSNRPSGVSNRFSGSKSGNTASLTISGLQAEDEADYYCSSYTSSS |
| | variable | TLYVFGTGTQLTVL |
| | region | |
| 639 | Anti FLT3 | EVQLVQSGAEVKKPGSSVKVSCKASGGTFSSYAISWVRQAPGQGLEW |
| | heavy chain | MGGIIPIFGTANYAQKFQRVTITADKSTSTAYMELSSLRSEDTAVYYCA |
| | | TFALFGFREQAFDIWGQGTTVTVSS |
| 640 | Anti FLT3 | DIQMTQSPSSLSASVGDRVTITCRASQSISSYLNWYQQKPGKAPKLLIY |
| | light chain | AASSLQSGVPSRFSGSGSGTDFTLTISSLQPEDLATYYCQQSYSTPFTFG |
| | | PGTKVDIK |
| 641 | Anti CD33 | EVQLQQSGPELVKPGASVKISCKASGYTFTDYNMHWVKQSHGKSLEW |
| | CAR 1 VH | IGYIYPYNGGTGYNQKFKSKATLTVDNSSSTAYMDVRSLTSEDSAVYY |
| | | CARGRPAMDYWGQGTSVTVS |
| 642 | Anti CD33 | DIVLTQSPASLAVSLGQRATISCRASESVDNYGISFMNWFQQKPGQPPK |
| | CAR 1 VL | LLIYAASNQGSGVPARFSGSGSGTDFSLNIHPMEEDDTAMYFCQQSKE |
| | | VPWTFGGGTKLEIK |
| 643 | Anti CD33 | QVQLQQSGPELVRPGTFVKISCKASGYTFTNYDINWVNQRPGQGLEWI |
| | CAR 2 VH | GWIYPGDGSTKYNEKFKAKATLTADKSSSTAYLQLNNLTSENSAVYFC |
| | | ASGYEDAMDYWGQGTSVTVSS |
| 644 | Anti CD33 | DIKMTQSPSSMYASLGERVIINCKASQDINSYLSWFQQKPGKSPKTLIY |
| | CAR 2 VL | RANRLVDGVPSRFSGSGSGQDYSLTISSLEYEDMGIYYCLQYDEFPLTF |
| | | GAGTKLELKR |
| 645 | Anti CD33 | EVKLQESGPELVKPGASVKMSCKASGYKFTDYVVHWLKQKPGQGLE |
| | CAR 3 VH | WIGYINPYNDGTKYNEKFKGKATLTSDKSSSTAYMEVSSLTSEDSAVY |
| | | YCARDYRYEVYGMDYWGQGTSVTVSS |
| 646 | Anti CD33 | DIVLTQSPTIMSASPGERVTMTCTASSSVNYIHWYQQKSGDSPLRWIFD |
| | CAR 3 VL | TSKVASGVPARFSGSGSGTSYSLTISTMEAEDAATYYCQQWRSYPLTF |
| | | GDGTRLELKRADAAPTVS |
| 647 | Anti CD33 | QVQLQQPGAEVVKPGASVKMSCKASGYTFTSYYIHWIKQTPGQGLEW |
| | CAR 4 VH | VGVIYPGNDDISYNQKFKGKATLTADKSSTTAYMQLSSLTSEDSAVYY |
| | | CAREVRLRYFDVWGAGTTVTVSS |
| 648 | Anti CD33 | NIMLTQSPSSLAVSAGEKVTMSCKSSQSVFFSSSQKNYLAWYQQIPGQS |
| | CAR 4 VL | PKLLIYWASTRESGVPDRFTGSGSGTDFTLTISSVQSEDLAIYYCHQYLS |
| | | SRTFGGGTKLEIKR |

TABLE 13
CAR hinge and transmembrane domain sequences

| SEQ ID NO | Name | Sequence |
|---|---|---|
| 649 | CD8 hinge | TTTPAPRPPTPAPTIASQPLSLRPEACRPAAGGAVHTRGLDFACD |
| 650 | Ig4 hinge | ESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWL |
| | | NGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLT |
| | | VDKSRWQEGNVFSCSVMHEALHNHYTQKSLSLSLGKM |
| 651 | IgD hinge | RWPESPKAQASSVPTAQPQAEGSLAKATTAPATTRNTGRGGEEKKKE |
| | | KEKEEQEERETKTPECPSHTQPLGVYLLTPAVQDLWLRDKATFTCFVV |
| | | GSDLKDAHLTWEVAGKVPTGGVEEGLLERHSNGSQSQHSRLTLPRSL |
| | | WNAGTSVTCTLNHPSLPPQRLMALREPAAQAPVKLSLNLLASSDPPEA |
| | | ASWLLCEVSGFSPPNILLMWLEDQREVNTSGFAPARPPPQPGSTTFWA |
| | | WSVLRVPAPPSPQPATYTCVVSHEDSRTLLNASRSLEVSYVTDH |
| 652 | CD8 | IYIWAPLAGTCGVLLLSLVITLYC |
| | transmembrane | |
| 653 | CD28 | FWVLVVVGGVLACYSLLVTVAFIIFWVRSKRSRGGHSDYAMNMTPRRP |
| | transmembrane | GPTRKHYQPYAPPRDFAAYRS |

TABLE 14
CAR costimulatory and primary signaling domain sequences

| SEQ ID NO | Name | Sequence |
|---|---|---|
| 654 | 4-1BB | KRGRKKLLYIFKQPFMRPVQTTQEEDGCSCRFPEEEEGGCEL |
| 655 | CD28 | RSKRSRLLHSDYMNMTPRRPGPTRKHYQPYAPPRDFAAYRS |
| 656 | ICOS | TKKKYSSSVHDPNGEYMFMRAVNTAKKSRLTDVTL |
| 657 | Mutant ICOS | TKKKYSSSVHDPNGEFMFMRAVNTAKKSRLTDVTL |
| 658 | CD27 | KRGRKKLLYIFKQPFMRPVQTTQEEDGCSCRFPEEEEGGCEL |
| 659 | CD3z | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR |
| 660 | Mutant CD3z | RVKFSRSADAPAYKQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR |

TABLE 15
full CAR sequences

| SEQ ID NO | Name | Sequence |
|---|---|---|
| 661 | Anti CD19 | MALPVTALLLPLALLLHAARPDIQMTQTTSSLSASLGDRVTISCRASQD |
| | CAR | ISKYLNWYQQKPDGTVKLLIYHTSRLHSGVPSRFSGSGSGTDYSLTISN |
| | | LEQEDIATYFCQQGNTLPYTFGGGTKLEITGGGGSGGGGSGGGGSEVK |
| | | LQESGPGLVAPSQSLSVTCTVSGVSLPDYGVSWIRQPPRKGLEWLGVI |
| | | WGSETTYYNSALKSRLTIIKDNSKSQVFLKMNSLQTDDTAIYYCAKHY |
| | | YYGGSYAMDYWGQGTSVTVSSTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPR |
| 662 | Anti CD19 | QVQLLESGAELVRPGSSVKISCKASGYAFSSYWMNWVKQRPGQGLEW |
| | CAR | IGQIYPGDGDTNYNGKFKGQATLTADKSSSTAYMQLSGLTSEDSAVYS |
| | | CARKTISSVVDFYFDYWGQGTTVTGGGSGGGSGGGSGGGSELVLTQS |
| | | PKFMSTSVGDRVSVTCKASQNVGTNVAWYQQKPGQSPKPLIYSATYR |
| | | NSGVPDRFTGSGSGTDFTLTITNVQSKDLADYFCQYNRYPYTSFFFTKL |
| | | EIKRRSKIEVMYPPPYLDNEKSNGTIIHVKGKHLCPSPLFPGPSKPFWVL |
| | | VVVGGVLACYSLLVTVAFIIFWVRSKRSRLLHSDYMNMTPRRPGPTRK |
| | | HYQPYAPPRDFAAYRSRVKFSRSADAPAYQQGQNQLYNELNLGRREE |
| | | YDVLDKRRGRDPEMGGKPRRKNPQEGLYNELQKDKMAEAYSEIGMK |
| | | GERRRGKGHDGLYQGLSTATKDTYDALHMQALPPR |
| 663 | Anti CD19 | DIQMTQTTSSLSASLGDRVTISCRASQDISKYLNWYQQKPDGTVKLLIY |
| | CAR | HTSRLHSGVPSRFSGSGSGTDYSLTISNLEQEDIATYFCQQGNTLPYTFG |
| | | GGTKLEITGSTSGSGKPGSGEGSTKGEVKLQESGPGLVAPSQSLSVTCT |
| | | VSGVSLPDYGVSWIRQPPRKGLEWLGVIWGSETTYYNSALKSRLTIIKD |
| | | NSKSQVFLKMNSLQTDDTAIYYCAKHYYYGGSYAMDYWGQGTSVTV |
| | | SSESKYGPPCPPCPMFWVLVVVGGVLACYSLLVTVAFIIFWVKRGRKK |
| | | LLYIFKQPFMRPVQTTQEEDGCSCRFEEEEGGCELRVKFSRSADAPAYQ |
| | | QGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHM |
| | | QALPPRL |
| 664 | Anti CD19 | DIQMTQTTSSLSASLGDRVTISCRASQDISKYLNWYQQKPDGTVKLLIY |
| | CAR | HTSRLHSGVPSRFSGSGSGTDYSLTISNLEQEDIATYFCQQGNTLPYTFG |
| | | GGTKLEITGSTSGSGKPGSGEGSTKGEVKLQESGPGLVAPSQSLSVTCT |
| | | VSGVSLPDYGVSWIRQPPRKGLEWLGVIWGSETTYYNSALKSRLTIIKD |
| | | NSKSQVFLKMNSLQTDDTAIYYCAKHYYYGGSYAMDYWGQGTSVTV |
| | | SSAAAIEVMYPPPYLDNEKSNGTIIHVKGKHLCPSPLFPGPSKPFWVLV |
| | | VVGGVLACYSLLVTVAFIIFWVRSKRSRLLHSDYMNMTPRRPGPTRKH |
| | | YQPYAPPRDFAAYRSRVKFSRSADAPAYQQGQNQLYNELNLGRREEY |
| | | DVLDKRRGRDPEMGGKPRRKNPQEGLYNELQKDKMAEAYSEIGMKG |
| | | ERRRGKGHDGLYQGLSTATKDTYDALHMQALPPR |
| 665 | Anti BCMA | MALPVTALLLPLALLLHAARPDIVLTQSPPSLAMSLGKRATISCRASES |
| | CAR | VTILGSHLIHWYQQKPGQPPTLLIQLASNVQTGVPARFSGSGSRTDFTL |
| | | TIDPVEEDDVAVYYCLQSRTIPRTFGGGTKLEIKGSTSGSGKPGSGEGST |
| | | KGQIQLVQSGPELKKPGETVKISCKASGYTFTDYSINWVKRAPGKGLK |
| | | WMGWINTETREPAYAYDFRGRFAFSLETSASTAYLQINNLKYEDTATY |
| | | FCALDYSYAMDYWGQGTSVTVSSAAATTTPAPRPPTPAPTIASQPLSLR |
| | | PEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTY |
| | | DALHMQALPPR |
| 706 | Anti CD19 | DIQMTQTTSSLSASLGDRVTISCRASQDISKYLNWYQQKPDGTVKLLIY |
| | CAR | HTSRLHSGVPSRFSGSGSGTDYSLTISNLEQEDIATYFCQQGNTLPYTFG |
| | | GGTKLEITGGGGSGGGGSGGGGSEVKLQESGPGLVAPSQSLSVTCTVS |
| | | GVSLPDYGVSWIRQPPRKGLEWLGVIWGSETTYYNSALKSRLTIIKDNS |
| | | KSQVFLKMNSLQTDDTAIYYCAKHYYYGGSYAMDYWGQGTSVTVSS |
| | | GSTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDG |
| | | CSCRFPEEEEGGCELRVKFSRSADAPAYQQGQNQLYNELNLGRREEYD |
| | | VLDKRRGRDPEMGGKPRRKNPQEGLYNELQKDKMAEAYSEIGMKGE |
| | | RRRGKGHDGLYQGLSTATKDTYDALHMQALPPR |

TABLE 16
TCR sequences

| SEQ ID NO | Name | Sequence |
|---|---|---|
| 666 | Anti MAGE | MKNQVEQSPQSLIILEGKNCTLQCNYTVSPFSNLRWYKQDTGRGPVSL |
| | A4 alpha | TIMTFSENTKSNGRYTATLDADTKQSSLHITASQLSDSASYICVVSGGT |
| | chain | DSWGKLQF |
| | variable | |
| | region 1 | |
| 667 | Anti MAGE | MKNQVEQSPQSLIILEGKNCTLQCNYTVSPFSNLRWYKQDTGRGPVSL |
| | A4 alpha | TIVTFSENTKSNGRYTATLDADTKQSSLHITASQLSDSASYICVVSGGT |
| | chain | DSWGKLQF |
| | variable | |
| | region 2 | |
| 668 | Anti MAGE | MKNQVEQSPQSLIILEGKNCTLQCNYTVSPFSNLRWYKQDTGRGPVSL |
| | A4 alpha | TILTFSENTKSNGRYTATLDADTKQSSLHITASQLSDSASYICVVSGGTD |
| | chain | SWGKLQF |
| | variable | |
| | region 3 | |
| 669 | Anti MAGE | MASLLFFCGAFYLLGTGSMDADVTQTPRNRITKTGKRIMLECSQTKGH |
| | A4 beta chain | DRMYWYRQDPGLGLRLIYYSFDVKDINKGEISDGYSVSRQAQAKFSLS |
| | variable | LESAIPNQTALYFCATSGQGAYNEQFF |
| | region 1 | |
| 670 | Anti MAGE | MASLLFFCGAFYLLGTGSMDADVTQTPRNRITKTGKRIMLECSQTKGH |
| | A4 beta chain | DRMYWYRQDPGLGLRLIYYSFDVKDINKGEISDGYSVSRQAQAKFSLS |
| | variable | LESAIPNQTALYFCATSGQGAYEEQFF |
| | region 2 | |
| 671 | Anti MAGE | MSLSSLLKVVTASLWLGPGIAQKITQTQPGMFVQEKEAVTLDCTYDTS |
| | A4 TCR 4 | DQSYGLFWYKQPSSGEMIFLIYQGSYDEQNATEGRYSLNFQKARKSAN |
| | alpha chain | LVISASQLGDSAMYFCAMSGDSAGNMLTFGGGTRLMVKPHIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSSDVPCDVKLVEKSF |
| | | ETDTNLNFQNLSVIGFRILLLKVAGFNLLMTLRLWSS |
| 672 | Anti MAGE | MASLLFFCGAFYLLGTGSMDADVTQTPRNRITKTGKRIMLECSQTKGH |
| | A4 TCR 4 | DRMYWYRQDPGLGLRLIYYSFDVKDINKGEISDGYSVSRQAQAKFSLS |
| | beta chain | LESAIPNQTALYFCATSDWDRSGDKETQYFGPGTRLLVLEDLNKVFPP |
| | | EVAVFEPSKAEIAHTQKATLVCLATGFFPDHVELSWWVNGKEVHSGV |
| | | STDPQPLKEQPALNDSRYCLSSRLRVSATFWQNPRNHFRCQVQFYGLS |
| | | ENDEWTQDRAKPVTQIVSAEAWGRADCGITSASYHQGVLSATILYEIL |
| | | LGKATLYAVLVSALVLMAMVKRKDF |
| 673 | Anti MAGE | MSLSSLLKVVTASLWLGPGIAQKITQTQPGMFVQEKEAVTLDCTYDTS |
| | A4 TCR 5 | DPSYGLFWYKQPSSGEMIFLIYQGSYDQQNATEGRYSLNFQKARKSAN |
| | alpha chain | LVISASQLGDSAMYFCAMSGGYTGGFKTIFGAGTRLFVKANIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSSDVPCDVKLVEKSF |
| | | ETDTNLNFQNLSVIGFRILLLKVAGFNLLMTLRLWSS |
| 674 | Anti MAGE | MGFRLLCCVAFCLLGAGPVDSGVTQTPKHLITATGQRVTLRCSPRSGD |
| | A4 TCR 5 | LSVYWYQQSLDQGLQFLIQYYNGEERAKGNILERFSAQQFPDLHSELN |
| | beta chain | LSSLELGDSALYFCASSGGDGDEQFFGPGTRLTVLEDLKNVFPPEVAVF |
| | | EPSKAEIAHTQKATLVCLATGFYPDHVELSWWVNGKEVHSGVSTDPQ |
| | | PLKEQPALNDSRYCLSSRLRVSATFWQNPRNHFRCQVQFYGLSENDE |
| | | WTQDRAKPVTQIVSAEAWGRADCGITSRSYHQGVLSATILYEILLGKA |
| | | TLYAVLVSALVLMAMVKRKDSRG |
| 675 | Anti MAGE | MTSIRAVFIFLWLQLDLVNGENVEQHPSTLSVQEGDSAVIKCTYSDSAS |
| | A4 TCR 6 | NYFPWYKQELGKRPQLIIDIRSNVGEKKDQRIAVTLNKTAKHFSLHITE |
| | alpha chain | TQPEDSAVYFCAASRGTGFQKLVFGTGTRLLVSPNIQNPDPAVYQLRD |
| | | SKSSDKSVCLFTDFDSQTNVSQSKDSDVYITDKTVLDMRSMDFKSNSA |
| | | VAWSNKSDFACANAFNNSIIPEDTFFPSSDVPCDVKLVEKSFETDTNLN |
| | | FQNLSVIGFRILLLKVAGFNLLMTLRLWSS |
| 676 | Anti MAGE | MGCRLLCCVVFCLLQAGPLDTAVSQTPKYLVTQMGNDKSIKCEQNLG |
| | A4 TCR 6 | HDTMYWYKQDSKKFLKIMFSYNNKELIINETVPNRFSPKSPDKAHLNL |
| | beta chain | HINSLELGDSAVYFCASSQFWDGAGDEQYFGPGTRLTVTEDLNKVFPP |
| | | EVAVFEPSKAEIAHTQKATLVCLATGFFPDHVELSWWVNGKEVHSGV |
| | | STDPQPLKEQPALNDSRYCLSSRLRVSATFWQNPRNHFRCQVQFYGLS |
| | | ENDEWTQDRAKPVTQIVSAEAWGRADCGITSASYHQGVLSATILYEIL |
| | | LGKATLYAVLVSALVLMAMVKRKDF |
| 677 | Anti MAGE | MSLSSLLKVVTASLWLGPGIAQKITQTQPGMFVQEKEAVTLDCTYDTS |
| | A4 TCR 7 | DQSYGLFWYKQPSSGEMIFLIYQGSYDEQNATEGRYSLNFQKARKSAN |
| | alpha chain | LVISASQLGDSAMYFCAMSGDSAGNMLTFGGGTRLMVKPHIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSPESSCDVKLVEKSFE |
| | | TDTNLNFQNLSVIGFRILLLKVAGFNLLMTLRLWSS |
| 678 | Anti MAGE | MASLLFFCGAFYLLGTGSMDADVTQTPRNRITKTGKRIMLECSQTKGH |
| | A4 TCR 7 | DRMYWYRQDPGLGLRLIYYSFDVKDINKGEISDGYSVSRQAQAKFSLS |
| | beta chain | LESAIPNQTALYFCATSDWDRSGDKETQYFGPGTRLLVLEDLNKVFPP |
| | | EVAVFEPSEAEISHTQKATLVCLATGFFPDHVELSWWVNGKEVHSGVS |
| | | TDPQPLKEQPALNDSRYCLSSRLRVSATFWQNPRNHFRCQVQFYGLSE |
| | | NDEWTQDRAKPVTQIVSAEAWGRADCGFTSVSYQQGVLSATILYEILL |
| | | GKATLYAVLVSALVLMAMVKRKDF |
| 679 | Anti MAGE | MSLSSLLKVVTASLWLGPGIAQKITQTQPGMFVQEKEAVTLDCTYDTS |
| | A4 TCR 8 | DPSYGLFWYKQPSSGEMIFLIYQGSYDQQNATEGRYSLNFQKARKSAN |
| | alpha chain | LVISASQLGDSAMYFCAMSGGYTGGFKTIFGAGTRLFVKANIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSPESSCDVKLVEKSFE |
| | | TDTNLNFQNLSVIGFRILLLKVAGFNLLMTLRLWSS |
| 680 | Anti MAGE | MGFRLLCCVAFCLLGAGPVDSGVTQTPKHLITATGQRVTLRCSPRSGD |
| | A4 TCR 8 | LSVYWYQQSLDQGLQFLIQYYNGEERAKGNILERFSAQQFPDLHSELN |
| | beta chain | LSSLELGDSALYFCASSGGDGDEQFFGPGTRLTVLEDLKNVFPPEVAVF |
| | | EPSEAEISHTQKATLVCLATGFYPDHVELSWWVNGKEVHSGVSTDPQP |
| | | LKEQPALNDSRYCLSSRLRVSATFWQNPRNHFRCQVQFYGLSENDEW |
| | | TQDRAKPVTQIVSAEAWGRADCGFTSESYQQGVLSATILYEILLGKAT |
| | | LYAVLVSALVLMAMVKRKDSRG |
| 681 | Anti MAGE | MTSIRAVFIFLWLQLDLVNGENVEQHPSTLSVQEGDSAVIKCTYSDSAS |
| | A4 TCR 9 | NYFPWYKQELGKRPQLIIDIRSNVGEKKDQRIAVTLNKTAKHFSLHITE |
| | alpha chain | TQPEDSAVYFCAASRGTGFQKLVFGTGTRLLVSPNIQNPDPAVYQLRD |
| | | SKSSDKSVCLFTDFDSQTNVSQSKDSDVYITDKTVLDMRSMDFKSNSA |
| | | VAWSNKSDFACANAFNNSIIPEDTFFPSPESSCDVKLVEKSFETDTNLNF |
| | | QNLSVIGFRILLLKVAGFNLLMTLRLWSS |
| 682 | Anti MAGE | MGCRLLCCVVFCLLQAGPLDTAVSQTPKYLVTQMGNDKSIKCEQNLG |
| | A4 TCR 9 | HDTMYWYKQDSKKFLKIMFSYNNKELIINETVPNRFSPKSPDKAHLNL |
| | beta chain | HINSLELGDSAVYFCASSQFWDGAGDEQYFGPGTRLTVTEDLNKVFPP |
| | | EVAVFEPSEAEISHTQKATLVCLATGFFPDHVELSWWVNGKEVHSGVS |
| | | TDPQPLKEQPALNDSRYCLSSRLRVSATFWQNPRNHFRCQVQFYGLSE |
| | | NDEWTQDRAKPVTQIVSAEAWGRADCGFTSVSYQQGVLSATILYEILL |
| | | GKATLYAVLVSALVLMAMVKRKD |
| 683 | Anti MAGE | MSLSSLLKVVTASLWLGPGIAQKITQTQPGMFVQEKEAVTLDCTYDTS |
| | A4 TCR 10 | DQSYGLFWYKQPSSGEMIFLIYQGSYDEQNATEGRYSLNFQKARKSAN |
| | alpha chain | LVISASQLGDSAMYFCAMSGDSAGNMLTFGGGTRLMVKPHIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSSDVPCDVKLVEKSF |
| | | ETDTNLNFQNLLVIVLRILLLKVAGFNLLMTLRLWSS |
| 684 | Anti MAGE | MASLLFFCGAFYLLGTGSMDADVTQTPRNRITKTGKRIMLECSQTKGH |
| | A4 TCR 10 | DRMYWYRQDPGLGLRLIYYSFDVKDINKGEISDGYSVSRQAQAKFSLS |
| | beta chain | LESAIPNQTALYFCATSDWDRSGDKETQYFGPGTRLLVLEDLNKVFPP |
| | | EVAVFEPSKAEIAHTQKATLVCLATGFFPDHVELSWWVNGKEVHSGV |
| | | STDPQPLKEQPALNDSRYCLSSRLRVSATFWQNPRNHFRCQVQFYGLS |
| | | ENDEWTQDRAKPVTQIVSAEAWGRADCGITSASYHQGVLSATILYEIL |
| | | LGKATLYAVLVSALVLMAMVKRKDF |
| 685 | Anti MAGE | MSLSSLLKVVTASLWLGPGIAQKITQTQPGMFVQEKEAVTLDCTYDTS |
| | A4 TCR 11 | DPSYGLFWYKQPSSGEMIFLIYQGSYDQQNATEGRYSLNFQKARKSAN |
| | alpha chain | LVISASQLGDSAMYFCAMSGGYTGGFKTIFGAGTRLFVKANIQNPDPA |
| | | VYQLRDSKSSDKSVCLFTDFDSQTNVSQSKDSDVYITDKTVLDMRSM |
| | | DFKSNSAVAWSNKSDFACANAFNNSIIPEDTFFPSSDVPCDVKLVEKSF |
| | | ETDTNLNFQNLLVIVLRILLLKVAGFNLLMTLRLWSS |
| 686 | Anti MAGE | MGFRLLCCVAFCLLGAGPVDSGVTQTPKHLITATGQRVTLRCSPRSGD |
| | A4 TCR 11 | LSVYWYQQSLDQGLQFLIQYYNGEERAKGNILERFSAQQFPDLHSELN |
| | beta chain | LSSLELGDSALYFCASSGGDGDEQFFGPGTRLTVLEDLKNVFPPEVAVF |
| | | EPSKAEIAHTQKATLVCLATGFYPDHVELSWWVNGKEVHSGVSTDPQ |
| | | PLKEQPALNDSRYCLSSRLRVSATFWQNPRNHFRCQVQFYGLSENDE |
| | | WTQDRAKPVTQIVSAEAWGRADCGITSASYHQGVLSATILYEILLGKA |
| | | TLYAVLVSALVLMAMVKRKDSRG |
| 687 | Anti MAGE | MTSIRAVFIFLWLQLDLVNGENVEQHPSTLSVQEGDSAVIKCTYSDSAS |
| | A4 TCR 12 | NYFPWYKQELGKRPQLIIDIRSNVGEKKDQRIAVTLNKTAKHFSLHITE |
| | alpha chain | TQPEDSAVYFCAASRGTGFQKLVFGTGTRLLVSPNIQNPDPAVYQLRD |
| | | SKSSDKSVCLFTDFDSQTNVSQSKDSDVYITDKTVLDMRSMDFKSNSA |
| | | VAWSNKSDFACANAFNNSIIPEDTFFPSSDVPCDVKLVEKSFETDTNLN |
| | | FQNLLVIVLRILLLKVAGFNLLMTLRLWSS |
| 688 | Anti MAGE | MGCRLLCCVVFCLLQAGPLDTAVSQTPKYLVTQMGNDKSIKCEQNLG |
| | A4 TCR 12 | HDTMYWYKQDSKKFLKIMFSYNNKELIINETVPNRFSPKSPDKAHLNL |
| | beta chain | HINSLELGDSAVYFCASSQFWDGAGDEQYFGPGTRLTVTEDLNKVFPP |
| | | EVAVFEPSKAEIAHTQKATLVCLATGFFPDHVELSWWVNGKEVHSGV |
| | | STDPQPLKEQPALNDSRYCLSSRLRVSATFWQNPRNHFRCQVQFYGLS |
| | | ENDEWTQDRAKPVTQIVSAEAWGRADCGITSASYHQGVLSATILYEIL |
| | | LGKATLYAVLVSALVLMAMVKRKD |
| 689 | Anti MAGE | MKNQVEQSPQSLIILEGKNCTLQCNYTVSPFSNLRWYKQDTGRGPVSL |
| | B2 alpha | TIMTFSENTKSNGRYTATLDADTKQSSLHITASQLSDSASYICVVSGGT |
| | chain | DSWGKLQF |
| | variable | |
| | region 1 | |
| 690 | Anti MAGE | MKNQVEQSPQSLIILEGKNCTLQCNYTVSPFSNLRWYKQDTGRGPVSL |
| | B2 alpha | TRMTFSENTKSNGRYTATLDADTKQSSLHITASQLSDSASYICVVSGGT |
| | chain | DSWGKLQF |
| | variable | |
| | region 2 | |
| 691 | Anti MAGE | MASLLFFCGAFYLLGTGSMDADVTQTPRNRITKTGKRIMLECSQTKGH |
| | B2 beta chain | DRMYWYRQDPGLGLRLIYYSFDVKDINKGEISDGYSVSRQAQAKFSLS |
| | variable | LESAIPNQTALYFCATSGQGAYNEQFF |
| | region 1 | |
| 692 | Anti MAGE | MASLLFFCGAFYLLGTGSMDADVTQTPRNRITKTGKRIMLECSQTKGR |
| | B2 beta chain | DRMYWYRQDPGLGLRLIYYSFDVKDINKGEISDGYSVSRQAQAKFSLS |
| | variable | LESAIPNQTALYFCATSGQGAYNEQFF |
| | region 2 | |
| 693 | Anti MAGE | MASLLFFCGAFYLLGTGSMDADVTQTPRNRITKTGKRIMLECSQTKGH |
| | B2 beta chain | DRMYWYRQDPGLGLRLIYYSFDVKDINKGEISDGYSVSRQAQAKFSLS |
| | variable | LESAIPNQTALYFCATSGQGAYEEQFF |
| | region 3 | |
| 694 | Anti MAGE | MASLLFFCGAFYLLGTGSMDADVTQTPRNRITKTGKRIMLECSQTKGH |
| | B2 beta chain | DRMYWYRQDPGLGLRLIYYSFDVKDINKGEISDGYSVSRQAQAKFSLS |
| | variable | LESAIPNQTALYFCATSGQGAYREQFF |
| | region 4 | |
| 695 | Anti MAGE | QKEVEQNSGPLSVPEGAIASLNCTYSDRGSQSFFWYRQYSGKSPELIMS |
| | A10 alpha | IYSNGDKEDGRFTAQLNKASQYVSLLIRDSQPSDSATYLCAVRGTGRR |
| | chain | ALTFGSGTRLQVQPNIQNPDPAVYQLRDSKSSDKSVCLFTDFDSQTNVS |
| | variable | QSKDSDVYITDKTVLDMRSMDFKSNSAVAWSNKSDFACANAFNNSIIP |
| | region 1 | EDTFFPSPESS |
| 696 | Anti MAGE | QKEVEQNSGPLSVPEGAIASLNCTYSDRGSASFFWYRQYSGKSPELIMS |
| | A10 alpha | IYSNGDKEDGRFTAQLNKASQYVSLLIRDSQPSDSATYLCAVRGTGRR |
| | chain | ALTFGSGTRLQVQPNIQNPDPAVYQLRDSKSSDKSVCLFTDFDSQTNVS |
| | variable | QSKDSDVYITDKTVLDMRSMDFKSNSAVAWSNKSDFACANAFNNSIIP |
| | region 2 | EDTFFPSPESS |
| 697 | Anti MAGE | QKEVEQNSGPLSVPEGAIASLNCTYSDRGSSSFFWYRQYSGKSPELIMSI |
| | A10 alpha | YSNGDKEDGRFTAQLNKASQYVSLLIRDSQPSDSATYLCAVRGTGRRA |
| | chain | LTFGSGTRLQVQPNIQNPDPAVYQLRDSKSSDKSVCLFTDFDSQTNVSQ |
| | variable | SKDSDVYITDKTVLDMRSMDFKSNSAVAWSNKSDFACANAFNNSIIPE |
| | region 3 | DTFFPSPESS |
| 698 | Anti MAGE | NAGVTQTPKFRVLKTGQSMTLLCAQDMNHEYMYWYRQDPGMGLRLI |
| | A10 beta | HYSVGEGTTAKGEVPDGYNVSRLKKQNFLLGLESAAPSQTSVYFCASS |
| | chain | FTDTQYFGPGTRLTVLEDLKNVFPPEVAVFEPSEAEISHTQKATLVCLA |
| | variable | TGFYPDHVELSWWVNGKEVHSGVSTDPQPLKEQPALNDSRYCLSSRL |
| | region 1 | RVSATFWQNPRNHFRCQVQFYGLSENDEWTQDRAKPVTQIVSAEAW |
| | | GRAD |
| 699 | Anti MAGE | NAGVTQTPKFRVLKTGQSMTLLCAQDMNHEYMYWYRQDPGMGLRLI |
| | A10 beta | HYSVSEGTTAKGEVPDGYNVSRLKKQNFLLGLESAAPSQTSVYFCASS |
| | chain | FTDTQYFGPGTRLTVLEDLKNVFPPEVAVFEPSEAEISHTQKATLVCLA |
| | variable | TGFYPDHVELSWWVNGKEVHSGVSTDPQPLKEQPALNDSRYCLSSRL |
| | region 2 | RVSATFWQNPRNHFRCQVQFYGLSENDEWTQDRAKPVTQIVSAEAW |
| | | GRAD |
| 700 | Anti MAGE | NAGVTQTPKFRVLKTGQSMTLLCAQDMNHEYMYWYRQDPGMGLRLI |
| | A10 beta | HYSVAEGTTAKGEVPDGYNVSRLKKQNFLLGLESAAPSQTSVYFCASS |
| | chain | FTDTQYFGPGTRLTVLEDLKNVFPPEVAVFEPSEAEISHTQKATLVCLA |
| | variable | TGFYPDHVELSWWVNGKEVHSGVSTDPQPLKEQPALNDSRYCLSSRL |
| | region 3 | RVSATFWQNPRNHFRCQVQFYGLSENDEWTQDRAKPVTQIVSAEAW |
| | | GRAD |
| 701 | Anti MAGE | NAGVTQTPKFRVLKTGQSMTLLCAQDMNHEYMYWYRQDPGMGLRLI |
| | A10 beta | HYSVFEGTTAKGEVPDGYNVSRLKKQNFLLGLESAAPSQTSVYFCASS |
| | chain | FTDTQYFGPGTRLTVLEDLKNVFPPEVAVFEPSEAEISHTQKATLVCLA |
| | variable | TGFYPDHVELSWWVNGKEVHSGVSTDPQPLKEQPALNDSRYCLSSRL |
| | region 4 | RVSATFWQNPRNHFRCQVQFYGLSENDEWTQDRAKPVTQIVSAEAW |
| | | GRAD |
| 702 | Anti MAGE | NAGVTQTPKFRVLKTGQSMTLLCAQDMNHDYMYWYRQDPGMGLRL |
| | A10 beta | IHYSVGEGTTAKGEVPDGYNVSRLKKQNFLLGLESAAPSQTSVYFCAS |
| | chain | SFTDTQYFGPGTRLTVLEDLKNVFPPEVAVFEPSEAEISHTQKATLVCL |
| | variable | ATGFYPDHVELSWWVNGKEVHSGVSTDPQPLKEQPALNDSRYCLSSR |
| | region 5 | LRVSATFWQNPRNHFRCQVQFYGLSENDEWTQDRAKPVTQIVSAEAW |
| | | GRAD |

TABLE 17
TFP sequences

| SEQ ID NO | Name | Sequence |
|---|---|---|
| 703 | Anti | EVQLVESGGGLVQPGGSLRLSCAAGGDWSANFMYWYRQAPGKQREL |
| | mesothelin | VARISGRGVVDYVEVKGRFTISRDNSKNTLYLQMNSLRAEDTAVYYC |
| | binder 1 | AVASYWGQGTLVTVSS |
| 704 | Anti | EVQLVESGGGLVQPGGSLRLSCAAGSTSSINTMYWYRQAPGKERELV |
| | mesothelin | AFISSGGSTNVRDSVKGRFTISRDNSKNTLYLQMNSLRAEDTAVYYCN |
| | binder 2 | TYIPYGGTLHDFWGQGTLVTVSS |
| 705 | Anti | QVQVESGGGVVQAGGSLRLSCAASGSTFSIRAMRWYRQAPGTERDLV |
| | mesothelin | AVIYGSSTYYADAVKGRFTISRDNSKNTLYLQMNSLRAEDTAVYYCN |
| | binder 3 | ADTIGTARDYWGQGTLVTVSS |

1. A method of treating a subject in need thereof, comprising administering to the subject an effective amount of:
a) an inducible cytokine prodrug, and
b) an adoptive cell therapy.

2. A method of treating a subject in need thereof, comprising administering to the subject an effective amount of:
a) an inducible cytokine prodrug, and
b) a polynucleotide encoding an antigen binding protein.

3. A method of treating a subject in need thereof, comprising administering to the subject an effective amount of:
a) a polynucleotide encoding an inducible cytokine prodrug, and
b) an adoptive cell therapy.

4. A method of treating a subject in need thereof, comprising administering to the subject an effective amount of:
a) a polynucleotide encoding an inducible cytokine prodrug, and
b) a polynucleotide encoding an antigen binding protein.

5. The method of claim 3 or claim 4, wherein the polynucleotide encoding an inducible cytokine prodrug is provided as a viral vector.

6. The method of claim 3 or claim 4, wherein the polynucleotide encoding an inducible cytokine prodrug is encapsulated in a viral particle.

7. The method of claim 3 or claim 4, wherein the polynucleotide encoding an inducible cytokine prodrug is encapsulated in a nanoparticle.

8. The method of claim 2 or claim 4, wherein the polynucleotide encoding an antigen binding protein is provided as a viral vector.

9. The method of any of claims 2, 4, or 8, wherein the polynucleotide encoding an antigen binding protein is encapsulated in a viral particle.

10. The method of claim 2 or claim 4, wherein the polynucleotide encoding an antigen binding protein is encapsulated in a nanoparticle.

11. The method of any of claims 2, or 4-10, wherein the antigen binding protein, when expressed in an immune cell, directs the immune cell and its immune effector functions to cells that express a desired antigen.

12. A method of treating a subject in need thereof, comprising administering to the subject an effective amount of:
a) A polynucleotide encoding an inducible cytokine prodrug and an antigen binding protein.

13. The method of claim 12, wherein the polynucleotide encoding an inducible cytokine prodrug and an antigen binding protein is provided as a viral vector.

14. The method of claim 12, wherein the polynucleotide encoding an inducible cytokine prodrug and an antigen binding protein is encapsulated in a viral particle.

15. The method of claim 12, wherein the polynucleotide encoding an inducible cytokine prodrug and an antigen binding protein is encapsulated in a nanoparticle.

16. A method of treating a subject in need thereof, comprising administering to the subject an effective amount of:
a) An immune cell comprising a polynucleotide encoding an inducible cytokine prodrug.

17. The method of any preceding claim, wherein the inducible cytokine prodrug comprises:
a) a cytokine polypeptide,
b) a protease cleavable linker, and
c) a blocking element,
wherein the inducible cytokine prodrug has attenuated biological activity and wherein cleavage of the protease cleavable linker by a protease produces a cytokine polypeptide with biological activity that is not attenuated.

18. The method of any of claims 1-16, wherein the inducible cytokine prodrug comprises
a) a inducible cytokine prodrug comprising
i) a cytokine polypeptide,
ii) a protease cleavable linker, and
iii) a blocking element, and
b) a second polypeptide
wherein the complex of the inducible cytokine prodrug and the second polypeptide has attenuated biological activity and wherein cleavage of the protease cleavable linker by a protease produces a cytokine polypeptide with biological activity that is not attenuated.

19. The method of claim 18, wherein the cytokine polypeptide is a cytokine subunit or a functional fragment thereof, and the second polypeptide comprises a second cytokine subunit or a functional fragment thereof, wherein the cytokine subunit or functional fragment thereof and the second cytokine subunit or functional fragment thereof are capable of associating to form a complex that has attenuated biological activity and wherein cleavage of the protease cleavable linker by a protease produces a cytokine with biological activity that is not attenuated.

20. The method of claim 19, wherein the first cytokine subunit or functional fragment thereof is IL-12 subunit p35 or a functional fragment thereof, and the second cytokine subunit or functional fragment thereof is IL-12 subunit p40 or a functional fragment thereof.

21. The method of claim 19, wherein the first cytokine subunit or functional fragment thereof is IL-12 subunit p40 or a functional fragment thereof, and the second cytokine subunit or functional fragment thereof is IL-12 subunit p35 or a functional fragment thereof.

22. The method of any of claims 19-20, wherein the second subunit protein is exogenous to the subject.

23. The method of any of claims 19-20, wherein the second subunit protein further comprises a linker and a half-life extension moiety.

24. The method of any of claims 1-16, wherein the inducible cytokine prodrug comprises
a) a inducible cytokine prodrug comprising
i) a cytokine polypeptide,
ii) a protease cleavable linker, and
iii) a first fragment of an antibody specific for the cytokine polypeptide, and
b) a second polypeptide comprising a second fragment of an antibody specific for the cytokine polypeptide,
wherein the first fragment of an antibody specific for the cytokine or cytokine polypeptide and second polypeptide comprising a second fragment of an antibody specific for the cytokine or cytokine polypeptide are capable of associating to form an antibody specific for the cytokine or cytokine polypeptide such that the biological activity of the complex is attenuated and wherein cleavage of the protease cleavable linker by a protease produces a cytokine with biological activity that is not attenuated.

25. The method of any preceding claim, wherein the second subunit protein is endogenous to the subject.

26. The method of claim 17 or claim 24, wherein the cytokine polypeptide is IL-2 or a mutein or functional fragment thereof.

27. The method of any of claims 17, 18, or 24, wherein the cytokine polypeptide is IL-12 subunit p35 or a mutein or functional fragment thereof.

28. The method of any of claims 17, 18, or 24, wherein the cytokine polypeptide is IL-12 subunit p40 or a mutein or functional fragment thereof.

29. The method of claim 17 or claim 24, wherein the cytokine polypeptide comprises IL-12 subunit p35 or a mutein or functional fragment thereof and IL-12 subunit p40 or a mutein or functional fragment thereof.

30. The method of claim 17 or claim 24, wherein the cytokine polypeptide can be defined by the formula [A1]-[L5]-[A2] wherein [A1] is IL-12 subunit p40 or a mutein or functional fragment thereof, [A2] is IL-12 subunit p35 or a mutein or functional fragment thereof, and [L5] is a polypeptide linker that is optionally protease cleavable.

31. The method of claim 17 or claim 24, wherein the cytokine polypeptide is IL-15 or a mutein, or functional fragment thereof.

32. The method of claim 17 or claim 24, wherein the cytokine polypeptide is IL-21 or a mutein, or functional fragment thereof.

33. The method of any of claims 17-32, wherein the protease cleavable linker comprises an amino acid sequence selected from SEQ ID NOs: 195-220 or an amino acid sequence that has at least about 90% identity to SEQ ID NOs: 195-220.

34. The method of any of claims 17-33, wherein the blocking element is selected from a steric blocking element, a specific blocking element, and the combination thereof.

35. The method of any of claims 17-34, wherein the protease cleavable linker comprises one cleavable moiety, which is a substrate for a protease.

36. The method of any of claims 17-34, wherein the protease cleavable linker comprises two or more cleavable moieties, each of which is a substrate for a protease.

37. The method of claims 17-36, wherein the protease cleavable linker comprises a first cleavable moiety comprising a first amino acid sequence that is a substrate for a first protease and a second cleavable moiety comprising a second amino acid sequence that is a substrate for a second protease.

38. The method of any of claims 17-35 or 37, wherein the protease cleavable linker further comprises a non-cleavable linker sequence.

39. The method of any of claims 17-35, 37, or 38, wherein the protease cleavable linker is cleaved with either (a) greater catalytic efficiency or (b) greater specificity or (c) both (a) and (b), by one or more proteases than a reference polypeptide sequence.

40. The method of claim 39, wherein the one or more proteases are selected from the group consisting of FAPα, CTSL1, an ADAM selected from ADAM 8, ADAM 9, ADAM 10, ADAM12 ADAM17, and ADAMTS1, and an MMP selected from MMP1, MMP2, MMP9 and MMP14.

41. The method of claim 39, wherein the one or more proteases are selected from MMP1, MMP2, MMP9, MMP4, cathepsin B, cathepsin C, cathepsin D, cathepsin E, cathepsin G, cathepsin K or cathepsin L.

42. The method of any of claims 39-41, wherein the reference polypeptide sequence is present in a naturally occurring polypeptide substrate for FAPα, CTSL1, an ADAM selected from ADAM 8, ADAM 9, ADAM 10, ADAM12 ADAM17, and ADAMTS1, and an MMP selected from MMP1, MMP2, MMP9 and MMP14, or a combination thereof.

43. The method of any of claims 17-42, wherein the blocking element comprises human serum albumin (HSA) or an anti-HSA antibody.

44. The method of any of claims 17-43, wherein the inducible cytokine prodrug further comprises one or more half-life extension domains.

45. The method of any of claims 17-44, wherein the inducible cytokine prodrug has a half-life of at least about 1 hour, 2 hours, 3 hours, 4 hours, 5 hours, 6 hours, 7 hours, 8 hours, 9 hours, 10 hours, 11 hours, 12 hours, 18 hours, or 24 hours.

46. The method of any of claims 1, 3, 5-7, or 17-45, wherein the adoptive cell therapy comprises TILs.

47. The method of any of claims 1, 3, 5-7, or 17-46, wherein the adoptive cell therapy comprises a T cell, an NK cell, or a NKT cell.

48. The method of any of claims 1, 3, 5-7, or 17-47, wherein the adoptive cell therapy comprises an immune cell that comprises an antigen binding protein.

49. The method of any of claims 2, 4-14, or 17-48, wherein the antigen binding protein confers specificity for an antigen selected from the group consisting of CD19, CD123, CD22, CD30, CD171, CS-1, CLL-1 (CLECL1), CD33, EGFRVIII, GD2, GD3, BCMA, Tn Ag, PSMA, ROR1, FLT3, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, FAP, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, fibronectin EDB (EDB-FN), 5T4 oncofetal antigen, and IGLL1.

50. The method of any of claims 2, 4-15, or 17-49, wherein the antigen binding protein is a CAR.

51. The method of any claim 50, wherein the CAR comprises a binding domain sequence selected from SEQ ID NO: 562-648.

52. The method of claim 50 or claim 51, wherein the CAR comprises a hinge domain sequence selected from SEQ ID NO: 649-651.

53. The method of any of claims 50-52, wherein the CAR comprises a transmembrane domain sequence selected from SEQ ID NO: 652-653.

54. The method of any of claims 50-53, wherein the CAR comprises a costimulatory domain sequence selected from SEQ ID NO: 654-658.

55. The method of any of claims 50-54, wherein the CAR comprises a primary signaling domain sequence selected from SEQ ID NO: 659-660.

56. The method of claim 50, wherein the CAR comprises a sequence selected from SEQ ID NO: 661-665.

57. The method of any of claims 2, 4-15, or 17-49, wherein the antigen binding protein is an exogenous TCR subunit.

58. The method of claim 57, wherein the TCR subunit comprises a sequence selected from SEQ ID NO: 666-702.

59. The method of any of claims 2, 4-15, or 17-49, wherein the antigen binding protein is a TFP.

60. The method of 59, wherein the TFP comprises a sequence selected from SEQ ID NO: 703-705.

61. The method of any of claims 1-48, 50, 51-57, or 59, wherein the subject has an autoimmune disease.

62. The method of claim 61, wherein the autoimmune disease is selected from the group consisting of graft versus host disease, multiple sclerosis, rheumatoid arthritis, myasthenia gravis, crohn's disease, and lupus.

63. The method of any of claims 1-48, 50-57, or 59, further comprising the step of selecting a subject at risk of graft rejection.

64. The method of any of claims 61-63, wherein adoptive cell therapy comprises Treg.

65. The method of any of claims 61-64, wherein the adoptive cell therapy comprises an immune cell that comprises a chimeric autoantibody receptor (CAAR).

66. The method of any of claims 1, 3, 5-7, or 17-65, wherein the adoptive cell therapy comprises autologous immune cells.

67. The method of any of claims 1, 3, 5-7, or 17-65, wherein the adoptive cell therapy comprises allogeneic immune cells.

68. The method of claim 67, wherein an endogenous TCR gene in the allogeneic immune cells has been disrupted.

69. The method of claim 67 or claim 68, wherein an endogenous MHC gene in the allogeneic immune cell has been disrupted.

70. The method of any of claims 17-70, wherein the inducible cytokine prodrug has a half-life of at least about 1 hour, or wherein the inducible cytokine prodrug has a half-life of at least about 2 hours, or wherein the inducible cytokine prodrug has a half-life of at least about 3 hours, or wherein the inducible cytokine prodrug has a half-life of at least about 4 hours, or wherein the inducible cytokine prodrug has a half-life of at least about 5 hours, or wherein the inducible cytokine prodrug has a half-life of at least about 6 hours, or wherein the inducible cytokine prodrug has a half-life of at least about 7 hours, or wherein the inducible cytokine prodrug has a half-life of at least about 8 hours, or wherein the inducible cytokine prodrug has a half-life of at least about 9 hours, or wherein the inducible cytokine prodrug has a half-life of at least about 10 hours, or wherein the inducible cytokine prodrug has a half-life of at least about 11 hours, or wherein the inducible cytokine prodrug has a half-life of at least about 12 hours, or wherein the inducible cytokine prodrug has a half-life of at least about 18 hours, or wherein the inducible cytokine prodrug has a half-life of at least about 24 hours.

71. The method of any of claims 17-70, wherein the subject is administered 1 dose of the inducible cytokine prodrug, or wherein the subject is administered 2 doses of the inducible cytokine prodrug, or wherein the subject is administered 3 doses of the inducible cytokine prodrug, or wherein the subject is administered 4 doses of the inducible cytokine prodrug, or wherein the subject is administered 5 or more doses of the inducible cytokine prodrug.

72. The method of any of claims 17-71, wherein the subject is administered 1 dose of the adoptive cell therapy, or wherein the subject is administered 2 doses of the adoptive cell therapy, or wherein the subject is administered 3 doses of the adoptive cell therapy, or wherein the subject is administered 4 doses of the adoptive cell therapy, or wherein the subject is administered 5 or more doses of the adoptive cell therapy.

73. The method of any of claims 17-72, wherein at least one dose of the inducible cytokine prodrug and at least one dose of the adoptive cell therapy are administered to the subject simultaneously.

74. The method of any of claims 17-73, wherein at least one dose of the inducible cytokine prodrug is administered before at least one dose of the immune cell.

75. The method of any of claims 17-74, wherein at least one dose of the inducible cytokine prodrug is administered about 1 day before at least one dose of the adoptive cell therapy, or wherein at least one dose of the inducible cytokine prodrug is administered about 2 days before at least one dose of the adoptive cell therapy, or wherein at least one dose of the inducible cytokine prodrug is administered about 3 days before at least one dose of the adoptive cell therapy, or wherein at least one dose of the inducible cytokine prodrug is administered about 4 days before at least one dose of the adoptive cell therapy, or wherein at least one dose of the inducible cytokine prodrug is administered about 5 days before at least one dose of the adoptive cell therapy, or wherein at least one dose of the inducible cytokine prodrug is administered about 6 days before at least one dose of the adoptive cell therapy, or wherein at least one dose of the inducible cytokine prodrug is administered about 7 days before at least one dose of the adoptive cell therapy, or wherein at least one dose of the inducible cytokine prodrug is administered about 2 weeks before at least one dose of the adoptive cell therapy, or wherein at least one dose of the inducible cytokine prodrug is administered about 3 weeks before at least one dose of the adoptive cell therapy, or wherein at least one dose of the inducible cytokine prodrug is administered about 4 weeks before at least one dose of the adoptive cell therapy, or wherein at least one dose of the inducible cytokine prodrug is administered about 5 weeks before at least one dose of the adoptive cell therapy, or wherein at least one dose of the inducible cytokine prodrug is administered about 6 weeks before at least one dose of the adoptive cell therapy.

76. The method of any of claims 17-75, wherein at least one dose of the inducible cytokine prodrug is administered after at least one dose of the adoptive cell therapy.

77. The method of any of claims 17-76, wherein at least one dose of the inducible cytokine prodrug is administered about 1 day after at least one dose of the adoptive cell therapy, or wherein at least one dose of the inducible cytokine prodrug is administered about 2 days after at least one dose of the adoptive cell therapy, or wherein at least one dose of the inducible cytokine prodrug is administered about 3 days after at least one dose of the adoptive cell therapy, or wherein at least one dose of the inducible cytokine prodrug is administered about 4 days after at least one dose of the adoptive cell therapy, or wherein at least one dose of the inducible cytokine prodrug is administered about 5 days after at least one dose of the adoptive cell therapy, or wherein at least one dose of the inducible cytokine prodrug is administered about 6 days after at least one dose of the adoptive cell therapy, or wherein at least one dose of the inducible cytokine prodrug is administered about 7 days after at least one dose of the adoptive cell therapy, or wherein at least one dose of the inducible cytokine prodrug is administered about 2 weeks after at least one dose of the adoptive cell therapy, or wherein at least one dose of the inducible cytokine prodrug is administered about 3 weeks after at least one dose of the adoptive cell therapy, or wherein at least one dose of the inducible cytokine prodrug is administered about 4 weeks after at least one dose of the adoptive cell therapy, or wherein at least one dose of the inducible cytokine prodrug is administered about 5 weeks after at least one dose of the adoptive cell therapy, or wherein at least one dose of the inducible cytokine prodrug is administered about 6 weeks after at least one dose of the adoptive cell therapy.

78. The method of any of claims 17-77, wherein at least one dose of the inducible cytokine prodrug is administered to a subject when the administered immune cell count has decreased by about 10%, or wherein at least one dose of the inducible cytokine prodrug is administered to a subject when the administered immune cell count has decreased by about 20%, or wherein at least one dose of the inducible cytokine prodrug is administered to a subject when the administered immune cell count has decreased by about 30%, or wherein at least one dose of the inducible cytokine prodrug is administered to a subject when the administered immune cell count has decreased by about 40%, or wherein at least one dose of the inducible cytokine prodrug is administered to a subject when the administered immune cell count has decreased by about 50%, or wherein at least one dose of the inducible cytokine prodrug is administered to a subject when the administered immune cell count has decreased by about 60%, or wherein at least one dose of the inducible cytokine prodrug is administered to a subject when the administered immune cell count has decreased by about 70%, or wherein at least one dose of the inducible cytokine prodrug is administered to a subject when the administered immune cell count has decreased by about 80%, or wherein at least one dose of the inducible cytokine prodrug is administered to a subject when the administered immune cell count has decreased by about 90%.

79. The method of any of claims 17-78, wherein at least one dose of the inducible cytokine prodrug is administered to a subject when administered immune cells are no longer detectable.

80. The method of any of claims 17-79, wherein the inducible cytokine prodrug and the immune cell have an overlap in the timing of their pharmacological or biological activities.

81. The method of any preceding claim, further comprising the step of administering a chemotherapeutic agent.

82. The method of claim 81, wherein the chemotherapeutic agent is selected from the group consisting of Adriamycin, Cerubidine, Bleomycin, Alkeran, Velban, Oncovin, Fluorouracil, Thiotepa, Methotrexate, Bisantrene, Noantrone, Thiguanine, Cytaribine, and Procarabizine.

83. The method of any preceding claim, further comprising the step of administering a checkpoint inhibitor.

84. The method of claim 83, wherein the checkpoint inhibitor is selected from the group consisting of an anti-PD-L1 agent, an anti-CTLA4 agent, an anti-PD-1 agent, an anti-CD47 agent, and an anti-GD2 agent.

85. The method of any preceding claim, wherein 2 or more inducible cytokine prodrugs comprising different cytokines, muteins, subunits, or functional fragments thereof are administered.

86. A pharmaceutical composition comprising:
a) A inducible cytokine prodrug comprising:
i) A cytokine polypeptide selected from the group IL-2, IL-12, IL-15, and IL-21, or a functional fragment, mutein or subunit thereof,
ii) A cleavable linker comprising an amino acid sequence selected from SEQ ID NOs: 195-220 or an amino acid sequence that has at least about 90% identity to SEQ ID NOs: 195-220, and
iii) A blocking element selected from a steric blocking element, a specific blocking element, and the combination thereof, and
b) An adoptive cell therapy.

87. The pharmaceutical composition of claim 86, wherein the inducible cytokine prodrug has attenuated biological activity and wherein cleavage of the protease cleavable linker by the protease produces a cytokine or a functional fragment thereof with biological activity that is not attenuated.

88. The pharmaceutical composition of any of claims 86-87, wherein the blocking element comprises human serum albumin (HSA) or an anti-HSA antibody.

89. The pharmaceutical composition of any of claims 86-88, wherein the inducible cytokine prodrug further comprises one or more half-life extension domains.

90. A polynucleotide encoding:
a) A inducible cytokine prodrug comprising:
i) A cytokine polypeptide selected from the group IL-2, IL-12, IL-15, and IL-21, or a functional fragment, mutein or subunit thereof,
ii) A cleavable linker comprising an amino acid sequence selected from SEQ ID NOs: 195-220 or an amino acid sequence that has at least about 90% identity to SEQ ID NOs: 195-220, and
iii) A blocking element selected from a steric blocking element, a specific blocking element, and the combination thereof, and
b) An antigen binding protein.

91. The polynucleotide of claim 90, wherein the inducible cytokine prodrug has attenuated biological activity and wherein cleavage of the protease cleavable linker by the protease produces a cytokine or a functional fragment thereof with biological activity that is not attenuated.

92. The polynucleotide of claim 90 or claim 91, wherein the blocking element comprises human serum albumin (HSA) or an anti-HSA antibody.

93. The polynucleotide of any of claims 90-92, wherein the inducible cytokine prodrug further comprises one or more half-life extension domains.

94. The polynucleotide of any of claims 90-93, wherein the antigen binding protein, when expressed in an immune cell, directs the immune cell and its immune effector functions to cells that express a desired antigen.

95. An immune cell comprising:
a) A polynucleotide encoding an inducible cytokine prodrug comprising:
i) A cytokine polypeptide selected from the group IL-2, IL-12, IL-15, and IL-21, or a functional fragment, mutein or subunit thereof,
ii) A protease cleavable linker comprising an amino acid sequence selected from SEQ ID NOs: 195-220 or an amino acid sequence that has at least about 90% identity to SEQ ID NOs: 195-220, and
iii) A blocking element selected from a steric blocking element, a specific blocking element, and the combination thereof, and
b) A polynucleotide encoding an antigen binding protein.

96. The immune cell of claim 95, wherein the inducible cytokine prodrug has attenuated biological activity and wherein cleavage of the protease cleavable linker by the protease produces a cytokine or a functional fragment thereof with biological activity that is not attenuated.

97. The immune cell of claim 95 or claim 96, wherein the blocking element comprises human serum albumin (HSA) or an anti-HSA antibody.

98. The immune cell of any of claims 95-97, wherein the inducible cytokine prodrug further comprises one or more half-life extension domains.

99. The immune cell of any of claims 95-98, wherein the antigen binding protein directs the immune cell and its immune effector functions to cells that express a desired antigen.

100. A method of making an immune cell comprising contacting an immune cell with a (one or more) polynucleotide encoding:
a) A inducible cytokine prodrug comprising:
i) A cytokine polypeptide selected from the group IL-2, IL-12, IL-15, and IL-21, or a functional fragment, mutein or subunit thereof,
ii) A protease cleavable linker comprising an amino acid sequence selected from SEQ ID NOs: 195-220 or an amino acid sequence that has at least about 90% identity to SEQ ID NOs: 195-220, and
iii) A blocking element selected from a steric blocking element, a specific blocking element, and the combination thereof.

101. The method of making an immune cell of claim 100, wherein the polynucleotide further encodes an antigen binding protein.

102. The method of making an immune cell of claim 100 or claim 101, wherein the inducible cytokine prodrug has attenuated biological activity and wherein cleavage of the protease cleavable linker by the protease produces a cytokine or a functional fragment thereof with biological activity that is not attenuated.

103. The method of making an immune cell of any of claims 100-102, wherein the blocking element comprises human serum albumin (HSA) or an anti-HSA antibody.

104. The method of making an immune cell of any of claims 100-103, wherein the inducible cytokine prodrug further comprises one or more half-life extension domains.

105. The immune cell of any one of claims 95-99, wherein the inducible cytokine prodrug is tethered to a membrane associated protein.

106. The immune cell of claim 105, wherein the inducible cytokine prodrug is tethered to the membrane associated protein with a spacer sequence.

107. The immune cell of claim 106, wherein the spacer sequence comprises a cleavage site for a protease.

108. The immune cell of any one of claims 105 or 106, wherein the spacer sequence is a protease cleavable linker.

109. The immune cell of any one of claims 95-99 or 105-108, wherein the membrane associated protein is a transmembrane domain.


SEQUENCE LISTING (710 sequences)

SEQ ID NO: 1 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 4 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 214 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 613 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 7 - protein, 598 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 8 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGDETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 589 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 895 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 218 aa, synthetic construct
QVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 15 - protein, 427 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPPLAQKLKSSPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 16 - protein, 901 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 17 - protein, 1365 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 18 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKDSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 19 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 20 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 21 - protein, 553 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 22 - protein, 553 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 23 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 24 - protein, 559 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 25 - protein, 591 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPGGPAGIGALFKSSFPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPGGPAGIGALFKSSFPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 26 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQTGGSLRLSCTTSGTIFSGYTMGWYRQAPGEQRELVAVISGGGDTNYADSVKGRFTISRDNTKDTMYLQMNSLKPEDTAVYYCYSREVTPPWKLYWGQGTQVTVSSAAAYPYDVPDYGSHHHHHH

SEQ ID NO: 27 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQEGGSLRLSCAASERIFSTDVMGWYRQAAEKQRELVAVVSARGTTNYLDAVKGRFTISRDNARNTLTLQMNDLKPEDTASYYCYVRETTSPWRIYWGQGTQVTVSSAAAYPYDVPDYGSHHHHHH

SEQ ID NO: 28 - protein, 445 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQAGGSLRLSCAASGSIFSANAMGWYRQAPGKQRELVAVISSGGSTNYADSVKGRFTISRDNAKNTVYLQMNSLKPEDTAVYYCMYSGSYYYTPNDYWGQGTQVTVSSAAAYPYDVPDYGSHHHHHH

SEQ ID NO: 29 - protein, 553 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGQAPRLLIYSASFRYSGVPSRFSGSGSGTDFTLTISSLQPEDFATYYCQQYYTYPYTFGGGTKVEIKHHHHHH

SEQ ID NO: 30 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 31 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPGPAGLYAQPGSGGGGSGGGGSGGGGSGGGGSGGGGSGGGGSQSVLTQPPSVSGAPGQRVTISCSGSRSNIGEETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 906 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 910 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGPALFKSSFPPGSGGGGSGGGGSGGGGSGGGGSGGGGSGGGGSQSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 488 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKAVGQDTQEVIVVPHSLPFKVVVISAILALVVLTIISLIILIMLWQKKPR

SEQ ID NO: 42 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 43 - protein, 609 aa, Homo sapiens [sequence omitted]

SEQ ID NO: 44 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPALFKSSFPPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYAESVKGRFTISRDNSKNTLYLQMNSLRAEDTAVYYCKTHGSHDNWGQGTMVTVSS

SEQ ID NO: 45 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 46 - protein, 8 aa, synthetic construct
KRALGLPG

SEQ ID NO: 47 - protein, 40 aa, synthetic construct
DEDEDEDEDEDEDEDERPLALWRSDRDRDRDRDRDRDRDR

SEQ ID NO: 48 - skipped

SEQ ID NO: 49 - protein, 5 aa, synthetic construct
LEATA

SEQ ID NO: 50 - protein, 10 aa, synthetic construct
GGAANLVRGG

SEQ ID NO: 51 - protein, 10 aa, synthetic construct
SGRIGFLRTA

SEQ ID NO: 52 - protein, 6 aa, synthetic construct
PLGLAG

SEQ ID NO: 53 - protein, 6 aa, synthetic construct
PLGLAX

SEQ ID NO: 54 - protein, 6 aa, synthetic construct
  MOD_RES 4: note = s-methyl cysteine
PLGCAG

SEQ ID NO: 55 - protein, 8 aa, synthetic construct
ESPAYYTA

SEQ ID NO: 56 - protein, 6 aa, synthetic construct
RLQLKL

SEQ ID NO: 57 - protein, 7 aa, synthetic construct
RLQLKAC

SEQ ID NO: 58 - protein, 7 aa, synthetic construct
  MOD_RES 3: note = citrulline
  MOD_RES 5: note = homophenylalanine
EPXGXYL

SEQ ID NO: 59 - protein, 5 aa, synthetic construct
SGRSA

SEQ ID NO: 60 - protein, 4 aa, synthetic construct
DAFK

SEQ ID NO: 61 - protein, 5 aa, synthetic construct
GGGRR

SEQ ID NO: 62 - protein, 4 aa, synthetic construct
GFLG

SEQ ID NO: 63 - protein, 4 aa, synthetic construct
ALAL

SEQ ID NO: 64-65 - skipped (2 entries)

SEQ ID NO: 66 - protein, 5 aa, synthetic construct
  MOD_RES 3: note = s-ethyl cysteine
PICFF

SEQ ID NO: 67 - protein, 8 aa, synthetic construct
GGPRGLPG

SEQ ID NO: 68 - protein, 6 aa, synthetic construct
HSSKLQ

SEQ ID NO: 69 - protein, 7 aa, synthetic construct
HSSKLQL

SEQ ID NO: 70 - protein, 9 aa, synthetic construct
HSSKLQEDA

SEQ ID NO: 71 - protein, 10 aa, synthetic construct
LVLASSSFGY

SEQ ID NO: 72 - protein, 10 aa, synthetic construct
GVSQNYPIVG

SEQ ID NO: 73 - protein, 10 aa, synthetic construct
GVVQASCRLA

SEQ ID NO: 74 - skipped

SEQ ID NO: 75 - protein, 6 aa, synthetic construct
DPRSFL

SEQ ID NO: 76 - protein, 6 aa, synthetic construct
PPRSFL

SEQ ID NO: 77 - protein, 4 aa, synthetic construct
DEVD

SEQ ID NO: 78 - protein, 5 aa, synthetic construct
DEVDP

SEQ ID NO: 79 - protein, 8 aa, synthetic construct
KGSGDVEG

SEQ ID NO: 80 - protein, 6 aa, synthetic construct
GWEHDG

SEQ ID NO: 81 - protein, 7 aa, synthetic construct
EDDDDKA

SEQ ID NO: 82 - protein, 9 aa, synthetic construct
KQEQNPGST

SEQ ID NO: 83 - protein, 6 aa, synthetic construct
GKAFRR

SEQ ID NO: 84 - protein, 4 aa, synthetic construct
DAFK

SEQ ID NO: 85 - protein, 4 aa, synthetic construct
DVLK

SEQ ID NO: 86 - protein, 4 aa, synthetic construct
DAFK

SEQ ID NO: 87 - protein, 7 aa, synthetic construct
ALLLALL

SEQ ID NO: 88 - protein, 7 aa, synthetic construct
GPLGVRG

SEQ ID NO: 89 - protein, 8 aa, synthetic construct
IPVSLRSG

SEQ ID NO: 90 - protein, 8 aa, synthetic construct
VPLSLYSG

SEQ ID NO: 91 - protein, 10 aa, synthetic construct
SGESPAYYTA

SEQ ID NO: 92-191 - skipped (100 entries)

SEQ ID NO: 192 - protein, 267 aa, synthetic construct
MDMRVPAQLLGLLLLWLRGARCEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 193 - protein, 269 aa, synthetic construct
MDMRVPAQLLGLLLLWLRGARCQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 194 - skipped

SEQ ID NO: 195 - protein, 8 aa, synthetic construct
GPAGLYAQ

SEQ ID NO: 196 - protein, 8 aa, synthetic construct
GPAGMKGL

SEQ ID NO: 197 - protein, 8 aa, synthetic construct
PGGPAGIG

SEQ ID NO: 198 - protein, 8 aa, synthetic construct
ALFKSSFP

SEQ ID NO: 199 - protein, 8 aa, synthetic construct
ALFFSSPP

SEQ ID NO: 200 - protein, 8 aa, synthetic construct
LAQRLRSS

SEQ ID NO: 201 - protein, 8 aa, synthetic construct
LAQKLKSS

SEQ ID NO: 202 - protein, 30 aa, synthetic construct
GALFKSSFPSGGGPAGLYAQGGSGKGGSGK

SEQ ID NO: 203 - protein, 30 aa, synthetic construct
RGSGGGPAGLYAQGSGGGPAGLYAQGGSGK

SEQ ID NO: 204 - protein, 30 aa, synthetic construct
KGGGPAGLYAQGPAGLYAQGPAGLYAQGSR

SEQ ID NO: 205 - protein, 30 aa, synthetic construct
RGGPAGLYAQGGPAGLYAQGGGPAGLYAQK

SEQ ID NO: 206 - protein, 30 aa, synthetic construct
KGGALFKSSFPGGPAGIGPLAQKLKSSGGS

SEQ ID NO: 207 - protein, 30 aa, synthetic construct
SGGPGGPAGIGALFKSSFPLAQKLKSSGGG

SEQ ID NO: 208 - protein, 30 aa, synthetic construct
RGPLAQKLKSSALFKSSFPGGPAGIGGGGK

SEQ ID NO: 209 - protein, 30 aa, synthetic construct
GGGALFKSSFPLAQKLKSSPGGPAGIGGGR

SEQ ID NO: 210 - protein, 30 aa, synthetic construct
RGPGGPAGIGPLAQKLKSSALFKSSFPGGG

SEQ ID NO: 211 - protein, 30 aa, synthetic construct
RGGPLAQKLKSSPGGPAGIGALFKSSFPGK

SEQ ID NO: 212 - protein, 30 aa, synthetic construct
RSGGPAGLYAQALFKSSFPLAQKLKSSGGG

SEQ ID NO: 213 - protein, 30 aa, synthetic construct
GGPLAQKLKSSALFKSSFPGPAGLYAQGGR

SEQ ID NO: 214 - protein, 30 aa, synthetic construct
GGALFKSSFPGPAGLYAQPLAQKLKSSGGK

SEQ ID NO: 215 - protein, 30 aa, synthetic construct
RGGALFKSSFPLAQKLKSSGPAGLYAQGGK

SEQ ID NO: 216 - protein, 30 aa, synthetic construct
RGGGPAGLYAQPLAQKLKSSALFKSSFPGG

SEQ ID NO: 217 - protein, 30 aa, synthetic construct
SGPLAQKLKSSGPAGLYAQALFKSSFPGSK

SEQ ID NO: 218 - protein, 30 aa, synthetic construct
KGGPGGPAGIGPLAQRLRSSALFKSSFPGR

SEQ ID NO: 219 - protein, 30 aa, synthetic construct
KSGPGGPAGIGALFFSSPPLAQKLKSSGGR

SEQ ID NO: 220 - protein, 30 aa, synthetic construct
SGGFPRSGGSFNPRTFGSKRKRRGSRGGGG

SEQ ID NO: 221-236 - skipped (16 entries)

SEQ ID NO: 237 - protein, 5 aa, synthetic construct
  VARIANT 3: note = any amino acid
LPXTG

SEQ ID NO: 238 - protein, 25 aa, synthetic construct
  VARIANT 6..25: note = GGGGS repeats may be deleted
GGGGSGGGGSGGGGSGGGGSGGGGS

SEQ ID NO: 239 - protein, 20 aa, synthetic construct
  VARIANT 5..20: note = GGGS repeats may be deleted
GGGSGGGSGGGSGGGSGGGS

SEQ ID NO: 240-257 - skipped (18 entries)

SEQ ID NO: 258 - protein, 8 aa, synthetic construct
GPLGLKAQ

SEQ ID NO: 259 - protein, 8 aa, synthetic construct
LPLGLKAQ

SEQ ID NO: 260 - protein, 8 aa, synthetic construct
SPLGLKAQ

SEQ ID NO: 261 - protein, 8 aa, synthetic construct
QPLGLKAQ

SEQ ID NO: 262 - protein, 8 aa, synthetic construct
KPLGLKAQ

SEQ ID NO: 263 - protein, 8 aa, synthetic construct
FPLGLKAQ

SEQ ID NO: 264 - protein, 8 aa, synthetic construct
HPLGLKAQ

SEQ ID NO: 265 - protein, 8 aa, synthetic construct
PPLGLKAQ

SEQ ID NO: 266 - protein, 8 aa, synthetic construct
APLGLKAQ

SEQ ID NO: 267 - protein, 8 aa, synthetic construct
DPLGLKAQ

SEQ ID NO: 268 - protein, 8 aa, synthetic construct
GPHGLKAQ

SEQ ID NO: 269 - protein, 8 aa, synthetic construct
GPSGLKAQ

SEQ ID NO: 270 - protein, 8 aa, synthetic construct
GPQGLKAQ

SEQ ID NO: 271 - protein, 8 aa, synthetic construct
GPPGLKAQ

SEQ ID NO: 272 - protein, 8 aa, synthetic construct
GPEGLKAQ

SEQ ID NO: 273 - protein, 8 aa, synthetic construct
GPFGLKAQ

SEQ ID NO: 274 - protein, 8 aa, synthetic construct
GPRGLKAQ

SEQ ID NO: 275 - protein, 8 aa, synthetic construct
GPGGLKAQ

SEQ ID NO: 276 - protein, 8 aa, synthetic construct
GPAGLKAQ

SEQ ID NO: 277 - protein, 8 aa, synthetic construct
LPAGLKGA

SEQ ID NO: 278 - protein, 8 aa, synthetic construct
GPANLVAQ

SEQ ID NO: 279 - protein, 8 aa, synthetic construct
GPAALVGA

SEQ ID NO: 280 - protein, 8 aa, synthetic construct
GPANLRAQ

SEQ ID NO: 281 - protein, 8 aa, synthetic construct
GPAGLRAQ

SEQ ID NO: 282 - protein, 8 aa, synthetic construct
GPAGLVAQ

SEQ ID NO: 283 - protein, 8 aa, synthetic construct
GPAGLRGA

SEQ ID NO: 284 - protein, 8 aa, synthetic construct
LPAGLVGA

SEQ ID NO: 285 - protein, 8 aa, synthetic construct
GPAGLKGA

SEQ ID NO: 286 - protein, 8 aa, synthetic construct
GPLALKAQ

SEQ ID NO: 287 - protein, 8 aa, synthetic construct
GPLNLKAQ

SEQ ID NO: 288 - protein, 8 aa, synthetic construct
GPLHLKAQ

SEQ ID NO: 289 - protein, 8 aa, synthetic construct
GPLYLKAQ

SEQ ID NO: 290 - protein, 8 aa, synthetic construct
GPLPLKAQ

SEQ ID NO: 291 - protein, 8 aa, synthetic construct
GPLELKAQ

SEQ ID NO: 292 - protein, 8 aa, synthetic construct
GPLRLKAQ

SEQ ID NO: 293 - protein, 8 aa, synthetic construct
GPLLLKAQ

SEQ ID NO: 294 - protein, 8 aa, synthetic construct
GPLSLKAQ

SEQ ID NO: 295 - protein, 8 aa, synthetic construct
GPLGLYAQ

SEQ ID NO: 296 - protein, 8 aa, synthetic construct
GPLGLFAQ

SEQ ID NO: 297 - protein, 8 aa, synthetic construct
GPLGLLAQ

SEQ ID NO: 298 - protein, 8 aa, synthetic construct
GPLGLHAQ

SEQ ID NO: 299 - protein, 8 aa, synthetic construct
GPLGLRAQ

SEQ ID NO: 300 - protein, 8 aa, synthetic construct
GPLGLAAQ

SEQ ID NO: 301 - protein, 8 aa, synthetic construct
GPLGLEAQ

SEQ ID NO: 302 - protein, 8 aa, synthetic construct
GPLGLGAQ

SEQ ID NO: 303 - protein, 8 aa, synthetic construct
GPLGLPAQ

SEQ ID NO: 304 - protein, 8 aa, synthetic construct
GPLGLQAQ

SEQ ID NO: 305 - protein, 8 aa, synthetic construct
GPLGLSAQ

SEQ ID NO: 306 - protein, 8 aa, synthetic construct
GPLGLVAQ

SEQ ID NO: 307 - protein, 8 aa, synthetic construct
GPLGLKLQ

SEQ ID NO: 308 - protein, 8 aa, synthetic construct
GPLGLKFQ

SEQ ID NO: 309 - protein, 8 aa, synthetic construct
GPLGLKEQ

SEQ ID NO: 310 - protein, 8 aa, synthetic construct
GPLGLKKQ

SEQ ID NO: 311 - protein, 8 aa, synthetic construct
GPLGLKQQ

SEQ ID NO: 312 - protein, 8 aa, synthetic construct
GPLGLKSQ

SEQ ID NO: 313 - protein, 8 aa, synthetic construct
GPLGLKGQ

SEQ ID NO: 314 - protein, 8 aa, synthetic construct
GPLGLKHQ

SEQ ID NO: 315 - protein, 8 aa, synthetic construct
GPLGLKPQ

SEQ ID NO: 316 - protein, 8 aa, synthetic construct
GPLGLKAG

SEQ ID NO: 317 - protein, 8 aa, synthetic construct
GPLGLKAF

SEQ ID NO: 318 - protein, 8 aa, synthetic construct
GPLGLKAP

SEQ ID NO: 319 - protein, 8 aa, synthetic construct
GPLGLKAL

SEQ ID NO: 320 - protein, 8 aa, synthetic construct
GPLGLKAE

SEQ ID NO: 321 - protein, 8 aa, synthetic construct
GPLGLKAA

SEQ ID NO: 322 - protein, 8 aa, synthetic construct
GPLGLKAH

SEQ ID NO: 323 - protein, 8 aa, synthetic construct
GPLGLKAK

SEQ ID NO: 324 - protein, 8 aa, synthetic construct
GPLGLKAS

SEQ ID NO: 325 - protein, 8 aa, synthetic construct
GPLGLFGA

SEQ ID NO: 326 - protein, 8 aa, synthetic construct
GPLGLQGA

SEQ ID NO: 327 - protein, 8 aa, synthetic construct
GPLGLVGA

SEQ ID NO: 328 - protein, 8 aa, synthetic construct
GPLGLAGA

SEQ ID NO: 329 - protein, 8 aa, synthetic construct
GPLGLLGA

SEQ ID NO: 330 - protein, 8 aa, synthetic construct
GPLGLRGA

SEQ ID NO: 331 - protein, 8 aa, synthetic construct
GPLGLYGA

SEQ ID NO: 332 - protein, 8 aa, synthetic construct
ALFKSSPP

SEQ ID NO: 333 - protein, 8 aa, synthetic construct
SPFRSSRQ

SEQ ID NO: 334 - protein, 8 aa, synthetic construct
KLFKSSPP

SEQ ID NO: 335 - protein, 8 aa, synthetic construct
HLFKSSPP

SEQ ID NO: 336 - protein, 8 aa, synthetic construct
SLFKSSPP

SEQ ID NO: 337 - protein, 8 aa, synthetic construct
QLFKSSPP

SEQ ID NO: 338 - protein, 8 aa, synthetic construct
LLFKSSPP

SEQ ID NO: 339 - protein, 8 aa, synthetic construct
PLFKSSPP

SEQ ID NO: 340 - protein, 8 aa, synthetic construct
FLFKSSPP

SEQ ID NO: 341 - protein, 8 aa, synthetic construct
GLFKSSPP

SEQ ID NO: 342 - protein, 8 aa, synthetic construct
VLFKSSPP

SEQ ID NO: 343 - protein, 8 aa, synthetic construct
ELFKSSPP

SEQ ID NO: 344 - protein, 8 aa, synthetic construct
AKFKSSPP

SEQ ID NO: 345 - protein, 8 aa, synthetic construct
AHFKSSPP

SEQ ID NO: 346 - protein, 8 aa, synthetic construct
AGFKSSPP

SEQ ID NO: 347 - protein, 8 aa, synthetic construct
APFKSSPP

SEQ ID NO: 348 - protein, 8 aa, synthetic construct
ANFKSSPP

SEQ ID NO: 349 - protein, 8 aa, synthetic construct
AFFKSSPP

SEQ ID NO: 350 - protein, 8 aa, synthetic construct
AAFKSSPP

SEQ ID NO: 351 - protein, 8 aa, synthetic construct
ASFKSSPP

SEQ ID NO: 352 - protein, 8 aa, synthetic construct
AEFKSSPP

SEQ ID NO: 353 - protein, 8 aa, synthetic construct
ALRKSSPP

SEQ ID NO: 354 - protein, 8 aa, synthetic construct
ALLKSSPP

SEQ ID NO: 355 - protein, 8 aa, synthetic construct
ALAKSSPP

SEQ ID NO: 356 - protein, 8 aa, synthetic construct
ALQKSSPP

SEQ ID NO: 357 - protein, 8 aa, synthetic construct
ALHKSSPP

SEQ ID NO: 358 - protein, 8 aa, synthetic construct
ALPKSSPP

SEQ ID NO: 359 - protein, 8 aa, synthetic construct
ALTKSSPP

SEQ ID NO: 360 - protein, 8 aa, synthetic construct
ALGKSSPP

SEQ ID NO: 361 - protein, 8 aa, synthetic construct
ALDKSSPP

SEQ ID NO: 362 - protein, 8 aa, synthetic construct
ALFHSSPP

SEQ ID NO: 363 - protein, 8 aa, synthetic construct
ALFTSSPP

SEQ ID NO: 364 - protein, 8 aa, synthetic construct
ALFASSPP

SEQ ID NO: 365 - protein, 8 aa, synthetic construct
ALFQSSPP

SEQ ID NO: 366 - protein, 8 aa, synthetic construct
ALFLSSPP

SEQ ID NO: 367 - protein, 8 aa, synthetic construct
ALFGSSPP

SEQ ID NO: 368 - protein, 8 aa, synthetic construct
ALFESSPP

SEQ ID NO: 369 - protein, 8 aa, synthetic construct
ALFPSSPP

SEQ ID NO: 370 - protein, 8 aa, synthetic construct
ALFKHSPP

SEQ ID NO: 371 - protein, 8 aa, synthetic construct
ALFKLSPP

SEQ ID NO: 372 - protein, 8 aa, synthetic construct
ALFKKSPP

SEQ ID NO: 373 - protein, 8 aa, synthetic construct
ALFKASPP

SEQ ID NO: 374 - protein, 8 aa, synthetic construct
ALFKISPP

SEQ ID NO: 375 - protein, 8 aa, synthetic construct
ALFKGSPP

SEQ ID NO: 376 - protein, 8 aa, synthetic construct
ALFKNSPP

SEQ ID NO: 377 - protein, 8 aa, synthetic construct
ALFKRSPP

SEQ ID NO: 378 - protein, 8 aa, synthetic construct
ALFKESPP

SEQ ID NO: 379 - protein, 8 aa, synthetic construct
ALFKFSPP

SEQ ID NO: 380 - protein, 8 aa, synthetic construct
ALFKPSPP

SEQ ID NO: 381 - protein, 8 aa, synthetic construct
ALFKSFPP

SEQ ID NO: 382 - protein, 8 aa, synthetic construct
ALFKSLPP

SEQ ID NO: 383 - protein, 8 aa, synthetic construct
ALFKSIPP

SEQ ID NO: 384 - protein, 8 aa, synthetic construct
ALFKSKPP

SEQ ID NO: 385 - protein, 8 aa, synthetic construct
ALFKSAPP

SEQ ID NO: 386 - protein, 8 aa, synthetic construct
ALFKSQPP

SEQ ID NO: 387 - protein, 8 aa, synthetic construct
ALFKSPPP

SEQ ID NO: 388 - protein, 8 aa, synthetic construct
ALFKSEPP

SEQ ID NO: 389 - protein, 8 aa, synthetic construct
ALFKSGPP

SEQ ID NO: 390 - protein, 8 aa, synthetic construct
ALFKSSLP

SEQ ID NO: 391 - protein, 8 aa, synthetic construct
ALFKSSGP

SEQ ID NO: 392 - protein, 8 aa, synthetic construct
ALFKSSSP

SEQ ID NO: 393 - protein, 8 aa, synthetic construct
ALFKSSVP

SEQ ID NO: 394 - protein, 8 aa, synthetic construct
ALFKSSHP

SEQ ID NO: 395 - protein, 8 aa, synthetic construct
ALFKSSAP

SEQ ID NO: 396 - protein, 8 aa, synthetic construct
ALFKSSNP

SEQ ID NO: 397 - protein, 8 aa, synthetic construct
ALFKSSKP

SEQ ID NO: 398 - protein, 8 aa, synthetic construct
ALFKSSEP

SEQ ID NO: 399 - protein, 8 aa, synthetic construct
ALFKSSPF

SEQ ID NO: 400 - protein, 8 aa, synthetic construct
ALFKSSPH

SEQ ID NO: 401 - protein, 8 aa, synthetic construct
ALFKSSPG

SEQ ID NO: 402 - protein, 8 aa, synthetic construct
ALFKSSPA

SEQ ID NO: 403 - protein, 8 aa, synthetic construct
ALFKSSPS

SEQ ID NO: 404 - protein, 8 aa, synthetic construct
ALFKSSPV

SEQ ID NO: 405 - protein, 8 aa, synthetic construct
ALFKSSPQ

SEQ ID NO: 406 - protein, 8 aa, synthetic construct
ALFKSSPK

SEQ ID NO: 407 - protein, 8 aa, synthetic construct
ALFKSSPL

SEQ ID NO: 408 - protein, 8 aa, synthetic construct
ALFKSSPD

SEQ ID NO: 409-561 - skipped (153 entries)

SEQ ID NO: 562 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 563 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 564 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 565 - protein, 242 aa, synthetic construct
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 566 - protein, 242 aa, synthetic construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 567 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 568 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 569 - protein, 247 aa, synthetic construct
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 570 - protein, 247 aa, synthetic construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 571 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 572 - protein, 247 aa, synthetic construct
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 573 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 574 - protein, 242 aa, synthetic construct
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 575 - protein, 244 aa, synthetic construct
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRS

SEQ ID NO: 576 - protein, 246 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSE

SEQ ID NO: 577 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 578 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 579 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 580 - protein, 111 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 581 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 582 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 583 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 584 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 585 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 586 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 587 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 588 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 589 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 590 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 591 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 592 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 593 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 594 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 595 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 596 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 597 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 598 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 599 - protein, 121 aa, synthetic construct
QVQLQQSGPGLVKPSQTLSLTCAISGDSVSSNSAAWNWIRQSPSRGLEWLGRTYYRSTWYNDYAVSVKSRITINPDTSKNQFSLQLNSVTPEDTAVYYCAREVSGTSAFDIWGQGTMVTVS

SEQ ID NO: 600 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 601 - protein, 126 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARASMTGGYSYGDAFDIWGQGTLVTVS

SEQ ID NO: 602 - protein, 107 aa, synthetic construct
AIRMTQSPSSLSASVGDRVTITCRASQSISSYLNWYQQKPGKAPKLLIYAASSLQSGVPSRFSGSGSGTDFSLTISSLQPEDSATYYCQQTYSTPLTFGQGTKVEIK

SEQ ID NO: 603 - protein, 122 aa, synthetic construct
QVQLQQSGPGLVEPSQTLSLTCAISGDSVSSDSVAWNWIRQSPSRGLEWLGRTYYRSTWYNDYAGSVKSRITINPDTSKNQFSLQLTSVTPEDTAVYYCTRSRHNTFRGMDVWGQGTTVTVS

SEQ ID NO: 604 - protein, 107 aa, synthetic construct
DIVMTQSPSSLSASVGDRVTITCRASQTISSYLNWYQQKPGKAPKLLIYDASSLQSGVPSRFSGSGSGTDFTLTINSLQPEDFATYYCQQSYTTPITFGQGTRLEIK

SEQ ID NO: 605 - protein, 122 aa, synthetic construct
QVQLQQSGPGLVEPSQTLSLTCAISGDSVSSNSAAWNWIRQSPSRGLEWLGRTYYRSTWYNDYAGSVKSRITINPDTSKNQFSLQLTSVTPEDTAVYYCTRSRHNTFRGMDVWGQGTLVTVS

SEQ ID NO: 606 - protein, 107 aa, synthetic construct
DIQLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 607 - protein, 118 aa, synthetic construct
QVQLQQSGPGLVEPSQTLSLTCAISGDSVSSDSVAWNWIRQSPSRGLEWLGRTYYRSTWYNDYAGSVKSRITINPDTSKNQFSLQLNSVTPEDTAVYYCARDRNGMDVWGQGTMVTVS

SEQ ID NO: 608 - protein, 107 aa, synthetic construct
DIVMTQSPSSLSASVGDRVTITCRASQSISSYLNWYQQKPGKAPKLLIYDASNLETGVPSRFSGSGSGTDFTFTITSLQPEDFATYYCQQSYTTPLTFGGGTKVEIK

SEQ ID NO: 609 - protein, 121 aa, synthetic construct
QVQLQQSGPGLVKPSQTLSLTCAISGDSVSSSSAAWNWIRQSPSRGLEWLGRTYYRSAWYNDYAVSVKSRITINPDTSKNQFSLQLNSVTPEDTAVYYCARESVLLDGMDVWGRGTTVTVS

SEQ ID NO: 610 - protein, 107 aa, synthetic construct
AIRMTQSPSTLSASVGDRVTITCRASQSISTYLNWYQQKAGKAPRLLIHDASSLQSGVPSRFSGSGSGTDFTLTISSLQPEDFATYYCQQSYSTPLTFGGGTKVEIK

SEQ ID NO: 611 - protein, 128 aa, synthetic construct
QVQLQQSGPGLVQPSQTLSLTCVISGDSVSSNSATWNWIRQSPSRGLEWLGRTYYRSKWYNDYAVSVKSRITINPDTSKNQFSLQLNSVTPEDTAVYYCARDGDGGSYYDYYYYGMDVWGQGTTVTVS

SEQ ID NO: 612 - protein, 107 aa, synthetic construct
DIQLTQSPSSLSTSVGDRVTITCRASQSISTYLNWYQQKPGKAPKLLIYAASNLQSGVPSRFSGSGSGTDFTLTISSLQPEDFATYFCQQSYTTPITFGQGTRLEIK

SEQ ID NO: 613 - protein, 118 aa, synthetic construct
QVQLQQSGPGLVKPSQTLSLTCAISGDSVSSNSAAWNWIRQSPSRGLEWLGRTYYRSAWYNDYAVSVKSRITINPDTSKNQFSLQLSSVTPEDTAVYYCARDVEGFDYWGQGTLVTVS

SEQ ID NO: 614 - protein, 107 aa, synthetic construct
DIVMTQTPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 615 - protein, 123 aa, synthetic construct
QVQLQQSGPGLVKPSQTLSLTCAISGDSVSGNRATWNWIRQSPSRGLEWLGRTYYRSAWYNDYAVSVKGRITFNPDTSKNQFSLQLNSVTPEDTAVYYCARGESGAAADAFDIWGQGTTVTVS

SEQ ID NO: 616 - protein, 107 aa, synthetic construct
DIQLTQSPPSLSASVGDRVTITCRASQSISSYLNWYQQKPGKAPKLLIYAASSLQSGVPSRFSGSGFGTDFTLTISSLQPEDFATYYCQQSYSTPQTFGQGTKVDIK

SEQ ID NO: 617 - protein, 118 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREDSGSGAFDIWGQGTLVTVS

SEQ ID NO: 618 - protein, 112 aa, synthetic construct
EIVLTQSPLSLPVTPGEPASISCRSSRSLLSYHGYNYLDWYLQKPGQSPQLLIFVGSNRAPGVPDRFSGSGSGTDFTLNISRVEAEDVGVYYCMQSLQTPRTFGQGTKVEIK

SEQ ID NO: 619 - protein, 121 aa, synthetic construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 620 - protein, 112 aa, synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 621 - protein, 124 aa, synthetic construct
EVQLQQSGPGLVKPSQTLSLTCAISGDSVSNNNAAWNWIRQSPSRGLEWLGRTYHRSTWYNDYVGSVKSRITINPDTSKNQFSLQLNSVTPEDTAVYYCARETDYGDYGAFDIWGQGTTVTVSS

SEQ ID NO: 622 - protein, 111 aa, synthetic construct
QSALTQPASVSGSPGQSITISCTGSRNDIGAYESVSWYQQHPGNAPKLIIHGVNNRPSGVFDRFSVSQSGNTASLTISGLQAEDEADYYCSSHTTTSTLYVFGTGTKVTVL

SEQ ID NO: 623 - protein, 121 aa, synthetic construct
EVQLQQSGPGLVNPSQTLSITCAISGDSVSSNSAAWNWIRQSPSRGLEWLGRTFYRSKWYNDYAVSVKGRITISPDTSKNQFSLQLNSVTPEDTAVYYCAGGDYYYGLDVWGQGTTVTVSS

SEQ ID NO: 624 - protein, 110 aa, synthetic construct
QSALTQPASVSGSPGQSITISCTGSSSDVGGYNSVSWYQQHPGKAPKLMIYEVINRPSGVSHRFSGSKSGNTASLTISGLQAEDEADYYCSSYTSSSTYVFGTGTKVTVL

SEQ ID NO: 625 - protein, 127 aa, synthetic construct
EVQLQQSGPGLVKPSQTLSLTCAISGDSVLSNSDTWNWIRQSPSRGLEWLGRTYHRSTWYDDYASSVRGRVSINVDTSKNQYSLQLNAVTPEDTGVYYCARDRLQDGNSWSDAFDVWGQGTMVTVSS

SEQ ID NO: 626 - protein, 111 aa, synthetic construct
QSALTQPASVSGSPGQSITISCTGSSSDIGGFNYVSWYQQHAGEAPKLMIYEVTNRPSGVSDRFSGSKSDNTASLTISGLQAEDEADYYCSSYASGSPLYVFGTGTKVTVL

SEQ ID NO: 627 - protein, 127 aa, synthetic construct; identical to SEQ ID NO: 625

SEQ ID NO: 628 - protein, 111 aa, synthetic construct
QSALTQPASVSGSPGQSITFSCTGTSSDIGGYNYVSWYQQHPGKAPKLMIYEVSNRPSGVSNRFSGTKSGNTASLTISGLQAEDEADYYCSSYTSSSTLYVFGTGTKLTVL

SEQ ID NO: 629 - protein, 127 aa, synthetic construct
QVQLQESGPGLVKPSQTLSLTCAISGDSVLSNSDTWNWIRQSPSRGLEWLGRTYHRSTWYDDYASSVRGRVSINVDTSKNQYSLQLNAVTPEDTGVYYCARDRLQDGNSWSDAFDVWGQGTMVTVSS

SEQ ID NO: 630 - protein, 111 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLYVFGTGTKVTVL

SEQ ID NO: 631 - protein, 127 aa, synthetic construct; identical to SEQ ID NO: 625

SEQ ID NO: 632 - protein, 111 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGTGTKVTVL

SEQ ID NO: 633 - protein, 127 aa, synthetic construct; identical to SEQ ID NO: 625

SEQ ID NO: 634 - protein, 111 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLYIFGTGTKVTVL

SEQ ID NO: 635 - protein, 127 aa, synthetic construct
EVQLQQSGPGLVKPSQTLPLTCAISGDSVLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 636 - protein, 111 aa, synthetic construct
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 637 - protein, 127 aa, synthetic construct
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 638 - protein, 111 aa, synthetic construct; identical to SEQ ID NO: 636

SEQ ID NO: 639 - protein, 121 aa, synthetic construct
EVQLVQSGAEVKKPGSSVKVSCKASGGTFSSYAISWVRQAPGQGLEWMGGIIPIFGTANYAQKFQRVTITADKSTSTAYMELSSLRSEDTAVYYCATFALFGFREQAFDIWGQGTTVTVSS

SEQ ID NO: 640 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 641 - protein, 115 aa, synthetic construct
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVS

SEQ ID NO: 642 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 643 - protein, 117 aa, synthetic construct
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSS

SEQ ID NO: 644 - protein, 108 aa, synthetic construct
DIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKR

SEQ ID NO: 645 - protein, 120 aa, synthetic construct
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSS

SEQ ID NO: 646 - protein, 115 aa, synthetic construct
DIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVS

SEQ ID NO: 647 - protein, 118 aa, synthetic construct
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 648 - protein, 113 aa, synthetic construct
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 649 - protein, 45 aa, synthetic construct
TTTPAPRPPTPAPTIASQPLSLRPEACRPAAGGAVHTRGLDFACD

SEQ ID NO: 650 - protein, 230 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 651 - protein, 282 aa, synthetic construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 652 - protein, 24 aa, synthetic construct
IYIWAPLAGTCGVLLLSLVITLYC

SEQ ID NO: 653 - protein, 68 aa, synthetic construct
FWVLVVVGGVLACYSLLVTVAFIIFWVRSKRSRGGHSDYMNMTPRRPGPTRKHYQPYAPPRDFAAYRS

SEQ ID NO: 654 - protein, 42 aa, synthetic construct
KRGRKKLLYIFKQPFMRPVQTTQEEDGCSCRFPEEEEGGCEL

SEQ ID NO: 655 - protein, 41 aa, synthetic construct
RSKRSRLLHSDYMNMTPRRPGPTRKHYQPYAPPRDFAAYRS

SEQ ID NO: 656 - protein, 35 aa, synthetic construct
TKKKYSSSVHDPNGEYMFMRAVNTAKKSRLTDVTL

SEQ ID NO: 657 - protein, 35 aa, synthetic construct
TKKKYSSSVHDPNGEFMFMRAVNTAKKSRLTDVTL

SEQ ID NO: 658 - protein, 42 aa, synthetic construct; identical to SEQ ID NO: 654

SEQ ID NO: 659 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 660 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 661 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 662 - protein, 464 aa, synthetic construct
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 663 - protein, 439 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 664 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 665 - protein, 493 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 666 - protein, 105 aa, synthetic construct
MKNQVEQSPQSLIILEGKNCTLQCNYTVSPFSNLRWYKQDTGRGPVSLTIMTFSENTKSNGRYTATLDADTKQSSLHITASQLSDSASYICVVSGGTDSWGKLQF

SEQ ID NO: 667 - protein, 105 aa, synthetic construct
MKNQVEQSPQSLIILEGKNCTLQCNYTVSPFSNLRWYKQDTGRGPVSLTIVTFSENTKSNGRYTATLDADTKQSSLHITASQLSDSASYICVVSGGTDSWGKLQF

SEQ ID NO: 668 - protein, 105 aa, synthetic construct
MKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQF

SEQ ID NO: 669 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYNEQFF

SEQ ID NO: 670 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFF

SEQ ID NO: 671 - protein, 276 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 672 - protein, 312 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 673 - protein, 277 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 674 - protein, 310 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSRSYHQGVLSATILYEILLGKATLYAVLVSALVLMAMVKRKDSRG

SEQ ID NO: 675 - protein, 273 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 676 - protein, 311 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 677 - protein, 276 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 678 - protein, 312 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDWDRSGDKETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 679 - protein, 277 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 680 - protein, 310 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 681 - protein, 273 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 682 - protein, 310 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKD

SEQ ID NO: 683 - protein, 276 aa, synthetic construct
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMSGDSAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 684 - protein, 312 aa, synthetic construct; identical to SEQ ID NO: 672

SEQ ID NO: 685 - protein, 277 aa, synthetic construct
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 686 - protein, 310 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 687 - protein, 273 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASRGTGFQKLVFGTGTRLLVSPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 688 - protein, 310 aa, synthetic construct
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQFWDGAGDEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKD

SEQ ID NO: 689 - protein, 105 aa, synthetic construct; identical to SEQ ID NO: 666

SEQ ID NO: 690 - protein, 105 aa, synthetic construct
MKNQVEQSPQSLIILEGKNCTLQCNYTVSPFSNLRWYKQDTGRGPVSLTRMTFSENTKSNGRYTATLDADTKQSSLHITASQLSDSASYICVVSGGTDSWGKLQF

SEQ ID NO: 691 - protein, 123 aa, synthetic construct; identical to SEQ ID NO: 669

SEQ ID NO: 692 - protein, 123 aa, synthetic construct
MASLLFFCGAFYLLGTGSMDADVTQTPRNRITKTGKRIMLECSQTKGRDRMYWYRQDPGLGLRLIYYSFDVKDINKGEISDGYSVSRQAQAKFSLSLESAIPNQTALYFCATSGQGAYNEQFF

SEQ ID NO: 693 - protein, 123 aa, synthetic construct; identical to SEQ ID NO: 670

SEQ ID NO: 694 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYREQFF

SEQ ID NO: 695 - protein, 205 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 696 - protein, 205 aa, synthetic construct
QKEVEQNSGPLSVPEGAIASLNCTYSDRGSASFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 697 - protein, 205 aa, synthetic construct
QKEVEQNSGPLSVPEGAIASLNCTYSDRGSSSFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 698 - protein, 240 aa, synthetic construct
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 699 - protein, 240 aa, synthetic construct
NAGVTQTPKFRVLKTGQSMTLLCAQDMNHEYMYWYRQDPGMGLRLIHYSVSEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 700 - protein, 240 aa, synthetic construct
NAGVTQTPKFRVLKTGQSMTLLCAQDMNHEYMYWYRQDPGMGLRLIHYSVAEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 701 - protein, 240 aa, synthetic construct
NAGVTQTPKFRVLKTGQSMTLLCAQDMNHEYMYWYRQDPGMGLRLIHYSVFEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 702 - protein, 240 aa, synthetic construct
NAGVTQTPKFRVLKTGQSMTLLCAQDMNHDYMYWYRQDPGMGLRLIHYSVGEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 703 - protein, 109 aa, synthetic construct
EVQLVESGGGLVQPGGSLRLSCAAGGDWSANFMYWYRQAPGKQRELVARISGRGVVDYVEVKGRFTISRDNSKNTLYLQMNSLRAEDTAVYYCAVASYWGQGTLVTVSS

SEQ ID NO: 704 - protein, 118 aa, synthetic construct
EVQLVESGGGLVQPGGSLRLSCAAGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 705 - protein, 115 aa, synthetic construct
QVQVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 706 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 707 - protein, 515 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGSSGGPALFKSSFPPGSGGGGSGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDPVYDLFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 708 - protein, 213 aa, synthetic construct
DIQLTQSPSTLSASVGDRVTITCRASQAISSWLAWYQQKPGKAPKLLIYDASSLESGVPSRFSGSGSGTEFTLTISSLQPDDFATYYCQQYDPLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 709 - protein, 7 aa, synthetic construct
PAGLYAQ

SEQ ID NO: 710 - protein, 5 aa, synthetic construct
QAPRL